(19) United States
(12) Patent Application Publication
(10) Pub. No.: US 2025/0276095 A1
(43) Pub. Date: Sep. 4, 2025

(54) ADENO-ASSOCIATED VIRUS COMPOSITIONS FOR THE TREATMENT OF DUCHENNE MUSCULAR DYSTROPHY

(71) Applicant: KATE THERAPEUTICS, INC., San Diego, CA (US)

(72) Inventors: Aboozar MONAVARFESHANI, San Diego, CA (US); Saurav SESHADRI, San Diego, CA (US); Mohammadsharif TABEBORDBAR, San Diego, CA (US); Shayan TABEBORDBAR, San Diego, CA (US); Danielle VELEZ, San Diego, CA (US); Simon YE, San Diego, CA (US)

(21) Appl. No.: 19/069,995

(22) Filed: Mar. 4, 2025

Related U.S. Application Data
(60) Provisional application No. 63/561,130, filed on Mar. 4, 2024.
(60) Provisional application No. 63/666,899, filed on Jul. 2, 2024.
(60) Provisional application No. 63/676,780, filed on Jul. 29, 2024.
(60) Provisional application No. 63/688,024, filed on Aug. 28, 2024.

(51) Int. Cl. A61K 48/00; A61P 21/00; C07K 14/005; C07K 14/47; C12N 15/86

(52) U.S. Cl. CPC A61K 48/0058; A61P 21/00; C07K 14/005; C07K 14/4708; C12N 15/86; C12N 2750/14122; C12N 2750/14143; C12N 2830/008; C12N 2830/42; C12N 2830/50

(57) ABSTRACT

The present invention provides novel gene therapy compositions for use in treatment of Duchenne Muscular Dystrophy (DMD).

### CLAIM OF PRIORITY

This application claims the benefit of U.S. Provisional Patent Application No. 63/561,130, filed Mar. 4, 2024; U.S. Provisional Patent Application No. 63/666,899, filed Jul. 2, 2024; U.S. Provisional Patent Application No. 63/676,780, filed Jul. 29, 2024; and U.S. Provisional Patent Application No. 63/688,024, filed Aug. 28, 2024; each of which is incorporated by reference herein in its entirety.

### SEQUENCE LISTING

This instant application contains a Sequence Listing which has been submitted electronically in XML file format and is hereby incorporated by reference in its entirety. The XML copy, created on Mar. 3, 2025, is named PAT059900-US-NP_SL.xml and is 2,260,685 bytes in size.

### FIELD OF DISCLOSURE

This disclosure relates to compositions for the treatment of Duchenne Muscular Dystrophy (DMD).

### BACKGROUND

Duchenne muscular dystrophy (DMD) is a severe type of muscular dystrophy, a group of rare genetic neuromuscular diseases, affecting approximately one in 3,600 to 9,200 live male births. DMD is the most common type of muscular dystrophy predominantly affecting men.

DMD causes progressive muscle weakness due to muscle fiber disarray, replacement with connective tissue or fat, and death. Voluntary muscles are affected first, especially those of the hips, pelvic area, thighs, calves, eventually progressing to the shoulders and neck, followed by arms, respiratory muscles, and other areas. Symptoms usually appear before age five, expressing as difficulty with motor skills including walking. As a result, falls may be frequent with walking becoming increasingly difficult and frequently impossible before age 13. Most men affected with DMD become essentially paralyzed from the neck down by the age of 21. Cardiomyopathy, particularly dilated cardiomyopathy, is also commonly seen in half of 18-year-olds with DMD. In late stages of the disease, respiratory impairment and swallowing impairment can occur. As a result, median life expectancy for men suffering from DMD is just 28-30 years, however comprehensive care may allow those suffering to live into their 30s or 40s.

DMD is caused by mutations and/or deletions in any of the 79 exons encoding the large dystrophin protein. While mutations causing DMD can affect any exon, exons 2-20 and 45-55 are common hotspots for large deletion and duplication mutations. The disorder follows an X-linked recessive inheritance pattern, with approximately two-thirds of cases inherited from the mother and one-third resulting from a new mutation.

Typical therapies for DMD are limited to management strategies, such as physical therapy, braces, and corrective surgery to alleviate symptoms. Assisted ventilation may be required in those with weakness of breathing muscles.

Gene therapies have been proposed for the treatment of DMD, including delandistrogene moxeparvovec-rokl, marketed under the brand name ELEVIDYS® by Sarepta Therapeutics. However, expression of the microdystrophin protein in skeletal muscles of patients injected with DMD gene therapies has been low and not uniform.

As a result, there exists a need for improved DMD gene therapies.

### SUMMARY

The present disclosure is directed to compositions (e.g., adeno-associated viral (AAV) compositions) and methods of use thereof for treating Duchenne Muscular Dystrophy (DMD).

Accordingly, aspects of the invention provide a formulation comprising an AAV particle comprising (e.g., encapsidating) a nucleic acid comprising a promoter and a sequence encoding microdystrophin.

Aspects of the invention provide a composition comprising an adeno-associated virus (AAV) particle comprising an engineered capsid protein comprising the amino acid sequence RGDR (SEQ ID NO: 2440). The capsid protein encapsidates a nucleic acid molecule (e.g., a cargo) comprising a promoter, a codon optimized microdystrophin sequence, for example a sequence having at least 95% sequence identity with a sequence selected from SEQ ID NO: 2405-2407. The nucleic acid may further comprise a dorsal root ganglia (DRG) de-targeting miRNA binding site. In aspects of the invention, the nucleic acid molecule may comprise a sequence having the having at least 95% sequence identity with a sequence selected from SEQ ID NO: 2405-2407 that encodes a codon optimized microdystrophin sequence.

The DRG de-targeting miRNA binding site may comprise a sequence selected from among SEQ ID NOs: 2399-2401.

The promoter may be a novel promoter that is exhibits specificity (e.g. high expression in) for muscle cells, for example myocytes. The promoter may comprise a sequence having at least 95% sequence identity a sequence selected from among SEQ ID NOs: 2402-2404.

For example, the nucleic acid molecule (e.g. cargo) may comprise, in order:
a first inverted terminal repeats (ITR) region;
a promoter;
an intron;
a sequence having at least 95% sequence identity with a sequence selected from any one of SEQ ID NO: 2405-2407;
a first DRG de-targeting miRNA binding site and preferably, a second DRG de-targeting miRNA binding site;
a poly-adenylation signal, preferably a shortened poly-adenylation signal; and
a second ITR region.

The ITR regions may be AAV2 ITR regions derived from an AAV2.

For example, the full nucleic acid molecule may comprise a sequence selected from any one of SEQ ID NOs: 2412-2438.

Advantageously, when administered to a subject with a mutation in the DMD gene that results in low or a lack of dystrophin protein expression, the composition is effective at restoring a healthy phenotype.

The engineered capsid protein may advantageously exhibit decreased liver tropism and/or increased muscle tropism.

For example, the capsid protein may comprise an amino acid sequence selected from Tables 1a in hypervariable region IV (HVR IV) relative to wild type AAV9. The capsid protein may comprise substitutions at amino acids 451-455 relative to a wild type AAV9 vector capsid selected from column 1 of Table 1b and a 7-mer insert selected from column 2 of Table 1b inserted after amino acid 455 relative to a wild type AAV9 vector. The capsid protein may further comprise a deletion of G267 relative to wild type AAV9 capsid or equivalent position in another AAV capsid.

Accordingly, aspects of the invention further provide methods and uses of the compositions of the invention (for example in the preparation of a medicament) for treating a subject suffering from DMD. The methods and uses may comprise providing to a subject a composition of the invention as described above.

Aspects of the present disclosure provide a recombinant nucleic acid comprising a muscle specific promoter; a sequence encoding microdystrophin; and at least one dorsal root ganglia (DRG) de-targeting miRNA binding site.

In some embodiments, the muscle specific promoter comprises a CK8 promoter, a desmin promoter, a Mb promoter, a MCK promoter, a MHCK7 promoter, a skeletal muscle alpha actin promoter, or a TTNI2 promoter. In some embodiments, the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404. In some embodiments, the promoter comprises or consists of the nucleic acid sequence of any one of SEQ ID NOs: 2402-2404. In some embodiments, the promoter comprises or consists of the nucleic acid sequence of SEQ ID NO: 2402. In some embodiments, the promoter comprises or consists of the nucleic acid sequence of SEQ ID NO: 2403. In some embodiments, the promoter comprises or consists of the nucleic acid sequence of SEQ ID NO: 2404.

In some embodiments, the sequence encoding microdystrophin is a sequence encoding human microdystrophin. In some embodiments, the sequence encoding microdystrophin encodes a microdystrophin protein comprising or consisting of an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 2439. In some embodiments, the sequence encoding microdystrophin encodes a microdystrophin protein comprising or consisting of the amino acid sequence of SEQ ID NO: 2439.

In some embodiments, the sequence encoding microdystrophin comprises or consists of a codon optimized sequence encoding microdystrophin. In some embodiments, the codon optimized sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407. In some embodiments, the codon optimized sequence encoding microdystrophin comprises or consists of the nucleic acid sequence of any one of SEQ ID NOs: 2405-2407. In some embodiments, the codon optimized sequence encoding microdystrophin comprises or consists of the nucleic acid sequence of SEQ ID NO: 2405. In some embodiments, the codon optimized sequence encoding microdystrophin comprises or consists of the nucleic acid sequence of SEQ ID NO: 2406. In some embodiments, the codon optimized sequence encoding microdystrophin comprises or consists of the nucleic acid sequence of SEQ ID NO: 2407.

In some embodiments, the at least one DRG de-targeting miRNA binding site comprises a binding site for miR-338, miR-138, or miR-9. In some embodiments, the at least one DRG de-targeting miRNA binding site comprises a binding site for miR-338-3p, miR-138-5p, or miR-9-5p. In some embodiments, the at least one DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401. In some embodiments, the at least one DRG de-targeting miRNA binding site comprises or consists of the nucleic acid sequence of any one of SEQ ID NOs: 2399-2401.

In some embodiments, the at least one DRG de-targeting miRNA binding site comprises a first and a second DRG de-targeting miRNA binding site. In some embodiments, each of the first and the second DRG de-targeting miRNA binding sites comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401. In some embodiments, each of the first and the second DRG de-targeting miRNA binding sites comprises or consists of the nucleic acid sequence of any one of SEQ ID NOs: 2399-2401. In some embodiments, each of the first and the second DRG de-targeting miRNA binding sites comprises or consists of the nucleic acid sequence of SEQ ID NO: 2399. In some embodiments, each of the first and the second DRG de-targeting miRNA binding sites comprises or consists of the nucleic acid sequence of SEQ ID NO: 2400. In some embodiments, each of the first and the second DRG de-targeting miRNA binding sites comprises or consists of the nucleic acid sequence of SEQ ID NO: 2401.

In some embodiments, the recombinant nucleic acid comprises, optionally from 5′ to 3′, a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In some embodiments, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In some embodiments, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In some embodiments, the recombinant nucleic acid further comprises an intron. In some embodiments, the intron comprises a simian virus 40 (SV40) intron sequence, a minute virus of mice (MVM) intron, a RK intron, a β-globin intron, and/or a chicken β-actin (CBA) intron, including functional variants or fragments thereof. In some embodiments, the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409. In some embodiments, the intron comprises or consists of the nucleic acid sequence of SEQ ID NO: 2409.

In some embodiments, the recombinant nucleic acid further comprises a polyadenylation (PolyA) signal. In some embodiments, the Poly A signal comprises a bovine growth hormone polyadenylation (bgh-Poly A) signal, a synthetic PolyA signal, or an SV40 Poly A signal. In some embodiments, the PolyA signal comprises the bgh-Poly A signal, and the bgh-Poly A signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410. In some embodiments, the PolyA signal comprises the bgh-Poly A signal, and the bgh-Poly A signal comprises or consists of the nucleic acid sequence of SEQ ID NO: 2410.

In some embodiments, the recombinant nucleic acid further comprises a 5′ inverted terminal repeat (ITR) and a 3′ ITR. In some embodiments, the 5′ ITR and/or the 3′ ITR is an adeno-associated virus 2 (AAV2) ITR, or a variant or fragment thereof.

In some embodiments, the 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408. In some embodiments, the 5′ ITR comprises or consists of the nucleic acid sequence of SEQ ID NO: 2408.

In some embodiments, the 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411. In some embodiments, the 3′ ITR comprises or consists of the nucleic acid sequence of SEQ ID NO: 2411.

In some embodiments, the recombinant nucleic acid comprises, optionally from 5′ to 3′, a 5′ ITR; a promoter; an intron; a sequence encoding microdystrophin; a first DRG de-targeting miRNA binding site; a second DRG de-targeting miRNA binding site; a PolyA signal; and a 3′ ITR.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:

2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:
2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:

2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:
2402; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:

2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:
2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:

2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:
2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO:

2411.

In some embodiments, the recombinant nucleic acid comprises a 5′ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a first DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ITR comprising or consisting of the nucleic acid sequence of SEQ ID NO: 2408; a promoter comprising or consisting of the nucleic acid sequence of SEQ ID NO: 2404; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprises or consists of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and the 3′ ITR comprising or consisting of a nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In some embodiments, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2412-2438. In some embodiments, the recombinant nucleic acid comprises or consists of the nucleic acid sequence of any one of SEQ ID NOs: 2412-2438.

In some embodiments, the recombinant nucleic acid comprises or consists of the nucleic acid sequence of SEQ ID NO: 2412. In some embodiments, the recombinant nucleic acid comprises or consists of the nucleic acid sequence of SEQ ID NO: 2413. In some embodiments, the recombinant nucleic acid comprises or consists of the nucleic acid sequence of SEQ ID NO: 2414. In some embodiments, the recombinant nucleic acid comprises or consists of the nucleic acid sequence of SEQ ID NO: 2415. In some embodiments, the recombinant nucleic acid comprises or consists of the nucleic acid sequence of SEQ ID NO: 2416. In some embodiments, the recombinant nucleic acid comprises or consists of the nucleic acid sequence of SEQ ID NO: 2417. In some embodiments, the recombinant nucleic acid comprises or consists of the nucleic acid sequence of SEQ ID NO: 2418. In some embodiments, the recombinant nucleic acid comprises or consists of the nucleic acid sequence of SEQ ID NO: 2419. In some embodiments, the recombinant nucleic acid comprises or consists of the nucleic acid sequence of SEQ ID NO: 2420. In some embodiments, the recombinant nucleic acid comprises or consists of the nucleic acid sequence of SEQ ID NO: 2421. In some embodiments, the recombinant nucleic acid comprises or consists of the nucleic acid sequence of SEQ ID NO: 2422. In some embodiments, the recombinant nucleic acid comprises or consists of the nucleic acid sequence of SEQ ID NO: 2423. In some embodiments, the recombinant nucleic acid comprises or consists of the nucleic acid sequence of SEQ ID NO: 2424. In some embodiments, the recombinant nucleic acid comprises or consists of the nucleic acid sequence of SEQ ID NO: 2425. In some embodiments, the recombinant nucleic acid comprises or consists of the nucleic acid sequence of SEQ ID NO: 2426. In some embodiments, the recombinant nucleic acid comprises or consists of the nucleic acid sequence of SEQ ID NO: 2427. In some embodiments, the recombinant nucleic acid comprises or consists of the nucleic acid sequence of SEQ ID NO: 2428. In some embodiments, the recombinant nucleic acid comprises or consists of the nucleic acid sequence of SEQ ID NO: 2429. In some embodiments, the recombinant nucleic acid comprises or consists of the nucleic acid sequence of SEQ ID NO: 2430. In some embodiments, the recombinant nucleic acid comprises or consists of the nucleic acid sequence of SEQ ID NO: 2431. In some embodiments, the recombinant nucleic acid comprises or consists of the nucleic acid sequence of SEQ ID NO: 2432. In some embodiments, the recombinant nucleic acid comprises or consists of the nucleic acid sequence of SEQ ID NO: 2433. In some embodiments, the recombinant nucleic acid comprises or consists of the nucleic acid sequence of SEQ ID NO: 2434. In some embodiments, the recombinant nucleic acid comprises or consists of the nucleic acid sequence of SEQ ID NO: 2435. In some embodiments, the recombinant nucleic acid comprises or consists of the nucleic acid sequence of SEQ ID NO: 2436. In some embodiments, the recombinant nucleic acid comprises or consists of the nucleic acid sequence of SEQ ID NO: 2437. In some embodiments, the recombinant nucleic acid comprises or consists of the nucleic acid sequence of SEQ ID NO: 2438.

Aspects of the present disclosure provide a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404. In some embodiments, the promoter comprises or consists of the nucleic acid sequence of any one of SEQ ID NOs: 2402-2404. In some embodiments, the promoter comprises or consists of the nucleic acid sequence of SEQ ID NO: 2402. In some embodiments, the promoter comprises or consists of the nucleic acid sequence of SEQ ID NO: 2403. In some embodiments, the promoter comprises or consists of the nucleic acid sequence of SEQ ID NO: 2404. In some embodiments, the recombinant nucleic acid comprising the promoter exhibits enhanced expression in skeletal muscle cells and/or cardiac muscle cells compared to non-skeletal muscle cells and/or non-cardiac muscle cells.

Aspects of the present disclosure provide a recombinant nucleic acid comprising a codon optimized sequence encoding microdystrophin that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407. In some embodiments, the codon optimized sequence encoding microdystrophin comprises or consists of the nucleic acid sequence of any one of SEQ ID NOs: 2405-2407. In some embodiments, the codon optimized sequence encoding microdystrophin comprises or consists of the nucleic acid sequence of SEQ ID NO: 2405. In some embodiments, the codon optimized sequence encoding microdystrophin comprises or consists of the nucleic acid sequence of SEQ ID NO: 2406. In some embodiments, the codon optimized sequence encoding microdystrophin comprises or consists of the nucleic acid sequence of SEQ ID NO: 2407.

Aspects of the present disclosure provide an adeno-associated virus (AAV) particle comprising any one of the recombinant nucleic acids described herein and a capsid protein. In some embodiments, the capsid protein is derived from a serotype selected from AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, and AAV7m8, or a derivative, hybrid or chimeric serotype thereof.

In some embodiments, the AAV particle has increased muscle tropism compared to a wild type AAV9 particle. In some embodiments, the AAV particle has reduced liver tropism compared to a wild type AAV9 particle.

In some embodiments, the capsid protein comprises an amino acid sequence of formula (I): X₁NX₂X₃X₄RGDRX₅X₆L (formula I; SEQ ID NO: 1), wherein each of X₁-X₆ is any amino acid.

In some embodiments, the amino acid sequence of formula (I) is in a hypervariable region IV (HVR IV) relative to a wild type AAV9 capsid. In some embodiments, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L (SEQ ID NO: 2441) is inserted after amino acid 455. In some embodiments, X₁ is A, I, F, G, H, L, M, Q, S, T, or V; X₂ is A, G, S, T, or Y; X₃ is S, N, G, or P; X₄ is A, G, H, I, M, S, T, or V; X₅ is A, G, or Q; and X₆ is A, I, L, M, N, S, or Y. In some embodiments, the amino acid sequence of formula (I) comprises of any one of SEQ ID NOs: 801-1599. In some embodiments, the amino acid sequence of formula (I) comprises of any one of SEQ ID NOs: 1600-2398. In some embodiments, the amino acid sequence of formula (I) comprises or consists of any one of SEQ ID NOs: 2-800.

In some embodiments, the amino acid sequence of formula (I) comprises or consists of SEQ ID NOs: 191, 198, 199, 215, 256, 379, 544, 550, or 748. In some embodiments, the amino acid sequence of formula (I) comprises or consists of SEQ ID NO: 191. In some embodiments, the amino acid sequence of formula (I) comprises or consists of SEQ ID NO: 198. In some embodiments, the amino acid sequence of formula (I) comprises or consists of SEQ ID NO: 199. In some embodiments, the amino acid sequence of formula (I) comprises or consists of SEQ ID NO: 215. In some embodiments, the amino acid sequence of formula (I) comprises or consists of SEQ ID NO: 256. In some embodiments, the amino acid sequence of formula (I) comprises or consists of SEQ ID NO: 379. In some embodiments, the amino acid sequence of formula (I) comprises or consists of SEQ ID NO: 544. In some embodiments, the amino acid sequence of formula (I) comprises or consists of SEQ ID NO: 550. In some embodiments, the amino acid sequence of formula (I) comprises or consists of SEQ ID NO: 748.

In some embodiments, the capsid protein further comprises a deletion of amino acid G267 relative to a wild type AAV9 vector. In some embodiments, the amino acid sequence of formula (I) comprises or consists of SEQ ID NO: 191 and the capsid protein further comprises a deletion of amino acid G267 relative to a wild type AAV9 vector. In some embodiments, the amino acid sequence of formula (I) comprises or consists of SEQ ID NO: 198 and the capsid protein further comprises a deletion of amino acid G267 relative to a wild type AAV9 vector. In some embodiments, the amino acid sequence of formula (I) comprises or consists of SEQ ID NO: 199 and the capsid protein further comprises a deletion of amino acid G267 relative to a wild type AAV9 vector. In some embodiments, the amino acid sequence of formula (I) comprises or consists of SEQ ID NO: 215 and the capsid protein further comprises a deletion of amino acid G267 relative to a wild type AAV9 vector. In some embodiments, the amino acid sequence of formula (I) comprises or consists of SEQ ID NO: 256 and the capsid protein further comprises a deletion of amino acid G267 relative to a wild type AAV9 vector. In some embodiments, the amino acid sequence of formula (I) comprises or consists of SEQ ID NO: 379 and the capsid protein further comprises a deletion of amino acid G267 relative to a wild type AAV9 vector. In some embodiments, the amino acid sequence of formula (I) comprises or consists of SEQ ID NO: 544 and the capsid protein further comprises a deletion of amino acid G267 relative to a wild type AAV9 vector. In some embodiments, the amino acid sequence of formula (I) comprises or consists of SEQ ID NO: 550 and the capsid protein further comprises a deletion of amino acid G267 relative to a wild type AAV9 vector. In some embodiments, the amino acid sequence of formula (I) comprises or consists of SEQ ID NO: 748 and the capsid protein further comprises a deletion of amino acid G267 relative to a wild type AAV9 vector.

Aspects of the present disclosure provide a pharmaceutical composition comprising any of the recombinant nucleic acids, the expression vectors (e.g., the AAV vectors), or the AAV particles described herein, and a pharmaceutically acceptable carrier.

Aspects of the present disclosure provide a method of treating DMD in a subject in need thereof, the method comprising administering to the subject an effective amount of any of the recombinant nucleic acids, the expression vectors (e.g., the AAV vectors), the AAV particles, or the pharmaceutical compositions described herein.

In some embodiments, the subject is a human subject. In some embodiments, the subject has at least one mutation in the dystrophin gene.

In some embodiments, the administering comprises intramuscular administration, subcutaneous injection, intravenous injection, or intravenous (IV) administration.

Aspects of the present disclosure provide uses of any of the recombinant nucleic acids, the expression vectors (e.g., the AAV vectors), the AAV particles, or the pharmaceutical compositions described herein for the manufacture of a medicament for treating DMD.

Aspects of the present disclosure provide any of the recombinant nucleic acids, the expression vectors (e.g., the AAV vectors), the AAV particles, or the pharmaceutical compositions described herein for use in treating DMD.

Aspects of the present disclosure provide a kit comprising any of the recombinant nucleic acids, the expression vectors (e.g., the AAV vectors), the AAV particles, or the pharmaceutical compositions described herein. In some embodiments, the kit further comprises instructions for use in treating Duchenne muscular dystrophy (DMD) in a subject in need thereof.

Aspects of the nucleic acid molecules (e.g., cargo) and capsid proteins are described in further detail below.

For sequences disclosed throughout this application, it is understood that nucleic acid molecules and peptides may comprise one or more substitutions, for example conservative substitutions, that allow sequences to continue to function. Accordingly, sequences may have at least 80%, 85%, 90%, 95%, 96%, 97%, 98%, or 99% sequence identity to the sequence disclosed.

A conservative substitution refers to amino acid substitutions that do not significantly affect or alter binding characteristics of a particular protein. Generally, conservative substitutions are ones in which a substituted amino acid residue is replaced with an amino acid residue having a similar side chain. For example, conservative substitutions may include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp.

### Codon Optimized Microdystrophin

The present invention provides novel engineered nucleic acids encoding microdystrophin proteins for use in gene therapy. The microdystrophin proteins of the invention are optimized to provide increased expression in muscle cells. Advantageously, microdystrophin proteins of the invention are substantially shorter than dystrophin, allowing for optimized packaging in expressing cassettes, including viral vectors.

Microdystrophin refers to a biologically active fragment of dystrophin, e.g., ΔR4-R23/ΔCT microdystrophin. Microdystrophin can be from any source, e.g., a human microdystrophin. The microdystrophin can comprise or consist of an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 2439. In some embodiments, microdystrophin comprises or consists of the amino acid sequence of SEQ ID NO: 2439.

Recombinant nucleic acids described herein can include a microdystrophin protein described herein or a variant thereof. For example, the recombinant nucleic acid comprises a microdystrophin comprising or consisting of an amino acid sequence of SEQ ID NO: 2439, or a variant thereof comprising 1, 2, 3, 4, 5, 6, 7, 8, 9, or 10 amino acid substitutions.

Microdystrophin protein sequence
(SEQ ID NO: 2439)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDILLKWQRLTEEQCLFSAWLSEKEDAVNKIHTTGFKDQNEMLSSL
QKLAVLKADLEKKKQSMGKLYSLKQDLLSTLKNKSVTQKTEAWLDNFARCWDNLVQKLEKSTAQISQAVTTTQPS
LTQTTVMETVTTVTTREQILVKHAQEELPPPPPQKKRTLERLQELQEATDELDLKLRQAEVIKGSWQPVGDLLID
SLQDHLEKVKALRGEIAPLKENVSHVNDLARQLTTLGIQLSPYNLSTLEDLNTRWKLLQVAVEDRVRQLHEAHRD
FGPASQHELSTSVQGPWERAISPNKVPYYINHETQTTCWDHPKMTELYQSLADLNNVRESAYRTAMKLRRLQKAL
CLDLLSLSAACDALDQHNLKQNDQPMDILQIINCLTTIYDRLEQEHNNLVNVPLCVDMCLNWLLNVYDTGRTGRI
RVLSFKTGIISLCKAHLEDKYRYLFKQVASSTGFCDQRRLGLLLHDSIQIPRQLGEVASFGGSNIEPSVRSCFQF
ANNKPEIEAALFLDWMRLEPQSMVWLPVLHRVAAAETAKHQAKCNICKECPIIGFRYRSLKHFNYDICQSCFFSG
RVAKGHKMHYPMVEYCTPTTSGEDVRDFAKVLKNKERTKRYFAKHPRMGYLPVQTVLEGDNMETDTM

Aspects of the invention provide a composition comprising a nucleic acid molecule comprising a sequence having at least 95% sequence identity with a sequence selected from SEQ ID NOs: 2405-2407, provided below:

| Name, | |
|---|---|
| SEQ ID | |
| NO | Optimized sequence |
| v1, | ATGCTGTGGTGGGAGGAAGTGGAGGACTGCTACGAGAGAGAGGACGTGCAGAAGAAGACCTTCACCA |
| SEQ ID | AGTGGGTGAACGCCCAGTTCAGCAAGTTCGGCAAGCAGCACATCGAGAACCTGTTCAGCGACCTGCA |
| NO: | GGACGGCAGAAGACTGCTGGACCTGCTGGAGGGCCTGACCGGCCAGAAGCTGCCTAAGGAGAAGGGC |
| 2405 | AGCACCAGAGTGCACGCTCTCAACAACGTGAACAAGGCCCTGAGAGTGCTGCAGAACAACAACGTGG |
| | ACCTTGTGAACATCGGAAGCACCGACATCGTGGATGGCAACCACAAGCTGACCCTGGGACTGATCTG |
| | GAACATCATCCTGCACTGGCAGGTGAAGAACGTCATGAAGAACATCATGGCCGGCCTGCAGCAGACC |
| | AACAGCGAAAAGATCCTGCTGAGCTGGGTCAGACAGAGCACCAGAAACTACCCTCAGGTGAACGTGA |
| | TCAACTTCACCACCAGCTGGAGCGACGGCCTGGCCCTGAACGCCCTGATCCACAGCCACAGACCCGA |
| | CCTGTTCGACTGGAACAGCGTGGTGTGCCAGCAGAGCGCCACCCAGAGACTGGAGCACGCCTTCAAC |
| | ATCGCCAGATACCAGCTGGGCATCGAGAAGCTGCTGGACCCCGAGGACGTGGATACCACCTACCCCG |
| | ACAAGAAGAGCATCCTGATGTACATCACCAGCCTGTTCCAGGTGCTGCCCCAGCAAGTGAGCATCGA |
| | GGCCATCCAGGAGGTGGAGATGCTGCCCAGACCTCCAAAGGTGACCAAGGAGGAGCACTTCCAGCTG |
| | CACCACCAGATGCACTACAGCCAGCAGATCACCGTGAGCCTGGCCCAGGGCTACGAGAGAACCAGCA |
| | GCCCCAAGCCCAGATTCAAGAGCTACGCCTACACCCAGGCCGCCTACGTGACCACCAGCGACCCCAC |
| | CAGAAGCCCCTTCCCCAGCCAGCACCTGGAGGCCCCCGAGGATAAGAGCTTCGGCAGCAGCCTGATG |
| | GAGAGCGAGGTGAACTTGGACAGATACCAGACCGCCCTGGAGGAGGTGCTGAGCTGGCTGCTGAGCG |
| | CCGAGGACACCCTTCAGGCACAGGGCGAGATCAGCAACGACGTGGAGGTGGTGAAGGACCAGTTCCA |
| | CACCCACGAGGGCTACATGATGGACCTGACCGCCCACCAGGGCAGAGTGGGTAACATCCTCCAGCTG |
| | GGAAGCAAGCTGATCGGCACCGGCAAGCTGAGCGAGGACGAGGAGACCGAGGTGCAAGAGCAAATGA |
| | ACCTGCTTAACAGCAGATGGGAGTGCCTGAGAGTGGCCAGCATGGAGAAGCAGAGCAACCTGCACAG |
| | AGTGCTGATGGACCTGCAGAACCAGAAGCTGAAGGAGCTGAACGACTGGCTGACCAAGACCGAGGAG |
| | AGAACCAGAAAGATGGAGGAGGAGCCCCTGGGCCCGGACCTGGAGGACCTGAAGCGGCAGGTGCAGC |
| | AGCACAAGGTCTTACAGGAGGACCTGGAGCAGGAGCAAGTGAGAGTGAACAGCCTGACCCACATGGT |
| | GGTGGTGGTGGATGAGAGCAGCGGCGACCACGCCACCGCCGCCCTGGAGGAGCAGCTGAAGGTGCTG |
| | GGCGACAGATGGGCCAACATCTGCAGATGGACCGAGGACAGATGGGTTCTGCTGCAGGACATCCTGC |
| | TGAAGTGGCAGAGACTGACCGAGGAACAGTGCCTGTTCAGCGCCTGGCTGAGTGAGAAGGAAGACGC |
| | TGTGAACAAGATCCACACCACCGGCTTCAAGGACCAGAACGAGATGCTGAGCAGCCTGCAGAAGCTG |
| | GCCGTGCTGAAGGCAGACCTGGAGAAGAAAAAGCAGTCCATGGGAAAGCTGTACAGCCTGAAGCAAG |
| | ACCTGCTGAGCACCCTGAAGAACAAGAGCGTGACCCAGAAGACCGAGGCCTGGCTGGACAACTTCGC |
| | CAGATGCTGGGACAACCTGGTTCAGAAGCTGGAGAAGAGCACCGCCCAGATCAGCCAGGCCGTGACC |
| | ACCACCCAGCCCAGCCTGACCCAGACCACCGTGATGGAGACCGTGACCACCGTGACCACCAGAGAGC |
| | AGATCCTGGTGAAGCACGCCCAGGAGGAGCTGCCCCCTCCTCCCCCGCAAAAGAAGAGAACCCTGGA |
| | GAGACTGCAGGAGCTGCAGGAGGCTACCGATGAGCTGGACCTGAAGCTGAGACAGGCCGAGGTGATC |
| | AAGGGCAGCTGGCAGCCCGTGGGTGACCTGCTTATCGACAGCCTGCAGGACCACCTAGAGAAGGTGA |
| | AGGCCCTGAGAGGCGAGATCGCCCCTCTGAAGGAGAACGTGAGCCACGTGAACGACCTGGCCAGACA |
| | GCTGACCACCCTGGGAATCCAGCTGAGCCCCTACAACCTGAGCACCCTGGAGGACCTGAACACGAGA |
| | TGGAAGCTGCTGCAAGTGGCCGTGGAGGACAGAGTCAGACAGTTGCACGAGGCTCACAGAGATTTCG |
| | GCCCCGCTAGTCAGCACTTCCTGAGCACCAGCGTGCAGGGCCCCTGGGAGAGAGCTATCAGCCCAAA |
| | CAAGGTGCCCTACTACATCAACCACGAGACCCAGACCACCTGCTGGGACCACCCCAAGATGACCGAG |
| | CTGTACCAGAGCCTGGCAGACCTCAACAACGTGAGATTCAGCGCCTACAGAACCGCCATGAAGCTGA |
| | GAAGACTGCAGAAGGCCCTGTGCCTGGACCTGCTGAGCCTGAGCGCCGCCTGCGACGCCCTGGACCA |
| | GCACAACCTGAAGCAGAACGACCAGCCCATGGACATCCTTCAGATCATCAACTGCCTGACCACCATC |
| | TACGACAGACTGGAGCAGGAACATAACAACCTGGTGAACGTGCCCCTGTGCGTGGACATGTGCCTGA |
| | ACTGGCTGCTGAACGTGTACGACACCGGCAGAACAGGCAGAATCAGAGTGCTGAGCTTCAAGACCGG |
| | CATCATCAGCCTGTGCAAGGCCCACCTGGAGGACAAGTACAGATACTTATTCAAGCAGGTGGCCAGC |
| | AGCACCGGCTTCTGCGATCAGAGAAGACTGGGCCTGCTGCTGCACGACAGCATCCAGATCCCCAGAC |
| | AGCTGGGCGAAGTGGCCAGCTTCGGCGGCAGCAACATCGAGCCCAGCGTGAGAAGCTGCTTCCAGTT |
| | CGCTAACAACAAGCCCGAGATCGAGGCCGCCCTGTTCTTGGACTGGATGAGACTGGAGCCCCAGAGC |
| | ATGGTGTGGCTGCCCGTGCTGCACAGAGTGGCCGCCGCCGAGACCGCCAAGCACCAGGCCAAGTGTA |
| | ACATCTGCAAGGAGTGCCCAATCATCGGCTTCAGATACAGAAGCCTGAAGCACTTCAACTACGACAT |
| | CTGCCAGAGCTGCTTCTTCAGCGGCAGAGTGGCCAAGGGCCACAAGATGCACTACCCCATGGTGGAG |
| | TACTGCACCCCCACCACCAGCGGCGAGGACGTGAGAGACTTCGCAAAGGTCCTGAAGAACAAGTTCA |
| | GAACCAAGAGATACTTCGCTAAGCACCCCAGAATGGGCTACCTGCCCGTGCAGACCGTGCTGGAGGG |
| | CGATAACATGGAGACCGACACCATGTGA |
| v2, | ATGCTGTGGTGGGAAGAGGTCGAGGACTGCTACGAGCGCGAAGATGTGCAGAAGAAAACTTTCACTA |
| SEQ ID | AATGGGTGAATGCCCAGTTCTCTAAGTTCGGAAAGCAGCACATCGAAAACCTGTTTTCCGATCTGCA |
| NO: | AGATGGCCGGCGGCTGCTGGACCTGCTGGAGGGGCTGACCGGACAGAAGCTGCCTAAGGAGAAAGGA |
| 2406 | TCTACCAGGGTCCATGCTCTGAATAACGTGAACAAGGCTCTGAGGGTGCTGCAGAACAATAACGTGG |
| | ACCTGGTGAACATTGGTTCCACAGACATCGTGGATGGAAACCACAAACTGACACTGGGACTGATCTG |
| | GAACATCATTCTGCATTGGCAAGTGAAGAACGTGATGAAGAACATCATGGCCGGACTGCAGCAGACG |
| | AACTCCGAGAAGATCCTGCTTAGCTGGGTGAGACAGAGTACCCGGAATTACCCACAGGTGAACGTGA |
| | TCAATTTCACAACCAGCTGGTCAGACGGCCTGGCTCTGAATGCCCTGATTCATAGCCACCGGCCAGA |
| | TCTGTTTGACTGGAACTCTGTCGTGTGTCAGCAGTCAGCCACCCAGAGGCTGGAGCACGCCTTCAAT |
| | ATTGCCCGCTATCAGCTGGGGATTGAGAAACTGCTGGACCCCGAGGATGTGGACACAACATACCCTG |
| | ACAAGAAGAGCATACTTATGTATATCACAAGCCTGTTCCAGGTGCTGCCACAGCAGGTCTCCATCGA |
| | GGCCATTCAAGAAGTGGAGATGCTGCCCAGGCCTCCTAAGGTCACCAAAGAGGAGCACTTTCAGCTC |
| | CATCATCAGATGCACTACAGCCAGCAAATTACCGTGTCCCTGGCCCAGGGCTACGAGAGGACAAGTT |
| | CCCCCAAGCCTAGATTTAAGAGCTATGCCTATACCCAGGCCGCCTACGTGACTACCTCCGATCCCAC |
| | CAGGAGCCCATTTCCCAGCCAGCACCTGGAGGCTCCCGAAGATAAGAGCTTTGGCAGCAGCCTGATG |
| | GAATCTGAGGTGAACCTGGACAGATACCAGACTGCGCTGGAGGAGGTGCTGTCCTGGCTGCTGAGCG |
| | CCGAGGACACCCTGCAGGCTCAGGGCGAGATTAGTAATGATGTGGAGGTGGTGAAGGATCAGTTTCA |
| | CACTCACGAAGGCTACATGATGGACCTGACCGCCCACCAGGGCAGGGTGGGGAACATTCTGCAGCTC |
| | GGGAGTAAGCTGATAGGGACCGGCAAACTGTCCGAGGATGAGGAAACCGAGGTGCAGGAGCAGATGA |
| | ACCTGCTTAACTCCCGGTGGGAGTGTCTGAGAGTGGCCAGCATGGAGAAGCAGAGCAATCTGCACAG |
| | AGTGCTGATGGATCTGCAGAATCAGAAGCTGAAGGAACTGAATGATTGGCTCACAAAGACAGAGGAG |
| | AGGACTAGAAAGATGGAGGAGGAGCCCCTGGGCCCTGACCTGGAGGACCTGAAGCGACAGGTGCAGC |
| | AGCACAAGGTGCTGCAGGAGGATCTGGAGCAAGAACAAGTGAGGGTGAACTCTCTGACTCATATGGT |
| | TGTGGTGGTGGACGAGTCTTCCGGAGACCACGCAACTGCCGCCCTCGAGGAGCAGCTGAAGGTGCTG |
| | GGCGATAGGTGGGCTAATATCTGTAGGTGGACCGAGGACAGATGGGTGCTGCTGCAGGATATCCTGC |
| | TGAAATGGCAGCGCCTGACAGAAGAGCAGTGTCTGTTCAGCGCATGGCTGAGCGAAAAGGAGGACGC |
| | TGTGAACAAGATCCACACTACAGGATTTAAAGACCAGAACGAGATGCTGAGTAGCCTGCAGAAGCTG |
| | GCTGTGCTGAAGGCTGATTTGGAGAAAAAGAAGCAGAGCATGGGCAAGCTGTACTCCCTGAAACAGG |
| | ACCTGCTGAGCACCCTGAAGAATAAGTCCGTGACCCAGAAAACCGAAGCTTGGCTGGACAACTTCGC |
| | GCGCTGCTGGGATAACCTGGTGCAGAAGCTGGAGAAGAGCACCGCCCAGATTTCTCAAGCAGTGACC |
| | ACAACCCAGCCCAGCCTGACCCAGACTACAGTAATGGAGACAGTCACTACCGTGACCACCCGGGAGC |
| | AGATCCTGGTGAAGCACGCCCAGGAAGAGCTGCCGCCCCCACCACCCCAGAAAAAGCGGACCCTGGA |
| | GCGCCTGCAGGAGCTCCAGGAGGCCACCGACGAACTGGATCTGAAGCTGAGGCAGGCCGAGGTGATT |
| | AAGGGATCCTGGCAGCCTGTGGGAGACCTGCTGATCGACAGCCTGCAGGATCATCTGGAGAAAGTCA |
| | AAGCTCTGAGAGGGGAGATCGCCCCACTGAAGGAGAACGTGAGCCACGTGAATGACCTGGCTAGACA |
| | GCTGACCACCTTGGGCATTCAGCTGAGTCCTTACAATCTGTCAACTCTGGAGGATCTGAACACCCGG |
| | TGGAAACTGCTGCAGGTGGCTGTGGAAGATCGGGTGCGCCAGTTACATGAGGCCCACAGGGACTTTG |
| | GCCCAGCCTCACAGCACTTTCTGTCAACCTCTGTGCAAGGACCTTGGGAGAGAGCAATCAGCCCAAA |
| | CAAGGTGCCATATTATATCAATCATGAGACTCAGACAACTTGCTGGGATCACCCCAAGATGACAGAA |
| | CTCTATCAGTCACTCGCCGATCTCAATAATGTACGGTTCTCTGCCTACCGGACAGCTATGAAGCTGC |
| | GGAGGCTGCAGAAGGCACTCTGTCTGGACCTGCTGAGTCTCTCTGCGGCCTGCGATGCCCTGGATCA |
| | ACATAACCTGAAGCAGAATGATCAGCCTATGGACATCCTGCAGATCATTAACTGTCTCACCACAATC |
| | TACGATCGGCTGGAACAGGAACATAACAACCTGGTGAATGTGCCACTGTGTGTGGACATGTGCCTGA |
| | ATTGGCTGCTCAACGTCTACGATACAGGCAGGACTGGCAGGATCCGCGTGCTGAGCTTTAAAACCGG |
| | CATCATCTCTCTGTGCAAAGCCCACCTGGAGGACAAATATCGGTACCTGTTTAAGCAGGTGGCCTCC |
| | AGCACAGGCTTCTGTGATCAGCGCCGGCTGGGCCTGCTGCTGCACGACAGCATTCAGATCCCTAGGC |
| | AGCTGGGCGAGGTGGCCTCCTTCGGCGGCTCAAACATTGAACCCAGCGTGAGATCTTGCTTTCAGTT |
| | TGCAAATAATAAACCCGAGATCGAGGCCGCCCTGTTCCTGGATTGGATGAGACTGGAGCCTCAGAGT |
| | ATGGTGTGGCTGCCAGTGCTGCATAGAGTGGCCGCCGCTGAGACGGCCAAGCACCAGGCAAAGTGCA |
| | ATATCTGCAAGGAATGCCCCATTATTGGCTTTCGATACCGGTCCTTGAAACACTTCAATTATGACAT |
| | CTGCCAGTCATGCTTCTTCAGCGGACGCGTGGCCAAGGGACACAAGATGCATTATCCTATGGTGGAA |
| | TACTGCACTCCAACTACTTCCGGCGAGGACGTGAGAGATTTCGCTAAAGTTCTTAAAAACAAATTTC |
| | GGACTAAGAGGTACTTCGCCAAGCACCCCCGCATGGGCTATCTGCCTGTGCAGACAGTGCTTGAAGG |
| | AGACAACATGGAAACCGATACAATGTGA |
| v3, | ATGCTGTGGTGGGAGGAGGTGGAGGATTGCTACGAGAGAGAGGACGTGCAGAAGAAAACCTTCACCA |
| SEQ ID | AATGGGTGAACGCCCAGTTCAGCAAGTTTGGCAAGCAGCACATCGAGAACCTGTTCAGCGATCTGCA |
| NO: | GGACGGCCGCAGGCTGCTGGATCTGCTGGAGGGTCTGACCGGCCAGAAACTGCCCAAAGAGAAGGGA |
| 2407 | TCAACCAGGGTGCACGCCCTGAATAATGTGAACAAGGCCCTGAGAGTGCTGCAAAATAATAATGTGG |
| | ATCTGGTGAACATCGGATCTACCGATATTGTGGACGGCAACCATAAGCTGACTCTGGGCCTGATCTG |
| | GAATATCATCCTGCACTGGCAGGTGAAAAATGTGATGAAAAACATCATGGCCGGGCTGCAGCAGACC |
| | AACTCTGAGAAGATCCTGCTGAGCTGGGTGAGGCAGTCTACAAGAAATTACCCCCAGGTGAACGTGA |
| | TCAATTTCACCACCAGCTGGTCCGACGGGCTGGCTCTGAATGCCCTGATTCACAGCCACCGGCCCGA |
| | CCTGTTCGACTGGAATTCCGTGGTGTGCCAGCAGAGCGCCACACAGAGACTGGAGCACGCTTTCAAT |
| | ATCGCTCGGTATCAGCTGGGCATCGAGAAGCTGCTGGACCCCGAGGACGTGGACACTACTTACCCCG |
| | ATAAAAAGAGCATCCTGATGTATATCACCTCCCTGTTCCAAGTGCTGCCCCAGCAGGTCTCCATCGA |
| | GGCCATCCAGGAGGTGGAAATGCTGCCCCGGCCCCCAAAGGTGACAAAGGAAGAACACTTCCAGCTC |
| | CACCACCAGATGCACTACTCTCAGCAGATCACCGTGAGCCTGGCCCAGGGGTACGAGCGGACCTCTT |
| | CTCCAAAGCCCCGCTTCAAGAGCTATGCCTATACCCAGGCCGCCTATGTGACCACCTCCGATCCCAC |
| | CCGCTCACCCTTCCCTTCTCAACACCTGGAAGCCCCAGAGGACAAGTCCTTCGGCAGTTCTCTGATG |
| | GAGTCTGAGGTGAACCTGGATAGATATCAGACTGCCCTGGAGGAGGTGCTGTCTTGGCTGCTCTCCG |
| | CCGAGGACACCCTGCAGGCACAGGGCGAGATCTCCAACGATGTGGAGGTGGTGAAGGACCAGTTCCA |
| | CACACACGAGGGCTATATGATGGACCTGACTGCCCATCAGGGCAGAGTGGGCAATATCCTGCAGCTG |
| | GGTTCCAAACTGATAGGCACCGGAAAGCTGAGCGAAGACGAGGAGACCGAGGTGCAGGAGCAGATGA |
| | ATCTGCTGAACTCCCGGTGGGAGTGCCTGCGAGTCGCCAGCATGGAAAAGCAGAGCAACCTGCACCG |
| | CGTGCTCATGGATCTGCAGAATCAGAAGCTGAAGGAGCTGAATGACTGGCTGACCAAGACCGAAGAG |
| | CGCACCAGGAAGATGGAGGAGGAGCCACTGGGCCCCGATCTGGAGGACCTGAAAAGACAGGTGCAGC |
| | AGCATAAAGTGCTGCAGGAGGACCTGGAGCAGGAACAAGTGAGAGTGAATTCTCTGACACACATGGT |
| | GGTGGTGGTGGACGAGAGCAGCGGCGACCACGCAACAGCCGCCCTGGAGGAGCAGCTGAAAGTGCTG |
| | GGAGACCGGTGGGCCAACATTTGCCGGTGGACCGAGGACCGCTGGGTGCTGCTGCAGGACATTCTGC |
| | TGAAGTGGCAGCGGCTGACCGAAGAGCAGTGCCTGTTCTCCGCCTGGCTGAGCGAGAAGGAGGACGC |
| | CGTGAATAAGATCCACACCACCGGGTTCAAGGACCAGAATGAGATGCTGTCCAGCCTGCAGAAGCTG |
| | GCCGTGCTGAAGGCCGATCTGGAAAAGAAGAAGCAGAGCATGGGAAAGCTGTATAGCCTGAAGCAGG |
| | ACCTGCTGTCTACTCTGAAAAACAAGTCAGTGACCCAGAAGACCGAGGCCTGGCTGGACAACTTCGC |
| | CCGCTGCTGGGACAACCTGGTGCAGAAGCTGGAAAAGAGCACCGCCCAGATCAGCCAGGCAGTGACT |
| | ACCACACAGCCCTCCCTGACACAGACTACTGTGATGGAGACCGTGACAACAGTGACCACAAGGGAGC |
| | AGATCCTGGTGAAGCACGCCCAGGAAGAACTGCCCCCTCCACCCCCTCAGAAGAAGAGAACTCTGGA |
| | AAGACTGCAGGAGCTGCAGGAGGCCACTGACGAGCTGGACCTGAAGCTGCGGCAGGCCGAGGTGATT |
| | AAGGGCAGCTGGCAGCCTGTGGGCGATCTGCTGATCGACAGCCTGCAGGACCACCTGGAGAAGGTGA |
| | AAGCCCTGCGGGGCGAGATCGCCCCTCTGAAGGAGAACGTCAGCCATGTGAATGACCTGGCCAGACA |
| | GCTGACCACACTGGGGATTCAGCTGTCTCCTTACAATCTGTCAACCCTGGAGGACCTGAACACTCGC |
| | TGGAAGCTGCTGCAGGTGGCCGTGGAGGACAGGGTGCGGCAGCTGCACGAAGCCCATCGCGACTTTG |
| | GGCCCGCCTCTCAGCACTTTCTGAGCACCTCCGTGCAGGGACCCTGGGAGCGGGCAATCAGCCCAAA |
| | CAAGGTGCCATACTACATCAACCACGAGACCCAGACCACCTGTTGGGATCACCCCAAGATGACAGAG |
| | CTGTACCAGTCTCTGGCTGACCTGAACAACGTGCGGTTCTCAGCCTACAGGACCGCCATGAAGCTGC |
| | GGAGACTGCAGAAGGCCCTGTGCCTGGATCTGCTGAGCCTCTCCGCTGCTTGTGACGCCCTGGATCA |
| | GCACAACCTGAAACAGAACGACCAGCCAATGGACATCCTGCAGATTATTAATTGCCTGACAACCATC |
| | TATGATCGGCTGGAACAGGAGCACAATAATCTGGTGAACGTGCCACTGTGCGTGGACATGTGCCTGA |
| | ATTGGCTGCTGAACGTGTACGACACAGGACGGACCGGCAGAATCAGAGTGCTGTCTTTTAAGACCGG |
| | GATCATCAGCCTGTGCAAAGCCCACCTGGAGGACAAGTACCGGTATCTGTTCAAACAAGTGGCCTCC |
| | AGCACCGGCTTTTGCGACCAGCGCAGGCTGGGCCTGCTGCTCCACGACTCCATCCAGATCCCTAGAC |
| | AGCTGGGCGAGGTGGCTTCTTTTGGCGGATCCAATATCGAGCCCAGTGTGCGGAGCTGTTTTCAGTT |
| | TGCTAATAATAAGCCTGAGATCGAGGCCGCTCTGTTCCTGGACTGGATGAGACTGGAGCCCCAGTCC |
| | ATGGTGTGGCTGCCAGTGCTGCACAGGGTCGCCGCCGCAGAGACTGCCAAGCACCAGGCCAAGTGCA |
| | ACATCTGCAAAGAATGCCCAATCATTGGCTTTAGATACCGGAGCCTCAAACACTTTAACTACGACAT |
| | CTGCCAGAGCTGCTTCTTCTCCGGCCGGGTGGCCAAGGGCCACAAGATGCACTACCCCATGGTGGAG |
| | TACTGTACCCCAACCACTTCCGGCGAGGACGTGAGAGATTTCGCCAAGGTGCTGAAGAACAAGTTCC |
| | GGACAAAGAGATACTTTGCAAAACACCCAAGAATGGGCTACCTGCCAGTGCAGACCGTGCTGGAGGG |
| | GGACAATATGGAGACAGACACAATGTGA |

In aspects of the invention, the nucleic acid molecule comprises a sequence having the sequence identity of SEQ ID NO: 2405, SEQ ID NO: 2406, or SEQ ID NO: 2407. In aspects of the invention, the nucleic acid molecule may comprise a sequence having at least 85%, 86%, 87%, 88% 89%, 90%, 91%, 92%, 93%, 94%, or 95% of the sequence identity of SEQ ID NO: 2405, SEQ ID NO: 2406, or SEQ ID NO: 2407.

Advantageously, microdystrophin proteins are substantially shorter than dystrophin, allowing for optimized packaging in expressing cassettes, including viral vectors.

Typical AAV vectors have a packaging capacity of ˜4.7 kb. Nucleic acids are almost universally packaged with two terminal ITRs, each about 0.14 kb in length. Additionally regulatory elements, for example promoter, intron, and poly (A) elements, may be about 0.6-1 kb. As a result, the maximal allowable cDNA length must be less than 3.8 kb. Gene therapies are restricted by the packaging capacity limit of adeno-associated viral (AAV) vectors. As a consequence, the size of the native dystrophin coding sequence prevents packaging of the transgene into traditional AAV serotypes, including AAV9.

Accordingly, in aspects of the invention, the nucleic acid molecule of the present invention may be suitably packaged in a vector.

For example, the vector may be an adeno-associated viral (AAV) vector. The nucleic acid molecule may comprise in order an inverted terminal repeats (ITR) region, a promoter, an intron, the sequence having at least 95% sequence identity with a sequence selected from SEQ ID NOs: 2405-2407, a poly adenylation signal, and an ITR region.

The AAV vector may comprise a capsid protein having at least one modification that results in reduced liver-tropism of the AAV vector and/or preferential targeting of the AAV vector to muscle tissue.

Advantageously, when administered to a subject with a mutation in the DMD gene, resulting in lack of dystrophin protein expression, the composition is effective at restoring a healthy phenotype.

Further advantageously, the microdystrophin protein expressed by the nucleic acid molecule may be smaller than 137.5 kilodaltons (for example, 137.32 kilodaltons). Aspects of the invention also provide methods of treating subjects suffering from DMD using compositions of the invention.

Methods of the invention comprise providing to the subject compositions of the invention described above, comprising a nucleic acid molecule comprising a sequence having at least 95% sequence identity with a sequence selected from SEQ ID NOs: 2405-2407.

DRG De-Targeting miRNAs

The present invention provides recombinant nucleic acids and recombinant expression vectors (e.g., AAV vectors) that encode a transgene (e.g., a microdystrophin) and the binding site(s) for specific micro RNAs (miRs) that direct the miRs to selectively inhibit expression of the same transgene (e.g., the microdystrophin). By the present invention, it was discovered that miR-338-3p, miR-138-5p, and miR-9-5p are highly expressed in dorsal root ganglial (DRG) cells and not expressed, or expressed at substantially low levels, in muscle cells (including skeletal and heart muscle cells).

Accordingly, aspects of the invention provide a recombinant nucleic acid and an engineered vector comprising a first nucleic acid sequence encoding a transgene (e.g., sequence encoding a microdystrophin) and a second nucleic acid sequence encoding for the binding site(s) of one or more miRs selected from among miR-338-3p, miR-138-5p, or miR-9-5p. In such instances, the second nucleic acid sequence can be referred to as DRG de-targeting miRNA binding site. Thus, as used herein, the term “DRG de-targeting binding site” refers to a nucleic acid sequence encoding for a binding site of a miR (e.g., miR-338-3p, miR-138-5p, and miR-9-5p) that is highly expressed in DRG cells but not expressed, or expressed at significantly lower levels, in muscle cells. The nucleic acid sequence encoding a binding site of a miR is sufficiently complementary with the nucleic acid sequence encoding the miR to effect base pairing (binding) between the DRG de-targeting binding site and the miR. Advantageously, the second nucleic acid sequence directs the miR, when present, to repress expression of the transgene. As a result, the selected miRs (miR-338-3p, miR-138-5p, miR-9-5p) in cells transduced by the vectors of the invention will be directed to inhibit expression of the transgene.

For example, the transgene (e.g., microdystrophin) may provide a therapeutic effect when expressed in skeletal and/or heart muscle but provide a toxic effect when expressed in DRG cells. Without being bound to a mechanism of action, transduction of both muscle cells and DRG cells by vectors of the invention results in transcription of both transgene mRNA and the binding site(s) of the select miR. In DRG cells with substantial endogenous expression of miR-338-3p, miR-138-5p, and miR-9-5p, transgene expression (e.g., microdystrophin expression) will be substantially repressed. In muscle cells with limited to no expression of miR-338-3p, miR-138-5p, miR-9-5p, transgene expression will not be substantially repressed. As a result, the therapeutic effect of the transgene will continue in muscle cells and the toxic effect in DRG cells will be abated.

In aspects of the invention, the second nucleic acid sequence may be operably linked to the first nucleic acid sequence, for example, the second nucleic acid sequence may be operably linked to the 3′ or 5′ end of the first nucleic acid sequence. Advantageously, this may result in increased transcription of both nucleic acid sequences.

The engineered vector may be a viral vector. For example, the vector may be an adeno-associated viral (AAV) vector. Further advantageously, the engineered vector may comprise a capsid protein with modified tropism for skeletal and/or heart muscle in comparison with the wild type AAV vector. For example, transduction and expression of the transgene may be increased in muscle tissue while limited expression of the transgene in off-target tissue types, for example, DRG may be observed.

The first nucleic acid sequence and second nucleic acid sequence may also be operably linked to tissue specific promoters. Advantageously, the first nucleic acid sequence may be operably linked to a muscle specific promoter. In aspects of the invention, the second nucleic acid is 3′ or 5′ of the first nucleic acid. For example, the second nucleic acid may be incorporated into a 3′ or 5′ untranslated region (UTR) of the transgene mRNA when transcribed.

In muscle tissue, where miR-338-3p, miR-138-5p, miR-9-5p expression is limited, the muscle specific promoter promotes transcription of the transgene and the miR binding site has limited to no effect due to the absence of the requisite miR. In cells expressing miR-338-3p, miR-138-5p, miR-9-5p, for example DRG, the muscle specific promoter limits transcription of the transgene while the miR binding site is still present in the transcript, inhibiting translation of any transgene mRNA transcribed.

Aspects of the invention also provide methods and uses of the recombinant nucleic acids and the recombinant expression vectors of the invention, for example, in therapeutic compositions. Accordingly, aspects of the invention provide a method or use of a recombinant nucleic acid or a recombinant expression vector, the method or use comprising providing to a subject a recombinant nucleic acid or a recombinant expression vector (e.g., an engineered vector (e.g., an AAV vector)) comprising a first nucleic acid sequence encoding a transgene (e.g., sequence encoding a microdystrophin protein) and a second nucleic acid sequence expressing the binding site of a miR selected from among miR-338-3p, miR-138-5p, or miR-9-5p. The second nucleic acid sequence thereby directs the miR, when present (for example endogenously), to repress expression of the transgene.

The transgene (e.g., microdystrophin) may be expressed in heart and/or skeletal muscle, thereby providing a therapeutic effect. The second nucleic acid sequence may be expressed in dorsal root ganglia cells and direct endogenous miR-338-3p, miR-138-5p, and/or miR-9-5p to repress expression of the transgene in dorsal root ganglia cells. For example, the transgene may provide a therapeutic effect when expressed in skeletal and/or heart muscle but provide a toxic effect when expressed in DRG cells.

Accordingly, in aspects of the invention, the DRG de-targeting miRNA may comprise a sequence have at least 90% or at least 95% of the sequence identity of one or more of:

(SEQ ID NO: 2399; miR-338-3p)
CAACAAAATCACTGATGCTGGA;
(SEQ ID NO: 2400; mir-138-5p)
CGGCCTGATTCACAACACCAGCT;
or
(SEQ ID NO: 2401; miR-9-5p)
TCATACAGCTAGATAACCAAAGA.

### Novel Promoters

The present invention provides novel promoters, that can be linked to a nucleic acid sequence encoding microdystrophin so that the transcription preferably occurs within myocytes and cardiomyocytes. Promoter regions enable the host cells to replicate the AAV delivered nucleic acid only in those cell types and tissues or organs in which the desired protein should be created. Here, the muscle specific promoter is included because it is principally desired that the proteins only be translated in myocytes and cardiomyocytes. Specificity of the cell type into which the nucleic acid is delivered and thus the protein translated is desired because of the adverse effects that may ensue from delivering the nucleic acid and having it translated in cells in which that nucleic acid and thus protein is not needed or not endogenously expressed.

Accordingly, aspects of the present invention provide a promoter having the sequence of any one of SEQ ID NOs: 2402-2404.

Promoter 1
(SEQ ID NO: 2402)
TAGTTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGCTAGA
CTAGCATGCTGCCCATGTAAGGAGGCAAGGCCTGGGGACACCCGAGATGCCTGGTTATAATTAACCCAGACATGT
GGCTGCCCCCCCCCCCCAACACCTGCTGCCTCTAAAAATAACCCTGCATGCCATGTTCCCGGCGAAGGGCCAGCT
GTCCCCCGCCAGCTAGACTCAGCTCTCAGGGGCCCCTCCCTGGGGACAGCCCCTCCTGGCTAGTCACACCCTGTA
GGCTCCTCTATATAACCCAGGGGCACAGGGGCTGCCCTCATTCTACCACCACCTCCACAGCACAGACAGACACTC
AGGAGCCAGCCAGC
Promoter 2
(SEQ ID NO: 2403)
CGTCTGTCTAATTCCAGCCAAGGGCATGGGGGAAAGGACAGAGCCCAGGGACCTGTGGCCTATTCTCTGAGAGGA
CGCTGGGCTAATAATAGGAGGGGAGATTAGGGAGGGATTTGACTGCATTGTTATCTCACAGGAATGGCGTTTGAG
AGCCTTGCTTCCAAGCTCTTCCAAGAGTCATGCCACCCCCCTCCAATCTCTGTGGAAGTGCCCACTGCCTTTCCC
GCTTCGTTCTCTTCTCTCTAAATCTGAGCCCACCCCACTCAGGGGTACAGGATCTGGTTCATTTATCTCCCAGAA
TGATGCCTTGGGTTGCGTCCCCTCTAAACTCTGGCACATTCCAGCCCCTCTTTGGGAGAAAGAACATCTCTTCTT
GCCCAGGCCCTAGTGAGACATGCATCGCAAGACATGCTCCCCCCTTCCTCCCCGCTGGGCTCCAGCTGCTGCCAG
CCCTCCAAGAAAGGAGAGGCCCTGAGTTGGGCTATTTTGGTATCTGGGGTGGGCACCCGCAGGGCTAAGGTTACC
TTGGTATGTTAAGGGCTCCCTGGGGCAGGACTATATAACCCCAGAGGGACTGCCCCATGCTGACTCCTTGCTTCT
TTCCTGCTCGAGCCGCTGCCTTGCCCCCCGGAGACTGAAGACA
Promoter 3
(SEQ ID NO: 2404)
CGTCTGTCTAATTCCAGCCAAGGGCATGGGGGAAAGGACAGAGCCCAGGGACCTGTGGCCTATTCTCTGAGAGGA
CGCTGGGCTAATAATAGGAGGGGAGATTAGGGAGGGATTTGACTGCATTGTTATCTCACAGGAATGGCGTTTGAG
AGCCTTGCTTCCAAGCTCTTCCAAGAGTCATGCCACCCCCCTCCAATCTCTGTGGAAGTGCCCACTGCCTTTCCC
GCTTCGTTCTCTTCTCTCGGTCCCATCCCTGACCCACTAGCCTGGTGCCGGGGGGAGACCAATTTCTGGAGGAAG
CTCTGGGTACCCAGTCTTGCCCTAAGCCTGCCTCTGAACAGGCTGTTCTTGTTTATGACCCCCGTCCCAGCAGAA
TAGTGATTGCTCATCCCTGCATGTGATTTGAGAGGGCCAGTCTTTCAGGCCTGGAGGAAACTCAAGCCCCACCAG
CCGCTTGGCATCGGAATGCTCAGCTGGTCCCCTCCTGGGGCTCATGTGACCAAGATCTAGCTATAAATATCAGAG
GGGCCTCAGACCAAGACAGAATTCGGGCACCAGGAGAAGGAAGCCAACAGGATCCGACCCGGTGTTTTGTGACAA
AGGCAAGACCCCCAGGTCTACTTAGAGCAACGTTAGTAGAGGA

In some embodiments, the recombinant nucleic acid comprises a muscle specific promoter. Non-limiting examples of muscle specific promoters include a CK8 promoter, a desmin promotor, a Mb promoter, a MCK promoter, a MHCK7 promoter, a skeletal muscle alpha actin promoter, and a TTNI2 promoter.

In some embodiments, the recombinant nucleic acid comprises or consists of the nucleic acid sequence of that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2404-2407. In some embodiments, the recombinant nucleic acid comprises or consists of the nucleic acid sequence of any one of SEQ ID NOs: 2402-2404.

In some embodiments, the recombinant nucleic acid comprises or consists of the nucleic acid sequence of SEQ ID NO: 2402. In some embodiments, the recombinant nucleic acid comprises or consists of the nucleic acid sequence of SEQ ID NO: 2403. In some embodiments, the recombinant nucleic acid comprises or consists of the nucleic acid sequence of SEQ ID NO: 2404.

### Engineered Capsid Proteins

The present invention also provides novel capsid protein variants for viral vectors that de-target liver tissue and target skeletal muscle and heart tissue at the same time. Aspects of the present invention provide adeno-associated virus (AAV) vectors comprising a capsid protein comprising an insert comprising the amino acid sequence RGDR (SEQ ID NO: 2440). In the capsid protein, RGDR (SEQ ID NO: 2440) can be inserted after amino acid 455 in reference to an AAV9 capsid or an equivalent position in another AAV capsid. AAV vectors can comprise the amino acid sequence X₁NX₂X₃X₄RGDRX₅X₆L (SEQ ID NO: 1), wherein each of X₁, X₂, X₃, X₄, X₅, and X₆ is any amino acid.

In aspects of the invention, X₁ is an amino acid selected from the group consisting of: A, I, F, G, H, L, M, Q, S, T, and V. In some aspects of the invention, X₁ may be an amino acid selected from the group consisting of: A, I, L, M, S, and V.

In aspects of the invention, X₂ is an amino acid selected from the group consisting of A, G, S, T, and Y.

In aspects of the invention, X₃ is an amino acid selected from the group consisting of S, N, G, and P. In some aspects of the invention, X₃ is S.

In aspects of the invention, X₄ is an amino acid selected from the group consisting of A, G, H, I, M, S, T, and V.

In aspects of the invention, X₅ is an amino acid selected from the group consisting of A, G, and Q.

In aspects of the invention, X₆ is an amino acid selected from the group consisting of A, I, L, M, N, Y. In some aspects of the invention, X₆ is selected from the group consisting of A, S, and Y.

In aspects of the invention, X₁ is located at amino acid 451, X₂ is located at amino acid 453, X₃ is located at amino acid 454, X₄ is located amino acid 455, and RGDRX₅X₆L (SEQ ID NO: 2441) is inserted after amino acid 455 in reference to an AAV9 capsid or an equivalent position in another AAV capsid.

In aspects of the invention, two amino acids from X₁, X₂, X₃, and X₄ are wild type amino acids in reference to an AAV9 capsid or an equivalent position in another AAV capsid and two amino acids from X₁, X₂, X₃, and X₄ are not wild type amino acids.

For example, capsid proteins variants of the invention can comprise a sequence as set forth in Table 1a, e.g., a capsid protein can comprise any one of SEQ ID NOs: 2-800. Notably, capsid protein variants on the invention comprise deletions, substitutions, and/or insertions relative to wild type viral vector capsids.

In aspects of the invention, the capsid protein comprises an amino acid sequence selected from Table 1a and the amino acid sequence is in hypervariable region IV (HVR IV) relative to wild type AAV9. The capsid protein variant can comprise substitutions at amino acids 451-455 relative to a wild type AAV9 capsid. For example, the substitutions at amino acids 451-455 relative to a wild type AAV9 capsid can be an amino acid sequence selected from column 1 of Table 1b, e.g., the substitutions relative to a wild type AAV9 capsid can be an amino acid sequence selected from any one of SEQ ID NOs: 801-1599. In aspects of the invention, the capsid protein variant can further comprise an insert. For example, the capsid protein can comprise a 7-mer insert selected from column 2 of Table 1b, e.g., the capsid protein variant comprises a 7-mer insert comprising an amino acid sequence selected from any one of SEQ ID NOs: 1600-2398. The insert can be in the location after amino acid 455 relative to a wild type AAV9 vector.

Advantageously, viral vectors comprising an amino acid sequence of the invention exhibit muscle tropism as compared to a wild type AAV vector (e.g., a wild type AAV9 vector).

In aspects of the invention, the capsid protein further comprises a deletion of G267 in reference to an AAV9 capsid or an equivalent position in another AAV capsid. Advantageously, the capsid protein comprising a deletion of G267 may exhibit reduced liver tropism as compared to a wild type AAV capsid protein.

As described, for the HVR IV variants, the 5 amino acids upstream are at positions 451-455, shown in column 1 of Table 1b. The 7-mer insert for HVR IV variants starts with “RGDR” (SEQ ID NO: 2440) and is inserted after amino acid 455, shown in column 2 of Table 1b.

In some embodiments, the capsid protein comprises, relative to the wild type AAV9 capsid protein, the amino acid sequence of LNASI (SEQ ID NO: 990) at positions 451-455, the amino acid sequence of RGDRQLL (SEQ ID NO: 1789) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNASM (SEQ ID NO: 997) at positions 451-455, the amino acid sequence of RGDRQAL (SEQ ID NO: 1796) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNASM (SEQ ID NO: 998) at positions 451-455, the amino acid sequence of RGDRQSL (SEQ ID NO: 1797) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNAST (SEQ ID NO: 1014) at positions 451-455, the amino acid sequence of RGDRQYL (SEQ ID NO: 1813) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNGST (SEQ ID NO: 1055) at positions 451-455, the amino acid sequence of RGDRQYL (SEQ ID NO: 1854) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNYST (SEQ ID NO: 1178) at positions 451-455, the amino acid sequence of RGDRQAL (SEQ ID NO: 1977) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of SNAST (SEQ ID NO: 1343) at positions 451-455, the amino acid sequence of RGDRQYL (SEQ ID NO: 2142) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of SNASV (SEQ ID NO: 1349) at positions 451-455, the amino acid sequence of RGDRQAL (SEQ ID NO: 2148) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of VNGST (SEQ ID NO: 1547) at positions 451-455, the amino acid sequence of RGDRQYL (SEQ ID NO: 2346) inserted after amino acid 455, a deletion of the amino acid G267, and the remainder of the capsid is otherwise identical to wild type AAV9.

TABLE 1a
HVR IV
Capsid Variants

| Amino Acid | SEQ ID |
|---|---|
| Sequence | NO: |
| ANASARGDRAAL | 2 |
| ANASARGDRGSL | 3 |
| ANASARGDRGYL | 4 |
| ANASARGDRQAL | 5 |
| ANASARGDRQSL | 6 |
| ANASARGDRQYL | 7 |
| ANASGRGDRAAL | 8 |
| ANASGRGDRASL | 9 |
| ANASGRGDRAYL | 10 |
| ANASGRGDRGAL | 11 |
| ANASGRGDRGSL | 12 |
| ANASGRGDRGYL | 13 |
| ANASGRGDRQAL | 14 |
| ANASGRGDRQSL | 15 |
| ANASGRGDRQYL | 16 |
| ANASMRGDRAAL | 17 |
| ANASMRGDRASL | 18 |
| ANASMRGDRAYL | 19 |
| ANASMRGDRQAL | 20 |
| ANASMRGDRQSL | 21 |
| ANASSRGDRGSL | 22 |
| ANASSRGDRGYL | 23 |
| ANASSRGDRQAL | 24 |
| ANASSRGDRQSL | 25 |
| ANASSRGDRQYL | 26 |
| ANASTRGDRASL | 27 |
| ANASTRGDRAYL | 28 |
| ANASTRGDRGAL | 29 |
| ANASTRGDRGSL | 30 |
| ANASTRGDRGYL | 31 |
| ANASTRGDRQAL | 32 |
| ANASTRGDRQSL | 33 |
| ANASTRGDRQYL | 34 |
| ANASVRGDRASL | 35 |
| ANASVRGDRGYL | 36 |
| ANASVRGDRQAL | 37 |
| ANASVRGDRQSL | 38 |
| ANASVRGDRQYL | 39 |
| ANGNFRGDRQNL | 40 |
| ANGNVRGDRGML | 41 |
| ANGSARGDRAYL | 42 |
| ANGSARGDRGSL | 43 |
| ANGSARGDRGYL | 44 |
| ANGSARGDRQAL | 45 |
| ANGSARGDRQYL | 46 |
| ANGSGRGDRAAL | 47 |
| ANGSGRGDRAYL | 48 |
| ANGSGRGDRGAL | 49 |
| ANGSGRGDRGSL | 50 |
| ANGSGRGDRGYL | 51 |
| ANGSGRGDRQAL | 52 |
| ANGSGRGDRQSL | 53 |
| ANGSGRGDRQYL | 54 |
| ANGSMRGDRGYL | 55 |
| ANGSMRGDRQAL | 56 |
| ANGSMRGDRQSL | 57 |
| ANGSMRGDRQYL | 58 |
| ANGSSRGDRAYL | 59 |
| ANGSSRGDRGSL | 60 |
| ANGSSRGDRGYL | 61 |
| ANGSSRGDRQAL | 62 |
| ANGSSRGDRQSL | 63 |
| ANGSSRGDRQYL | 64 |
| ANGSTRGDRAAL | 65 |
| ANGSTRGDRAYL | 66 |
| ANGSTRGDRGAL | 67 |
| ANGSTRGDRGYL | 68 |
| ANGSTRGDRQAL | 69 |
| ANGSTRGDRQSL | 70 |
| ANGSTRGDRQYL | 71 |
| ANGSVRGDRAYL | 72 |
| ANGSVRGDRGYL | 73 |
| ANGSVRGDRQAL | 74 |
| ANGSVRGDRQYL | 75 |
| ANSGMRGDRGAL | 76 |
| ANSSARGDRGAL | 77 |
| ANSSARGDRGSL | 78 |
| ANSSARGDRGYL | 79 |
| ANSSARGDRQAL | 80 |
| ANSSARGDRQSL | 81 |
| ANSSARGDRQYL | 82 |
| ANSSGRGDRAAL | 83 |
| ANSSGRGDRASL | 84 |
| ANSSGRGDRAYL | 85 |
| ANSSGRGDRGAL | 86 |
| ANSSGRGDRGSL | 87 |
| ANSSGRGDRGYL | 88 |
| ANSSGRGDRQAL | 89 |
| ANSSGRGDRQSL | 90 |
| ANSSGRGDRQYL | 91 |
| ANSSMRGDRAYL | 92 |
| ANSSMRGDRQAL | 93 |
| ANSSMRGDRQSL | 94 |
| ANSSMRGDRQYL | 95 |
| ANSSSRGDRGSL | 96 |
| ANSSSRGDRGYL | 97 |
| ANSSSRGDRQAL | 98 |
| ANSSSRGDRQSL | 99 |
| ANSSSRGDRQYL | 100 |
| ANSSTRGDRAAL | 101 |
| ANSSTRGDRGSL | 102 |
| ANSSTRGDRQAL | 103 |
| ANSSTRGDRQSL | 104 |
| ANSSTRGDRQYL | 105 |
| ANSSVRGDRAYL | 106 |
| ANSSVRGDRGYL | 107 |
| ANSSVRGDRQAL | 108 |
| ANSSVRGDRQSL | 109 |
| ANSSVRGDRQYL | 110 |
| ANTSARGDRAYL | 111 |
| ANTSARGDRGAL | 112 |
| ANTSARGDRGSL | 113 |
| ANTSARGDRGYL | 114 |
| ANTSARGDRQAL | 115 |
| ANTSARGDRQYL | 116 |
| ANTSGRGDRASL | 117 |
| ANTSGRGDRGAL | 118 |
| ANTSGRGDRGSL | 119 |
| ANTSGRGDRGYL | 120 |
| ANTSGRGDRQAL | 121 |
| ANTSGRGDRQSL | 122 |
| ANTSGRGDRQYL | 123 |
| ANTSMRGDRAYL | 124 |
| ANTSMRGDRQAL | 125 |
| ANTSMRGDRQSL | 126 |
| ANTSMRGDRQYL | 127 |
| ANTSSRGDRGSL | 128 |
| ANTSSRGDRQAL | 129 |
| ANTSSRGDRQYL | 130 |
| ANTSTRGDRAAL | 131 |
| ANTSTRGDRQAL | 132 |
| ANTSTRGDRQSL | 133 |
| ANTSTRGDRQYL | 134 |
| ANTSVRGDRAYL | 135 |
| ANTSVRGDRGSL | 136 |
| ANTSVRGDRQAL | 137 |
| ANTSVRGDRQSL | 138 |
| ANTSVRGDRQYL | 139 |
| ANYPHRGDRGAL | 140 |
| ANYSARGDRAAL | 141 |
| ANYSARGDRASL | 142 |
| ANYSARGDRGAL | 143 |
| ANYSARGDRGSL | 144 |
| ANYSARGDRQAL | 145 |
| ANYSARGDRQSL | 146 |
| ANYSGRGDRAAL | 147 |
| ANYSGRGDRASL | 148 |
| ANYSGRGDRGAL | 149 |
| ANYSGRGDRGSL | 150 |
| ANYSGRGDRQAL | 151 |
| ANYSGRGDRQSL | 152 |
| ANYSGRGDRQYL | 153 |
| ANYSMRGDRAAL | 154 |
| ANYSMRGDRASL | 155 |
| ANYSMRGDRAYL | 156 |
| ANYSMRGDRQAL | 157 |
| ANYSMRGDRQSL | 158 |
| ANYSSRGDRGAL | 159 |
| ANYSSRGDRGSL | 160 |
| ANYSSRGDRQAL | 161 |
| ANYSSRGDRQSL | 162 |
| ANYSSRGDRQYL | 163 |
| ANYSTRGDRQAL | 164 |
| ANYSTRGDRQSL | 165 |
| ANYSTRGDRQYL | 166 |
| ANYSVRGDRQAL | 167 |
| ANYSVRGDRQSL | 168 |
| ANYSVRGDRQYL | 169 |
| FNSGMRGDRGAL | 170 |
| GNSGMRGDRGAL | 171 |
| HNSGMRGDRGAL | 172 |
| LNASARGDRAAL | 173 |
| LNASARGDRASL | 174 |
| LNASARGDRAYL | 175 |
| LNASARGDRGAL | 176 |
| LNASARGDRGSL | 177 |
| LNASARGDRGYL | 178 |
| LNASARGDRQAL | 179 |
| LNASARGDRQSL | 180 |
| LNASARGDRQYL | 181 |
| LNASGRGDRAAL | 182 |
| LNASGRGDRASL | 183 |
| LNASGRGDRAYL | 184 |
| LNASGRGDRGAL | 185 |
| LNASGRGDRGSL | 186 |
| LNASGRGDRGYL | 187 |
| LNASGRGDRQAL | 188 |
| LNASGRGDRQSL | 189 |
| LNASGRGDRQYL | 190 |
| LNASIRGDRQLL | 191 |
| LNASMRGDRAAL | 192 |
| LNASMRGDRASL | 193 |
| LNASMRGDRAYL | 194 |
| LNASMRGDRGAL | 195 |
| LNASMRGDRGSL | 196 |
| LNASMRGDRGYL | 197 |
| LNASMRGDRQAL | 198 |
| LNASMRGDRQSL | 199 |
| LNASMRGDRQYL | 200 |
| LNASSRGDRAAL | 201 |
| LNASSRGDRASL | 202 |
| LNASSRGDRGAL | 203 |
| LNASSRGDRGSL | 204 |
| LNASSRGDRQAL | 205 |
| LNASSRGDRQSL | 206 |
| LNASSRGDRQYL | 207 |
| LNASTRGDRAAL | 208 |
| LNASTRGDRASL | 209 |
| LNASTRGDRGAL | 210 |
| LNASTRGDRGSL | 211 |
| LNASTRGDRGYL | 212 |
| LNASTRGDRQAL | 213 |
| LNASTRGDRQSL | 214 |
| LNASTRGDRQYL | 215 |
| LNASVRGDRAAL | 216 |
| LNASVRGDRASL | 217 |
| LNASVRGDRGAL | 218 |
| LNASVRGDRGSL | 219 |
| LNASVRGDRQAL | 220 |
| LNASVRGDRQSL | 221 |
| LNASVRGDRQYL | 222 |
| LNGNFRGDRQNL | 223 |
| LNGNVRGDRGML | 224 |
| LNGSARGDRAAL | 225 |
| LNGSARGDRAYL | 226 |
| LNGSARGDRGSL | 227 |
| LNGSARGDRGYL | 228 |
| LNGSARGDRQAL | 229 |
| LNGSARGDRQSL | 230 |
| LNGSARGDRQYL | 231 |
| LNGSGRGDRAAL | 232 |
| LNGSGRGDRAYL | 233 |
| LNGSGRGDRGAL | 234 |
| LNGSGRGDRGSL | 235 |
| LNGSGRGDRGYL | 236 |
| LNGSGRGDRQAL | 237 |
| LNGSGRGDRQYL | 238 |
| LNGSMRGDRASL | 239 |
| LNGSMRGDRGYL | 240 |
| LNGSMRGDRQAL | 241 |
| LNGSMRGDRQSL | 242 |
| LNGSMRGDRQYL | 243 |
| LNGSSRGDRAAL | 244 |
| LNGSSRGDRAYL | 245 |
| LNGSSRGDRGAL | 246 |
| LNGSSRGDRQYL | 247 |
| LNGSTRGDRAAL | 248 |
| LNGSTRGDRASL | 249 |
| LNGSTRGDRAYL | 250 |
| LNGSTRGDRGAL | 251 |
| LNGSTRGDRGSL | 252 |
| LNGSTRGDRGYL | 253 |
| LNGSTRGDRQAL | 254 |
| LNGSTRGDRQSL | 255 |
| LNGSTRGDRQYL | 256 |
| LNGSVRGDRASL | 257 |
| LNGSVRGDRAYL | 258 |
| LNGSVRGDRGYL | 259 |
| LNGSVRGDRQAL | 260 |
| LNGSVRGDRQSL | 261 |
| LNGSVRGDRQYL | 262 |
| LNSGMRGDRGAL | 263 |
| LNSSARGDRASL | 264 |
| LNSSARGDRAYL | 265 |
| LNSSARGDRGAL | 266 |
| LNSSARGDRGIL | 267 |
| LNSSARGDRGSL | 268 |
| LNSSARGDRGYL | 269 |
| LNSSARGDRQAL | 270 |
| LNSSARGDRQSL | 271 |
| LNSSARGDRQYL | 272 |
| LNSSGRGDRAAL | 273 |
| LNSSGRGDRASL | 274 |
| LNSSGRGDRAYL | 275 |
| LNSSGRGDRGAL | 276 |
| LNSSGRGDRGSL | 277 |
| LNSSGRGDRGYL | 278 |
| LNSSGRGDRQAL | 279 |
| LNSSGRGDRQSL | 280 |
| LNSSGRGDRQYL | 281 |
| LNSSMRGDRAAL | 282 |
| LNSSMRGDRASL | 283 |
| LNSSMRGDRAYL | 284 |
| LNSSMRGDRGAL | 285 |
| LNSSMRGDRGSL | 286 |
| LNSSMRGDRQAL | 287 |
| LNSSMRGDRQSL | 288 |
| LNSSMRGDRQYL | 289 |
| LNSSSRGDRAAL | 290 |
| LNSSSRGDRGAL | 291 |
| LNSSSRGDRGSL | 292 |
| LNSSSRGDRQAL | 293 |
| LNSSSRGDRQSL | 294 |
| LNSSSRGDRQYL | 295 |
| LNSSTRGDRAAL | 296 |
| LNSSTRGDRASL | 297 |
| LNSSTRGDRGAL | 298 |
| LNSSTRGDRGSL | 299 |
| LNSSTRGDRQAL | 300 |
| LNSSTRGDRQSL | 301 |
| LNSSTRGDRQYL | 302 |
| LNSSVRGDRAAL | 303 |
| LNSSVRGDRASL | 304 |
| LNSSVRGDRGAL | 305 |
| LNSSVRGDRGSL | 306 |
| LNSSVRGDRGYL | 307 |
| LNSSVRGDRQAL | 308 |
| LNSSVRGDRQSL | 309 |
| LNSSVRGDRQYL | 310 |
| LNTSARGDRAAL | 311 |
| LNTSARGDRASL | 312 |
| LNTSARGDRAYL | 313 |
| LNTSARGDRGAL | 314 |
| LNTSARGDRGSL | 315 |
| LNTSARGDRGYL | 316 |
| LNTSARGDRQAL | 317 |
| LNTSARGDRQSL | 318 |
| LNTSARGDRQYL | 319 |
| LNTSGRGDRAAL | 320 |
| LNTSGRGDRASL | 321 |
| LNTSGRGDRAYL | 322 |
| LNTSGRGDRGAL | 323 |
| LNTSGRGDRGSL | 324 |
| LNTSGRGDRGYL | 325 |
| LNTSGRGDRQAL | 326 |
| LNTSGRGDRQSL | 327 |
| LNTSGRGDRQYL | 328 |
| LNTSMRGDRAAL | 329 |
| LNTSMRGDRASL | 330 |
| LNTSMRGDRAYL | 331 |
| LNTSMRGDRGAL | 332 |
| LNTSMRGDRGSL | 333 |
| LNTSMRGDRQAL | 334 |
| LNTSMRGDRQSL | 335 |
| LNTSMRGDRQYL | 336 |
| LNTSSRGDRAAL | 337 |
| LNTSSRGDRGSL | 338 |
| LNTSSRGDRQAL | 339 |
| LNTSSRGDRQSL | 340 |
| LNTSSRGDRQYL | 341 |
| LNTSTRGDRAAL | 342 |
| LNTSTRGDRASL | 343 |
| LNTSTRGDRGAL | 344 |
| LNTSTRGDRGSL | 345 |
| LNTSTRGDRQAL | 346 |
| LNTSTRGDRQSL | 347 |
| LNTSTRGDRQYL | 348 |
| LNTSVRGDRAAL | 349 |
| LNTSVRGDRASL | 350 |
| LNTSVRGDRAYL | 351 |
| LNTSVRGDRGAL | 352 |
| LNTSVRGDRGSL | 353 |
| LNTSVRGDRQAL | 354 |
| LNTSVRGDRQSL | 355 |
| LNYPHRGDRGAL | 356 |
| LNYSARGDRGAL | 357 |
| LNYSARGDRGSL | 358 |
| LNYSARGDRQAL | 359 |
| LNYSARGDRQSL | 360 |
| LNYSARGDRQYL | 361 |
| LNYSGRGDRAAL | 362 |
| LNYSGRGDRASL | 363 |
| LNYSGRGDRGAL | 364 |
| LNYSGRGDRGSL | 365 |
| LNYSGRGDRQAL | 366 |
| LNYSGRGDRQSL | 367 |
| LNYSGRGDRQYL | 368 |
| LNYSMRGDRQAL | 369 |
| LNYSMRGDRQSL | 370 |
| LNYSSRGDRGAL | 371 |
| LNYSSRGDRGSL | 372 |
| LNYSSRGDRQAL | 373 |
| LNYSSRGDRQSL | 374 |
| LNYSSRGDRQYL | 375 |
| LNYSTRGDRASL | 376 |
| LNYSTRGDRGAL | 377 |
| LNYSTRGDRGSL | 378 |
| LNYSTRGDRQAL | 379 |
| LNYSTRGDRQSL | 380 |
| LNYSTRGDRQYL | 381 |
| LNYSVRGDRQAL | 382 |
| LNYSVRGDRQSL | 383 |
| MNASARGDRAAL | 384 |
| MNASARGDRAYL | 385 |
| MNASARGDRGSL | 386 |
| MNASARGDRGYL | 387 |
| MNASARGDRQAL | 388 |
| MNASARGDRQSL | 389 |
| MNASARGDRQYL | 390 |
| MNASGRGDRAAL | 391 |
| MNASGRGDRASL | 392 |
| MNASGRGDRAYL | 393 |
| MNASGRGDRGSL | 394 |
| MNASGRGDRQAL | 395 |
| MNASGRGDRQSL | 396 |
| MNASGRGDRQYL | 397 |
| MNASIRGDRQLL | 398 |
| MNASMRGDRAAL | 399 |
| MNASMRGDRAYL | 400 |
| MNASMRGDRGAL | 401 |
| MNASMRGDRQAL | 402 |
| MNASMRGDRQSL | 403 |
| MNASSRGDRAAL | 404 |
| MNASTRGDRAAL | 405 |
| MNASTRGDRASL | 406 |
| MNASTRGDRAYL | 407 |
| MNASTRGDRGAL | 408 |
| MNASTRGDRGSL | 409 |
| MNASTRGDRQAL | 410 |
| MNASTRGDRQSL | 411 |
| MNASTRGDRQYL | 412 |
| MNASVRGDRAAL | 413 |
| MNASVRGDRASL | 414 |
| MNASVRGDRAYL | 415 |
| MNASVRGDRQAL | 416 |
| MNASVRGDRQSL | 417 |
| MNASVRGDRQYL | 418 |
| MNGNFRGDRQNL | 419 |
| MNGSARGDRAYL | 420 |
| MNGSARGDRGYL | 421 |
| MNGSARGDRQAL | 422 |
| MNGSARGDRQSL | 423 |
| MNGSARGDRQYL | 424 |
| MNGSGRGDRAAL | 425 |
| MNGSGRGDRAYL | 426 |
| MNGSGRGDRGAL | 427 |
| MNGSGRGDRGSL | 428 |
| MNGSGRGDRGYL | 429 |
| MNGSGRGDRQAL | 430 |
| MNGSGRGDRQSL | 431 |
| MNGSGRGDRQYL | 432 |
| MNGSMRGDRQAL | 433 |
| MNGSMRGDRQSL | 434 |
| MNGSMRGDRQYL | 435 |
| MNGSSRGDRQYL | 436 |
| MNGSTRGDRAYL | 437 |
| MNGSTRGDRGAL | 438 |
| MNGSTRGDRGYL | 439 |
| MNGSTRGDRQAL | 440 |
| MNGSTRGDRQSL | 441 |
| MNGSTRGDRQYL | 442 |
| MNGSVRGDRGYL | 443 |
| MNGSVRGDRQAL | 444 |
| MNGSVRGDRQYL | 445 |
| MNSGMRGDRGAL | 446 |
| MNSSARGDRAAL | 447 |
| MNSSARGDRAYL | 448 |
| MNSSARGDRGSL | 449 |
| MNSSARGDRGYL | 450 |
| MNSSARGDRQAL | 451 |
| MNSSARGDRQSL | 452 |
| MNSSARGDRQYL | 453 |
| MNSSGRGDRAAL | 454 |
| MNSSGRGDRASL | 455 |
| MNSSGRGDRAYL | 456 |
| MNSSGRGDRGAL | 457 |
| MNSSGRGDRGSL | 458 |
| MNSSGRGDRQAL | 459 |
| MNSSGRGDRQSL | 460 |
| MNSSGRGDRQYL | 461 |
| MNSSMRGDRAAL | 462 |
| MNSSMRGDRASL | 463 |
| MNSSMRGDRAYL | 464 |
| MNSSMRGDRGSL | 465 |
| MNSSMRGDRQAL | 466 |
| MNSSMRGDRQSL | 467 |
| MNSSMRGDRQYL | 468 |
| MNSSSRGDRAAL | 469 |
| MNSSTRGDRAAL | 470 |
| MNSSTRGDRASL | 471 |
| MNSSTRGDRQAL | 472 |
| MNSSTRGDRQSL | 473 |
| MNSSTRGDRQYL | 474 |
| MNSSVRGDRAAL | 475 |
| MNSSVRGDRAYL | 476 |
| MNSSVRGDRQAL | 477 |
| MNSSVRGDRQSL | 478 |
| MNSSVRGDRQYL | 479 |
| MNTSARGDRAYL | 480 |
| MNTSGRGDRAAL | 481 |
| MNTSGRGDRQAL | 482 |
| MNTSGRGDRQYL | 483 |
| MNTSMRGDRAYL | 484 |
| MNTSTRGDRQYL | 485 |
| MNYPHRGDRGAL | 486 |
| MNYSARGDRAAL | 487 |
| MNYSARGDRGYL | 488 |
| MNYSARGDRQAL | 489 |
| MNYSARGDRQSL | 490 |
| MNYSGRGDRAAL | 491 |
| MNYSGRGDRASL | 492 |
| MNYSGRGDRGSL | 493 |
| MNYSGRGDRQAL | 494 |
| MNYSGRGDRQSL | 495 |
| MNYSSRGDRQAL | 496 |
| MNYSTRGDRAAL | 497 |
| MNYSTRGDRQAL | 498 |
| MNYSTRGDRQSL | 499 |
| MNYSTRGDRQYL | 500 |
| NNSGMRGDRGAL | 501 |
| QNSGMRGDRGAL | 502 |
| SNASARGDRAAL | 503 |
| SNASARGDRASL | 504 |
| SNASARGDRAYL | 505 |
| SNASARGDRGAL | 506 |
| SNASARGDRGSL | 507 |
| SNASARGDRGYL | 508 |
| SNASARGDRQAL | 509 |
| SNASARGDRQSL | 510 |
| SNASARGDRQYL | 511 |
| SNASGRGDRAAL | 512 |
| SNASGRGDRASL | 513 |
| SNASGRGDRAYL | 514 |
| SNASGRGDRGAL | 515 |
| SNASGRGDRGSL | 516 |
| SNASGRGDRGYL | 517 |
| SNASGRGDRQAL | 518 |
| SNASGRGDRQSL | 519 |
| SNASGRGDRQYL | 520 |
| SNASIRGDRQLL | 521 |
| SNASMRGDRAAL | 522 |
| SNASMRGDRASL | 523 |
| SNASMRGDRGAL | 524 |
| SNASMRGDRGSL | 525 |
| SNASMRGDRQAL | 526 |
| SNASMRGDRQSL | 527 |
| SNASSRGDRAAL | 528 |
| SNASSRGDRASL | 529 |
| SNASSRGDRAYL | 530 |
| SNASSRGDRGAL | 531 |
| SNASSRGDRGSL | 532 |
| SNASSRGDRQAL | 533 |
| SNASSRGDRQSL | 534 |
| SNASSRGDRQYL | 535 |
| SNASTRGDRAAL | 536 |
| SNASTRGDRASL | 537 |
| SNASTRGDRAYL | 538 |
| SNASTRGDRGAL | 539 |
| SNASTRGDRGSL | 540 |
| SNASTRGDRGYL | 541 |
| SNASTRGDRQAL | 542 |
| SNASTRGDRQSL | 543 |
| SNASTRGDRQYL | 544 |
| SNASVRGDRAAL | 545 |
| SNASVRGDRASL | 546 |
| SNASVRGDRAYL | 547 |
| SNASVRGDRGSL | 548 |
| SNASVRGDRGYL | 549 |
| SNASVRGDRQAL | 550 |
| SNASVRGDRQSL | 551 |
| SNASVRGDRQYL | 552 |
| SNGNFRGDRQNL | 553 |
| SNGNVRGDRGML | 554 |
| SNGSARGDRGYL | 555 |
| SNGSARGDRQAL | 556 |
| SNGSARGDRQSL | 557 |
| SNGSARGDRQYL | 558 |
| SNGSGRGDRASL | 559 |
| SNGSGRGDRAYL | 560 |
| SNGSGRGDRGAL | 561 |
| SNGSGRGDRGSL | 562 |
| SNGSGRGDRGYL | 563 |
| SNGSGRGDRQAL | 564 |
| SNGSGRGDRQSL | 565 |
| SNGSGRGDRQYL | 566 |
| SNGSMRGDRAYL | 567 |
| SNGSMRGDRGYL | 568 |
| SNGSMRGDRQAL | 569 |
| SNGSMRGDRQSL | 570 |
| SNGSMRGDRQYL | 571 |
| SNGSSRGDRGYL | 572 |
| SNGSSRGDRQYL | 573 |
| SNGSTRGDRAAL | 574 |
| SNGSTRGDRASL | 575 |
| SNGSTRGDRGAL | 576 |
| SNGSTRGDRGYL | 577 |
| SNGSTRGDRQAL | 578 |
| SNGSTRGDRQSL | 579 |
| SNGSTRGDRQYL | 580 |
| SNGSVRGDRGYL | 581 |
| SNGSVRGDRQAL | 582 |
| SNGSVRGDRQSL | 583 |
| SNGSVRGDRQYL | 584 |
| SNSGMRGDRGAL | 585 |
| SNSSARGDRAYL | 586 |
| SNSSARGDRGAL | 587 |
| SNSSARGDRGIL | 588 |
| SNSSARGDRGSL | 589 |
| SNSSARGDRGYL | 590 |
| SNSSARGDRQAL | 591 |
| SNSSARGDRQSL | 592 |
| SNSSARGDRQYL | 593 |
| SNSSGRGDRAAL | 594 |
| SNSSGRGDRAYL | 595 |
| SNSSGRGDRGAL | 596 |
| SNSSGRGDRGSL | 597 |
| SNSSGRGDRGYL | 598 |
| SNSSGRGDRQAL | 599 |
| SNSSGRGDRQSL | 600 |
| SNSSGRGDRQYL | 601 |
| SNSSMRGDRAAL | 602 |
| SNSSMRGDRASL | 603 |
| SNSSMRGDRAYL | 604 |
| SNSSMRGDRGSL | 605 |
| SNSSMRGDRQAL | 606 |
| SNSSMRGDRQSL | 607 |
| SNSSMRGDRQYL | 608 |
| SNSSSRGDRAAL | 609 |
| SNSSSRGDRGSL | 610 |
| SNSSSRGDRQAL | 611 |
| SNSSSRGDRQSL | 612 |
| SNSSSRGDRQYL | 613 |
| SNSSTRGDRAAL | 614 |
| SNSSTRGDRASL | 615 |
| SNSSTRGDRGSL | 616 |
| SNSSTRGDRGYL | 617 |
| SNSSTRGDRQAL | 618 |
| SNSSTRGDRQSL | 619 |
| SNSSTRGDRQYL | 620 |
| SNSSVRGDRQAL | 621 |
| SNSSVRGDRQSL | 622 |
| SNSSVRGDRQYL | 623 |
| SNTSARGDRAAL | 624 |
| SNTSARGDRGAL | 625 |
| SNTSARGDRGSL | 626 |
| SNTSARGDRGYL | 627 |
| SNTSARGDRQAL | 628 |
| SNTSARGDRQSL | 629 |
| SNTSARGDRQYL | 630 |
| SNTSGRGDRAAL | 631 |
| SNTSGRGDRASL | 632 |
| SNTSGRGDRGAL | 633 |
| SNTSGRGDRGSL | 634 |
| SNTSGRGDRGYL | 635 |
| SNTSGRGDRQAL | 636 |
| SNTSGRGDRQSL | 637 |
| SNTSGRGDRQYL | 638 |
| SNTSMRGDRAYL | 639 |
| SNTSMRGDRGSL | 640 |
| SNTSMRGDRQAL | 641 |
| SNTSMRGDRQSL | 642 |
| SNTSSRGDRQAL | 643 |
| SNTSTRGDRQAL | 644 |
| SNTSTRGDRQSL | 645 |
| SNTSTRGDRQYL | 646 |
| SNTSVRGDRAYL | 647 |
| SNTSVRGDRQAL | 648 |
| SNTSVRGDRQSL | 649 |
| SNTSVRGDRQYL | 650 |
| SNYPHRGDRGAL | 651 |
| SNYSARGDRAAL | 652 |
| SNYSARGDRASL | 653 |
| SNYSARGDRGAL | 654 |
| SNYSARGDRGSL | 655 |
| SNYSARGDRQAL | 656 |
| SNYSARGDRQSL | 657 |
| SNYSARGDRQYL | 658 |
| SNYSGRGDRAAL | 659 |
| SNYSGRGDRGAL | 660 |
| SNYSGRGDRGSL | 661 |
| SNYSGRGDRQAL | 662 |
| SNYSGRGDRQSL | 663 |
| SNYSMRGDRAAL | 664 |
| SNYSMRGDRASL | 665 |
| SNYSMRGDRGSL | 666 |
| SNYSMRGDRQAL | 667 |
| SNYSMRGDRQSL | 668 |
| SNYSSRGDRGAL | 669 |
| SNYSSRGDRGSL | 670 |
| SNYSSRGDRQAL | 671 |
| SNYSSRGDRQSL | 672 |
| SNYSTRGDRAAL | 673 |
| SNYSTRGDRGAL | 674 |
| SNYSTRGDRGSL | 675 |
| SNYSTRGDRQAL | 676 |
| SNYSTRGDRQSL | 677 |
| SNYSTRGDRQYL | 678 |
| SNYSVRGDRQAL | 679 |
| SNYSVRGDRQSL | 680 |
| SNYSVRGDRQYL | 681 |
| TNSGMRGDRGAL | 682 |
| VNASARGDRAAL | 683 |
| VNASARGDRGAL | 684 |
| VNASARGDRGSL | 685 |
| VNASARGDRGYL | 686 |
| VNASARGDRQAL | 687 |
| VNASARGDRQYL | 688 |
| VNASGRGDRAAL | 689 |
| VNASGRGDRAYL | 690 |
| VNASGRGDRGAL | 691 |
| VNASGRGDRGSL | 692 |
| VNASGRGDRGYL | 693 |
| VNASGRGDRQAL | 694 |
| VNASGRGDRQYL | 695 |
| VNASMRGDRAYL | 696 |
| VNASMRGDRQAL | 697 |
| VNASMRGDRQSL | 698 |
| VNASSRGDRGSL | 699 |
| VNASSRGDRQAL | 700 |
| VNASSRGDRQYL | 701 |
| VNASTRGDRAAL | 702 |
| VNASTRGDRGAL | 703 |
| VNASTRGDRGSL | 704 |
| VNASTRGDRGYL | 705 |
| VNASTRGDRQAL | 706 |
| VNASTRGDRQSL | 707 |
| VNASTRGDRQYL | 708 |
| VNASVRGDRGSL | 709 |
| VNASVRGDRGYL | 710 |
| VNASVRGDRQAL | 711 |
| VNASVRGDRQSL | 712 |
| VNASVRGDRQYL | 713 |
| VNGNFRGDRQNL | 714 |
| VNGNVRGDRGML | 715 |
| VNGSARGDRAYL | 716 |
| VNGSARGDRGAL | 717 |
| VNGSARGDRGSL | 718 |
| VNGSARGDRGYL | 719 |
| VNGSARGDRQAL | 720 |
| VNGSARGDRQSL | 721 |
| VNGSARGDRQYL | 722 |
| VNGSGRGDRAAL | 723 |
| VNGSGRGDRASL | 724 |
| VNGSGRGDRAYL | 725 |
| VNGSGRGDRGAL | 726 |
| VNGSGRGDRGSL | 727 |
| VNGSGRGDRGYL | 728 |
| VNGSGRGDRQAL | 729 |
| VNGSGRGDRQSL | 730 |
| VNGSGRGDRQYL | 731 |
| VNGSMRGDRGYL | 732 |
| VNGSMRGDRQAL | 733 |
| VNGSMRGDRQSL | 734 |
| VNGSMRGDRQYL | 735 |
| VNGSSRGDRAYL | 736 |
| VNGSSRGDRGAL | 737 |
| VNGSSRGDRGSL | 738 |
| VNGSSRGDRQAL | 739 |
| VNGSSRGDRQSL | 740 |
| VNGSSRGDRQYL | 741 |
| VNGSTRGDRAAL | 742 |
| VNGSTRGDRAYL | 743 |
| VNGSTRGDRGAL | 744 |
| VNGSTRGDRGYL | 745 |
| VNGSTRGDRQAL | 746 |
| VNGSTRGDRQSL | 747 |
| VNGSTRGDRQYL | 748 |
| VNGSVRGDRAYL | 749 |
| VNGSVRGDRQAL | 750 |
| VNGSVRGDRQSL | 751 |
| VNGSVRGDRQYL | 752 |
| VNSGMRGDRGAL | 753 |
| VNSSARGDRGAL | 754 |
| VNSSARGDRGSL | 755 |
| VNSSARGDRGYL | 756 |
| VNSSARGDRQAL | 757 |
| VNSSARGDRQSL | 758 |
| VNSSARGDRQYL | 759 |
| VNSSGRGDRAAL | 760 |
| VNSSGRGDRAYL | 761 |
| VNSSGRGDRGAL | 762 |
| VNSSGRGDRQAL | 763 |
| VNSSGRGDRQSL | 764 |
| VNSSGRGDRQYL | 765 |
| VNSSMRGDRAYL | 766 |
| VNSSMRGDRQAL | 767 |
| VNSSMRGDRQSL | 768 |
| VNSSMRGDRQYL | 769 |
| VNSSSRGDRGSL | 770 |
| VNSSSRGDRQAL | 771 |
| VNSSSRGDRQSL | 772 |
| VNSSTRGDRGAL | 773 |
| VNSSTRGDRQAL | 774 |
| VNSSTRGDRQSL | 775 |
| VNSSTRGDRQYL | 776 |
| VNSSVRGDRAYL | 777 |
| VNSSVRGDRQAL | 778 |
| VNSSVRGDRQSL | 779 |
| VNTSARGDRAYL | 780 |
| VNTSGRGDRAAL | 781 |
| VNTSGRGDRGAL | 782 |
| VNTSGRGDRGSL | 783 |
| VNTSGRGDRGYL | 784 |
| VNTSGRGDRQAL | 785 |
| VNTSGRGDRQSL | 786 |
| VNTSGRGDRQYL | 787 |
| VNTSTRGDRAYL | 788 |
| VNYPHRGDRGAL | 789 |
| VNYSARGDRGAL | 790 |
| VNYSARGDRGSL | 791 |
| VNYSARGDRQAL | 792 |
| VNYSARGDRQSL | 793 |
| VNYSGRGDRAAL | 794 |
| VNYSGRGDRGAL | 795 |
| VNYSGRGDRQAL | 796 |
| VNYSGRGDRQSL | 797 |
| VNYSGRGDRQYL | 798 |
| VNYSSRGDRGSL | 799 |
| VNYSSRGDRGYL | 800 |

TABLE 1b
Split Amino Acid Sequences
from Table 1a

| 5 aa | SEQ ID | | SEQ ID |
|---|---|---|---|
| Substitution | NO: | 7 aa Insert | NO: |
| ANASA | 801 | RGDRAAL | 1600 |
| ANASA | 802 | RGDRGSL | 1601 |
| ANASA | 803 | RGDRGYL | 1602 |
| ANASA | 804 | RGDRQAL | 1603 |
| ANASA | 805 | RGDRQSL | 1604 |
| ANASA | 806 | RGDRQYL | 1605 |
| ANASG | 807 | RGDRAAL | 1606 |
| ANASG | 808 | RGDRASL | 1607 |
| ANASG | 809 | RGDRAYL | 1608 |
| ANASG | 810 | RGDRGAL | 1609 |
| ANASG | 811 | RGDRGSL | 1610 |
| ANASG | 812 | RGDRGYL | 1611 |
| ANASG | 813 | RGDRQAL | 1612 |
| ANASG | 814 | RGDRQSL | 1613 |
| ANASG | 815 | RGDRQYL | 1614 |
| ANASM | 816 | RGDRAAL | 1615 |
| ANASM | 817 | RGDRASL | 1616 |
| ANASM | 818 | RGDRAYL | 1617 |
| ANASM | 819 | RGDRQAL | 1618 |
| ANASM | 820 | RGDRQSL | 1619 |
| ANASS | 821 | RGDRGSL | 1620 |
| ANASS | 822 | RGDRGYL | 1621 |
| ANASS | 823 | RGDRQAL | 1622 |
| ANASS | 824 | RGDRQSL | 1623 |
| ANASS | 825 | RGDRQYL | 1624 |
| ANAST | 826 | RGDRASL | 1625 |
| ANAST | 827 | RGDRAYL | 1626 |
| ANAST | 828 | RGDRGAL | 1627 |
| ANAST | 829 | RGDRGSL | 1628 |
| ANAST | 830 | RGDRGYL | 1629 |
| ANAST | 831 | RGDRQAL | 1630 |
| ANAST | 832 | RGDRQSL | 1631 |
| ANAST | 833 | RGDRQYL | 1632 |
| ANASV | 834 | RGDRASL | 1633 |
| ANASV | 835 | RGDRGYL | 1634 |
| ANASV | 836 | RGDRQAL | 1635 |
| ANASV | 837 | RGDRQSL | 1636 |
| ANASV | 838 | RGDRQYL | 1637 |
| ANGNF | 839 | RGDRQNL | 1638 |
| ANGNV | 840 | RGDRGML | 1639 |
| ANGSA | 841 | RGDRAYL | 1640 |
| ANGSA | 842 | RGDRGSL | 1641 |
| ANGSA | 843 | RGDRGYL | 1642 |
| ANGSA | 844 | RGDRQAL | 1643 |
| ANGSA | 845 | RGDRQYL | 1644 |
| ANGSG | 846 | RGDRAAL | 1645 |
| ANGSG | 847 | RGDRAYL | 1646 |
| ANGSG | 848 | RGDRGAL | 1647 |
| ANGSG | 849 | RGDRGSL | 1648 |
| ANGSG | 850 | RGDRGYL | 1649 |
| ANGSG | 851 | RGDRQAL | 1650 |
| ANGSG | 852 | RGDRQSL | 1651 |
| ANGSG | 853 | RGDRQYL | 1652 |
| ANGSM | 854 | RGDRGYL | 1653 |
| ANGSM | 855 | RGDRQAL | 1654 |
| ANGSM | 856 | RGDRQSL | 1655 |
| ANGSM | 857 | RGDRQYL | 1656 |
| ANGSS | 858 | RGDRAYL | 1657 |
| ANGSS | 859 | RGDRGSL | 1658 |
| ANGSS | 860 | RGDRGYL | 1659 |
| ANGSS | 861 | RGDRQAL | 1660 |
| ANGSS | 862 | RGDRQSL | 1661 |
| ANGSS | 863 | RGDRQYL | 1662 |
| ANGST | 864 | RGDRAAL | 1663 |
| ANGST | 865 | RGDRAYL | 1664 |
| ANGST | 866 | RGDRGAL | 1665 |
| ANGST | 867 | RGDRGYL | 1666 |
| ANGST | 868 | RGDRQAL | 1667 |
| ANGST | 869 | RGDRQSL | 1668 |
| ANGST | 870 | RGDRQYL | 1669 |
| ANGSV | 871 | RGDRAYL | 1670 |
| ANGSV | 872 | RGDRGYL | 1671 |
| ANGSV | 873 | RGDRQAL | 1672 |
| ANGSV | 874 | RGDRQYL | 1673 |
| ANSGM | 875 | RGDRGAL | 1674 |
| ANSSA | 876 | RGDRGAL | 1675 |
| ANSSA | 877 | RGDRGSL | 1676 |
| ANSSA | 878 | RGDRGYL | 1677 |
| ANSSA | 879 | RGDRQAL | 1678 |
| ANSSA | 880 | RGDRQSL | 1679 |
| ANSSA | 881 | RGDRQYL | 1680 |
| ANSSG | 882 | RGDRAAL | 1681 |
| ANSSG | 883 | RGDRASL | 1682 |
| ANSSG | 884 | RGDRAYL | 1683 |
| ANSSG | 885 | RGDRGAL | 1684 |
| ANSSG | 886 | RGDRGSL | 1685 |
| ANSSG | 887 | RGDRGYL | 1686 |
| ANSSG | 888 | RGDRQAL | 1687 |
| ANSSG | 889 | RGDRQSL | 1688 |
| ANSSG | 890 | RGDRQYL | 1689 |
| ANSSM | 891 | RGDRAYL | 1690 |
| ANSSM | 892 | RGDRQAL | 1691 |
| ANSSM | 893 | RGDRQSL | 1692 |
| ANSSM | 894 | RGDRQYL | 1693 |
| ANSSS | 895 | RGDRGSL | 1694 |
| ANSSS | 896 | RGDRGYL | 1695 |
| ANSSS | 897 | RGDRQAL | 1696 |
| ANSSS | 898 | RGDRQSL | 1697 |
| ANSSS | 899 | RGDRQYL | 1698 |
| ANSST | 900 | RGDRAAL | 1699 |
| ANSST | 901 | RGDRGSL | 1700 |
| ANSST | 902 | RGDRQAL | 1701 |
| ANSST | 903 | RGDRQSL | 1702 |
| ANSST | 904 | RGDRQYL | 1703 |
| ANSSV | 905 | RGDRAYL | 1704 |
| ANSSV | 906 | RGDRGYL | 1705 |
| ANSSV | 907 | RGDRQAL | 1706 |
| ANSSV | 908 | RGDRQSL | 1707 |
| ANSSV | 909 | RGDRQYL | 1708 |
| ANTSA | 910 | RGDRAYL | 1709 |
| ANTSA | 911 | RGDRGAL | 1710 |
| ANTSA | 912 | RGDRGSL | 1711 |
| ANTSA | 913 | RGDRGYL | 1712 |
| ANTSA | 914 | RGDRQAL | 1713 |
| ANTSA | 915 | RGDRQYL | 1714 |
| ANTSG | 916 | RGDRASL | 1715 |
| ANTSG | 917 | RGDRGAL | 1716 |
| ANTSG | 918 | RGDRGSL | 1717 |
| ANTSG | 919 | RGDRGYL | 1718 |
| ANTSG | 920 | RGDRQAL | 1719 |
| ANTSG | 921 | RGDRQSL | 1720 |
| ANTSG | 922 | RGDRQYL | 1721 |
| ANTSM | 923 | RGDRAYL | 1722 |
| ANTSM | 924 | RGDRQAL | 1723 |
| ANTSM | 925 | RGDRQSL | 1724 |
| ANTSM | 926 | RGDRQYL | 1725 |
| ANTSS | 927 | RGDRGSL | 1726 |
| ANTSS | 928 | RGDRQAL | 1727 |
| ANTSS | 929 | RGDRQYL | 1728 |
| ANTST | 930 | RGDRAAL | 1729 |
| ANTST | 931 | RGDRQAL | 1730 |
| ANTST | 932 | RGDRQSL | 1731 |
| ANTST | 933 | RGDRQYL | 1732 |
| ANTSV | 934 | RGDRAYL | 1733 |
| ANTSV | 935 | RGDRGSL | 1734 |
| ANTSV | 936 | RGDRQAL | 1735 |
| ANTSV | 937 | RGDRQSL | 1736 |
| ANTSV | 938 | RGDRQYL | 1737 |
| ANYPH | 939 | RGDRGAL | 1738 |
| ANYSA | 940 | RGDRAAL | 1739 |
| ANYSA | 941 | RGDRASL | 1740 |
| ANYSA | 942 | RGDRGAL | 1741 |
| ANYSA | 943 | RGDRGSL | 1742 |
| ANYSA | 944 | RGDRQAL | 1743 |
| ANYSA | 945 | RGDRQSL | 1744 |
| ANYSG | 946 | RGDRAAL | 1745 |
| ANYSG | 947 | RGDRASL | 1746 |
| ANYSG | 948 | RGDRGAL | 1747 |
| ANYSG | 949 | RGDRGSL | 1748 |
| ANYSG | 950 | RGDRQAL | 1749 |
| ANYSG | 951 | RGDRQSL | 1750 |
| ANYSG | 952 | RGDRQYL | 1751 |
| ANYSM | 953 | RGDRAAL | 1752 |
| ANYSM | 954 | RGDRASL | 1753 |
| ANYSM | 955 | RGDRAYL | 1754 |
| ANYSM | 956 | RGDRQAL | 1755 |
| ANYSM | 957 | RGDRQSL | 1756 |
| ANYSS | 958 | RGDRGAL | 1757 |
| ANYSS | 959 | RGDRGSL | 1758 |
| ANYSS | 960 | RGDRQAL | 1759 |
| ANYSS | 961 | RGDRQSL | 1760 |
| ANYSS | 962 | RGDRQYL | 1761 |
| ANYST | 963 | RGDRQAL | 1762 |
| ANYST | 964 | RGDRQSL | 1763 |
| ANYST | 965 | RGDRQYL | 1764 |
| ANYSV | 966 | RGDRQAL | 1765 |
| ANYSV | 967 | RGDRQSL | 1766 |
| ANYSV | 968 | RGDRQYL | 1767 |
| FNSGM | 969 | RGDRGAL | 1768 |
| GNSGM | 970 | RGDRGAL | 1769 |
| HNSGM | 971 | RGDRGAL | 1770 |
| LNASA | 972 | RGDRAAL | 1771 |
| LNASA | 973 | RGDRASL | 1772 |
| LNASA | 974 | RGDRAYL | 1773 |
| LNASA | 975 | RGDRGAL | 1774 |
| LNASA | 976 | RGDRGSL | 1775 |
| LNASA | 977 | RGDRGYL | 1776 |
| LNASA | 978 | RGDRQAL | 1777 |
| LNASA | 979 | RGDRQSL | 1778 |
| LNASA | 980 | RGDRQYL | 1779 |
| LNASG | 981 | RGDRAAL | 1780 |
| LNASG | 982 | RGDRASL | 1781 |
| LNASG | 983 | RGDRAYL | 1782 |
| LNASG | 984 | RGDRGAL | 1783 |
| LNASG | 985 | RGDRGSL | 1784 |
| LNASG | 986 | RGDRGYL | 1785 |
| LNASG | 987 | RGDRQAL | 1786 |
| LNASG | 988 | RGDRQSL | 1787 |
| LNASG | 989 | RGDRQYL | 1788 |
| LNASI | 990 | RGDRQLL | 1789 |
| LNASM | 991 | RGDRAAL | 1790 |
| LNASM | 992 | RGDRASL | 1791 |
| LNASM | 993 | RGDRAYL | 1792 |
| LNASM | 994 | RGDRGAL | 1793 |
| LNASM | 995 | RGDRGSL | 1794 |
| LNASM | 996 | RGDRGYL | 1795 |
| LNASM | 997 | RGDRQAL | 1796 |
| LNASM | 998 | RGDRQSL | 1797 |
| LNASM | 999 | RGDRQYL | 1798 |
| LNASS | 1000 | RGDRAAL | 1799 |
| LNASS | 1001 | RGDRASL | 1800 |
| LNASS | 1002 | RGDRGAL | 1801 |
| LNASS | 1003 | RGDRGSL | 1802 |
| LNASS | 1004 | RGDRQAL | 1803 |
| LNASS | 1005 | RGDRQSL | 1804 |
| LNASS | 1006 | RGDRQYL | 1805 |
| LNAST | 1007 | RGDRAAL | 1806 |
| LNAST | 1008 | RGDRASL | 1807 |
| LNAST | 1009 | RGDRGAL | 1808 |
| LNAST | 1010 | RGDRGSL | 1809 |
| LNAST | 1011 | RGDRGYL | 1810 |
| LNAST | 1012 | RGDRQAL | 1811 |
| LNAST | 1013 | RGDRQSL | 1812 |
| LNAST | 1014 | RGDRQYL | 1813 |
| LNASV | 1015 | RGDRAAL | 1814 |
| LNASV | 1016 | RGDRASL | 1815 |
| LNASV | 1017 | RGDRGAL | 1816 |
| LNASV | 1018 | RGDRGSL | 1817 |
| LNASV | 1019 | RGDRQAL | 1818 |
| LNASV | 1020 | RGDRQSL | 1819 |
| LNASV | 1021 | RGDRQYL | 1820 |
| LNGNF | 1022 | RGDRQNL | 1821 |
| LNGNV | 1023 | RGDRGML | 1822 |
| LNGSA | 1024 | RGDRAAL | 1823 |
| LNGSA | 1025 | RGDRAYL | 1824 |
| LNGSA | 1026 | RGDRGSL | 1825 |
| LNGSA | 1027 | RGDRGYL | 1826 |
| LNGSA | 1028 | RGDRQAL | 1827 |
| LNGSA | 1029 | RGDRQSL | 1828 |
| LNGSA | 1030 | RGDRQYL | 1829 |
| LNGSG | 1031 | RGDRAAL | 1830 |
| LNGSG | 1032 | RGDRAYL | 1831 |
| LNGSG | 1033 | RGDRGAL | 1832 |
| LNGSG | 1034 | RGDRGSL | 1833 |
| LNGSG | 1035 | RGDRGYL | 1834 |
| LNGSG | 1036 | RGDRQAL | 1835 |
| LNGSG | 1037 | RGDRQYL | 1836 |
| LNGSM | 1038 | RGDRASL | 1837 |
| LNGSM | 1039 | RGDRGYL | 1838 |
| LNGSM | 1040 | RGDRQAL | 1839 |
| LNGSM | 1041 | RGDRQSL | 1840 |
| LNGSM | 1042 | RGDRQYL | 1841 |
| LNGSS | 1043 | RGDRAAL | 1842 |
| LNGSS | 1044 | RGDRAYL | 1843 |
| LNGSS | 1045 | RGDRGAL | 1844 |
| LNGSS | 1046 | RGDRQYL | 1845 |
| LNGST | 1047 | RGDRAAL | 1846 |
| LNGST | 1048 | RGDRASL | 1847 |
| LNGST | 1049 | RGDRAYL | 1848 |
| LNGST | 1050 | RGDRGAL | 1849 |
| LNGST | 1051 | RGDRGSL | 1850 |
| LNGST | 1052 | RGDRGYL | 1851 |
| LNGST | 1053 | RGDRQAL | 1852 |
| LNGST | 1054 | RGDRQSL | 1853 |
| LNGST | 1055 | RGDRQYL | 1854 |
| LNGSV | 1056 | RGDRASL | 1855 |
| LNGSV | 1057 | RGDRAYL | 1856 |
| LNGSV | 1058 | RGDRGYL | 1857 |
| LNGSV | 1059 | RGDRQAL | 1858 |
| LNGSV | 1060 | RGDRQSL | 1859 |
| LNGSV | 1061 | RGDRQYL | 1860 |
| LNSGM | 1062 | RGDRGAL | 1861 |
| LNSSA | 1063 | RGDRASL | 1862 |
| LNSSA | 1064 | RGDRAYL | 1863 |
| LNSSA | 1065 | RGDRGAL | 1864 |
| LNSSA | 1066 | RGDRGIL | 1865 |
| LNSSA | 1067 | RGDRGSL | 1866 |
| LNSSA | 1068 | RGDRGYL | 1867 |
| LNSSA | 1069 | RGDRQAL | 1868 |
| LNSSA | 1070 | RGDRQSL | 1869 |
| LNSSA | 1071 | RGDRQYL | 1870 |
| LNSSG | 1072 | RGDRAAL | 1871 |
| LNSSG | 1073 | RGDRASL | 1872 |
| LNSSG | 1074 | RGDRAYL | 1873 |
| LNSSG | 1075 | RGDRGAL | 1874 |
| LNSSG | 1076 | RGDRGSL | 1875 |
| LNSSG | 1077 | RGDRGYL | 1876 |
| LNSSG | 1078 | RGDRQAL | 1877 |
| LNSSG | 1079 | RGDRQSL | 1878 |
| LNSSG | 1080 | RGDRQYL | 1879 |
| LNSSM | 1081 | RGDRAAL | 1880 |
| LNSSM | 1082 | RGDRASL | 1881 |
| LNSSM | 1083 | RGDRAYL | 1882 |
| LNSSM | 1084 | RGDRGAL | 1883 |
| LNSSM | 1085 | RGDRGSL | 1884 |
| LNSSM | 1086 | RGDRQAL | 1885 |
| LNSSM | 1087 | RGDRQSL | 1886 |
| LNSSM | 1088 | RGDRQYL | 1887 |
| LNSSS | 1089 | RGDRAAL | 1888 |
| LNSSS | 1090 | RGDRGAL | 1889 |
| LNSSS | 1091 | RGDRGSL | 1890 |
| LNSSS | 1092 | RGDRQAL | 1891 |
| LNSSS | 1093 | RGDRQSL | 1892 |
| LNSSS | 1094 | RGDRQYL | 1893 |
| LNSST | 1095 | RGDRAAL | 1894 |
| LNSST | 1096 | RGDRASL | 1895 |
| LNSST | 1097 | RGDRGAL | 1896 |
| LNSST | 1098 | RGDRGSL | 1897 |
| LNSST | 1099 | RGDRQAL | 1898 |
| LNSST | 1100 | RGDRQSL | 1899 |
| LNSST | 1101 | RGDRQYL | 1900 |
| LNSSV | 1102 | RGDRAAL | 1901 |
| LNSSV | 1103 | RGDRASL | 1902 |
| LNSSV | 1104 | RGDRGAL | 1903 |
| LNSSV | 1105 | RGDRGSL | 1904 |
| LNSSV | 1106 | RGDRGYL | 1905 |
| LNSSV | 1107 | RGDRQAL | 1906 |
| LNSSV | 1108 | RGDRQSL | 1907 |
| LNSSV | 1109 | RGDRQYL | 1908 |
| LNTSA | 1110 | RGDRAAL | 1909 |
| LNTSA | 1111 | RGDRASL | 1910 |
| LNTSA | 1112 | RGDRAYL | 1911 |
| LNTSA | 1113 | RGDRGAL | 1912 |
| LNTSA | 1114 | RGDRGSL | 1913 |
| LNTSA | 1115 | RGDRGYL | 1914 |
| LNTSA | 1116 | RGDRQAL | 1915 |
| LNTSA | 1117 | RGDRQSL | 1916 |
| LNTSA | 1118 | RGDRQYL | 1917 |
| LNTSG | 1119 | RGDRAAL | 1918 |
| LNTSG | 1120 | RGDRASL | 1919 |
| LNTSG | 1121 | RGDRAYL | 1920 |
| LNTSG | 1122 | RGDRGAL | 1921 |
| LNTSG | 1123 | RGDRGSL | 1922 |
| LNTSG | 1124 | RGDRGYL | 1923 |
| LNTSG | 1125 | RGDRQAL | 1924 |
| LNTSG | 1126 | RGDRQSL | 1925 |
| LNTSG | 1127 | RGDRQYL | 1926 |
| LNTSM | 1128 | RGDRAAL | 1927 |
| LNTSM | 1129 | RGDRASL | 1928 |
| LNTSM | 1130 | RGDRAYL | 1929 |
| LNTSM | 1131 | RGDRGAL | 1930 |
| LNTSM | 1132 | RGDRGSL | 1931 |
| LNTSM | 1133 | RGDRQAL | 1932 |
| LNTSM | 1134 | RGDRQSL | 1933 |
| LNTSM | 1135 | RGDRQYL | 1934 |
| LNTSS | 1136 | RGDRAAL | 1935 |
| LNTSS | 1137 | RGDRGSL | 1936 |
| LNTSS | 1138 | RGDRQAL | 1937 |
| LNTSS | 1139 | RGDRQSL | 1938 |
| LNTSS | 1140 | RGDRQYL | 1939 |
| LNTST | 1141 | RGDRAAL | 1940 |
| LNTST | 1142 | RGDRASL | 1941 |
| LNTST | 1143 | RGDRGAL | 1942 |
| LNTST | 1144 | RGDRGSL | 1943 |
| LNTST | 1145 | RGDRQAL | 1944 |
| LNTST | 1146 | RGDRQSL | 1945 |
| LNTST | 1147 | RGDRQYL | 1946 |
| LNTSV | 1148 | RGDRAAL | 1947 |
| LNTSV | 1149 | RGDRASL | 1948 |
| LNTSV | 1150 | RGDRAYL | 1949 |
| LNTSV | 1151 | RGDRGAL | 1950 |
| LNTSV | 1152 | RGDRGSL | 1951 |
| LNTSV | 1153 | RGDRQAL | 1952 |
| LNTSV | 1154 | RGDRQSL | 1953 |
| LNYPH | 1155 | RGDRGAL | 1954 |
| LNYSA | 1156 | RGDRGAL | 1955 |
| LNYSA | 1157 | RGDRGSL | 1956 |
| LNYSA | 1158 | RGDRQAL | 1957 |
| LNYSA | 1159 | RGDRQSL | 1958 |
| LNYSA | 1160 | RGDRQYL | 1959 |
| LNYSG | 1161 | RGDRAAL | 1960 |
| LNYSG | 1162 | RGDRASL | 1961 |
| LNYSG | 1163 | RGDRGAL | 1962 |
| LNYSG | 1164 | RGDRGSL | 1963 |
| LNYSG | 1165 | RGDRQAL | 1964 |
| LNYSG | 1166 | RGDRQSL | 1965 |
| LNYSG | 1167 | RGDRQYL | 1966 |
| LNYSM | 1168 | RGDRQAL | 1967 |
| LNYSM | 1169 | RGDRQSL | 1968 |
| LNYSS | 1170 | RGDRGAL | 1969 |
| LNYSS | 1171 | RGDRGSL | 1970 |
| LNYSS | 1172 | RGDRQAL | 1971 |
| LNYSS | 1173 | RGDRQSL | 1972 |
| LNYSS | 1174 | RGDRQYL | 1973 |
| LNYST | 1175 | RGDRASL | 1974 |
| LNYST | 1176 | RGDRGAL | 1975 |
| LNYST | 1177 | RGDRGSL | 1976 |
| LNYST | 1178 | RGDRQAL | 1977 |
| LNYST | 1179 | RGDRQSL | 1978 |
| LNYST | 1180 | RGDRQYL | 1979 |
| LNYSV | 1181 | RGDRQAL | 1980 |
| LNYSV | 1182 | RGDRQSL | 1981 |
| MNASA | 1183 | RGDRAAL | 1982 |
| MNASA | 1184 | RGDRAYL | 1983 |
| MNASA | 1185 | RGDRGSL | 1984 |
| MNASA | 1186 | RGDRGYL | 1985 |
| MNASA | 1187 | RGDRQAL | 1986 |
| MNASA | 1188 | RGDRQSL | 1987 |
| MNASA | 1189 | RGDRQYL | 1988 |
| MNASG | 1190 | RGDRAAL | 1989 |
| MNASG | 1191 | RGDRASL | 1990 |
| MNASG | 1192 | RGDRAYL | 1991 |
| MNASG | 1193 | RGDRGSL | 1992 |
| MNASG | 1194 | RGDRQAL | 1993 |
| MNASG | 1195 | RGDRQSL | 1994 |
| MNASG | 1196 | RGDRQYL | 1995 |
| MNASI | 1197 | RGDRQLL | 1996 |
| MNASM | 1198 | RGDRAAL | 1997 |
| MNASM | 1199 | RGDRAYL | 1998 |
| MNASM | 1200 | RGDRGAL | 1999 |
| MNASM | 1201 | RGDRQAL | 2000 |
| MNASM | 1202 | RGDRQSL | 2001 |
| MNASS | 1203 | RGDRAAL | 2002 |
| MNAST | 1204 | RGDRAAL | 2003 |
| MNAST | 1205 | RGDRASL | 2004 |
| MNAST | 1206 | RGDRAYL | 2005 |
| MNAST | 1207 | RGDRGAL | 2006 |
| MNAST | 1208 | RGDRGSL | 2007 |
| MNAST | 1209 | RGDRQAL | 2008 |
| MNAST | 1210 | RGDRQSL | 2009 |
| MNAST | 1211 | RGDRQYL | 2010 |
| MNASV | 1212 | RGDRAAL | 2011 |
| MNASV | 1213 | RGDRASL | 2012 |
| MNASV | 1214 | RGDRAYL | 2013 |
| MNASV | 1215 | RGDRQAL | 2014 |
| MNASV | 1216 | RGDRQSL | 2015 |
| MNASV | 1217 | RGDRQYL | 2016 |
| MNGNF | 1218 | RGDRQNL | 2017 |
| MNGSA | 1219 | RGDRAYL | 2018 |
| MNGSA | 1220 | RGDRGYL | 2019 |
| MNGSA | 1221 | RGDRQAL | 2020 |
| MNGSA | 1222 | RGDRQSL | 2021 |
| MNGSA | 1223 | RGDRQYL | 2022 |
| MNGSG | 1224 | RGDRAAL | 2023 |
| MNGSG | 1225 | RGDRAYL | 2024 |
| MNGSG | 1226 | RGDRGAL | 2025 |
| MNGSG | 1227 | RGDRGSL | 2026 |
| MNGSG | 1228 | RGDRGYL | 2027 |
| MNGSG | 1229 | RGDRQAL | 2028 |
| MNGSG | 1230 | RGDRQSL | 2029 |
| MNGSG | 1231 | RGDRQYL | 2030 |
| MNGSM | 1232 | RGDRQAL | 2031 |
| MNGSM | 1233 | RGDRQSL | 2032 |
| MNGSM | 1234 | RGDRQYL | 2033 |
| MNGSS | 1235 | RGDRQYL | 2034 |
| MNGST | 1236 | RGDRAYL | 2035 |
| MNGST | 1237 | RGDRGAL | 2036 |
| MNGST | 1238 | RGDRGYL | 2037 |
| MNGST | 1239 | RGDRQAL | 2038 |
| MNGST | 1240 | RGDRQSL | 2039 |
| MNGST | 1241 | RGDRQYL | 2040 |
| MNGSV | 1242 | RGDRGYL | 2041 |
| MNGSV | 1243 | RGDRQAL | 2042 |
| MNGSV | 1244 | RGDRQYL | 2043 |
| MNSGM | 1245 | RGDRGAL | 2044 |
| MNSSA | 1246 | RGDRAAL | 2045 |
| MNSSA | 1247 | RGDRAYL | 2046 |
| MNSSA | 1248 | RGDRGSL | 2047 |
| MNSSA | 1249 | RGDRGYL | 2048 |
| MNSSA | 1250 | RGDRQAL | 2049 |
| MNSSA | 1251 | RGDRQSL | 2050 |
| MNSSA | 1252 | RGDRQYL | 2051 |
| MNSSG | 1253 | RGDRAAL | 2052 |
| MNSSG | 1254 | RGDRASL | 2053 |
| MNSSG | 1255 | RGDRAYL | 2054 |
| MNSSG | 1256 | RGDRGAL | 2055 |
| MNSSG | 1257 | RGDRGSL | 2056 |
| MNSSG | 1258 | RGDRQAL | 2057 |
| MNSSG | 1259 | RGDRQSL | 2058 |
| MNSSG | 1260 | RGDRQYL | 2059 |
| MNSSM | 1261 | RGDRAAL | 2060 |
| MNSSM | 1262 | RGDRASL | 2061 |
| MNSSM | 1263 | RGDRAYL | 2062 |
| MNSSM | 1264 | RGDRGSL | 2063 |
| MNSSM | 1265 | RGDRQAL | 2064 |
| MNSSM | 1266 | RGDRQSL | 2065 |
| MNSSM | 1267 | RGDRQYL | 2066 |
| MNSSS | 1268 | RGDRAAL | 2067 |
| MNSST | 1269 | RGDRAAL | 2068 |
| MNSST | 1270 | RGDRASL | 2069 |
| MNSST | 1271 | RGDRQAL | 2070 |
| MNSST | 1272 | RGDRQSL | 2071 |
| MNSST | 1273 | RGDRQYL | 2072 |
| MNSSV | 1274 | RGDRAAL | 2073 |
| MNSSV | 1275 | RGDRAYL | 2074 |
| MNSSV | 1276 | RGDRQAL | 2075 |
| MNSSV | 1277 | RGDRQSL | 2076 |
| MNSSV | 1278 | RGDRQYL | 2077 |
| MNTSA | 1279 | RGDRAYL | 2078 |
| MNTSG | 1280 | RGDRAAL | 2079 |
| MNTSG | 1281 | RGDRQAL | 2080 |
| MNTSG | 1282 | RGDRQYL | 2081 |
| MNTSM | 1283 | RGDRAYL | 2082 |
| MNTST | 1284 | RGDRQYL | 2083 |
| MNYPH | 1285 | RGDRGAL | 2084 |
| MNYSA | 1286 | RGDRAAL | 2085 |
| MNYSA | 1287 | RGDRGYL | 2086 |
| MNYSA | 1288 | RGDRQAL | 2087 |
| MNYSA | 1289 | RGDRQSL | 2088 |
| MNYSG | 1290 | RGDRAAL | 2089 |
| MNYSG | 1291 | RGDRASL | 2090 |
| MNYSG | 1292 | RGDRGSL | 2091 |
| MNYSG | 1293 | RGDRQAL | 2092 |
| MNYSG | 1294 | RGDRQSL | 2093 |
| MNYSS | 1295 | RGDRQAL | 2094 |
| MNYST | 1296 | RGDRAAL | 2095 |
| MNYST | 1297 | RGDRQAL | 2096 |
| MNYST | 1298 | RGDRQSL | 2097 |
| MNYST | 1299 | RGDRQYL | 2098 |
| NNSGM | 1300 | RGDRGAL | 2099 |
| QNSGM | 1301 | RGDRGAL | 2100 |
| SNASA | 1302 | RGDRAAL | 2101 |
| SNASA | 1303 | RGDRASL | 2102 |
| SNASA | 1304 | RGDRAYL | 2103 |
| SNASA | 1305 | RGDRGAL | 2104 |
| SNASA | 1306 | RGDRGSL | 2105 |
| SNASA | 1307 | RGDRGYL | 2106 |
| SNASA | 1308 | RGDRQAL | 2107 |
| SNASA | 1309 | RGDRQSL | 2108 |
| SNASA | 1310 | RGDRQYL | 2109 |
| SNASG | 1311 | RGDRAAL | 2110 |
| SNASG | 1312 | RGDRASL | 2111 |
| SNASG | 1313 | RGDRAYL | 2112 |
| SNASG | 1314 | RGDRGAL | 2113 |
| SNASG | 1315 | RGDRGSL | 2114 |
| SNASG | 1316 | RGDRGYL | 2115 |
| SNASG | 1317 | RGDRQAL | 2116 |
| SNASG | 1318 | RGDRQSL | 2117 |
| SNASG | 1319 | RGDRQYL | 2118 |
| SNASI | 1320 | RGDRQLL | 2119 |
| SNASM | 1321 | RGDRAAL | 2120 |
| SNASM | 1322 | RGDRASL | 2121 |
| SNASM | 1323 | RGDRGAL | 2122 |
| SNASM | 1324 | RGDRGSL | 2123 |
| SNASM | 1325 | RGDRQAL | 2124 |
| SNASM | 1326 | RGDRQSL | 2125 |
| SNASS | 1327 | RGDRAAL | 2126 |
| SNASS | 1328 | RGDRASL | 2127 |
| SNASS | 1329 | RGDRAYL | 2128 |
| SNASS | 1330 | RGDRGAL | 2129 |
| SNASS | 1331 | RGDRGSL | 2130 |
| SNASS | 1332 | RGDRQAL | 2131 |
| SNASS | 1333 | RGDRQSL | 2132 |
| SNASS | 1334 | RGDRQYL | 2133 |
| SNAST | 1335 | RGDRAAL | 2134 |
| SNAST | 1336 | RGDRASL | 2135 |
| SNAST | 1337 | RGDRAYL | 2136 |
| SNAST | 1338 | RGDRGAL | 2137 |
| SNAST | 1339 | RGDRGSL | 2138 |
| SNAST | 1340 | RGDRGYL | 2139 |
| SNAST | 1341 | RGDRQAL | 2140 |
| SNAST | 1342 | RGDRQSL | 2141 |
| SNAST | 1343 | RGDRQYL | 2142 |
| SNASV | 1344 | RGDRAAL | 2143 |
| SNASV | 1345 | RGDRASL | 2144 |
| SNASV | 1346 | RGDRAYL | 2145 |
| SNASV | 1347 | RGDRGSL | 2146 |
| SNASV | 1348 | RGDRGYL | 2147 |
| SNASV | 1349 | RGDRQAL | 2148 |
| SNASV | 1350 | RGDRQSL | 2149 |
| SNASV | 1351 | RGDRQYL | 2150 |
| SNGNF | 1352 | RGDRQNL | 2151 |
| SNGNV | 1353 | RGDRGML | 2152 |
| SNGSA | 1354 | RGDRGYL | 2153 |
| SNGSA | 1355 | RGDRQAL | 2154 |
| SNGSA | 1356 | RGDRQSL | 2155 |
| SNGSA | 1357 | RGDRQYL | 2156 |
| SNGSG | 1358 | RGDRASL | 2157 |
| SNGSG | 1359 | RGDRAYL | 2158 |
| SNGSG | 1360 | RGDRGAL | 2159 |
| SNGSG | 1361 | RGDRGSL | 2160 |
| SNGSG | 1362 | RGDRGYL | 2161 |
| SNGSG | 1363 | RGDRQAL | 2162 |
| SNGSG | 1364 | RGDRQSL | 2163 |
| SNGSG | 1365 | RGDRQYL | 2164 |
| SNGSM | 1366 | RGDRAYL | 2165 |
| SNGSM | 1367 | RGDRGYL | 2166 |
| SNGSM | 1368 | RGDRQAL | 2167 |
| SNGSM | 1369 | RGDRQSL | 2168 |
| SNGSM | 1370 | RGDRQYL | 2169 |
| SNGSS | 1371 | RGDRGYL | 2170 |
| SNGSS | 1372 | RGDRQYL | 2171 |
| SNGST | 1373 | RGDRAAL | 2172 |
| SNGST | 1374 | RGDRASL | 2173 |
| SNGST | 1375 | RGDRGAL | 2174 |
| SNGST | 1376 | RGDRGYL | 2175 |
| SNGST | 1377 | RGDRQAL | 2176 |
| SNGST | 1378 | RGDRQSL | 2177 |
| SNGST | 1379 | RGDRQYL | 2178 |
| SNGSV | 1380 | RGDRGYL | 2179 |
| SNGSV | 1381 | RGDRQAL | 2180 |
| SNGSV | 1382 | RGDRQSL | 2181 |
| SNGSV | 1383 | RGDRQYL | 2182 |
| SNSGM | 1384 | RGDRGAL | 2183 |
| SNSSA | 1385 | RGDRAYL | 2184 |
| SNSSA | 1386 | RGDRGAL | 2185 |
| SNSSA | 1387 | RGDRGIL | 2186 |
| SNSSA | 1388 | RGDRGSL | 2187 |
| SNSSA | 1389 | RGDRGYL | 2188 |
| SNSSA | 1390 | RGDRQAL | 2189 |
| SNSSA | 1391 | RGDRQSL | 2190 |
| SNSSA | 1392 | RGDRQYL | 2191 |
| SNSSG | 1393 | RGDRAAL | 2192 |
| SNSSG | 1394 | RGDRAYL | 2193 |
| SNSSG | 1395 | RGDRGAL | 2194 |
| SNSSG | 1396 | RGDRGSL | 2195 |
| SNSSG | 1397 | RGDRGYL | 2196 |
| SNSSG | 1398 | RGDRQAL | 2197 |
| SNSSG | 1399 | RGDRQSL | 2198 |
| SNSSG | 1400 | RGDRQYL | 2199 |
| SNSSM | 1401 | RGDRAAL | 2200 |
| SNSSM | 1402 | RGDRASL | 2201 |
| SNSSM | 1403 | RGDRAYL | 2202 |
| SNSSM | 1404 | RGDRGSL | 2203 |
| SNSSM | 1405 | RGDRQAL | 2204 |
| SNSSM | 1406 | RGDRQSL | 2205 |
| SNSSM | 1407 | RGDRQYL | 2206 |
| SNSSS | 1408 | RGDRAAL | 2207 |
| SNSSS | 1409 | RGDRGSL | 2208 |
| SNSSS | 1410 | RGDRQAL | 2209 |
| SNSSS | 1411 | RGDRQSL | 2210 |
| SNSSS | 1412 | RGDRQYL | 2211 |
| SNSST | 1413 | RGDRAAL | 2212 |
| SNSST | 1414 | RGDRASL | 2213 |
| SNSST | 1415 | RGDRGSL | 2214 |
| SNSST | 1416 | RGDRGYL | 2215 |
| SNSST | 1417 | RGDRQAL | 2216 |
| SNSST | 1418 | RGDRQSL | 2217 |
| SNSST | 1419 | RGDRQYL | 2218 |
| SNSSV | 1420 | RGDRQAL | 2219 |
| SNSSV | 1421 | RGDRQSL | 2220 |
| SNSSV | 1422 | RGDRQYL | 2221 |
| SNTSA | 1423 | RGDRAAL | 2222 |
| SNTSA | 1424 | RGDRGAL | 2223 |
| SNTSA | 1425 | RGDRGSL | 2224 |
| SNTSA | 1426 | RGDRGYL | 2225 |
| SNTSA | 1427 | RGDRQAL | 2226 |
| SNTSA | 1428 | RGDRQSL | 2227 |
| SNTSA | 1429 | RGDRQYL | 2228 |
| SNTSG | 1430 | RGDRAAL | 2229 |
| SNTSG | 1431 | RGDRASL | 2230 |
| SNTSG | 1432 | RGDRGAL | 2231 |
| SNTSG | 1433 | RGDRGSL | 2232 |
| SNTSG | 1434 | RGDRGYL | 2233 |
| SNTSG | 1435 | RGDRQAL | 2234 |
| SNTSG | 1436 | RGDRQSL | 2235 |
| SNTSG | 1437 | RGDRQYL | 2236 |
| SNTSM | 1438 | RGDRAYL | 2237 |
| SNTSM | 1439 | RGDRGSL | 2238 |
| SNTSM | 1440 | RGDRQAL | 2239 |
| SNTSM | 1441 | RGDRQSL | 2240 |
| SNTSS | 1442 | RGDRQAL | 2241 |
| SNTST | 1443 | RGDRQAL | 2242 |
| SNTST | 1444 | RGDRQSL | 2243 |
| SNTST | 1445 | RGDRQYL | 2244 |
| SNTSV | 1446 | RGDRAYL | 2245 |
| SNTSV | 1447 | RGDRQAL | 2246 |
| SNTSV | 1448 | RGDRQSL | 2247 |
| SNTSV | 1449 | RGDRQYL | 2248 |
| SNYPH | 1450 | RGDRGAL | 2249 |
| SNYSA | 1451 | RGDRAAL | 2250 |
| SNYSA | 1452 | RGDRASL | 2251 |
| SNYSA | 1453 | RGDRGAL | 2252 |
| SNYSA | 1454 | RGDRGSL | 2253 |
| SNYSA | 1455 | RGDRQAL | 2254 |
| SNYSA | 1456 | RGDRQSL | 2255 |
| SNYSA | 1457 | RGDRQYL | 2256 |
| SNYSG | 1458 | RGDRAAL | 2257 |
| SNYSG | 1459 | RGDRGAL | 2258 |
| SNYSG | 1460 | RGDRGSL | 2259 |
| SNYSG | 1461 | RGDRQAL | 2260 |
| SNYSG | 1462 | RGDRQSL | 2261 |
| SNYSM | 1463 | RGDRAAL | 2262 |
| SNYSM | 1464 | RGDRASL | 2263 |
| SNYSM | 1465 | RGDRGSL | 2264 |
| SNYSM | 1466 | RGDRQAL | 2265 |
| SNYSM | 1467 | RGDRQSL | 2266 |
| SNYSS | 1468 | RGDRGAL | 2267 |
| SNYSS | 1469 | RGDRGSL | 2268 |
| SNYSS | 1470 | RGDRQAL | 2269 |
| SNYSS | 1471 | RGDRQSL | 2270 |
| SNYST | 1472 | RGDRAAL | 2271 |
| SNYST | 1473 | RGDRGAL | 2272 |
| SNYST | 1474 | RGDRGSL | 2273 |
| SNYST | 1475 | RGDRQAL | 2274 |
| SNYST | 1476 | RGDRQSL | 2275 |
| SNYST | 1477 | RGDRQYL | 2276 |
| SNYSV | 1478 | RGDRQAL | 2277 |
| SNYSV | 1479 | RGDRQSL | 2278 |
| SNYSV | 1480 | RGDRQYL | 2279 |
| TNSGM | 1481 | RGDRGAL | 2280 |
| VNASA | 1482 | RGDRAAL | 2281 |
| VNASA | 1483 | RGDRGAL | 2282 |
| VNASA | 1484 | RGDRGSL | 2283 |
| VNASA | 1485 | RGDRGYL | 2284 |
| VNASA | 1486 | RGDRQAL | 2285 |
| VNASA | 1487 | RGDRQYL | 2286 |
| VNASG | 1488 | RGDRAAL | 2287 |
| VNASG | 1489 | RGDRAYL | 2288 |
| VNASG | 1490 | RGDRGAL | 2289 |
| VNASG | 1491 | RGDRGSL | 2290 |
| VNASG | 1492 | RGDRGYL | 2291 |
| VNASG | 1493 | RGDRQAL | 2292 |
| VNASG | 1494 | RGDRQYL | 2293 |
| VNASM | 1495 | RGDRAYL | 2294 |
| VNASM | 1496 | RGDRQAL | 2295 |
| VNASM | 1497 | RGDRQSL | 2296 |
| VNASS | 1498 | RGDRGSL | 2297 |
| VNASS | 1499 | RGDRQAL | 2298 |
| VNASS | 1500 | RGDRQYL | 2299 |
| VNAST | 1501 | RGDRAAL | 2300 |
| VNAST | 1502 | RGDRGAL | 2301 |
| VNAST | 1503 | RGDRGSL | 2302 |
| VNAST | 1504 | RGDRGYL | 2303 |
| VNAST | 1505 | RGDRQAL | 2304 |
| VNAST | 1506 | RGDRQSL | 2305 |
| VNAST | 1507 | RGDRQYL | 2306 |
| VNASV | 1508 | RGDRGSL | 2307 |
| VNASV | 1509 | RGDRGYL | 2308 |
| VNASV | 1510 | RGDRQAL | 2309 |
| VNASV | 1511 | RGDRQSL | 2310 |
| VNASV | 1512 | RGDRQYL | 2311 |
| VNGNF | 1513 | RGDRQNL | 2312 |
| VNGNV | 1514 | RGDRGML | 2313 |
| VNGSA | 1515 | RGDRAYL | 2314 |
| VNGSA | 1516 | RGDRGAL | 2315 |
| VNGSA | 1517 | RGDRGSL | 2316 |
| VNGSA | 1518 | RGDRGYL | 2317 |
| VNGSA | 1519 | RGDRQAL | 2318 |
| VNGSA | 1520 | RGDRQSL | 2319 |
| VNGSA | 1521 | RGDRQYL | 2320 |
| VNGSG | 1522 | RGDRAAL | 2321 |
| VNGSG | 1523 | RGDRASL | 2322 |
| VNGSG | 1524 | RGDRAYL | 2323 |
| VNGSG | 1525 | RGDRGAL | 2324 |
| VNGSG | 1526 | RGDRGSL | 2325 |
| VNGSG | 1527 | RGDRGYL | 2326 |
| VNGSG | 1528 | RGDRQAL | 2327 |
| VNGSG | 1529 | RGDRQSL | 2328 |
| VNGSG | 1530 | RGDRQYL | 2329 |
| VNGSM | 1531 | RGDRGYL | 2330 |
| VNGSM | 1532 | RGDRQAL | 2331 |
| VNGSM | 1533 | RGDRQSL | 2332 |
| VNGSM | 1534 | RGDRQYL | 2333 |
| VNGSS | 1535 | RGDRAYL | 2334 |
| VNGSS | 1536 | RGDRGAL | 2335 |
| VNGSS | 1537 | RGDRGSL | 2336 |
| VNGSS | 1538 | RGDRQAL | 2337 |
| VNGSS | 1539 | RGDRQSL | 2338 |
| VNGSS | 1540 | RGDRQYL | 2339 |
| VNGST | 1541 | RGDRAAL | 2340 |
| VNGST | 1542 | RGDRAYL | 2341 |
| VNGST | 1543 | RGDRGAL | 2342 |
| VNGST | 1544 | RGDRGYL | 2343 |
| VNGST | 1545 | RGDRQAL | 2344 |
| VNGST | 1546 | RGDRQSL | 2345 |
| VNGST | 1547 | RGDRQYL | 2346 |
| VNGSV | 1548 | RGDRAYL | 2347 |
| VNGSV | 1549 | RGDRQAL | 2348 |
| VNGSV | 1550 | RGDRQSL | 2349 |
| VNGSV | 1551 | RGDRQYL | 2350 |
| VNSGM | 1552 | RGDRGAL | 2351 |
| VNSSA | 1553 | RGDRGAL | 2352 |
| VNSSA | 1554 | RGDRGSL | 2353 |
| VNSSA | 1555 | RGDRGYL | 2354 |
| VNSSA | 1556 | RGDRQAL | 2355 |
| VNSSA | 1557 | RGDRQSL | 2356 |
| VNSSA | 1558 | RGDRQYL | 2357 |
| VNSSG | 1559 | RGDRAAL | 2358 |
| VNSSG | 1560 | RGDRAYL | 2359 |
| VNSSG | 1561 | RGDRGAL | 2360 |
| VNSSG | 1562 | RGDRQAL | 2361 |
| VNSSG | 1563 | RGDRQSL | 2362 |
| VNSSG | 1564 | RGDRQYL | 2363 |
| VNSSM | 1565 | RGDRAYL | 2364 |
| VNSSM | 1566 | RGDRQAL | 2365 |
| VNSSM | 1567 | RGDRQSL | 2366 |
| VNSSM | 1568 | RGDRQYL | 2367 |
| VNSSS | 1569 | RGDRGSL | 2368 |
| VNSSS | 1570 | RGDRQAL | 2369 |
| VNSSS | 1571 | RGDRQSL | 2370 |
| VNSST | 1572 | RGDRGAL | 2371 |
| VNSST | 1573 | RGDRQAL | 2372 |
| VNSST | 1574 | RGDRQSL | 2373 |
| VNSST | 1575 | RGDRQYL | 2374 |
| VNSSV | 1576 | RGDRAYL | 2375 |
| VNSSV | 1577 | RGDRQAL | 2376 |
| VNSSV | 1578 | RGDRQSL | 2377 |
| VNTSA | 1579 | RGDRAYL | 2378 |
| VNTSG | 1580 | RGDRAAL | 2379 |
| VNTSG | 1581 | RGDRGAL | 2380 |
| VNTSG | 1582 | RGDRGSL | 2381 |
| VNTSG | 1583 | RGDRGYL | 2382 |
| VNTSG | 1584 | RGDRQAL | 2383 |
| VNTSG | 1585 | RGDRQSL | 2384 |
| VNTSG | 1586 | RGDRQYL | 2385 |
| VNTST | 1587 | RGDRAYL | 2386 |
| VNYPH | 1588 | RGDRGAL | 2387 |
| VNYSA | 1589 | RGDRGAL | 2388 |
| VNYSA | 1590 | RGDRGSL | 2389 |
| VNYSA | 1591 | RGDRQAL | 2390 |
| VNYSA | 1592 | RGDRQSL | 2391 |
| VNYSG | 1593 | RGDRAAL | 2392 |
| VNYSG | 1594 | RGDRGAL | 2393 |
| VNYSG | 1595 | RGDRQAL | 2394 |
| VNYSG | 1596 | RGDRQSL | 2395 |
| VNYSG | 1597 | RGDRQYL | 2396 |
| VNYSS | 1598 | RGDRGSL | 2397 |
| VNYSS | 1599 | RGDRGYL | 2398 |

### BRIEF DESCRIPTION OF THE DRAWINGS

FIGS. 1A-1B show results from analysis of microdystrophin expression in quadriceps from B10-mdx mice dosed with either Composition 1 (4E13 vg/kg) or Reference AAV composition (1.33E14 vg/kg). FIG. 1A shows images of FLAG immunofluorescence of microdystrophin expression in B10-mdx mouse quadriceps following administration of Composition 1 or Reference AAV composition. FIG. 1B shows results from capillary electrophoresis quantifying microdystrophin expression in B10-mdx mouse quadriceps following administration of Composition 1 or Reference AAV composition.

FIGS. 2A-2B show results from analysis of microdystrophin expression in triceps from B10-mdx mice dosed with either Composition 1 (4E13 vg/kg) or Reference AAV composition (1.33E14 vg/kg). FIG. 2A shows images of FLAG immunofluorescence of microdystrophin expression in B10-mdx mouse triceps following administration of Composition 1 or Reference AAV composition. FIG. 2B shows results from capillary electrophoresis quantifying microdystrophin expression in B10-mdx mouse triceps following administration of Composition 1 or Reference AAV composition.

FIGS. 3A-3B show results from analysis of microdystrophin expression in heart tissue from B10-mdx mice dosed with either Composition 1 (4E13 vg/kg) or Reference AAV composition (1.33E14 vg/kg). FIG. 3A shows images of FLAG immunofluorescence of microdystrophin expression in B10-mdx mouse hearts following administration of Composition 1 or Reference AAV composition. FIG. 3B shows results from capillary electrophoresis quantifying microdystrophin expression in B10-mouse hearts following administration of Composition 1 or Reference AAV composition.

FIGS. 4A-4C show results from analysis of microdystrophin expression and muscle physiology in B10-mdx mice dosed with either Composition 1 (4E13 vg/kg) or Reference AAV composition (1.33E14 vg/kg). FIG. 4A shows a graph of specific force of muscles from B10-mice following administration of Composition 1 or Reference AAV composition.

FIG. 4B shows a graph of percent force drop after eccentric damage, which demonstrates protection against eccentric damage, in muscles from B10-mice following administration of Composition 1 or Reference AAV composition. FIG. 4C shows results from capillary electrophoresis quantifying microdystrophin expression in mouse EDL muscle following administration of Composition 1 or Reference AAV composition.

FIGS. 5A-5B show results from analysis of microdystrophin expression in heart left ventricles from NHP dosed with either Composition 1 (4E13 vg/kg) or Reference AAV composition (1.33E14 vg/kg). FIG. 5A shows images of FLAG immunofluorescence of microdystrophin expression in NHP heart left ventricles following administration of Composition 1 or Reference AAV composition. FIG. 5B shows results from capillary electrophoresis quantifying microdystrophin expression in NHP heart left ventricles following administration of Composition 1 or Reference AAV composition.

FIGS. 6A-6B show results from analysis of microdystrophin expression in heart right ventricles from NHP dosed with either Composition 1 (4E13 vg/kg) or Reference AAV composition (1.33E14 vg/kg). FIG. 6A shows images of FLAG immunofluorescence of microdystrophin expression in NHP heart right ventricles following administration of Composition 1 or Reference AAV composition. FIG. 6B shows results from capillary electrophoresis quantifying microdystrophin expression in NHP heart right ventricles following administration of Composition 1 or Reference AAV composition.

FIGS. 7A-7B show results from analysis of microdystrophin expression in heart interventricular septum from NHP dosed with either Composition 1 (4E13 vg/kg) or Reference AAV composition (1.33E14 vg/kg). FIG. 7A shows images of FLAG immunofluorescence of microdystrophin expression in NHP heart interventricular septum following administration of Composition 1 or Reference AAV composition. FIG. 7B shows results from capillary electrophoresis quantifying microdystrophin expression in NHP heart interventricular septum following administration of Composition 1 or Reference AAV composition.

FIGS. 8A-8B show results from analysis of microdystrophin expression in gastrocnemius from NHP dosed with either Composition 1 (4E13 vg/kg) or Reference AAV composition (1.33E14 vg/kg). FIG. 8A shows images of FLAG immunofluorescence of microdystrophin expression in NHP gastrocnemius following administration of Composition 1 or Reference AAV composition. FIG. 8B shows results from capillary electrophoresis quantifying microdystrophin expression in NHP gastrocnemius following administration of Composition 1 or Reference AAV composition.

FIGS. 9A-9B show results from analysis of microdystrophin expression in pectoralis major from NHP dosed with either Composition 1 (4E13 vg/kg) or Reference AAV composition (1.33E14 vg/kg). FIG. 9A shows images of FLAG immunofluorescence of microdystrophin expression in NHP pectoralis major following administration of Composition 1 or Reference AAV composition. FIG. 9B shows results from capillary electrophoresis quantifying microdystrophin expression in NHP pectoralis major following administration of Composition 1 or Reference AAV composition.

FIGS. 10A-10B show results from analysis of microdystrophin expression in diaphragm from NHP dosed with either Composition 1 (4E13 vg/kg) or Reference AAV composition (1.33E14 vg/kg). FIG. 10A shows images of FLAG immunofluorescence of microdystrophin expression in NHP diaphragm following administration of Composition 1 or Reference AAV composition. FIG. 10B shows results from capillary electrophoresis quantifying microdystrophin expression in NHP diaphragm following administration of Composition 1 or Reference AAV composition.

FIG. 11 shows a graph of vector genome detected in the liver of NHPs.

FIG. 12 shows representative images of FLAG immunofluorescence of diaphragm, quadriceps, heart, and extensor digitorum longus (EDL) tissue of D2-mdx mice treated with Composition 1. Mice were dosed with either a low (1E13 vg/kg), a mid (2E13 vg/kg), or high (4E13 vg/kg) dose of Composition 1. WT mice and D2-mdx mice were treated with vehicle as a control. Tissues were analyzed 7 weeks post-injection.

FIGS. 13A-13B show results from capillary electrophoresis analysis of microdystrophin expression in D2-mdx mouse heart at 7 weeks (FIG. 13A) and 22 weeks (FIG. 13B) post-injection of Composition 1. Mice were dosed with either a low (1E13 vg/kg), a mid (2E13 vg/kg), or high (4E13 vg/kg) dose of Composition 1. WT mice and D2-mdx mice were treated with vehicle as a control.

FIGS. 14A-14B show results from capillary electrophoresis analysis of microdystrophin expression in D2-mdx mouse skeletal muscle at 7 weeks (FIG. 13A) and 22 weeks (FIG. 13B) post-injection of Composition 1 or Reference AAV composition. Mice were dosed with either a low (1E13 vg/kg), a mid (2E13 vg/kg), or high (4E13 vg/kg) dose of Composition 1. WT mice and D2-mdx mice were treated with vehicle as a control.

FIGS. 15A-15B show a graph of body weight of D2-mdx mice dosed with either a low (1E13 vg/kg), a mid (2E13 vg/kg), or high (4E13 vg/kg) dose of Composition 1. Body weight was measured pre-dose and monitored twice weekly throughout the 7 weeks (FIG. 15A) and 22 weeks (FIG. 15B) study cohorts. WT mice and D2-mdx mice were treated with vehicle as a control. Error bars are mean±standard deviation.

FIGS. 16A-16B show a graph of body weight and tibialis anterior (TA) muscle weight of D2-mdx mice dosed with either a low (1E13 vg/kg), a mid (2E13 vg/kg), or high (4E13 vg/kg) dose of Composition 1. Body weight and TA weight was measured pre-dose and monitored twice weekly throughout the 7 weeks (FIG. 16A) and 22 weeks (FIG. 16B) study cohorts. WT mice and D2-mdx mice were treated with vehicle as a control. Error bars are mean±standard deviation. Asterisks indicate statistical differences from mdx-vehicle mice (*p<0.05; ** p<0.01; **** p<0.0001).

FIGS. 17A-17E show results from physiological analysis of D2-mdx mice dosed with either a low (1E13 vg/kg), a mid (2E13 vg/kg), or high (4E13 vg/kg) dose of Composition 1. WT mice and D2-mdx mice were treated with vehicle as a control. Tissues were analyzed 7 weeks post-injection. Error bars are mean±standard deviation. Asterisks, indicate statistical differences from mdx-vehicle mice (*p<0.05; ** P<0.01; *** p<0.001; **** p<0.0001; lack of asterisks indicates non-significance). (#), indicate WT-vehicle statistical differences from mdx-vehicle mice. (*), indicate WT-vehicle and Composition 1 statistical differences from mdx-vehicle mice. FIG. 17A shows graphs of anatomical properties of EDL muscle, including EDL weight (left panel), optimal length (Lo; middle panel), and cross-sectional area (CSA; right panel). FIG. 17B shows graphs of quantitative measurements of muscle contractility in EDL muscle, including specific twitch force (sPt; left panel) and specific tetanic force (sPo; right panel). FIG. 17C shows a graph of eccentric contraction of EDL muscle. FIG. 17D shows a graph of the stress-strain relationship to evaluate the elastic properties of EDL muscle. FIG. 17E shows a graph of the stress relaxation rate to evaluate the viscous properties of EDL muscle.

FIGS. 18A-18E show results from physiological analysis of D2-mdx mice dosed with either a low (1E13 vg/kg), a mid (2E13 vg/kg), or high (4E13 vg/kg) dose of Composition 1. WT mice and D2-mdx mice were treated with vehicle as a control. Tissues were analyzed 22 weeks post-injection. Error bars are mean±standard deviation. Asterisks, indicate statistical differences from mdx-vehicle mice (*p<0.05; ** P<0.01; *** p<0.001; **** p<0.0001; lack of asterisks indicates non-significance). (#), indicate WT-vehicle statistical differences from mdx-vehicle mice. (†), indicate WT-vehicle and Composition 1 statistical differences from mdx-vehicle mice. FIG. 18A shows graphs of anatomical properties of EDL muscle, including EDL weight (left panel), optimal length (Lo; middle panel), and cross-sectional area (CSA; right panel). FIG. 18B shows graphs of quantitative measurements of muscle contractility in EDL muscle, including specific twitch force (sPt; left panel) and specific tetanic force (sPo; right panel). FIG. 18C shows a graph of eccentric contraction of EDL muscle. FIG. 18D shows a graph of the stress-strain relationship to evaluate the elastic properties of EDL muscle. FIG. 18E shows a graph of the stress relaxation rate to evaluate the viscous properties of EDL muscle.

FIGS. 19A-19C show graphs of mRNA levels of microdystrophin-FLAG in heart (FIG. 19A), skeletal muscle (FIG. 19B), and off-target tissues (FIG. 19C) from NHPs dosed with 4E13 vg/kg of Composition 1. Tissues were analyzed 28 days post-injection. The transcript copy was normalized to nhpRPL30 (n=3/group). Error bars are mean±standard deviation. Avg.: average value; Sk.: skeletal; DRG: dorsal root ganglion.

FIG. 20 shows representative images of FLAG immunofluorescence of heart and skeletal muscle from NHPs dosed with 4E13 vg/kg of Composition 1. Tissues were analyzed 28 days post-injection.

FIGS. 21A-21B show results from capillary electrophoresis analysis of microdystrophin expression in heart (FIG. 21A) and skeletal muscle (FIG. 21B) from NHPs dosed with 4E13 vg/kg of Composition 1. Tissues were analyzed at 28 days or 90-days post-injection. The amount of the microdystrophin expressed in tissues was calculated by interpolation to the full-length dystrophin standard curve derived from normal human heart or skeletal muscle samples.

FIGS. 22A-22B show results from capillary electrophoresis analysis of microdystrophin expression from an endogenous coding sequence or a codon optimized microdystrophin sequence (v1, v2, or v3) in C2C12 derived myotubes.

FIGS. 23A-23B show results from capillary electrophoresis analysis of microdystrophin expression from an endogenous coding sequence or a codon optimized microdystrophin sequence (v1, v2, or v3) in human primary myotubes.

FIGS. 24A-24C show results from analysis of microdystrophin expression in gastrocnemius from NHP dosed with MyoAAV-LD A- or AAV9-CBh-uDys-FLAG. FIG. 24A shows images of FLAG immunofluorescence of microdystrophin expression in NHP gastrocnemius following administration of MyoAAV-LD A- or AAV9-CBh-uDys-FLAG.

FIG. 24B shows results from capillary electrophoresis quantifying microdystrophin expression in NHP gastrocnemius following administration of MyoAAV-LD A- or AAV9-CBh-uDys-FLAG. FIG. 24C shows a graph of mRNA levels of microdystrophin in gastrocnemius from NHP dosed with MyoAAV-LD A- or AAV9-CBh-uDys-FLAG.]

FIGS. 25A-25C show results from analysis of microdystrophin expression in triceps from NHP dosed with MyoAAV-LD A- or AAV9-CBh-uDys-FLAG. FIG. 25A shows images of FLAG immunofluorescence of microdystrophin expression in NHP triceps following administration of MyoAAV-LD A- or AAV9-CBh-uDys-FLAG. FIG. 25B shows results from capillary electrophoresis quantifying microdystrophin expression in NHP triceps following administration of MyoAAV-LD A- or AAV9-CBh-uDys-FLAG. FIG. 25C shows a graph of mRNA levels of microdystrophin in triceps from NHP dosed with MyoAAV-LD A- or AAV9-CBh-uDys-FLAG.

FIGS. 26A-26C show results from analysis of microdystrophin expression in biceps brachii from NHP dosed with MyoAAV-LD A- or AAV9-CBh-uDys-FLAG. FIG. 26A shows images of FLAG immunofluorescence of microdystrophin expression in NHP biceps brachii following administration of MyoAAV-LD A- or AAV9-CBh-uDys-FLAG. FIG. 26B shows results from capillary electrophoresis quantifying microdystrophin expression in NHP biceps brachii following administration of MyoAAV-LD A- or AAV9-CBh-uDys-FLAG. FIG. 26C shows a graph of mRNA levels of microdystrophin in biceps brachii from NHP dosed with MyoAAV-LD A- or AAV9-CBh-uDys-FLAG.

FIGS. 27A-27C show results from analysis of microdystrophin expression in pectoralis major from NHP dosed with MyoAAV-LD A- or AAV9-CBh-uDys-FLAG. FIG. 27A shows images of FLAG immunofluorescence of microdystrophin expression in NHP pectoralis major following administration of MyoAAV-LD A- or AAV9-CBh-uDys-FLAG. FIG. 27B shows results from capillary electrophoresis quantifying microdystrophin expression in NHP pectoralis major brachii following administration of MyoAAV-LD A- or AAV9-CBh-uDys-FLAG. FIG. 27C shows a graph of mRNA levels of microdystrophin in pectoralis major from NHP dosed with MyoAAV-LD A- or AAV9-CBh-uDys-FLAG.

FIGS. 28A-28B show results from analysis of muscle physiology in B10-mdx mice dosed with DMD tool compound (2E13 vg/kg). FIG. 28A shows a graph of specific force following administration of the DMD tool compound to WT and mdx mice. FIG. 28B shows a graph of protection against eccentric damage following administration of the DMD tool compound to WT and mdx mice. *: WT significantly different from mdx, #: Mdx significantly different from mdx+tool compound, ‡: WT significantly different from mdx+tool compound.

### DETAILED DESCRIPTION

The present invention provides novel engineered nucleic acids encoding microdystrophin proteins for use in gene therapy.

Specifically, aspects of the invention provide a formulation comprising an AAV particle comprising (e.g., encapsidating) a nucleic acid comprising a promoter and a sequence encoding microdystrophin.

Aspects of the invention provide a composition comprising an adeno-associated virus (AAV) particle comprising an engineered capsid protein comprising the amino acid sequence RGDR (SEQ ID NO: 2440). The capsid protein encapsidates a nucleic acid molecule (e.g., a cargo) comprising a promoter, a codon optimized microdystrophin sequence, for example, a sequence having at least 95% sequence identity with a sequence selected from any one of SEQ ID NOs: 2405-2407. The nucleic acid may further comprise a dorsal root ganglia (DRG) de-targeting miRNA binding site.

### DMD Gene

The DMD gene is the largest known human gene. The most common mutations that cause DMD are large deletion mutations of one or more exons (60-70%), however duplication mutations (5-10%), and single nucleotide variants (25-35%), can also cause pathogenic dystrophin variants. In DMD, mutations often lead to a frame shift resulting in a premature stop codon and a truncated, non-functional or unstable protein. Nonsense point mutations can also result in premature termination codons with the same result. While mutations causing DMD can affect any exon, exons 2-20 and 45-55 are common hotspots for large deletion and duplication mutations.

Full-length dystrophin is a large (427 kDa) protein comprising a number of subdomains that contribute to its function. These subdomains include, in order from the amino-terminus toward the carboxy-terminus, the N-terminal actin-binding domain, a central so-called “rod” domain, a cysteine-rich domain and lastly a carboxy-terminal domain or region. The rod domain is comprised of 4 proline-rich hinge domains (abbreviated H), and 24 spectrin-like repeats (abbreviated R) in the following order: a first hinge domain (H1), 3 spectrin-like repeats (R1, R2, R3), a second hinge domain (H2), 16 more spectrin-like repeats (R4, R5, R6, R7, R8, R9, R10, R11, R12, R13, R14, R15, R16, R17, R18, R19), a third hinge domain (H3), 5 more spectrin-like repeats (R20, R21, R22, R23, R24), and a fourth hinge domain (H4) (including the WW domain). Following the rod domain are the cysteine-rich domain, and the COOH (C)-terminal (CT) domain.

### Optimized Nucleic Acid Sequences

Optimized nucleic acid sequences of the invention may include codon-optimization, which refers to a gene coding sequence that has been optimized to increase expression by substituting one or more codons normally present in a coding sequence with a codon for the same amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wild type gene in an otherwise similar cell.

Sequence identity for a nucleic acid or amino acid sequence, for example an optimized nucleic acid, refers has the standard meaning in the art.

A percent nucleic acid sequence identity refers to the percentage of nucleotide residues in the candidate sequence that are identical with the nucleotides in the polynucleotide specifically disclosed herein. The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

Aa number of different programs can be used to identify whether a polynucleotide or polypeptide has sequence identity or similarity to a known sequence. Sequence identity or similarity may be determined using standard techniques known in the art, including, but not limited to the BLAST algorithm, described in Altschul et al., J Mol. Biol. 215:403 (1990) and Karlin et al., Proc. Natl. Acad. Sci. USA 90:5873 (1993).

### Micro RNA

MicroRNAs are small, single-stranded, non-coding RNA molecules. miRNAs base-pair to complementary sequences in mRNA molecules, thereby producing post-transcriptional regulation of gene expression. Typically, miRNA molecules silence messenger RNA (mRNA) translation by facilitating a process that leads to cleavage of mRNA strand into two pieces or destabilization of the mRNA by shortening its poly (A) tail. miRNAs are typically ˜22 nt and are expressed in a cell and tissue type specific manner.

miRNAs resemble small interfering RNAs (siRNAs), however miRNAs derive from regions of RNA transcripts that fold back on themselves to form short hairpins. Animal miRNAs are initially transcribed as part of one arm of an RNA stem-loop that in turn forms part of a several hundred nucleotide-long miRNA precursor termed a pri-miRNA. A single pri-miRNA may contain from one to six miRNA precursors. These hairpin loop structures are typically composed of about 70 nucleotides each. Each hairpin is flanked by sequences necessary for efficient processing.

The pre-miRNA hairpin is typically cleaved by the RNase enzyme Dicer. The RNase interacts with 5′ and 3′ ends of the hairpin and cuts away the loop joining the 3′ and 5′ arms, resulting in a miRNA: miRNA duplex about 22 nucleotides in length. Overall hairpin length and loop size influence the efficiency of Dicer processing. Although either strand of the duplex may potentially act as a functional miRNA, only one strand is generally incorporated into the RNA-induced silencing complex (RISC) where the miRNA and its mRNA target interact.

The present invention provides miR-338-3p, miR-138-5p, and miR-9-5p functional sites that inhibit expression of a transgene to selectively inhibit expression of the transgene in the dorsal root ganglia (DRG).

### I. Recombinant Nucleic Acids

Aspects of the present disclosure provide a recombinant nucleic acid comprising a muscle specific promoter, a sequence encoding microdystrophin, and at least one DRG de-targeting miRNA binding site. In some embodiments, the recombinant nucleic acid can further comprise an intron and/or a Poly A signal. Alternatively, or in addition to, the recombinant nucleic acid can further comprise a 5′ inverted terminal repeat (ITR) and a 3′ ITR.

Various promoters, including inducible promoters and constitutive promoters, can be used to drive expression from the recombinant nucleic acids described herein. Non-limiting examples of promoters that can be used in the recombinant nucleic acids disclosed herein include a cytomegalovirus (CMV) promoter, a chicken β-actin (CBA) promoter, an alpha-1 antitrypsin (hAAT) promoter, and any promoter derived from an immunoglobulin gene, SV40, or other tissue specific genes.

In some embodiments, the promoter is tissue-specific such that, in a multi-cellular organism, the promoter drives expression only in a subset of specific cells. For example, the promoter is a muscle specific promoter. Non-limiting examples of muscle specific promoters include a CK8 promoter, a desmin promoter, a Mb promoter, a MCK promoter, a MHCK7 promoter, a skeletal muscle alpha actin promoter, or a TTNI2 promoter.

In some embodiments, the promoter is a novel promoter described herein. For example, the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404. In another example, the promoter comprises or consists of the nucleic acid sequence of any one of SEQ ID NOs: 2402-2404. In some embodiments, the recombinant nucleic acid comprises or consists of the nucleic acid sequence of SEQ ID NO: 2402. In some embodiments, the recombinant nucleic acid comprises or consists of the nucleic acid sequence of SEQ ID NO: 2403. In some embodiments, the recombinant nucleic acid comprises or consists of the nucleic acid sequence of SEQ ID NO: 2404.

Recombinant nucleic acids described herein comprise a sequence encoding microdystrophin (e.g., a sequence encoding human microdystrophin). Upon administration of any of the recombinant nucleic acids described herein, microdystrophin protein (e.g., any biologically active fragment of dystrophin, e.g., ΔR4-R23/ΔCT microdystrophin) is expressed in muscle cells or muscle tissue to produce a therapeutic effect.

In some embodiments, the microdystrophin protein can comprise or consist of an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 2439. In some embodiments, the microdystrophin protein comprises or consists of the amino acid sequence of SEQ ID NO: 2439.

The sequence encoding microdystrophin can be an endogenous sequence encoding micodystrophin or a codon optimized sequence encoding microdystrophin such as the codon optimized sequences encoding microdystrophin provided as SEQ ID NOs: 2405-2407.

In some embodiments, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405. In some embodiments, the sequence encoding microdystrophin comprises or consists of the nucleic acid sequence of SEQ ID NO: 2405.

In some embodiments, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406. In some embodiments, the sequence encoding microdystrophin comprises or consists of the nucleic acid sequence of SEQ ID NO: 2406.

In some embodiments, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407. In some embodiments, the sequence encoding microdystrophin comprises or consists of the nucleic acid sequence of SEQ ID NO: 2407.

Any of the sequences encoding microdystrophin described herein can produce a microdystrophin protein that retains its biological activity (e.g., muscle membrane stabilization).

For example, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405, and microdystrophin retains biological activity (e.g., muscle membrane stabilization). In another example, the sequence encoding microdystrophin comprises or consists of the nucleic acid sequence of SEQ ID NO: 2405, and microdystrophin retains biological activity (e.g., muscle membrane stabilization).

In another example, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406, and microdystrophin retains biological activity (e.g., muscle membrane stabilization). In another example, the sequence encoding microdystrophin comprises or consists of the nucleic acid sequence of SEQ ID NO: 2406, and microdystrophin retains biological activity (e.g., muscle membrane stabilization).

In yet another example, the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407, and microdystrophin retains biological activity (e.g., muscle membrane stabilization). In another example, the sequence encoding microdystrophin comprises or consists of the nucleic acid sequence of SEQ ID NO: 2407, and microdystrophin retains biological activity (e.g., muscle membrane stabilization).

Recombinant nucleic acids described herein can comprise at least one DRG de-targeting miRNA binding site, e.g., at least one, at least two, at least three, at least four, at least five, or more DRG de-targeting miRNA binding sites.

Any DRG de-targeting miRNA binding site suitable for binding a DRG de-targeting miRNA can be included in the recombinant nucleic acids described herein. In some embodiments, the DRG de-targeting miRNA binding site can comprise or consist of a binding site for miR-338 (e.g., miR-338-3p), miR-138 (e.g., miR-138-5p), or miR-9 (e.g., miR-9-5p).

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401. In some embodiments, the DRG de-targeting miRNA binding site comprises or consists of the nucleic acid sequence of any one of SEQ ID NOs: 2399-2401.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399. In some embodiments, the DRG de-targeting miRNA binding site comprises or consists of the nucleic acid sequence of SEQ ID NOs: 2399.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400. In some embodiments, the DRG de-targeting miRNA binding site comprises or consists of the nucleic acid sequence of SEQ ID NOs: 2400.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401. In some embodiments, the DRG de-targeting miRNA binding site comprises or consists of the nucleic acid sequence of SEQ ID NOs: 2401.

In some embodiments, when the recombinant nucleic acid comprises more than one DRG de-targeting miRNA binding site, the more than one DRG de-targeting miRNA binding site can have the same sequence or a different sequence.

For example, when the recombinant nucleic acid comprises a first and a second DRG de-targeting miRNA binding site, each of the DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of any one of SEQ ID NOs: 2399-2401.

Alternatively, the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 2399 and SEQ ID NO: 2400, respectively, or vice versa; the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 2399 and SEQ ID NO: 2401, respectively, or vice versa; or the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 2400 and SEQ ID NO: 2401, respectively, or vice versa.

The at least one DRG de-targeting miRNA binding site can be positioned in the recombinant nucleic acids described herein at any location suitable for binding a DRG de-targeting miRNA. For example, the DRG de-targeting miRNA binding site can be positioned upstream or downstream of the sequence encoding microdystrophin. In another example, when the recombinant nucleic acid comprises more than one DRG de-targeting miRNA binding site, each of the DRG de-targeting miRNA binding sites can be positioned upstream or downstream of the sequence encoding microdystrophin. Alternatively, one or more of the DRG de-targeting miRNA binding sites can be positioned upstream of the sequence encoding microdystrophin and one or more of the DRG de-targeting miRNA binding sites can be positioned downstream of the sequence encoding microdystrophin.

Recombinant nucleic acids described herein can further comprise an intron to increase overall efficiency and output of gene expression. Preferably, the intron is positioned between the promoter and transgene (e.g., microdystrophin transgene) in a 5′ to 3′ orientation. Non-limiting examples of introns include a simian virus 40 (SV40) intron sequence, a minute virus of mice (MVM) intron, a RK intron, a β-globin intron, and/or a chicken β-actin (CBA) intron, including functional variants or fragments thereof.

In some embodiments, the intron is a SV40 intron, including functional variants or fragments thereof. In some embodiments, the SV40 intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409. In some embodiments, the SV40 intron comprises or consists of a nucleic acid sequence comprising at least one, two, or three, but no more than four, modifications, e.g., substitutions, relative to the nucleic acid sequence of SEQ ID NO: 2409. In some embodiments, the SV40 intron comprises or consists of the nucleic acid sequence of SEQ ID NO: 2409.

Recombinant nucleic acids described herein can further comprise a polyadenylation (PolyA) signal sequence or functional fragment thereof, e.g., a fragment capable of measurably increasing expression as compared to expression in its absence. Non-limiting examples of PolyA signal sequences include a bovine growth hormone polyadenylation (bgh-PolyA) signal, a synthetic PolyA signal, and an SV40 Poly A signal.

In some embodiments, the Poly A signal comprises a bgh-PolyA signal, and the bgh-PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410. In some embodiments, the PolyA signal comprises a bgh-PolyA signal, and the bgh-PolyA signal comprises or consists of the nucleic acid sequence of SEQ ID NO: 2410.

Recombinant nucleic acids described herein can comprise one or more Poly A signal sequences or functional fragments thereof. In some embodiments, the recombinant nucleic acid described herein comprises a poly A sequence between the 3′ end of a sequence encoding the microdystrophin transgene and the 5′ end of the 3′ ITR.

Recombinant nucleic acids described herein can comprise one or more ITRs, e.g., two ITRs, with one upstream and the other downstream of a sequence encoding microdystrophin and/or the other elements discussed above (e.g., a promoter, at least one DRG de-targeting miRNA binding site).

An ITR sequence may be wild type, or it may comprise one or more mutations, e.g., by the insertion, deletion, or substitution of nucleotides, as long as the sequences retain one or more function of a wild type ITR, such as providing for functional rescue, replication and packaging. In some embodiments, a recombinant nucleic acid as described herein can comprise two ITR sequences, each of which is wild type, variant, or modified ITR sequences. In some embodiments, a recombinant nucleic acid as described herein can comprise two ITR sequences, which are different (e.g., one wild type ITR sequence and one variant or modified ITR sequence). For example, the “left” or 5′ ITR is a modified ITR sequence that allows for production of self-complementary genomes, and the “right” or 3′ ITR is a wild type ITR sequence. In some embodiments, the “right” or 3′ ITR is a modified ITR sequence that allows for the production of self-complementary genomes, and the “left” or 5′ ITR is a wild type ITR sequence.

In some embodiments, a recombinant nucleic acid can comprise ITR sequences from any AAV serotype suitable for replication and packaging of the virus. Non-limiting examples AAV serotypes include AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, AAV7m8, or any derivative, hybrid or chimeric serotype thereof.

In some embodiments, the recombinant nucleic acid comprises one or more ITRs derived from AAV2. The nucleotides in an ITR sequence may be in a forward or reverse orientation. In some embodiments, the ITR sequences are both wild type, variant, or modified AAV2 ITR sequences. In some embodiments, the ITR sequences are different (e.g., one ITR is a wild type AAV2 sequence and one ITR is variant or modified AAV2 ITR sequence). In some embodiments, the “left” or 5′ ITR is a modified AAV2 ITR sequence that allows for production of self-complementary genomes, and the “right” or 3′ ITR is a wild type AAV2 ITR sequence. In some embodiments, the “right” or 3′ ITR is a modified AAV2 ITR sequence that allows for the production of self-complementary genomes, and the “left” or 5′ ITR is a wild type AAV2 ITR sequence. In some embodiments, the 5′ ITR is an AAV2 5′ ITR as described in McCarty, D. M., et al., Gene Ther (2001), which is incorporated by reference in its entirety. In some embodiments, the 3′ ITR is an AAV2 3′ ITR as described in Rolling, F. & Samulski, R. J. Molecular biotechnology (1995), which is incorporated by reference in its entirety. In some embodiments, the 3′ ITR may comprise a deletion of the terminal resolution site (dTR), which inhibits Rep protein nicking of the single stranded viral genome. The presence of the dTR in the 3′ ITR increases self-complementary binding of the viral genome to itself, which it may do because of its small size that allows for a double-stranded viral genome to be packaged within a viral capsid.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408. In some embodiments, the recombinant nucleic acid comprises a 5′ ITR comprising or consisting of the nucleic acid sequence of SEQ ID NO: 2408.

In some embodiments, the recombinant nucleic acid comprises a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411. In some embodiments, the recombinant nucleic acid comprises a 3′ ITR comprising or consisting of the nucleic acid sequence of SEQ ID NO: 2411.

### Example Recombinant Nucleic Acids

Non-limiting examples of recombinant nucleic acids for use in methods described herein are provided below. In some examples, the recombinant nucleic acid comprises, optionally from 5′ to 3′, a muscle specific promoter; a sequence encoding microdystrophin; and at least one DRG de-targeting miRNA binding site.

For example, the recombinant nucleic acid comprises, optionally from 5′ to 3′, a muscle specific promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In another example, the recombinant nucleic acid comprises a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

In some examples, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2405; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2405;

and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2406; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2399.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2400.

In another example, the recombinant nucleic acid comprises a promoter comprising or consisting of the nucleic acid sequence of SEQ ID NO: 2402; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2403; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In another example, the recombinant nucleic acid comprises a promoter comprising or consisting of the nucleic acid sequence of SEQ ID NO: 2404; a sequence encoding microdystrophin comprising or consisting of the nucleic acid sequence of SEQ ID NO: 2407; and at least one DRG de-targeting miRNA binding site comprising or consisting of the nucleic acid sequence of SEQ ID NO: 2401.

In some examples, the recombinant nucleic acid comprises, optionally from 5′ to 3′, a 5′ ITR; a muscle specific promoter; an intron; a sequence encoding microdystrophin; a first DRG de-targeting miRNA binding site; a second DRG de-targeting miRNA binding site; a Poly A signal; and a 3′ ITR.

For example, the recombinant nucleic acid comprises a 5′ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408; a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404; an intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409; a sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a first DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2404; an intron comprises or consists of the nucleic acid sequence of SEQ ID NO: 2409; a sequence encoding microdystrophin comprising or consisting of the nucleic acid sequence of SEQ ID NO: 2405; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprises or consists of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2406; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2399; a second DRG de-targeting miRNA binding site comprising or consisting of the nucleic acid sequence of SEQ ID NO: 2399; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and the 3′ ITR comprising or consisting of a nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2400; a second DRG de-targeting miRNA binding site comprising or consisting of the nucleic acid sequence of SEQ ID NO: 2400; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ ITR comprising or consisting of the nucleic acid sequence of SEQ ID NO: 2408; a promoter comprising or consisting of the nucleic acid sequence of SEQ ID NO: 2402; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2403; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises a 5′ITR comprising or consisting of the nucleic acid sequence of SEQ ID NO: 2408; a promoter comprising or consisting of the nucleic acid sequence of SEQ ID NO: 2404; an intron comprising or consisting of the nucleic acid sequence of SEQ ID NO: 2409; a sequence encoding microdystrophin comprising or consisting of the nucleic acid sequence of SEQ ID NO: 2407; a first DRG de-targeting miRNA binding site comprising or consisting of the nucleic acid sequence of SEQ ID NO: 2401; a second DRG de-targeting miRNA binding site comprising or consisting of the nucleic acid sequence of SEQ ID NO: 2401; a PolyA signal comprising or consisting of the nucleic acid sequence of SEQ ID NO: 2410; and a 3′ ITR comprising or consisting of the nucleic acid sequence of SEQ ID NO: 2411.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2412. In another example, the recombinant nucleic acid comprises or consists of the nucleic acid sequence of SEQ ID NO: 2412.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2413. In another example, the recombinant nucleic acid comprises or consists of the nucleic acid sequence of SEQ ID NO: 2413.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2414. In another example, the recombinant nucleic acid comprises or consists of the nucleic acid sequence of SEQ ID NO: 2414.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2415. In another example, the recombinant nucleic acid comprises or consists of the nucleic acid sequence of SEQ ID NO: 2415.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2416. In another example, the recombinant nucleic acid comprises or consists of the nucleic acid sequence of SEQ ID NO: 2416.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2417. In another example, the recombinant nucleic acid comprises or consists of the nucleic acid sequence of SEQ ID NO: 2417.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2418. In another example, the recombinant nucleic acid comprises or consists of the nucleic acid sequence of SEQ ID NO: 2418.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2419. In another example, the recombinant nucleic acid comprises or consists of the nucleic acid sequence of SEQ ID NO: 2419.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2420. In another example, the recombinant nucleic acid comprises or consists of the nucleic acid sequence of SEQ ID NO: 2420.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2421. In another example, the recombinant nucleic acid comprises or consists of the nucleic acid sequence of SEQ ID NO: 2421.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2422. In another example, the recombinant nucleic acid comprises or consists of the nucleic acid sequence of SEQ ID NO: 2422.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2423. In another example, the recombinant nucleic acid comprises or consists of the nucleic acid sequence of SEQ ID NO: 2423.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2424. In another example, the recombinant nucleic acid comprises or consists of the nucleic acid sequence of SEQ ID NO: 2424.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2425. In another example, the recombinant nucleic acid comprises or consists of the nucleic acid sequence of SEQ ID NO: 2425.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2426. In another example, the recombinant nucleic acid comprises or consists of the nucleic acid sequence of SEQ ID NO: 2426.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2427. In another example, the recombinant nucleic acid comprises or consists of the nucleic acid sequence of SEQ ID NO: 2427.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2428. In another example, the recombinant nucleic acid comprises or consists of the nucleic acid sequence of SEQ ID NO: 2428.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429. In another example, the recombinant nucleic acid comprises or consists of the nucleic acid sequence of SEQ ID NO: 2429.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2430. In another example, the recombinant nucleic acid comprises or consists of the nucleic acid sequence of SEQ ID NO: 2430.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2431. In another example, the recombinant nucleic acid comprises or consists of the nucleic acid sequence of SEQ ID NO: 2431.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2432. In another example, the recombinant nucleic acid comprises or consists of the nucleic acid sequence of SEQ ID NO: 2432.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2433. In another example, the recombinant nucleic acid comprises or consists of the nucleic acid sequence of SEQ ID NO: 2433.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2434. In another example, the recombinant nucleic acid comprises or consists of the nucleic acid sequence of SEQ ID NO: 2434.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2435. In another example, the recombinant nucleic acid comprises or consists of the nucleic acid sequence of SEQ ID NO: 2435.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2436. In another example, the recombinant nucleic acid comprises or consists of the nucleic acid sequence of SEQ ID NO: 2436.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2437. In another example, the recombinant nucleic acid comprises or consists of the nucleic acid sequence of SEQ ID NO: 2437.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2438. In another example, the recombinant nucleic acid comprises or consists of the nucleic acid sequence of SEQ ID NO: 2438.

### II. Recombinant Expression Vectors

Any of the recombinant nucleic acids described herein can be delivered to a cell (e.g., a muscle cell), a tissue (e.g., muscle tissue), or subject (e.g., human subject) without or with the use of a delivery system (e.g., recombinant expression vector (e.g., viral vector (e.g., AAV (e.g., rAAV))).

### (a) Recombinant Expression Vectors

Any of the recombinant nucleic acids described herein can be included in a recombinant expression vector (e.g., a viral vector (e.g., an AAV vector (e.g., arAAV vector))). Recombinant expression vectors can be delivered into a cell, a tissue, or a subject using any method known in the art or described herein. A recombinant expression vector can be an organic or inorganic molecule. A recombinant expression vector can be a small molecule (i.e., <5 kD) or a macromolecule (i.e., >5 kD). The recombinant expression vector can comprise DNA, RNA, or both DNA and RNA. The recombinant expression vector can be a DNA vector, a circular vector, or a plasmid. The recombinant expression vector can be double stranded or single stranded.

In some embodiments, a recombinant expression vector (e.g., a viral vector (e.g., an AAV (e.g., a rAAV))) disclosed herein can exhibit higher expression of the recombinant nucleic acid (e.g., expression of microdystrophin) in a specific tissue type (e.g., muscle) as compared to the expression of the same vector in a different tissue type (e.g., liver). For example, the recombinant expression vector can exhibit higher expression of microdystrophin in a skeletal muscle tissue or cell as compared to the expression of the same vector in a non-skeletal muscle tissue (e.g., heart, liver) or cell (e.g., heart cell, liver cell). In some embodiments, the vector exhibits at least about 1.2 fold, at least about 1.5 fold, at least about 1.75 fold, at least about 2 fold, at least about 2.5 fold, at least about 3 fold, at least about 4 fold higher expression of the recombinant nucleic acid (e.g., expression of microdystrophin) in a skeletal muscle tissue or cell as compared to the expression of the same vector in a non-skeletal muscle tissue (e.g., heart, liver) or cell (e.g., heart cell, liver cell).

In some embodiments, the recombinant expression vector is a viral vector. Non-limiting examples of viral vectors for use in compositions and methods described herein include retroviral vectors, adenoviral vectors, lentiviral vectors, adeno-associated viral (AAVs) vectors, herpes simplex viral (HSV) vectors, alphaviral vectors, pox viral vectors, murine leukemia viral vectors, and hybrid viral vectors.

### (b) Adeno Associated Virus Vectors

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

Previous approaches to identify AAV sequences correlated with tropism have relied upon the comparison of highly related extant serotypes with distinct characteristics, random domain swaps between unrelated serotypes, or consideration of higher-order structure, to identify motifs that define liver tropism. For example, mapping determinants of AAV tropism have been carried out by comparing highly related serotypes. One such example is the single-amino acid change (E531K) between AAV1 and AAV6 that improves murine liver transduction in AAV1. See, e.g., Wu et al. (2006) J. Virol., 80 (22): 11393-7, incorporated by reference herein. Another example is a reciprocal domain swap between AAV2 and AAV8 that alters tropism, but fails to define any robust specific tissue-targeting motifs. See, e.g., Raupp et al. (201) J. Virol., 86 (17): 9396-408, incorporated by reference herein. Further, global consideration of structure has only highlighted gross differences between better- or worse-liver-transducers that are more observational than useful in practice. Nam et al (2007) J. Virol., 81 (22): 12260-71.

AAVs exhibiting modified tissue tropism that may be used with the present invention are described in U.S. Pat. Nos. 9,695,220, 9,719,070; 10,119,125; 10,526,584; U.S. Patent Application Publication No. 2018-0369414; U.S. Patent Application Publication No. 2020-0123504; U.S. Patent Application Publication No. 2020-0318082; PCT International Patent Application Publication No. WO 2015/054653; PCT International Patent Application Publication No. WO 2016/179496; PCT International Patent Application Publication No. WO 2017/100791; and PCT International Patent Application Publication No. WO 2019/217911, the entirety of the contents of each of which are incorporated by reference herein.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV1, AAV2, AAV3, AAV4, AAV5, AAV6, AAV7, AAV8, AAV9 or any combinations thereof. In some embodiments, the AAV can be AAV1, AAV2, AAV5, AAV9 or any combination thereof. One can select the AAV of the AAV with regard to the cells to be targeted; e.g., one can select AAV serotypes 1, 2, 5, 9 or a hybrid capsid AAV1, AAV2, AAV5, AAV9 or any combination thereof for targeting brain and/or neuronal cells; and one can select AAV4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV1, AAV2, AAV5 or any combination thereof. In some embodiments, an AAV vector or system thereof capable of producing AAV particles capable of targeting cardiac tissue can be configured to generate an AAV particle having an AAV4 serotype. In some embodiments, an AAV vector or system thereof capable of producing AAV particles capable of targeting the liver can be configured to generate an AAV having an AAV8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild type AAV of any serotype can be generated via a method described herein and determined to have a particular cell-specific tropism, which can be the same or different as that of the reference wild type AAV serotype. In some embodiments, the cell, tissue, and/or specificity of the wild type serotype can be enhanced (e.g., made more selective or specific for a particular cell type that the serotype is already biased towards). For example, wild type AAV9 is biased towards muscle and brain in humans (see, e.g., Srivastava. 2017. Curr. Opin. Virol. 21:75-80.) By including an engineered AAV capsid and/or capsid protein variant of wild type AAV9 as described herein, the tropism for nervous cells might be reduced or eliminated and/or the muscle specificity increased such that the nervous specificity appears reduced in comparison, thus enhancing the specificity for muscle as compared to the wild type AAV9. As previously mentioned, inclusion of an engineered capsid and/or capsid protein variant of a wild type AAV serotype can have a different tropism than the wild type reference AAV serotype. For example, an engineered AAV capsid and/or capsid protein variant of AAV9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild type hybrid AAV particles suffer the same specificity issues as with the non-hybrid wild type serotypes previously discussed.

Advantages achieved by the wild type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild type serotype that the engineered AAV capsid is a variant of. For example, a hybrid AAV can be produced that includes an engineered AAV capsid that is a variant of an AAV9 serotype that is used to package a genome that contains components (e.g., rep elements) from an AAV2 serotype. As with wild type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as International Patent Application Publications WO WO 2021/050974 and WO 2021/077000 and PCT International Application No. WO 2022/020616, the contents of which are incorporated by reference herein.

Additional AAV vectors are described in International Patent Application Publication WO 2019/2071632, the contents of which are incorporated by reference herein.

Further AAV vectors are described in International Patent Application Publications WO 2020/086881 and WO 2020/235543, the contents of each of which are incorporated by reference herein.

Further AAV vectors are described in International Patent Application Publications WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### III. Viral Particles and Capsid Proteins

Any of the recombinant nucleic acids or recombinant expression vectors (e.g., recombinant expression vector (e.g., viral vector (e.g., AAV (e.g., rAAV))) described herein can be packaged or encapsulated in a viral particle (e.g., AAV particle). Viral particles for use in compositions and methods described herein can include any viral capsid protein (e.g., AAV capsid protein or variant thereof) known in the art or described herein.

In some embodiments, a viral particle (e.g., an AAV particle (e.g., arAAV particle)) disclosed herein can exhibit higher expression of the recombinant nucleic acid (e.g., expression of microdystrophin) in a specific tissue type as compared to the expression of the same viral particle in a different tissue type. For example, the viral particle can exhibit higher expression of microdystrophin in a skeletal muscle tissue or cell as compared to the expression of the same viral particle in a non-skeletal muscle tissue (e.g., heart, liver) or cell (e.g., heart cell, liver cell). In some embodiments, the viral particle exhibits at least about 1.2 fold, at least about 1.5 fold, at least about 1.75 fold, at least about 2 fold, at least about 2.5 fold, at least about 3 fold, at least about 4 fold higher expression of the recombinant nucleic acid (e.g., expression of microdystrophin) in a skeletal muscle tissue or cell as compared to the expression of the same viral vector in a non-skeletal muscle tissue (e.g., heart, liver) or cell (e.g., heart cell, liver cell).

Viral particles described herein can include any virus suitable for delivery of a transgene, e.g., retroviruses, adenovirus, lentivirus, AAV, and murine leukemia viruses. In some embodiments, the viral particle is a recombinant adenovirus comprising a recombinant nucleic acid or a recombinant expression vector described herein. In some embodiments, the viral particle is a recombinant AAV comprising a recombinant nucleic acid or a recombinant AAV vector described herein. The AAV particle can be a scAAV or a ssAVV.

AAV particles described herein can include one or more AAV capsid proteins. In some embodiments, the one or more AAV capsid proteins can be from one or more AAV serotypes. Non-limiting examples of AAV serotypes include AAV1, AAV2, AAV3, AAV4, AAV5, AAV6, AAV7, AAV8, AAV9, AAV10, AAV11, AAV12, AAVrh8, AAVfh10, AAV-DJ, AAV-DJ/8, AAV-PHP.B, AAV-PHP.B2, AAV-PHP.B3, AAV-PHP.A, AAV-PHP.eB, AAV-PHP.S, and functional variants of any AAV serotypes.

In some embodiments, the AAV particle comprises a capsid protein comprising an amino acid sequence of formula (I) X₁NX₂X₃X₄RGDRX₅X₆L (SEQ ID NO: 1), wherein each of X₁-X₆ is any amino acid. In some embodiments, X₁ is A, I, F, G, H, L, M, Q, S, T, or V. In some embodiments, X₂ is A, G, S, T, or Y. In some embodiments, X₃ is S, N, G, or P. In some embodiments, X₄ is A, G, H, I, M, S, T, or V. In some embodiments, X₅ is A, G, or Q. In some embodiments, X₆ is A, I, L, M, N, S, or Y.

The amino acid sequence of formula (I) can be in a hypervariable region IV (HVR IV) relative to a wild type AAV9 capsid. For example, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L (SEQ ID NO: 2441) is inserted after amino acid 455.

In some embodiments, the AAV particle comprises a capsid protein comprising an amino acid sequence of any one of SEQ ID NOs: 801-1599. In some embodiments, the AAV particle comprises a capsid protein comprising an amino acid sequence of any one of SEQ ID NOs: 1600-2398. In some embodiments, the AAV particle comprises or consists of a capsid protein comprising an amino acid sequence of any one of SEQ ID NOs: 2-800. In some embodiments, the AAV particle comprises a capsid protein comprising an amino acid sequence comprising or consisting of SEQ ID NOs: 191, 198, 199, 215, 256, 379, 544, 550, or 748. In some embodiments, the AAV particle comprises a capsid protein comprising an amino acid sequence comprising or consisting of SEQ ID NO: 191. In some embodiments, the AAV particle comprises a capsid protein comprising an amino acid sequence comprising or consisting of SEQ ID NO: 198. In some embodiments, the AAV particle comprises a capsid protein comprising an amino acid sequence comprising or consisting of SEQ ID NO: 199. In some embodiments, the AAV particle comprises a capsid protein comprising an amino acid sequence comprising or consisting of SEQ ID NO: 215. In some embodiments, the AAV particle comprises a capsid protein comprising an amino acid sequence comprising or consisting of SEQ ID NO: 256. In some embodiments, the AAV particle comprises a capsid protein comprising an amino acid sequence comprising or consisting of SEQ ID NO: 379. In some embodiments, the AAV particle comprises a capsid protein comprising an amino acid sequence comprising or consisting of SEQ ID NO: 544. In some embodiments, the AAV particle comprises a capsid protein comprising an amino acid sequence comprising or consisting of SEQ ID NO: 550. In some embodiments, the AAV particle comprises a capsid protein comprising an amino acid sequence comprising or consisting of SEQ ID NO: 748.

Any of the capsid proteins described herein can further include a deletion. For example, the capsid protein can further comprise a deletion of amino acid G267 relative to a wild type AAV9 vector.

The capsid protein is the shell or coating of the virus that enables its delivery into the host. Without the protein, the nucleic acids would be destroyed by the host without entering into the host cells and beginning transcription and translation. The capsid protein may be in the natural conformation of a naturally occurring AAV, or it may be modified.

In certain example embodiments, the AAV capsid protein is an engineered AAV capsid protein having reduced or eliminated uptake in a non-muscle cell as compared to a corresponding wild type AAV capsid polypeptide.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter.

Described herein are various embodiments of engineered viral capsids, such as adeno-associated virus (AAV) capsids, that can be engineered to confer cell-specific tropism, such as muscle specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-specific tropism, reduced immunogenicity, or both to the engineered viral particle. The engineered viral capsids described herein can include one or more engineered viral capsid proteins described herein. The engineered viral capsids described herein can include one or more engineered viral capsid proteins described herein that can contain a muscle-specific targeting moiety containing or composed of an n-mer motif described elsewhere herein.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered viral capsids can be variants of wild type viral capsid. For example, in some embodiments, the engineered AAV capsids can be variants of wild type AAV capsids. In some embodiments, the wild type AAV capsids can be composed of VP1, VP2, VP3 capsid proteins or a combination thereof. In other words, the engineered AAV capsids can include one or more variants of a wild type VP1, wild type VP2, and/or wild type VP3 capsid proteins. In some embodiments, the serotype of the reference wild type AAV capsid can be AAV1, AAV2, AAV3, AAV4, AAV5, AAV6, AAV8, AAV9 or any combination thereof. In some embodiments, the serotype of the wild type AAV capsid can be AAV9. The engineered AAV capsids can have a different tropism than that of the reference wild type AAV capsid.

The engineered viral capsid can contain 1-60 engineered capsid proteins. In some embodiments, the engineered viral capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered viral capsid can contain 0-59 wild type viral capsid proteins. In some embodiments, the engineered viral capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild type viral capsid proteins.

In some embodiments, the engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV capsid can contain 0-59 wild type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild type AAV capsid proteins.

In some embodiments, the engineered viral capsid protein can have an n-mer amino acid motif, where n can be at least 3 amino acids. In some embodiments, n can be 3, 4, 5, 6, 7, 8, 9, 10, 11, 12, 13, 14, or 15 amino acids. In some embodiments, an engineered AAV capsid can have a 6-mer or 7-mer amino acid motif. In some embodiments, the n-mer amino acid motif can be inserted between two amino acids in the wild type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild type AAV viral protein contains an eight-stranded beta-barrel motif (betaB to betaI) and an alpha-helix (alphaA) that are conserved in autonomous parvovirus capsids (see, e.g., DiMattia et al. 2012. J. Virol. 86 (12): 6947-6958). Structural variable regions (VRs) occur in the surface loops that connect the beta-strands, which cluster to produce local variations in the capsid surface. AAVs have 12 variable regions (also referred to as hypervariable regions) (see, e.g., Weitzman and Linden. 2011. “Adeno-Associated Virus Biology.” In Snyder, R. O., Moullier, P. (eds.) Totowa, NJ: Humana Press). In some embodiments, one or more n-mer motifs can be inserted between two amino acids in one or more of the 12 variable regions in the wild type AVV capsid proteins. In some embodiments, the one or more n-mer motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid protein. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer inserted between amino acids 588 and 589 of an AAV9 viral protein. In some embodiments, the engineered capsid can have a 7-mer motif inserted between amino acids 588 and 589 of an AAV9 viral protein. In other embodiments, the motif inserted is a 10-mer motif, with replacement of amino acids 586-88 and an insertion before 589. It will be appreciated that n-mers can be inserted in analogous positions in AAV viral proteins of other serotypes. In some embodiments as previously discussed, the n-mer(s) can be inserted between any two contiguous amino acids within the AAV viral protein and in some embodiments the insertion is made in a variable region.

In some embodiments, the first 1, 2, 3, or 4 amino acids of an n-mer motif can replace 1, 2, 3, or 4 amino acids of a polypeptide into which it is inserted and preceding the insertion site. In some embodiments, the amino acids of the n-mer motif that replace 1 or more amino acids of the polypeptide into which the n-mer motif is inserted come before or immediately before an “RGDG” (SEQ ID NO: 2440) in an n-mer motif. For example, in one or more of the 10-mer inserts, the first three amino acids shown can replace 1-3 amino acids into a polypeptide to which they may be inserted. Using an AAV as another non-limiting example, one or more of the n-mer motifs can be inserted into, e.g., and AAV9 capsid prolylpeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the n-mer motif is inserted are replaced by the n-mer motif.

In some embodiments, the AAV capsids or other viral capsids or compositions can be muscle specific. In some embodiments, muscle-specificity of the engineered AAV or other viral capsid or other composition is conferred by a muscle specific n-mer motif incorporated in the engineered AAV or other viral capsid or other composition described herein. While not intending to be bound by theory, it is believed that the n-mer motif confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a muscle cell. In some embodiments, the cell surface receptor is AAV receptor (AAVR). In some embodiments, the cell surface receptor is a muscle cell specific AAV receptor. In some embodiments, the cell surface receptor or other molecule is a cell surface receptor or other molecule selectively expressed on the surface of a muscle cell. In some embodiments, the cell surface receptor or molecule is an integrin or dimer thereof. In some embodiments, the cell surface receptor or molecule is an Vb6 integrin heterodimer.

In some embodiments, a muscle specific engineered viral particle or other composition described herein containing the muscle-specific capsid, n-mer motif, or muscle-specific targeting moiety described herein can have an increased uptake, delivery rate, transduction rate, efficiency, amount, or a combination thereof in a muscle cell as compared to other cells types and/or other virus particles (including but not limited to AAVs) and other compositions that do not contain the muscle-specific n-mer motif of the present invention.

### IV. Pharmaceutical Compositions

Any of the recombinant nucleic acids, the expression vectors (e.g., AAV vectors), or the AAV particles described herein can be included in a pharmaceutical composition comprising a pharmaceutically acceptable carrier.

Some embodiments of the invention may include any acceptable form of providing the AAV vector to a subject. For example, the AAV vector may be provided to the subject in the form of a composition or formulation comprising the AAV vector. The expression vector (e.g., AAV vector) of this invention can be formulated and administered to treat a variety of disease states by any means that produces contact of the active ingredient with the agent's site of action in the body of the subject. The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more muscle-specific targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, when engineered AAV capsid particles are included in the formulation, the formulation can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### (a) Pharmaceutically Acceptable Carriers and Auxiliary Ingredients and Agents

Any recombinant nucleic acids, expression vectors (e.g., AAV vectors), or AAV particles described herein can be included in a pharmaceutical composition comprising a pharmaceutically acceptable carrier.

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain antioxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### V. Methods of Treatment and Use

Aspects of the present disclosure provide methods for treating DMD in a subject in need thereof using any of the recombinant nucleic acids, the expression vectors (e.g., the AAV vectors), the AAV particles, or pharmaceutical compositions thereof described herein.

### (a) Subjects

A subject can be any subject for whom diagnosis, treatment, or therapy is desired. A subject can be a human or a non-human primate. A subject can be any age. In some embodiments, the subject is a human subject that is any age (e.g., a human subject that is between the ages of 2 and 5).

A subject (e.g., human subject) to be treated by the methods described herein can be a subject having, suspected of having, or at risk for having DMD. A subject (e.g., human subject) suspected of having DMD might show one or more symptoms of DMD, e.g., muscle weakness, muscle wasting, muscle pain, enlarged muscles, walking abnormalities, or difficulty breathing. A subject (e.g., human subject) at risk for DMD can be a subject having one or more risk factors for DMD, e.g., one or more mutations in a dystrophin gene. A subject (e.g., human subject) who needs the treatment described herein can be identified by routine medical examination, e.g., laboratory tests (e.g., serum creatine kinase (CK) blood tests), genetic testing (e.g., testing for one or more mutations in a dystrophin gene), or muscle biopsy.

Any of the methods described herein can further comprise a step of identifying a subject (e.g., a human subject) for treatment based on identifying one or more mutations in a dystrophin gene. In some embodiments, presence of one or more mutations in a dystrophin gene is determined in a biological sample (e.g., a blood sample) obtained from a subject (e.g., a human subject).

### (b) Administration

Methods described herein encompass administering an effective amount of a recombinant nucleic acid, an expression vector (e.g., an AAV vector), an AAV particle, or a pharmaceutical composition thereof to a subject, e.g., a subject in need thereof, for example, a human subject, by a variety of methods. The route of administration can be intramuscular administration, subcutaneous injection, intravenous injection, or intravenous (IV) administration.

An effective amount refers to the amount of a recombinant nucleic acid, an expression vector (e.g., an AAV vector), an AAV particle, or a pharmaceutical composition thereof needed to prevent or alleviate at least one or more signs or symptoms of DMD, and relates to a sufficient amount of a recombinant nucleic acid, an expression vector (e.g., an AAV vector), an AAV particle, or pharmaceutical compositions thereof that provides the desired effect, e.g., to treat a subject (e.g., human subject) having DMD. An effective amount also can include an amount sufficient to prevent or delay the development of a symptom of DMD, alter the course of a symptom of DMD (e.g., slow the progression of a symptom of DMD), or reverse a symptom of DMD.

Methods described herein comprise administering (e.g., intramuscularly or intravenously) to a subject (e.g., a human subject) a recombinant nucleic acid, an expression vector (e.g., an AAV vector), an AAV particle, or a pharmaceutical composition described herein one or more times. In some embodiments, an effective amount of a recombinant nucleic acid, an expression vector (e.g., an AAV vector), an AAV particle, or a pharmaceutical composition thereof is administered to a subject (e.g., a human subject) no more than once or more than once (e.g., administered twice, administered three times, or administered four times).

Any of the methods for treating DMD described herein can be used in combination therapies. For example, any of the methods for treating DMD described herein can be co-used with other therapeutic agents for treating DMD, or for enhancing efficacy of the compositions described herein (e.g., the recombinant nucleic acids, the expression vectors (e.g., the AAV vectors), or pharmaceutical compositions) and/or reducing side effects of the compositions described herein (e.g., the recombinant nucleic acids, the expression vectors (e.g., the AAV vectors), or pharmaceutical compositions).

Any of the recombinant nucleic acids, the expression vectors (e.g., the AAV vectors), or pharmaceutical compositions described herein can be used as a medicament or in the manufacture of a medicament for the treatment of DMD, or ameliorating one or more symptoms thereof in a subject.

### VI. Kits for Therapeutic Use

The present disclosure provides kits for treating DMD. Such kits can include any of the recombinant nucleic acids, AAV vectors, AAV particles, or pharmaceutical compositions thereof described herein. In some embodiments, the kit can additionally comprise instructions for practicing any of the methods described herein. The instructions may include information as to dosage, dosing schedule, and route of administration. Instructions supplied in the kits of the disclosure can be written instructions on a label or a package insert.

### OTHER EMBODIMENTS

Without further elaboration, it is believed that one skilled in the art can, based on the above description, utilize the present disclosure to its fullest extent. The specific embodiments herein are to be construed as merely illustrative, and not limitative of the remainder of the disclosure in any way whatsoever. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

In any of the aspects and embodiments described herein, the nucleic acid sequences contemplated can be DNA, RNA, or modified versions thereof. Modified nucleic acids can be distinguished from naturally occurring nucleic acids by modifications to the backbone of the polynucleotide chain, for example, peptide nucleic acids (PNA), morpholinos, locked nucleic acids (LNA), glycol nucleic acids (GNA) and threose nucleic acid (TNA). Modified nucleic acids can also include analogs with modifications to the four nucleobases. In some embodiments, the nucleic acids are PNAs. In some embodiments, the nucleic acids are LNAs. In some embodiments, the nucleic acids are morpholinos. In some embodiments, the nucleic acids are in a single-stranded form. In some embodiments, the nucleic acids are in double-stranded form. In some embodiments, the nucleic acids are linear. In some embodiments, the nucleic acids are circular. In some embodiments, the nucleic acids are plasmids.

Non-limiting embodiments of the present disclosure include:

Embodiment 1 is a composition comprising an adeno-associated virus (AAV) particle comprising an engineered capsid protein comprising the amino acid sequence RGDR (SEQ ID NO: 2440); and a nucleic acid molecule comprising a promoter; a sequence having at least 95% sequence identity with a sequence selected from SEQ ID NOs: 2405-2407; and a dorsal root ganglia (DRG) de-targeting miRNA binding site.

Embodiment 2 is the composition of Embodiment 1, wherein the nucleic acid molecule comprises a sequence having the sequence identity with a sequence selected from SEQ ID NOs: 2405-2407.

Embodiment 3 is the composition of Embodiment 2, wherein the DRG de-targeting miRNA binding site comprises a sequence selected from among SEQ ID NOs: 2399-2401.

Embodiment 4 is the composition of Embodiment 3, wherein the promoter comprises the sequence having at least 95% sequence identity a sequence selected from among SEQ ID NOs: 2402-2404.

Embodiment 5 is the composition of Embodiment 4, wherein the nucleic acid molecule comprises in order: a first inverted terminal repeats (ITR) region; a promoter; an intron; the sequence having at least 95% sequence identity with a sequence selected from SEQ ID NOs: 2405-2407; a first DRG de-targeting miRNA binding site; a second DRG de-targeting miRNA binding site; a poly adenylation signal; and a second ITR region.

Embodiment 6 is the composition of Embodiment 5, wherein when administered to a subject with a mutation in the DMD gene, resulting in lack of dystrophin protein expression, the composition is effective at restoring a healthy phenotype.

Embodiment 7 is the composition of Embodiment 6, wherein the nucleic acid molecule comprises a sequence selected from among of SEQ ID NOs: 2412-2438.

Embodiment 8 is the composition of Embodiment 1, wherein the capsid protein comprises an amino acid sequence selected from Table 1a in hypervariable region IV (HVR IV) relative to wild type AAV9, wherein the capsid protein comprises substitutions at amino acids 451-455 relative to a wild type AAV9 vector capsid selected from column 1 of Table 1b; and a 7-mer insert selected from column 2 of Table 1b inserted after amino acid 455 relative to a wild type AAV9 vector.

Embodiment 9 is the composition of Embodiment 8, wherein the capsid protein further comprises a deletion of G267 relative to wild type AAV9 capsid or equivalent position in another AAV capsid.

Embodiment 10 is the composition of Embodiment 9, wherein the AAV vector exhibits reduced liver tropism and increased muscle tropism as compared to a wild type AAV vector.

Embodiment 11 is a method of treating a subject suffering from DMD, the method comprising providing to the subject a composition comprising an adeno-associated virus (AAV) particle comprising an engineered capsid protein comprising the amino acid sequence RGDR (SEQ ID NO: 2440); and a nucleic acid molecule comprising a promoter; a sequence having at least 95% sequence identity with a sequence selected from SEQ ID NOs: 2405-2407; and a dorsal root ganglia (DRG) de-targeting miRNA binding site.

Embodiment 12 is the method of Embodiment 11, wherein the nucleic acid molecule comprises a sequence having the sequence identity with a sequence selected from SEQ ID NOs: 2405-2407.

Embodiment 13 is the method of Embodiment 12, wherein the DRG de-targeting miRNA binding site comprises a sequence selected from among SEQ ID NOs: 2399-2401.

Embodiment 14 is the method of Embodiment 13, wherein the promoter comprises the sequence having at least 95% sequence identity a sequence selected from among SEQ ID NOs: 2402-2404.

Embodiment 15 is the method of Embodiment 14, wherein the nucleic acid molecule comprises in order: a first inverted terminal repeats (ITR) region; a promoter; an intron; the sequence having at least 95% sequence identity with a sequence selected from SEQ ID NOs: 2405-2407; a first DRG de-targeting miRNA binding site; a second DRG de-targeting miRNA binding site; a poly adenylation signal; and a second ITR region.

Embodiment 16 is the method of Embodiment 15, wherein when administered to a subject with a mutation in the DMD gene, resulting in lack of dystrophin protein expression, the method is effective at restoring a healthy phenotype.

Embodiment 17 is the method of Embodiment 16, wherein the nucleic acid molecule comprises a sequence selected from among of SEQ ID NOs: 2412-2438

Embodiment 18 is the method of Embodiment 11, wherein the capsid protein comprises an amino acid sequence selected from Table 1a in hypervariable region IV (HVR IV) relative to wild type AAV9, wherein the capsid protein comprises substitutions at amino acids 451-455 relative to a wild type AAV9 vector capsid selected from column 1 of Table 1b; and a 7-mer insert selected from column 2 of Table 1b inserted after amino acid 455 relative to a wild type AAV9 vector.

Embodiment 19 is the method of Embodiment 18, wherein the capsid protein further comprises a deletion of G267 relative to wild type AAV9 capsid or equivalent position in another AAV capsid.

Embodiment 20 is the method of Embodiment 19, wherein the AAV vector exhibits reduced liver tropism and increased muscle tropism as compared to a wild type AAV vector. Embodiment 21 is a composition comprising a nucleic acid molecule comprising a sequence having at least 95% sequence identity with a sequence selected from any one of SEQ ID NOs: 2405-2407.

Embodiment 22 is the composition of Embodiment 21, wherein the nucleic acid molecule comprises a sequence having the sequence identity of SEQ ID NO: 2405.

Embodiment 23 is the composition of Embodiment 21, wherein the nucleic acid molecule comprises a sequence having the sequence identity of SEQ ID NO: 2406.

Embodiment 24 is the composition of Embodiment 21, wherein the nucleic acid molecule comprises a sequence having the sequence identity of SEQ ID NO: 2407.

Embodiment 25 is the composition of Embodiment 21, wherein the nucleic acid molecule is packaged in a vector.

Embodiment 26 is the composition of Embodiment 25, wherein the vector is an adeno-associated viral (AAV) vector.

Embodiment 27 is the composition of Embodiment 26, wherein the nucleic acid molecule comprises, in order, an inverted terminal repeats (ITR) region; a promoter; an intron; the sequence having at least 95% sequence identity with a sequence selected from any one of SEQ ID NOs: 2405-2407; a poly adenylation signal; and an ITR region.

Embodiment 28 is the composition of Embodiment 26, wherein each AAV vector comprises a capsid protein having at least one modification that results in reduced liver-tropism of the AAV vector and/or preferential targeting of the AAV vector to muscle tissue.

Embodiment 29 is the composition of Embodiment 21, wherein, when administered to a subject with a mutation in the DMD gene, which results in lack of dystrophin protein expression, the composition is effective at restoring a healthy phenotype.

Embodiment 30 is the composition of Embodiment 21, wherein a microdystrophin protein expressed by the nucleic acid molecule is smaller than 137.5 kilodaltons.

Embodiment 31 is a method of treating a subject suffering from DMD, the method comprising providing to the subject a composition comprising a nucleic acid molecule comprising a sequence having at least 95% sequence identity with a sequence selected from any one of SEQ ID NOs: 2405-2407.

Embodiment 32 is the method of Embodiment 31, wherein the nucleic acid molecule comprises a sequence having the sequence identity of SEQ ID NO: 2405.

Embodiment 33 is the method of Embodiment 31, wherein the nucleic acid molecule comprises a sequence having the sequence identity of SEQ ID NO: 2406.

Embodiment 34 is the method of Embodiment 31, wherein the nucleic acid molecule comprises a sequence having the sequence identity of SEQ ID NO: 2407.

Embodiment 35 is the method of Embodiment 31, wherein the nucleic acid molecule is packaged is in a vector.

Embodiment 36 is the method of Embodiment 35, wherein the vector is an adeno-associated viral (AAV) vector.

Embodiment 37 is the method of Embodiment 36, wherein the nucleic acid molecule comprises, in order, an inverted terminal repeats (ITR) region; a promoter; an intron; the sequence having at least 95% sequence identity with a sequence selected from SEQ ID NO: 2405-2407; a poly adenylation signal; and an ITR region.

Embodiment 38 is the method of Embodiment 36, wherein each AAV vector comprises a capsid protein having at least one modification that results in reduced liver-tropism of the AAV vector and/or preferential targeting of the AAV vector to muscle tissue.

Embodiment 39 is the method of Embodiment 31, wherein the providing step comprises providing to a subject having a mutation in the DMD gene, which results in lack of dystrophin protein expression, the composition is effective at restoring a healthy phenotype. Embodiment 40 is the method of Embodiment 31, wherein the protein expressed by the nucleic acid molecule is smaller than 137.5 kilodaltons.

### EXAMPLES

In order that the invention described may be more fully understood, the following examples are set forth. The examples described in this application are offered to illustrate the methods and compositions provided herein and are not to be construed in any way as limiting their scope.

### Example 1: Exemplary Recombinant Nucleic Acids and AAV Particles

Exemplary AAV particles were engineered comprising the capsid protein MyoAAV-LD B comprising an RGDR (SEQ ID NO: 2440) motif and a recombinant nucleic acid (also referred to as cargo) described herein.

The capsid protein MyoAAV-LD A can be described relative to a wild type AAV9, specifically the capsid protein MyoAAV-LD A includes the amino acid sequence of LNASM (SEQ ID NO: 998) at positions 451-455, the amino acid sequence of RGDRQSL (SEQ ID NO: 1797) inserted after amino acid 455, a deletion of the amino acid G267 relative to the wild type AAV9 capsid protein, and the remainder of the capsid is otherwise identical to wild type AAV9.

The capsid protein MyoAAV-LD B can be described relative to a wild type AAV9, specifically the capsid protein MyoAAV-LD B includes the amino acid sequence of LNAST (SEQ ID NO: 1014) at positions 451-455, the amino acid sequence of RGDRQYL (SEQ ID NO: 1854) inserted after amino acid 455, a deletion of the amino acid G267 relative to the wild type AAV9 capsid protein, and the remainder of the capsid is otherwise identical to wild type AAV9.

The capsid encapsidated a cargo nucleic acid comprising a novel promoter comprising a sequence selected from any one of SEQ ID NO: 2402-2404, a codon optimized microdystrophin comprising a sequence selected from any one of SEQ ID NO: 2405-2407, and a first and a second DRG binding sites comprising a sequence selected from any one of SEQ ID NOs: 2399-2401.

Exemplary recombinant nucleic acids are described below:

Nucleic acid 1
(SEQ ID NO: 2412)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAAGTGGAGGACTGCTACGAGAGAGAGGACGTGCAGAAGAAGACCTTCACC
AAGTGGGTGAACGCCCAGTTCAGCAAGTTCGGCAAGCAGCACATCGAGAACCTGTTCAGCGACCTGCAGGACGGC
AGAAGACTGCTGGACCTGCTGGAGGGCCTGACCGGCCAGAAGCTGCCTAAGGAGAAGGGCAGCACCAGAGTGCAC
GCTCTCAACAACGTGAACAAGGCCCTGAGAGTGCTGCAGAACAACAACGTGGACCTTGTGAACATCGGAAGCACC
GACATCGTGGATGGCAACCACAAGCTGACCCTGGGACTGATCTGGAACATCATCCTGCACTGGCAGGTGAAGAAC
GTCATGAAGAACATCATGGCCGGCCTGCAGCAGACCAACAGCGAAAAGATCCTGCTGAGCTGGGTCAGACAGAGC
ACCAGAAACTACCCTCAGGTGAACGTGATCAACTTCACCACCAGCTGGAGCGACGGCCTGGCCCTGAACGCCCTG
ATCCACAGCCACAGACCCGACCTGTTCGACTGGAACAGCGTGGTGTGCCAGCAGAGCGCCACCCAGAGACTGGAG
CACGCCTTCAACATCGCCAGATACCAGCTGGGCATCGAGAAGCTGCTGGACCCCGAGGACGTGGATACCACCTAC
CCCGACAAGAAGAGCATCCTGATGTACATCACCAGCCTGTTCCAGGTGCTGCCCCAGCAAGTGAGCATCGAGGCC
ATCCAGGAGGTGGAGATGCTGCCCAGACCTCCAAAGGTGACCAAGGAGGAGCACTTCCAGCTGCACCACCAGATG
CACTACAGCCAGCAGATCACCGTGAGCCTGGCCCAGGGCTACGAGAGAACCAGCAGCCCCAAGCCCAGATTCAAG
AGCTACGCCTACACCCAGGCCGCCTACGTGACCACCAGCGACCCCACCAGAAGCCCCTTCCCCAGCCAGCACCTG
GAGGCCCCCGAGGATAAGAGCTTCGGCAGCAGCCTGATGGAGAGCGAGGTGAACTTGGACAGATACCAGACCGCC
CTGGAGGAGGTGCTGAGCTGGCTGCTGAGCGCCGAGGACACCCTTCAGGCACAGGGCGAGATCAGCAACGACGTG
GAGGTGGTGAAGGACCAGTTCCACACCCACGAGGGCTACATGATGGACCTGACCGCCCACCAGGGCAGAGTGGGT
AACATCCTCCAGCTGGGAAGCAAGCTGATCGGCACCGGCAAGCTGAGCGAGGACGAGGAGACCGAGGTGCAAGAG
CAAATGAACCTGCTTAACAGCAGATGGGAGTGCCTGAGAGTGGCCAGCATGGAGAAGCAGAGCAACCTGCACAGA
GTGCTGATGGACCTGCAGAACCAGAAGCTGAAGGAGCTGAACGACTGGCTGACCAAGACCGAGGAGAGAACCAGA
AAGATGGAGGAGGAGCCCCTGGGCCCGGACCTGGAGGACCTGAAGCGGCAGGTGCAGCAGCACAAGGTCTTACAG
GAGGACCTGGAGCAGGAGCAAGTGAGAGTGAACAGCCTGACCCACATGGTGGTGGTGGTGGATGAGAGCAGCGGC
GACCACGCCACCGCCGCCCTGGAGGAGCAGCTGAAGGTGCTGGGCGACAGATGGGCCAACATCTGCAGATGGACC
GAGGACAGATGGGTTCTGCTGCAGGACATCCTGCTGAAGTGGCAGAGACTGACCGAGGAACAGTGCCTGTTCAGC
GCCTGGCTGAGTGAGAAGGAAGACGCTGTGAACAAGATCCACACCACCGGCTTCAAGGACCAGAACGAGATGCTG
AGCAGCCTGCAGAAGCTGGCCGTGCTGAAGGCAGACCTGGAGAAGAAAAAGCAGTCCATGGGAAAGCTGTACAGC
CTGAAGCAAGACCTGCTGAGCACCCTGAAGAACAAGAGCGTGACCCAGAAGACCGAGGCCTGGCTGGACAACTTC
GCCAGATGCTGGGACAACCTGGTTCAGAAGCTGGAGAAGAGCACCGCCCAGATCAGCCAGGCCGTGACCACCACC
CAGCCCAGCCTGACCCAGACCACCGTGATGGAGACCGTGACCACCGTGACCACCAGAGAGCAGATCCTGGTGAAG
CACGCCCAGGAGGAGCTGCCCCCTCCTCCCCCGCAAAAGAAGAGAACCCTGGAGAGACTGCAGGAGCTGCAGGAG
GCTACCGATGAGCTGGACCTGAAGCTGAGACAGGCCGAGGTGATCAAGGGCAGCTGGCAGCCCGTGGGTGACCTG
CTTATCGACAGCCTGCAGGACCACCTAGAGAAGGTGAAGGCCCTGAGAGGCGAGATCGCCCCTCTGAAGGAGAAC
GTGAGCCACGTGAACGACCTGGCCAGACAGCTGACCACCCTGGGAATCCAGCTGAGCCCCTACAACCTGAGCACC
CTGGAGGACCTGAACACGAGATGGAAGCTGCTGCAAGTGGCCGTGGAGGACAGAGTCAGACAGTTGCACGAGGCT
CACAGAGATTTCGGCCCCGCTAGTCAGCACTTCCTGAGCACCAGCGTGCAGGGCCCCTGGGAGAGAGCTATCAGC
CCAAACAAGGTGCCCTACTACATCAACCACGAGACCCAGACCACCTGCTGGGACCACCCCAAGATGACCGAGCTG
TACCAGAGCCTGGCAGACCTCAACAACGTGAGATTCAGCGCCTACAGAACCGCCATGAAGCTGAGAAGACTGCAG
AAGGCCCTGTGCCTGGACCTGCTGAGCCTGAGCGCCGCCTGCGACGCCCTGGACCAGCACAACCTGAAGCAGAAC
GACCAGCCCATGGACATCCTTCAGATCATCAACTGCCTGACCACCATCTACGACAGACTGGAGCAGGAACATAAC
AACCTGGTGAACGTGCCCCTGTGCGTGGACATGTGCCTGAACTGGCTGCTGAACGTGTACGACACCGGCAGAACA
GGCAGAATCAGAGTGCTGAGCTTCAAGACCGGCATCATCAGCCTGTGCAAGGCCCACCTGGAGGACAAGTACAGA
TACTTATTCAAGCAGGTGGCCAGCAGCACCGGCTTCTGCGATCAGAGAAGACTGGGCCTGCTGCTGCACGACAGC
ATCCAGATCCCCAGACAGCTGGGCGAAGTGGCCAGCTTCGGCGGCAGCAACATCGAGCCCAGCGTGAGAAGCTGC
TTCCAGTTCGCTAACAACAAGCCCGAGATCGAGGCCGCCCTGTTCTTGGACTGGATGAGACTGGAGCCCCAGAGC
ATGGTGTGGCTGCCCGTGCTGCACAGAGTGGCCGCCGCCGAGACCGCCAAGCACCAGGCCAAGTGTAACATCTGC
AAGGAGTGCCCAATCATCGGCTTCAGATACAGAAGCCTGAAGCACTTCAACTACGACATCTGCCAGAGCTGCTTC
TTCAGCGGCAGAGTGGCCAAGGGCCACAAGATGCACTACCCCATGGTGGAGTACTGCACCCCCACCACCAGCGGC
GAGGACGTGAGAGACTTCGCAAAGGTCCTGAAGAACAAGTTCAGAACCAAGAGATACTTCGCTAAGCACCCCAGA
ATGGGCTACCTGCCCGTGCAGACCGTGCTGGAGGGCGATAACATGGAGACCGACACCATGTGATAGATTCCAACA
AAATCACTGATGCTGGAAGATCAGCAACAAAATCACTGATGCTGGAAGACGCAGGAATAAAATATCTTTATTTTC
ATTACATCTGTGTGTTGGTTTTTTGTGTGAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGTGAGCGAGCG
AGCGCGC

| Nucleic acid | |
|---|---|
| cargo 1 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | TAGTTATTAATAGTAATCAATTACGGGGTCATTAGTTCATAGCCCATATATGGAGTTC |
| promoter | CGCGTTACATAACTTACGGTAAATGGCCCGCCTGGCTGACCGCCCAACGACCCCCGCC |
| | CATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGT |
| | CATCGCTATTACCATGCTAGACTAGCATGCTGCCCATGTAAGGAGGCAAGGCCTGGGG |
| | ACACCCGAGATGCCTGGTTATAATTAACCCAGACATGTGGCTGCCCCCCCCCCCCAAC |
| | ACCTGCTGCCTCTAAAAATAACCCTGCATGCCATGTTCCCGGCGAAGGGCCAGCTGTC |
| | CCCCGCCAGCTAGACTCAGCTCTCAGGGGCCCCTCCCTGGGGACAGCCCCTCCTGGCT |
| | AGTCACACCCTGTAGGCTCCTCTATATAACCCAGGGGCACAGGGGCTGCCCTCATTCT |
| | ACCACCACCTCCACAGCACAGACAGACACTCAGGAGCCAGCCAGC (SEQ ID NO: |
| | 2402) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAGGAAGTGGAGGACTGCTACGAGAGAGAGGACGTGCAGAAGAAGA |
| optimized | CCTTCACCAAGTGGGTGAACGCCCAGTTCAGCAAGTTCGGCAAGCAGCACATCGAGAA |
| microdystrophin | CCTGTTCAGCGACCTGCAGGACGGCAGAAGACTGCTGGACCTGCTGGAGGGCCTGACC |
| coding | GGCCAGAAGCTGCCTAAGGAGAAGGGCAGCACCAGAGTGCACGCTCTCAACAACGTGA |
| sequence | ACAAGGCCCTGAGAGTGCTGCAGAACAACAACGTGGACCTTGTGAACATCGGAAGCAC |
| | CGACATCGTGGATGGCAACCACAAGCTGACCCTGGGACTGATCTGGAACATCATCCTG |
| | CACTGGCAGGTGAAGAACGTCATGAAGAACATCATGGCCGGCCTGCAGCAGACCAACA |
| | GCGAAAAGATCCTGCTGAGCTGGGTCAGACAGAGCACCAGAAACTACCCTCAGGTGAA |
| | CGTGATCAACTTCACCACCAGCTGGAGCGACGGCCTGGCCCTGAACGCCCTGATCCAC |
| | AGCCACAGACCCGACCTGTTCGACTGGAACAGCGTGGTGTGCCAGCAGAGCGCCACCC |
| | AGAGACTGGAGCACGCCTTCAACATCGCCAGATACCAGCTGGGCATCGAGAAGCTGCT |
| | GGACCCCGAGGACGTGGATACCACCTACCCCGACAAGAAGAGCATCCTGATGTACATC |
| | ACCAGCCTGTTCCAGGTGCTGCCCCAGCAAGTGAGCATCGAGGCCATCCAGGAGGTGG |
| | AGATGCTGCCCAGACCTCCAAAGGTGACCAAGGAGGAGCACTTCCAGCTGCACCACCA |
| | GATGCACTACAGCCAGCAGATCACCGTGAGCCTGGCCCAGGGCTACGAGAGAACCAGC |
| | AGCCCCAAGCCCAGATTCAAGAGCTACGCCTACACCCAGGCCGCCTACGTGACCACCA |
| | GCGACCCCACCAGAAGCCCCTTCCCCAGCCAGCACCTGGAGGCCCCCGAGGATAAGAG |
| | CTTCGGCAGCAGCCTGATGGAGAGCGAGGTGAACTTGGACAGATACCAGACCGCCCTG |
| | GAGGAGGTGCTGAGCTGGCTGCTGAGCGCCGAGGACACCCTTCAGGCACAGGGCGAGA |
| | TCAGCAACGACGTGGAGGTGGTGAAGGACCAGTTCCACACCCACGAGGGCTACATGAT |
| | GGACCTGACCGCCCACCAGGGCAGAGTGGGTAACATCCTCCAGCTGGGAAGCAAGCTG |
| | ATCGGCACCGGCAAGCTGAGCGAGGACGAGGAGACCGAGGTGCAAGAGCAAATGAACC |
| | TGCTTAACAGCAGATGGGAGTGCCTGAGAGTGGCCAGCATGGAGAAGCAGAGCAACCT |
| | GCACAGAGTGCTGATGGACCTGCAGAACCAGAAGCTGAAGGAGCTGAACGACTGGCTG |
| | ACCAAGACCGAGGAGAGAACCAGAAAGATGGAGGAGGAGCCCCTGGGCCCGGACCTGG |
| | AGGACCTGAAGCGGCAGGTGCAGCAGCACAAGGTCTTACAGGAGGACCTGGAGCAGGA |
| | GCAAGTGAGAGTGAACAGCCTGACCCACATGGTGGTGGTGGTGGATGAGAGCAGCGGC |
| | GACCACGCCACCGCCGCCCTGGAGGAGCAGCTGAAGGTGCTGGGCGACAGATGGGCCA |
| | ACATCTGCAGATGGACCGAGGACAGATGGGTTCTGCTGCAGGACATCCTGCTGAAGTG |
| | GCAGAGACTGACCGAGGAACAGTGCCTGTTCAGCGCCTGGCTGAGTGAGAAGGAAGAC |
| | GCTGTGAACAAGATCCACACCACCGGCTTCAAGGACCAGAACGAGATGCTGAGCAGCC |
| | TGCAGAAGCTGGCCGTGCTGAAGGCAGACCTGGAGAAGAAAAAGCAGTCCATGGGAAA |
| | GCTGTACAGCCTGAAGCAAGACCTGCTGAGCACCCTGAAGAACAAGAGCGTGACCCAG |
| | AAGACCGAGGCCTGGCTGGACAACTTCGCCAGATGCTGGGACAACCTGGTTCAGAAGC |
| | TGGAGAAGAGCACCGCCCAGATCAGCCAGGCCGTGACCACCACCCAGCCCAGCCTGAC |
| | CCAGACCACCGTGATGGAGACCGTGACCACCGTGACCACCAGAGAGCAGATCCTGGTG |
| | AAGCACGCCCAGGAGGAGCTGCCCCCTCCTCCCCCGCAAAAGAAGAGAACCCTGGAGA |
| | GACTGCAGGAGCTGCAGGAGGCTACCGATGAGCTGGACCTGAAGCTGAGACAGGCCGA |
| | GGTGATCAAGGGCAGCTGGCAGCCCGTGGGTGACCTGCTTATCGACAGCCTGCAGGAC |
| | CACCTAGAGAAGGTGAAGGCCCTGAGAGGCGAGATCGCCCCTCTGAAGGAGAACGTGA |
| | GCCACGTGAACGACCTGGCCAGACAGCTGACCACCCTGGGAATCCAGCTGAGCCCCTA |
| | CAACCTGAGCACCCTGGAGGACCTGAACACGAGATGGAAGCTGCTGCAAGTGGCCGTG |
| | GAGGACAGAGTCAGACAGTTGCACGAGGCTCACAGAGATTTCGGCCCCGCTAGTCAGC |
| | ACTTCCTGAGCACCAGCGTGCAGGGCCCCTGGGAGAGAGCTATCAGCCCAAACAAGGT |
| | GCCCTACTACATCAACCACGAGACCCAGACCACCTGCTGGGACCACCCCAAGATGACC |
| | GAGCTGTACCAGAGCCTGGCAGACCTCAACAACGTGAGATTCAGCGCCTACAGAACCG |
| | CCATGAAGCTGAGAAGACTGCAGAAGGCCCTGTGCCTGGACCTGCTGAGCCTGAGCGC |
| | CGCCTGCGACGCCCTGGACCAGCACAACCTGAAGCAGAACGACCAGCCCATGGACATC |
| | CTTCAGATCATCAACTGCCTGACCACCATCTACGACAGACTGGAGCAGGAACATAACA |
| | ACCTGGTGAACGTGCCCCTGTGCGTGGACATGTGCCTGAACTGGCTGCTGAACGTGTA |
| | CGACACCGGCAGAACAGGCAGAATCAGAGTGCTGAGCTTCAAGACCGGCATCATCAGC |
| | CTGTGCAAGGCCCACCTGGAGGACAAGTACAGATACTTATTCAAGCAGGTGGCCAGCA |
| | GCACCGGCTTCTGCGATCAGAGAAGACTGGGCCTGCTGCTGCACGACAGCATCCAGAT |
| | CCCCAGACAGCTGGGCGAAGTGGCCAGCTTCGGCGGCAGCAACATCGAGCCCAGCGTG |
| | AGAAGCTGCTTCCAGTTCGCTAACAACAAGCCCGAGATCGAGGCCGCCCTGTTCTTGG |
| | ACTGGATGAGACTGGAGCCCCAGAGCATGGTGTGGCTGCCCGTGCTGCACAGAGTGGC |
| | CGCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGCT |
| | TCTTCAGCGGCAGAGTGGCCAAGGGCCACAAGATGCACTACCCCATGGTGGAGTACTG |
| | CACCCCCACCACCAGCGGCGAGGACGTGAGAGACTTCGCAAAGGTCCTGAAGAACAAG |
| | TTCAGAACCAAGAGATACTTCGCTAAGCACCCCAGAATGGGCTACCTGCCCGTGCAGA |
| | CCGTGCTGGAGGGCGATAACATGGAGACCGACACCATGTGA (SEQ ID NO: |
| | 2405) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 2
(SEQ ID NO: 2413)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 2 | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | CGTCTGTCTAATTCCAGCCAAGGGCATGGGGGAAAGGACAGAGCCCAGGGACCTGTG |
| promoter | GCCTATTCTCTGAGAGGACGCTGGGCTAATAATAGGAGGGGAGATTAGGGAGGGATT |
| | TGACTGCATTGTTATCTCACAGGAATGGCGTTTGAGAGCCTTGCTTCCAAGCTCTTC |
| | CAAGAGTCATGCCACCCCCCTCCAATCTCTGTGGAAGTGCCCACTGCCTTTCCCGCT |
| | TCGTTCTCTTCTCTCTAAATCTGAGCCCACCCCACTCAGGGGTACAGGATCTGGTTC |
| | ATTTATCTCCCAGAATGATGCCTTGGGTTGCGTCCCCTCTAAACTCTGGCACATTCC |
| | AGCCCCTCTTTGGGAGAAAGAACATCTCTTCTTGCCCAGGCCCTAGTGAGACATGCA |
| | TCGCAAGACATGCTCCCCCCTTCCTCCCCGCTGGGCTCCAGCTGCTGCCAGCCCTCC |
| | AAGAAAGGAGAGGCCCTGAGTTGGGCTATTTTGGTATCTGGGGTGGGCACCCGCAGG |
| | GCTAAGGTTACCTTGGTATGTTAAGGGCTCCCTGGGGCAGGACTATATAACCCCAGA |
| | GGGACTGCCCCATGCTGACTCCTTGCTTCTTTCCTGCTCGAGCCGCTGCCTTGCCCC |
| | CCGGAGACTGAAGACA (SEQ ID NO: 2403) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAGGAAGTGGAGGACTGCTACGAGAGAGAGGACGTGCAGAAGAAG |
| optimized | ACCTTCACCAAGTGGGTGAACGCCCAGTTCAGCAAGTTCGGCAAGCAGCACATCGAG |
| microdystrophin | AACCTGTTCAGCGACCTGCAGGACGGCAGAAGACTGCTGGACCTGCTGGAGGGCCTG |
| coding | ACCGGCCAGAAGCTGCCTAAGGAGAAGGGCAGCACCAGAGTGCACGCTCTCAACAAC |
| sequence | GTGAACAAGGCCCTGAGAGTGCTGCAGAACAACAACGTGGACCTTGTGAACATCGGA |
| | AGCACCGACATCGTGGATGGCAACCACAAGCTGACCCTGGGACTGATCTGGAACATC |
| | ATCCTGCACTGGCAGGTGAAGAACGTCATGAAGAACATCATGGCCGGCCTGCAGCAG |
| | ACCAACAGCGAAAAGATCCTGCTGAGCTGGGTCAGACAGAGCACCAGAAACTACCCT |
| | CAGGTGAACGTGATCAACTTCACCACCAGCTGGAGCGACGGCCTGGCCCTGAACGCC |
| | CTGATCCACAGCCACAGACCCGACCTGTTCGACTGGAACAGCGTGGTGTGCCAGCAG |
| | AGCGCCACCCAGAGACTGGAGCACGCCTTCAACATCGCCAGATACCAGCTGGGCATC |
| | GAGAAGCTGCTGGACCCCGAGGACGTGGATACCACCTACCCCGACAAGAAGAGCATC |
| | CTGATGTACATCACCAGCCTGTTCCAGGTGCTGCCCCAGCAAGTGAGCATCGAGGCC |
| | ATCCAGGAGGTGGAGATGCTGCCCAGACCTCCAAAGGTGACCAAGGAGGAGCACTTC |
| | CAGCTGCACCACCAGATGCACTACAGCCAGCAGATCACCGTGAGCCTGGCCCAGGGC |
| | TACGAGAGAACCAGCAGCCCCAAGCCCAGATTCAAGAGCTACGCCTACACCCAGGCC |
| | GCCTACGTGACCACCAGCGACCCCACCAGAAGCCCCTTCCCCAGCCAGCACCTGGAG |
| | GCCCCCGAGGATAAGAGCTTCGGCAGCAGCCTGATGGAGAGCGAGGTGAACTTGGAC |
| | AGATACCAGACCGCCCTGGAGGAGGTGCTGAGCTGGCTGCTGAGCGCCGAGGACACC |
| | CTTCAGGCACAGGGCGAGATCAGCAACGACGTGGAGGTGGTGAAGGACCAGTTCCAC |
| | ACCCACGAGGGCTACATGATGGACCTGACCGCCCACCAGGGCAGAGTGGGTAACATC |
| | CTCCAGCTGGGAAGCAAGCTGATCGGCACCGGCAAGCTGAGCGAGGACGAGGAGACC |
| | GAGGTGCAAGAGCAAATGAACCTGCTTAACAGCAGATGGGAGTGCCTGAGAGTGGCC |
| | AGCATGGAGAAGCAGAGCAACCTGCACAGAGTGCTGATGGACCTGCAGAACCAGAAG |
| | CTGAAGGAGCTGAACGACTGGCTGACCAAGACCGAGGAGAGAACCAGAAAGATGGAG |
| | GAGGAGCCCCTGGGCCCGGACCTGGAGGACCTGAAGCGGCAGGTGCAGCAGCACAAG |
| | GTCTTACAGGAGGACCTGGAGCAGGAGCAAGTGAGAGTGAACAGCCTGACCCACATG |
| | GTGGTGGTGGTGGATGAGAGCAGCGGCGACCACGCCACCGCCGCCCTGGAGGAGCAG |
| | CTGAAGGTGCTGGGCGACAGATGGGCCAACATCTGCAGATGGACCGAGGACAGATGG |
| | GTTCTGCTGCAGGACATCCTGCTGAAGTGGCAGAGACTGACCGAGGAACAGTGCCTG |
| | TTCAGCGCCTGGCTGAGTGAGAAGGAAGACGCTGTGAACAAGATCCACACCACCGGC |
| | TTCAAGGACCAGAACGAGATGCTGAGCAGCCTGCAGAAGCTGGCCGTGCTGAAGGCA |
| | GACCTGGAGAAGAAAAAGCAGTCCATGGGAAAGCTGTACAGCCTGAAGCAAGACCTG |
| | CTGAGCACCCTGAAGAACAAGAGCGTGACCCAGAAGACCGAGGCCTGGCTGGACAAC |
| | TTCGCCAGATGCTGGGACAACCTGGTTCAGAAGCTGGAGAAGAGCACCGCCCAGATC |
| | AGCCAGGCCGTGACCACCACCCAGCCCAGCCTGACCCAGACCACCGTGATGGAGACC |
| | GTGACCACCGTGACCACCAGAGAGCAGATCCTGGTGAAGCACGCCCAGGAGGAGCTG |
| | CCCCCTCCTCCCCCGCAAAAGAAGAGAACCCTGGAGAGACTGCAGGAGCTGCAGGAG |
| | GCTACCGATGAGCTGGACCTGAAGCTGAGACAGGCCGAGGTGATCAAGGGCAGCTGG |
| | CAGCCCGTGGGTGACCTGCTTATCGACAGCCTGCAGGACCACCTAGAGAAGGTGAAG |
| | GCCCTGAGAGGCGAGATCGCCCCTCTGAAGGAGAACGTGAGCCACGTGAACGACCTG |
| | GCCAGACAGCTGACCACCCTGGGAATCCAGCTGAGCCCCTACAACCTGAGCACCCTG |
| | GAGGACCTGAACACGAGATGGAAGCTGCTGCAAGTGGCCGTGGAGGACAGAGTCAGA |
| | CAGTTGCACGAGGCTCACAGAGATTTCGGCCCCGCTAGTCAGCACTTCCTGAGCACC |
| | AGCGTGCAGGGCCCCTGGGAGAGAGCTATCAGCCCAAACAAGGTGCCCTACTACATC |
| | AACCACGAGACCCAGACCACCTGCTGGGACCACCCCAAGATGACCGAGCTGTACCAG |
| | AGCCTGGCAGACCTCAACAACGTGAGATTCAGCGCCTACAGAACCGCCATGAAGCTG |
| | AGAAGACTGCAGAAGGCCCTGTGCCTGGACCTGCTGAGCCTGAGCGCCGCCTGCGAC |
| | GCCCTGGACCAGCACAACCTGAAGCAGAACGACCAGCCCATGGACATCCTTCAGATC |
| | ATCAACTGCCTGACCACCATCTACGACAGACTGGAGCAGGAACATAACAACCTGGTG |
| | AACGTGCCCCTGTGCGTGGACATGTGCCTGAACTGGCTGCTGAACGTGTACGACACC |
| | GGCAGAACAGGCAGAATCAGAGTGCTGAGCTTCAAGACCGGCATCATCAGCCTGTGC |
| | AAGGCCCACCTGGAGGACAAGTACAGATACTTATTCAAGCAGGTGGCCAGCAGCACC |
| | GGCTTCTGCGATCAGAGAAGACTGGGCCTGCTGCTGCACGACAGCATCCAGATCCCC |
| | AGACAGCTGGGCGAAGTGGCCAGCTTCGGCGGCAGCAACATCGAGCCCAGCGTGAGA |
| | AGCTGCTTCCAGTTCGCTAACAACAAGCCCGAGATCGAGGCCGCCCTGTTCTTGGAC |
| | TGGATGAGACTGGAGCCCCAGAGCATGGTGTGGCTGCCCGTGCTGCACAGAGTGGCC |
| | GCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGC |
| | TTCTTCAGCGGCAGAGTGGCCAAGGGCCACAAGATGCACTACCCCATGGTGGAGTAC |
| | TGCACCCCCACCACCAGCGGCGAGGACGTGAGAGACTTCGCAAAGGTCCTGAAGAAC |
| | AAGTTCAGAACCAAGAGATACTTCGCTAAGCACCCCAGAATGGGCTACCTGCCCGTG |
| | CAGACCGTGCTGGAGGGCGATAACATGGAGACCGACACCATGTGA (SEQ ID NO: |
| | 2405) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 3
(SEQ ID NO: 2414)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 3 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | CGTCTGTCTAATTCCAGCCAAGGGCATGGGGGAAAGGACAGAGCCCAGGGACCTGTGG |
| promoter | CCTATTCTCTGAGAGGACGCTGGGCTAATAATAGGAGGGGAGATTAGGGAGGGATTTG |
| | ACTGCATTGTTATCTCACAGGAATGGCGTTTGAGAGCCTTGCTTCCAAGCTCTTCCAA |
| | GAGTCATGCCACCCCCCTCCAATCTCTGTGGAAGTGCCCACTGCCTTTCCCGCTTCGT |
| | TCTCTTCTCTCGGTCCCATCCCTGACCCACTAGCCTGGTGCCGGGGGGAGACCAATTT |
| | CTGGAGGAAGCTCTGGGTACCCAGTCTTGCCCTAAGCCTGCCTCTGAACAGGCTGTTC |
| | TTGTTTATGACCCCCGTCCCAGCAGAATAGTGATTGCTCATCCCTGCATGTGATTTGA |
| | GAGGGCCAGTCTTTCAGGCCTGGAGGAAACTCAAGCCCCACCAGCCGCTTGGCATCGG |
| | AATGCTCAGCTGGTCCCCTCCTGGGGCTCATGTGACCAAGATCTAGCTATAAATATCA |
| | GAGGGGCCTCAGACCAAGACAGAATTCGGGCACCAGGAGAAGGAAGCCAACAGGATCC |
| | GACCCGGTGTTTTGTGACAAAGGCAAGACCCCCAGGTCTACTTAGAGCAACGTTAGTA |
| | GAGGA (SEQ ID NO: 2404) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAGGAAGTGGAGGACTGCTACGAGAGAGAGGACGTGCAGAAGAAGA |
| optimized | CCTTCACCAAGTGGGTGAACGCCCAGTTCAGCAAGTTCGGCAAGCAGCACATCGAGAA |
| microdystrophin | CCTGTTCAGCGACCTGCAGGACGGCAGAAGACTGCTGGACCTGCTGGAGGGCCTGACC |
| coding | GGCCAGAAGCTGCCTAAGGAGAAGGGCAGCACCAGAGTGCACGCTCTCAACAACGTGA |
| sequence | ACAAGGCCCTGAGAGTGCTGCAGAACAACAACGTGGACCTTGTGAACATCGGAAGCAC |
| | CGACATCGTGGATGGCAACCACAAGCTGACCCTGGGACTGATCTGGAACATCATCCTG |
| | CACTGGCAGGTGAAGAACGTCATGAAGAACATCATGGCCGGCCTGCAGCAGACCAACA |
| | GCGAAAAGATCCTGCTGAGCTGGGTCAGACAGAGCACCAGAAACTACCCTCAGGTGAA |
| | CGTGATCAACTTCACCACCAGCTGGAGCGACGGCCTGGCCCTGAACGCCCTGATCCAC |
| | AGCCACAGACCCGACCTGTTCGACTGGAACAGCGTGGTGTGCCAGCAGAGCGCCACCC |
| | AGAGACTGGAGCACGCCTTCAACATCGCCAGATACCAGCTGGGCATCGAGAAGCTGCT |
| | GGACCCCGAGGACGTGGATACCACCTACCCCGACAAGAAGAGCATCCTGATGTACATC |
| | ACCAGCCTGTTCCAGGTGCTGCCCCAGCAAGTGAGCATCGAGGCCATCCAGGAGGTGG |
| | AGATGCTGCCCAGACCTCCAAAGGTGACCAAGGAGGAGCACTTCCAGCTGCACCACCA |
| | GATGCACTACAGCCAGCAGATCACCGTGAGCCTGGCCCAGGGCTACGAGAGAACCAGC |
| | AGCCCCAAGCCCAGATTCAAGAGCTACGCCTACACCCAGGCCGCCTACGTGACCACCA |
| | GCGACCCCACCAGAAGCCCCTTCCCCAGCCAGCACCTGGAGGCCCCCGAGGATAAGAG |
| | CTTCGGCAGCAGCCTGATGGAGAGCGAGGTGAACTTGGACAGATACCAGACCGCCCTG |
| | GAGGAGGTGCTGAGCTGGCTGCTGAGCGCCGAGGACACCCTTCAGGCACAGGGCGAGA |
| | TCAGCAACGACGTGGAGGTGGTGAAGGACCAGTTCCACACCCACGAGGGCTACATGAT |
| | GGACCTGACCGCCCACCAGGGCAGAGTGGGTAACATCCTCCAGCTGGGAAGCAAGCTG |
| | ATCGGCACCGGCAAGCTGAGCGAGGACGAGGAGACCGAGGTGCAAGAGCAAATGAACC |
| | TGCTTAACAGCAGATGGGAGTGCCTGAGAGTGGCCAGCATGGAGAAGCAGAGCAACCT |
| | GCACAGAGTGCTGATGGACCTGCAGAACCAGAAGCTGAAGGAGCTGAACGACTGGCTG |
| | ACCAAGACCGAGGAGAGAACCAGAAAGATGGAGGAGGAGCCCCTGGGCCCGGACCTGG |
| | AGGACCTGAAGCGGCAGGTGCAGCAGCACAAGGTCTTACAGGAGGACCTGGAGCAGGA |
| | GCAAGTGAGAGTGAACAGCCTGACCCACATGGTGGTGGTGGTGGATGAGAGCAGCGGC |
| | GACCACGCCACCGCCGCCCTGGAGGAGCAGCTGAAGGTGCTGGGCGACAGATGGGCCA |
| | ACATCTGCAGATGGACCGAGGACAGATGGGTTCTGCTGCAGGACATCCTGCTGAAGTG |
| | GCAGAGACTGACCGAGGAACAGTGCCTGTTCAGCGCCTGGCTGAGTGAGAAGGAAGAC |
| | GCTGTGAACAAGATCCACACCACCGGCTTCAAGGACCAGAACGAGATGCTGAGCAGCC |
| | TGCAGAAGCTGGCCGTGCTGAAGGCAGACCTGGAGAAGAAAAAGCAGTCCATGGGAAA |
| | GCTGTACAGCCTGAAGCAAGACCTGCTGAGCACCCTGAAGAACAAGAGCGTGACCCAG |
| | AAGACCGAGGCCTGGCTGGACAACTTCGCCAGATGCTGGGACAACCTGGTTCAGAAGC |
| | TGGAGAAGAGCACCGCCCAGATCAGCCAGGCCGTGACCACCACCCAGCCCAGCCTGAC |
| | CCAGACCACCGTGATGGAGACCGTGACCACCGTGACCACCAGAGAGCAGATCCTGGTG |
| | AAGCACGCCCAGGAGGAGCTGCCCCCTCCTCCCCCGCAAAAGAAGAGAACCCTGGAGA |
| | GACTGCAGGAGCTGCAGGAGGCTACCGATGAGCTGGACCTGAAGCTGAGACAGGCCGA |
| | GGTGATCAAGGGCAGCTGGCAGCCCGTGGGTGACCTGCTTATCGACAGCCTGCAGGAC |
| | CACCTAGAGAAGGTGAAGGCCCTGAGAGGCGAGATCGCCCCTCTGAAGGAGAACGTGA |
| | GCCACGTGAACGACCTGGCCAGACAGCTGACCACCCTGGGAATCCAGCTGAGCCCCTA |
| | CAACCTGAGCACCCTGGAGGACCTGAACACGAGATGGAAGCTGCTGCAAGTGGCCGTG |
| | GAGGACAGAGTCAGACAGTTGCACGAGGCTCACAGAGATTTCGGCCCCGCTAGTCAGC |
| | ACTTCCTGAGCACCAGCGTGCAGGGCCCCTGGGAGAGAGCTATCAGCCCAAACAAGGT |
| | GCCCTACTACATCAACCACGAGACCCAGACCACCTGCTGGGACCACCCCAAGATGACC |
| | GAGCTGTACCAGAGCCTGGCAGACCTCAACAACGTGAGATTCAGCGCCTACAGAACCG |
| | CCATGAAGCTGAGAAGACTGCAGAAGGCCCTGTGCCTGGACCTGCTGAGCCTGAGCGC |
| | CGCCTGCGACGCCCTGGACCAGCACAACCTGAAGCAGAACGACCAGCCCATGGACATC |
| | CTTCAGATCATCAACTGCCTGACCACCATCTACGACAGACTGGAGCAGGAACATAACA |
| | ACCTGGTGAACGTGCCCCTGTGCGTGGACATGTGCCTGAACTGGCTGCTGAACGTGTA |
| | CGACACCGGCAGAACAGGCAGAATCAGAGTGCTGAGCTTCAAGACCGGCATCATCAGC |
| | CTGTGCAAGGCCCACCTGGAGGACAAGTACAGATACTTATTCAAGCAGGTGGCCAGCA |
| | GCACCGGCTTCTGCGATCAGAGAAGACTGGGCCTGCTGCTGCACGACAGCATCCAGAT |
| | CCCCAGACAGCTGGGCGAAGTGGCCAGCTTCGGCGGCAGCAACATCGAGCCCAGCGTG |
| | AGAAGCTGCTTCCAGTTCGCTAACAACAAGCCCGAGATCGAGGCCGCCCTGTTCTTGG |
| | ACTGGATGAGACTGGAGCCCCAGAGCATGGTGTGGCTGCCCGTGCTGCACAGAGTGGC |
| | CGCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGCT |
| | TCTTCAGCGGCAGAGTGGCCAAGGGCCACAAGATGCACTACCCCATGGTGGAGTACTG |
| | CACCCCCACCACCAGCGGCGAGGACGTGAGAGACTTCGCAAAGGTCCTGAAGAACAAG |
| | TTCAGAACCAAGAGATACTTCGCTAAGCACCCCAGAATGGGCTACCTGCCCGTGCAGA |
| | CCGTGCTGGAGGGCGATAACATGGAGACCGACACCATGTGA (SEQ ID NO: |
| | 2405) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 4
(SEQ ID NO: 2415)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAAGTGGAGGACTGCTACGAGAGAGAGGACGTGCAGAAGAAGACCTTCACC
AAGTGGGTGAACGCCCAGTTCAGCAAGTTCGGCAAGCAGCACATCGAGAACCTGTTCAGCGACCTGCAGGACGGC
AGAAGACTGCTGGACCTGCTGGAGGGCCTGACCGGCCAGAAGCTGCCTAAGGAGAAGGGCAGCACCAGAGTGCAC
GCTCTCAACAACGTGAACAAGGCCCTGAGAGTGCTGCAGAACAACAACGTGGACCTTGTGAACATCGGAAGCACC
GACATCGTGGATGGCAACCACAAGCTGACCCTGGGACTGATCTGGAACATCATCCTGCACTGGCAGGTGAAGAAC
GTCATGAAGAACATCATGGCCGGCCTGCAGCAGACCAACAGCGAAAAGATCCTGCTGAGCTGGGTCAGACAGAGC
ACCAGAAACTACCCTCAGGTGAACGTGATCAACTTCACCACCAGCTGGAGCGACGGCCTGGCCCTGAACGCCCTG
ATCCACAGCCACAGACCCGACCTGTTCGACTGGAACAGCGTGGTGTGCCAGCAGAGCGCCACCCAGAGACTGGAG
CACGCCTTCAACATCGCCAGATACCAGCTGGGCATCGAGAAGCTGCTGGACCCCGAGGACGTGGATACCACCTAC
CCCGACAAGAAGAGCATCCTGATGTACATCACCAGCCTGTTCCAGGTGCTGCCCCAGCAAGTGAGCATCGAGGCC
ATCCAGGAGGTGGAGATGCTGCCCAGACCTCCAAAGGTGACCAAGGAGGAGCACTTCCAGCTGCACCACCAGATG
CACTACAGCCAGCAGATCACCGTGAGCCTGGCCCAGGGCTACGAGAGAACCAGCAGCCCCAAGCCCAGATTCAAG
AGCTACGCCTACACCCAGGCCGCCTACGTGACCACCAGCGACCCCACCAGAAGCCCCTTCCCCAGCCAGCACCTG
GAGGCCCCCGAGGATAAGAGCTTCGGCAGCAGCCTGATGGAGAGCGAGGTGAACTTGGACAGATACCAGACCGCC
CTGGAGGAGGTGCTGAGCTGGCTGCTGAGCGCCGAGGACACCCTTCAGGCACAGGGCGAGATCAGCAACGACGTG
GAGGTGGTGAAGGACCAGTTCCACACCCACGAGGGCTACATGATGGACCTGACCGCCCACCAGGGCAGAGTGGGT
AACATCCTCCAGCTGGGAAGCAAGCTGATCGGCACCGGCAAGCTGAGCGAGGACGAGGAGACCGAGGTGCAAGAG
CAAATGAACCTGCTTAACAGCAGATGGGAGTGCCTGAGAGTGGCCAGCATGGAGAAGCAGAGCAACCTGCACAGA
GTGCTGATGGACCTGCAGAACCAGAAGCTGAAGGAGCTGAACGACTGGCTGACCAAGACCGAGGAGAGAACCAGA
AAGATGGAGGAGGAGCCCCTGGGCCCGGACCTGGAGGACCTGAAGCGGCAGGTGCAGCAGCACAAGGTCTTACAG
GAGGACCTGGAGCAGGAGCAAGTGAGAGTGAACAGCCTGACCCACATGGTGGTGGTGGTGGATGAGAGCAGCGGC
GACCACGCCACCGCCGCCCTGGAGGAGCAGCTGAAGGTGCTGGGCGACAGATGGGCCAACATCTGCAGATGGACC
GAGGACAGATGGGTTCTGCTGCAGGACATCCTGCTGAAGTGGCAGAGACTGACCGAGGAACAGTGCCTGTTCAGC
GCCTGGCTGAGTGAGAAGGAAGACGCTGTGAACAAGATCCACACCACCGGCTTCAAGGACCAGAACGAGATGCTG
AGCAGCCTGCAGAAGCTGGCCGTGCTGAAGGCAGACCTGGAGAAGAAAAAGCAGTCCATGGGAAAGCTGTACAGC
CTGAAGCAAGACCTGCTGAGCACCCTGAAGAACAAGAGCGTGACCCAGAAGACCGAGGCCTGGCTGGACAACTTC
GCCAGATGCTGGGACAACCTGGTTCAGAAGCTGGAGAAGAGCACCGCCCAGATCAGCCAGGCCGTGACCACCACC
CAGCCCAGCCTGACCCAGACCACCGTGATGGAGACCGTGACCACCGTGACCACCAGAGAGCAGATCCTGGTGAAG
CACGCCCAGGAGGAGCTGCCCCCTCCTCCCCCGCAAAAGAAGAGAACCCTGGAGAGACTGCAGGAGCTGCAGGAG
GCTACCGATGAGCTGGACCTGAAGCTGAGACAGGCCGAGGTGATCAAGGGCAGCTGGCAGCCCGTGGGTGACCTG
CTTATCGACAGCCTGCAGGACCACCTAGAGAAGGTGAAGGCCCTGAGAGGCGAGATCGCCCCTCTGAAGGAGAAC
GTGAGCCACGTGAACGACCTGGCCAGACAGCTGACCACCCTGGGAATCCAGCTGAGCCCCTACAACCTGAGCACC
CTGGAGGACCTGAACACGAGATGGAAGCTGCTGCAAGTGGCCGTGGAGGACAGAGTCAGACAGTTGCACGAGGCT
CACAGAGATTTCGGCCCCGCTAGTCAGCACTTCCTGAGCACCAGCGTGCAGGGCCCCTGGGAGAGAGCTATCAGC
CCAAACAAGGTGCCCTACTACATCAACCACGAGACCCAGACCACCTGCTGGGACCACCCCAAGATGACCGAGCTG
TACCAGAGCCTGGCAGACCTCAACAACGTGAGATTCAGCGCCTACAGAACCGCCATGAAGCTGAGAAGACTGCAG
AAGGCCCTGTGCCTGGACCTGCTGAGCCTGAGCGCCGCCTGCGACGCCCTGGACCAGCACAACCTGAAGCAGAAC
GACCAGCCCATGGACATCCTTCAGATCATCAACTGCCTGACCACCATCTACGACAGACTGGAGCAGGAACATAAC
AACCTGGTGAACGTGCCCCTGTGCGTGGACATGTGCCTGAACTGGCTGCTGAACGTGTACGACACCGGCAGAACA
GGCAGAATCAGAGTGCTGAGCTTCAAGACCGGCATCATCAGCCTGTGCAAGGCCCACCTGGAGGACAAGTACAGA
TACTTATTCAAGCAGGTGGCCAGCAGCACCGGCTTCTGCGATCAGAGAAGACTGGGCCTGCTGCTGCACGACAGC
ATCCAGATCCCCAGACAGCTGGGCGAAGTGGCCAGCTTCGGCGGCAGCAACATCGAGCCCAGCGTGAGAAGCTGC
TTCCAGTTCGCTAACAACAAGCCCGAGATCGAGGCCGCCCTGTTCTTGGACTGGATGAGACTGGAGCCCCAGAGC
ATGGTGTGGCTGCCCGTGCTGCACAGAGTGGCCGCCGCCGAGACCGCCAAGCACCAGGCCAAGTGTAACATCTGC
AAGGAGTGCCCAATCATCGGCTTCAGATACAGAAGCCTGAAGCACTTCAACTACGACATCTGCCAGAGCTGCTTC
TTCAGCGGCAGAGTGGCCAAGGGCCACAAGATGCACTACCCCATGGTGGAGTACTGCACCCCCACCACCAGCGGC
GAGGACGTGAGAGACTTCGCAAAGGTCCTGAAGAACAAGTTCAGAACCAAGAGATACTTCGCTAAGCACCCCAGA
ATGGGCTACCTGCCCGTGCAGACCGTGCTGGAGGGCGATAACATGGAGACCGACACCATGTGATAGATTCCGGCC
TGATTCACAACACCAGCTAGATCAGCGGCCTGATTCACAACACCAGCTAGACGCAGGAATAAAATATCTTTATTT
TCATTACATCTGTGTGTTGGTTTTTTGTGTGAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCAGTGAGCGAG
CGAGCGCGC

| Nucleic acid | |
|---|---|
| 4 cargo | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | TAGTTATTAATAGTAATCAATTACGGGGTCATTAGTTCATAGCCCATATATGGAGTT |
| promoter | CCGCGTTACATAACTTACGGTAAATGGCCCGCCTGGCTGACCGCCCAACGACCCCCG |
| | CCCATTGACGTCAATAATGACGTATGTTCCCATAGTAACGCCAATAGGGACTTTCCA |
| | TTGACGTCAATGGGTGGAGTATTTACGGTAAACTGCCCACTTGGCAGTACATCAAGT |
| | GTATCATATGCCAAGTACGCCCCCTATTGACGTCAATGACGGTAAATGGCCCGCCTG |
| | GCATTATGCCCAGTACATGACCTTATGGGACTTTCCTACTTGGCAGTACATCTACGT |
| | ATTAGTCATCGCTATTACCATGCTAGACTAGCATGCTGCCCATGTAAGGAGGCAAGG |
| | CCTGGGGACACCCGAGATGCCTGGTTATAATTAACCCAGACATGTGGCTGCCCCCCC |
| | CCCCCAACACCTGCTGCCTCTAAAAATAACCCTGCATGCCATGTTCCCGGCGAAGGG |
| | CCAGCTGTCCCCCGCCAGCTAGACTCAGCTCTCAGGGGCCCCTCCCTGGGGACAGCC |
| | CCTCCTGGCTAGTCACACCCTGTAGGCTCCTCTATATAACCCAGGGGCACAGGGGCT |
| | GCCCTCATTCTACCACCACCTCCACAGCACAGACAGACACTCAGGAGCCAGCCAGC |
| | (SEQ ID NO: 2402) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAGGAAGTGGAGGACTGCTACGAGAGAGAGGACGTGCAGAAGAAG |
| optimized | ACCTTCACCAAGTGGGTGAACGCCCAGTTCAGCAAGTTCGGCAAGCAGCACATCGAG |
| microdystrophin | AACCTGTTCAGCGACCTGCAGGACGGCAGAAGACTGCTGGACCTGCTGGAGGGCCTG |
| coding | ACCGGCCAGAAGCTGCCTAAGGAGAAGGGCAGCACCAGAGTGCACGCTCTCAACAAC |
| sequence | GTGAACAAGGCCCTGAGAGTGCTGCAGAACAACAACGTGGACCTTGTGAACATCGGA |
| | AGCACCGACATCGTGGATGGCAACCACAAGCTGACCCTGGGACTGATCTGGAACATC |
| | ATCCTGCACTGGCAGGTGAAGAACGTCATGAAGAACATCATGGCCGGCCTGCAGCAG |
| | ACCAACAGCGAAAAGATCCTGCTGAGCTGGGTCAGACAGAGCACCAGAAACTACCCT |
| | CAGGTGAACGTGATCAACTTCACCACCAGCTGGAGCGACGGCCTGGCCCTGAACGCC |
| | CTGATCCACAGCCACAGACCCGACCTGTTCGACTGGAACAGCGTGGTGTGCCAGCAG |
| | AGCGCCACCCAGAGACTGGAGCACGCCTTCAACATCGCCAGATACCAGCTGGGCATC |
| | GAGAAGCTGCTGGACCCCGAGGACGTGGATACCACCTACCCCGACAAGAAGAGCATC |
| | CTGATGTACATCACCAGCCTGTTCCAGGTGCTGCCCCAGCAAGTGAGCATCGAGGCC |
| | ATCCAGGAGGTGGAGATGCTGCCCAGACCTCCAAAGGTGACCAAGGAGGAGCACTTC |
| | CAGCTGCACCACCAGATGCACTACAGCCAGCAGATCACCGTGAGCCTGGCCCAGGGC |
| | TACGAGAGAACCAGCAGCCCCAAGCCCAGATTCAAGAGCTACGCCTACACCCAGGCC |
| | GCCTACGTGACCACCAGCGACCCCACCAGAAGCCCCTTCCCCAGCCAGCACCTGGAG |
| | GCCCCCGAGGATAAGAGCTTCGGCAGCAGCCTGATGGAGAGCGAGGTGAACTTGGAC |
| | AGATACCAGACCGCCCTGGAGGAGGTGCTGAGCTGGCTGCTGAGCGCCGAGGACACC |
| | CTTCAGGCACAGGGCGAGATCAGCAACGACGTGGAGGTGGTGAAGGACCAGTTCCAC |
| | ACCCACGAGGGCTACATGATGGACCTGACCGCCCACCAGGGCAGAGTGGGTAACATC |
| | CTCCAGCTGGGAAGCAAGCTGATCGGCACCGGCAAGCTGAGCGAGGACGAGGAGACC |
| | GAGGTGCAAGAGCAAATGAACCTGCTTAACAGCAGATGGGAGTGCCTGAGAGTGGCC |
| | AGCATGGAGAAGCAGAGCAACCTGCACAGAGTGCTGATGGACCTGCAGAACCAGAAG |
| | CTGAAGGAGCTGAACGACTGGCTGACCAAGACCGAGGAGAGAACCAGAAAGATGGAG |
| | GAGGAGCCCCTGGGCCCGGACCTGGAGGACCTGAAGCGGCAGGTGCAGCAGCACAAG |
| | GTCTTACAGGAGGACCTGGAGCAGGAGCAAGTGAGAGTGAACAGCCTGACCCACATG |
| | GTGGTGGTGGTGGATGAGAGCAGCGGCGACCACGCCACCGCCGCCCTGGAGGAGCAG |
| | CTGAAGGTGCTGGGCGACAGATGGGCCAACATCTGCAGATGGACCGAGGACAGATGG |
| | GTTCTGCTGCAGGACATCCTGCTGAAGTGGCAGAGACTGACCGAGGAACAGTGCCTG |
| | TTCAGCGCCTGGCTGAGTGAGAAGGAAGACGCTGTGAACAAGATCCACACCACCGGC |
| | TTCAAGGACCAGAACGAGATGCTGAGCAGCCTGCAGAAGCTGGCCGTGCTGAAGGCA |
| | GACCTGGAGAAGAAAAAGCAGTCCATGGGAAAGCTGTACAGCCTGAAGCAAGACCTG |
| | CTGAGCACCCTGAAGAACAAGAGCGTGACCCAGAAGACCGAGGCCTGGCTGGACAAC |
| | TTCGCCAGATGCTGGGACAACCTGGTTCAGAAGCTGGAGAAGAGCACCGCCCAGATC |
| | AGCCAGGCCGTGACCACCACCCAGCCCAGCCTGACCCAGACCACCGTGATGGAGACC |
| | GTGACCACCGTGACCACCAGAGAGCAGATCCTGGTGAAGCACGCCCAGGAGGAGCTG |
| | CCCCCTCCTCCCCCGCAAAAGAAGAGAACCCTGGAGAGACTGCAGGAGCTGCAGGAG |
| | GCTACCGATGAGCTGGACCTGAAGCTGAGACAGGCCGAGGTGATCAAGGGCAGCTGG |
| | CAGCCCGTGGGTGACCTGCTTATCGACAGCCTGCAGGACCACCTAGAGAAGGTGAAG |
| | GCCCTGAGAGGCGAGATCGCCCCTCTGAAGGAGAACGTGAGCCACGTGAACGACCTG |
| | GCCAGACAGCTGACCACCCTGGGAATCCAGCTGAGCCCCTACAACCTGAGCACCCTG |
| | GAGGACCTGAACACGAGATGGAAGCTGCTGCAAGTGGCCGTGGAGGACAGAGTCAGA |
| | CAGTTGCACGAGGCTCACAGAGATTTCGGCCCCGCTAGTCAGCACTTCCTGAGCACC |
| | AGCGTGCAGGGCCCCTGGGAGAGAGCTATCAGCCCAAACAAGGTGCCCTACTACATC |
| | AACCACGAGACCCAGACCACCTGCTGGGACCACCCCAAGATGACCGAGCTGTACCAG |
| | AGCCTGGCAGACCTCAACAACGTGAGATTCAGCGCCTACAGAACCGCCATGAAGCTG |
| | AGAAGACTGCAGAAGGCCCTGTGCCTGGACCTGCTGAGCCTGAGCGCCGCCTGCGAC |
| | GCCCTGGACCAGCACAACCTGAAGCAGAACGACCAGCCCATGGACATCCTTCAGATC |
| | ATCAACTGCCTGACCACCATCTACGACAGACTGGAGCAGGAACATAACAACCTGGTG |
| | AACGTGCCCCTGTGCGTGGACATGTGCCTGAACTGGCTGCTGAACGTGTACGACACC |
| | GGCAGAACAGGCAGAATCAGAGTGCTGAGCTTCAAGACCGGCATCATCAGCCTGTGC |
| | AAGGCCCACCTGGAGGACAAGTACAGATACTTATTCAAGCAGGTGGCCAGCAGCACC |
| | GGCTTCTGCGATCAGAGAAGACTGGGCCTGCTGCTGCACGACAGCATCCAGATCCCC |
| | AGACAGCTGGGCGAAGTGGCCAGCTTCGGCGGCAGCAACATCGAGCCCAGCGTGAGA |
| | AGCTGCTTCCAGTTCGCTAACAACAAGCCCGAGATCGAGGCCGCCCTGTTCTTGGAC |
| | TGGATGAGACTGGAGCCCCAGAGCATGGTGTGGCTGCCCGTGCTGCACAGAGTGGCC |
| | GCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGC |
| | TTCTTCAGCGGCAGAGTGGCCAAGGGCCACAAGATGCACTACCCCATGGTGGAGTAC |
| | TGCACCCCCACCACCAGCGGCGAGGACGTGAGAGACTTCGCAAAGGTCCTGAAGAAC |
| | AAGTTCAGAACCAAGAGATACTTCGCTAAGCACCCCAGAATGGGCTACCTGCCCGTG |
| | CAGACCGTGCTGGAGGGCGATAACATGGAGACCGACACCATGTGA (SEQ ID NO: |
| | 2405) |
| First DRG de- | CGGCCTGATTCACAACACCAGCT (SEQ ID NO: 2400) |
| targeting | |
| miRNA binding | |
| site | |
| Second DRG | CGGCCTGATTCACAACACCAGCT (SEQ ID NO: 2400) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 5
(SEQ ID NO: 2412)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 5 | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | CGTCTGTCTAATTCCAGCCAAGGGCATGGGGGAAAGGACAGAGCCCAGGGACCTGTG |
| promoter | GCCTATTCTCTGAGAGGACGCTGGGCTAATAATAGGAGGGGAGATTAGGGAGGGATT |
| | TGACTGCATTGTTATCTCACAGGAATGGCGTTTGAGAGCCTTGCTTCCAAGCTCTTC |
| | CAAGAGTCATGCCACCCCCCTCCAATCTCTGTGGAAGTGCCCACTGCCTTTCCCGCT |
| | TCGTTCTCTTCTCTCTAAATCTGAGCCCACCCCACTCAGGGGTACAGGATCTGGTTC |
| | ATTTATCTCCCAGAATGATGCCTTGGGTTGCGTCCCCTCTAAACTCTGGCACATTCC |
| | AGCCCCTCTTTGGGAGAAAGAACATCTCTTCTTGCCCAGGCCCTAGTGAGACATGCA |
| | TCGCAAGACATGCTCCCCCCTTCCTCCCCGCTGGGCTCCAGCTGCTGCCAGCCCTCC |
| | AAGAAAGGAGAGGCCCTGAGTTGGGCTATTTTGGTATCTGGGGTGGGCACCCGCAGG |
| | GCTAAGGTTACCTTGGTATGTTAAGGGCTCCCTGGGGCAGGACTATATAACCCCAGA |
| | GGGACTGCCCCATGCTGACTCCTTGCTTCTTTCCTGCTCGAGCCGCTGCCTTGCCCC |
| | CCGGAGACTGAAGACA (SEQ ID NO: 2403) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAGGAAGTGGAGGACTGCTACGAGAGAGAGGACGTGCAGAAGAAG |
| optimized | ACCTTCACCAAGTGGGTGAACGCCCAGTTCAGCAAGTTCGGCAAGCAGCACATCGAG |
| microdystrophin | AACCTGTTCAGCGACCTGCAGGACGGCAGAAGACTGCTGGACCTGCTGGAGGGCCTG |
| coding | ACCGGCCAGAAGCTGCCTAAGGAGAAGGGCAGCACCAGAGTGCACGCTCTCAACAAC |
| sequence | GTGAACAAGGCCCTGAGAGTGCTGCAGAACAACAACGTGGACCTTGTGAACATCGGA |
| | AGCACCGACATCGTGGATGGCAACCACAAGCTGACCCTGGGACTGATCTGGAACATC |
| | ATCCTGCACTGGCAGGTGAAGAACGTCATGAAGAACATCATGGCCGGCCTGCAGCAG |
| | ACCAACAGCGAAAAGATCCTGCTGAGCTGGGTCAGACAGAGCACCAGAAACTACCCT |
| | CAGGTGAACGTGATCAACTTCACCACCAGCTGGAGCGACGGCCTGGCCCTGAACGCC |
| | CTGATCCACAGCCACAGACCCGACCTGTTCGACTGGAACAGCGTGGTGTGCCAGCAG |
| | AGCGCCACCCAGAGACTGGAGCACGCCTTCAACATCGCCAGATACCAGCTGGGCATC |
| | GAGAAGCTGCTGGACCCCGAGGACGTGGATACCACCTACCCCGACAAGAAGAGCATC |
| | CTGATGTACATCACCAGCCTGTTCCAGGTGCTGCCCCAGCAAGTGAGCATCGAGGCC |
| | ATCCAGGAGGTGGAGATGCTGCCCAGACCTCCAAAGGTGACCAAGGAGGAGCACTTC |
| | CAGCTGCACCACCAGATGCACTACAGCCAGCAGATCACCGTGAGCCTGGCCCAGGGC |
| | TACGAGAGAACCAGCAGCCCCAAGCCCAGATTCAAGAGCTACGCCTACACCCAGGCC |
| | GCCTACGTGACCACCAGCGACCCCACCAGAAGCCCCTTCCCCAGCCAGCACCTGGAG |
| | GCCCCCGAGGATAAGAGCTTCGGCAGCAGCCTGATGGAGAGCGAGGTGAACTTGGAC |
| | AGATACCAGACCGCCCTGGAGGAGGTGCTGAGCTGGCTGCTGAGCGCCGAGGACACC |
| | CTTCAGGCACAGGGCGAGATCAGCAACGACGTGGAGGTGGTGAAGGACCAGTTCCAC |
| | ACCCACGAGGGCTACATGATGGACCTGACCGCCCACCAGGGCAGAGTGGGTAACATC |
| | CTCCAGCTGGGAAGCAAGCTGATCGGCACCGGCAAGCTGAGCGAGGACGAGGAGACC |
| | GAGGTGCAAGAGCAAATGAACCTGCTTAACAGCAGATGGGAGTGCCTGAGAGTGGCC |
| | AGCATGGAGAAGCAGAGCAACCTGCACAGAGTGCTGATGGACCTGCAGAACCAGAAG |
| | CTGAAGGAGCTGAACGACTGGCTGACCAAGACCGAGGAGAGAACCAGAAAGATGGAG |
| | GAGGAGCCCCTGGGCCCGGACCTGGAGGACCTGAAGCGGCAGGTGCAGCAGCACAAG |
| | GTCTTACAGGAGGACCTGGAGCAGGAGCAAGTGAGAGTGAACAGCCTGACCCACATG |
| | GTGGTGGTGGTGGATGAGAGCAGCGGCGACCACGCCACCGCCGCCCTGGAGGAGCAG |
| | CTGAAGGTGCTGGGCGACAGATGGGCCAACATCTGCAGATGGACCGAGGACAGATGG |
| | GTTCTGCTGCAGGACATCCTGCTGAAGTGGCAGAGACTGACCGAGGAACAGTGCCTG |
| | TTCAGCGCCTGGCTGAGTGAGAAGGAAGACGCTGTGAACAAGATCCACACCACCGGC |
| | TTCAAGGACCAGAACGAGATGCTGAGCAGCCTGCAGAAGCTGGCCGTGCTGAAGGCA |
| | GACCTGGAGAAGAAAAAGCAGTCCATGGGAAAGCTGTACAGCCTGAAGCAAGACCTG |
| | CTGAGCACCCTGAAGAACAAGAGCGTGACCCAGAAGACCGAGGCCTGGCTGGACAAC |
| | TTCGCCAGATGCTGGGACAACCTGGTTCAGAAGCTGGAGAAGAGCACCGCCCAGATC |
| | AGCCAGGCCGTGACCACCACCCAGCCCAGCCTGACCCAGACCACCGTGATGGAGACC |
| | GTGACCACCGTGACCACCAGAGAGCAGATCCTGGTGAAGCACGCCCAGGAGGAGCTG |
| | CCCCCTCCTCCCCCGCAAAAGAAGAGAACCCTGGAGAGACTGCAGGAGCTGCAGGAG |
| | GCTACCGATGAGCTGGACCTGAAGCTGAGACAGGCCGAGGTGATCAAGGGCAGCTGG |
| | CAGCCCGTGGGTGACCTGCTTATCGACAGCCTGCAGGACCACCTAGAGAAGGTGAAG |
| | GCCCTGAGAGGCGAGATCGCCCCTCTGAAGGAGAACGTGAGCCACGTGAACGACCTG |
| | GCCAGACAGCTGACCACCCTGGGAATCCAGCTGAGCCCCTACAACCTGAGCACCCTG |
| | GAGGACCTGAACACGAGATGGAAGCTGCTGCAAGTGGCCGTGGAGGACAGAGTCAGA |
| | CAGTTGCACGAGGCTCACAGAGATTTCGGCCCCGCTAGTCAGCACTTCCTGAGCACC |
| | AGCGTGCAGGGCCCCTGGGAGAGAGCTATCAGCCCAAACAAGGTGCCCTACTACATC |
| | AACCACGAGACCCAGACCACCTGCTGGGACCACCCCAAGATGACCGAGCTGTACCAG |
| | AGCCTGGCAGACCTCAACAACGTGAGATTCAGCGCCTACAGAACCGCCATGAAGCTG |
| | AGAAGACTGCAGAAGGCCCTGTGCCTGGACCTGCTGAGCCTGAGCGCCGCCTGCGAC |
| | GCCCTGGACCAGCACAACCTGAAGCAGAACGACCAGCCCATGGACATCCTTCAGATC |
| | ATCAACTGCCTGACCACCATCTACGACAGACTGGAGCAGGAACATAACAACCTGGTG |
| | AACGTGCCCCTGTGCGTGGACATGTGCCTGAACTGGCTGCTGAACGTGTACGACACC |
| | GGCAGAACAGGCAGAATCAGAGTGCTGAGCTTCAAGACCGGCATCATCAGCCTGTGC |
| | AAGGCCCACCTGGAGGACAAGTACAGATACTTATTCAAGCAGGTGGCCAGCAGCACC |
| | GGCTTCTGCGATCAGAGAAGACTGGGCCTGCTGCTGCACGACAGCATCCAGATCCCC |
| | AGACAGCTGGGCGAAGTGGCCAGCTTCGGCGGCAGCAACATCGAGCCCAGCGTGAGA |
| | AGCTGCTTCCAGTTCGCTAACAACAAGCCCGAGATCGAGGCCGCCCTGTTCTTGGAC |
| | TGGATGAGACTGGAGCCCCAGAGCATGGTGTGGCTGCCCGTGCTGCACAGAGTGGCC |
| | GCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGC |
| | TTCTTCAGCGGCAGAGTGGCCAAGGGCCACAAGATGCACTACCCCATGGTGGAGTAC |
| | TGCACCCCCACCACCAGCGGCGAGGACGTGAGAGACTTCGCAAAGGTCCTGAAGAAC |
| | AAGTTCAGAACCAAGAGATACTTCGCTAAGCACCCCAGAATGGGCTACCTGCCCGTG |
| | CAGACCGTGCTGGAGGGCGATAACATGGAGACCGACACCATGTGA (SEQ ID NO: |
| | 2405) |
| First DRG de- | CAACAAAATCACTGATGCTGGA (SEQ ID NO: 2400) |
| targeting | |
| miRNA binding | |
| site | |
| Second DRG | CAACAAAATCACTGATGCTGGA (SEQ ID NO: 2400) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 6
(SEQ ID NO: 2417)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 6 | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | CGTCTGTCTAATTCCAGCCAAGGGCATGGGGGAAAGGACAGAGCCCAGGGACCTGTG |
| promoter | GCCTATTCTCTGAGAGGACGCTGGGCTAATAATAGGAGGGGAGATTAGGGAGGGATT |
| | TGACTGCATTGTTATCTCACAGGAATGGCGTTTGAGAGCCTTGCTTCCAAGCTCTTC |
| | CAAGAGTCATGCCACCCCCCTCCAATCTCTGTGGAAGTGCCCACTGCCTTTCCCGCT |
| | TCGTTCTCTTCTCTCGGTCCCATCCCTGACCCACTAGCCTGGTGCCGGGGGGAGACC |
| | AATTTCTGGAGGAAGCTCTGGGTACCCAGTCTTGCCCTAAGCCTGCCTCTGAACAGG |
| | CTGTTCTTGTTTATGACCCCCGTCCCAGCAGAATAGTGATTGCTCATCCCTGCATGT |
| | GATTTGAGAGGGCCAGTCTTTCAGGCCTGGAGGAAACTCAAGCCCCACCAGCCGCTT |
| | GGCATCGGAATGCTCAGCTGGTCCCCTCCTGGGGCTCATGTGACCAAGATCTAGCTA |
| | TAAATATCAGAGGGGCCTCAGACCAAGACAGAATTCGGGCACCAGGAGAAGGAAGCC |
| | AACAGGATCCGACCCGGTGTTTTGTGACAAAGGCAAGACCCCCAGGTCTACTTAGAG |
| | CAACGTTAGTAGAGGA (SEQ ID NO: 2404) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAGGAAGTGGAGGACTGCTACGAGAGAGAGGACGTGCAGAAGAAG |
| optimized | ACCTTCACCAAGTGGGTGAACGCCCAGTTCAGCAAGTTCGGCAAGCAGCACATCGAG |
| microdystroph | AACCTGTTCAGCGACCTGCAGGACGGCAGAAGACTGCTGGACCTGCTGGAGGGCCTG |
| in coding | ACCGGCCAGAAGCTGCCTAAGGAGAAGGGCAGCACCAGAGTGCACGCTCTCAACAAC |
| sequence | GTGAACAAGGCCCTGAGAGTGCTGCAGAACAACAACGTGGACCTTGTGAACATCGGA |
| | AGCACCGACATCGTGGATGGCAACCACAAGCTGACCCTGGGACTGATCTGGAACATC |
| | ATCCTGCACTGGCAGGTGAAGAACGTCATGAAGAACATCATGGCCGGCCTGCAGCAG |
| | ACCAACAGCGAAAAGATCCTGCTGAGCTGGGTCAGACAGAGCACCAGAAACTACCCT |
| | CAGGTGAACGTGATCAACTTCACCACCAGCTGGAGCGACGGCCTGGCCCTGAACGCC |
| | CTGATCCACAGCCACAGACCCGACCTGTTCGACTGGAACAGCGTGGTGTGCCAGCAG |
| | AGCGCCACCCAGAGACTGGAGCACGCCTTCAACATCGCCAGATACCAGCTGGGCATC |
| | GAGAAGCTGCTGGACCCCGAGGACGTGGATACCACCTACCCCGACAAGAAGAGCATC |
| | CTGATGTACATCACCAGCCTGTTCCAGGTGCTGCCCCAGCAAGTGAGCATCGAGGCC |
| | ATCCAGGAGGTGGAGATGCTGCCCAGACCTCCAAAGGTGACCAAGGAGGAGCACTTC |
| | CAGCTGCACCACCAGATGCACTACAGCCAGCAGATCACCGTGAGCCTGGCCCAGGGC |
| | TACGAGAGAACCAGCAGCCCCAAGCCCAGATTCAAGAGCTACGCCTACACCCAGGCC |
| | GCCTACGTGACCACCAGCGACCCCACCAGAAGCCCCTTCCCCAGCCAGCACCTGGAG |
| | GCCCCCGAGGATAAGAGCTTCGGCAGCAGCCTGATGGAGAGCGAGGTGAACTTGGAC |
| | AGATACCAGACCGCCCTGGAGGAGGTGCTGAGCTGGCTGCTGAGCGCCGAGGACACC |
| | CTTCAGGCACAGGGCGAGATCAGCAACGACGTGGAGGTGGTGAAGGACCAGTTCCAC |
| | ACCCACGAGGGCTACATGATGGACCTGACCGCCCACCAGGGCAGAGTGGGTAACATC |
| | CTCCAGCTGGGAAGCAAGCTGATCGGCACCGGCAAGCTGAGCGAGGACGAGGAGACC |
| | GAGGTGCAAGAGCAAATGAACCTGCTTAACAGCAGATGGGAGTGCCTGAGAGTGGCC |
| | AGCATGGAGAAGCAGAGCAACCTGCACAGAGTGCTGATGGACCTGCAGAACCAGAAG |
| | CTGAAGGAGCTGAACGACTGGCTGACCAAGACCGAGGAGAGAACCAGAAAGATGGAG |
| | GAGGAGCCCCTGGGCCCGGACCTGGAGGACCTGAAGCGGCAGGTGCAGCAGCACAAG |
| | GTCTTACAGGAGGACCTGGAGCAGGAGCAAGTGAGAGTGAACAGCCTGACCCACATG |
| | GTGGTGGTGGTGGATGAGAGCAGCGGCGACCACGCCACCGCCGCCCTGGAGGAGCAG |
| | CTGAAGGTGCTGGGCGACAGATGGGCCAACATCTGCAGATGGACCGAGGACAGATGG |
| | GTTCTGCTGCAGGACATCCTGCTGAAGTGGCAGAGACTGACCGAGGAACAGTGCCTG |
| | TTCAGCGCCTGGCTGAGTGAGAAGGAAGACGCTGTGAACAAGATCCACACCACCGGC |
| | TTCAAGGACCAGAACGAGATGCTGAGCAGCCTGCAGAAGCTGGCCGTGCTGAAGGCA |
| | GACCTGGAGAAGAAAAAGCAGTCCATGGGAAAGCTGTACAGCCTGAAGCAAGACCTG |
| | CTGAGCACCCTGAAGAACAAGAGCGTGACCCAGAAGACCGAGGCCTGGCTGGACAAC |
| | TTCGCCAGATGCTGGGACAACCTGGTTCAGAAGCTGGAGAAGAGCACCGCCCAGATC |
| | AGCCAGGCCGTGACCACCACCCAGCCCAGCCTGACCCAGACCACCGTGATGGAGACC |
| | GTGACCACCGTGACCACCAGAGAGCAGATCCTGGTGAAGCACGCCCAGGAGGAGCTG |
| | CCCCCTCCTCCCCCGCAAAAGAAGAGAACCCTGGAGAGACTGCAGGAGCTGCAGGAG |
| | GCTACCGATGAGCTGGACCTGAAGCTGAGACAGGCCGAGGTGATCAAGGGCAGCTGG |
| | CAGCCCGTGGGTGACCTGCTTATCGACAGCCTGCAGGACCACCTAGAGAAGGTGAAG |
| | GCCCTGAGAGGCGAGATCGCCCCTCTGAAGGAGAACGTGAGCCACGTGAACGACCTG |
| | GCCAGACAGCTGACCACCCTGGGAATCCAGCTGAGCCCCTACAACCTGAGCACCCTG |
| | GAGGACCTGAACACGAGATGGAAGCTGCTGCAAGTGGCCGTGGAGGACAGAGTCAGA |
| | CAGTTGCACGAGGCTCACAGAGATTTCGGCCCCGCTAGTCAGCACTTCCTGAGCACC |
| | AGCGTGCAGGGCCCCTGGGAGAGAGCTATCAGCCCAAACAAGGTGCCCTACTACATC |
| | AACCACGAGACCCAGACCACCTGCTGGGACCACCCCAAGATGACCGAGCTGTACCAG |
| | AGCCTGGCAGACCTCAACAACGTGAGATTCAGCGCCTACAGAACCGCCATGAAGCTG |
| | AGAAGACTGCAGAAGGCCCTGTGCCTGGACCTGCTGAGCCTGAGCGCCGCCTGCGAC |
| | GCCCTGGACCAGCACAACCTGAAGCAGAACGACCAGCCCATGGACATCCTTCAGATC |
| | ATCAACTGCCTGACCACCATCTACGACAGACTGGAGCAGGAACATAACAACCTGGTG |
| | AACGTGCCCCTGTGCGTGGACATGTGCCTGAACTGGCTGCTGAACGTGTACGACACC |
| | GGCAGAACAGGCAGAATCAGAGTGCTGAGCTTCAAGACCGGCATCATCAGCCTGTGC |
| | AAGGCCCACCTGGAGGACAAGTACAGATACTTATTCAAGCAGGTGGCCAGCAGCACC |
| | GGCTTCTGCGATCAGAGAAGACTGGGCCTGCTGCTGCACGACAGCATCCAGATCCCC |
| | AGACAGCTGGGCGAAGTGGCCAGCTTCGGCGGCAGCAACATCGAGCCCAGCGTGAGA |
| | AGCTGCTTCCAGTTCGCTAACAACAAGCCCGAGATCGAGGCCGCCCTGTTCTTGGAC |
| | TGGATGAGACTGGAGCCCCAGAGCATGGTGTGGCTGCCCGTGCTGCACAGAGTGGCC |
| | GCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGC |
| | TTCTTCAGCGGCAGAGTGGCCAAGGGCCACAAGATGCACTACCCCATGGTGGAGTAC |
| | TGCACCCCCACCACCAGCGGCGAGGACGTGAGAGACTTCGCAAAGGTCCTGAAGAAC |
| | AAGTTCAGAACCAAGAGATACTTCGCTAAGCACCCCAGAATGGGCTACCTGCCCGTG |
| | CAGACCGTGCTGGAGGGCGATAACATGGAGACCGACACCATGTGA (SEQ ID NO: |
| | 2405) |
| First DRG de- | CGGCCTGATTCACAACACCAGCT (SEQ ID NO: 2400) |
| targeting | |
| miRNA binding | |
| site | |
| Second DRG | CGGCCTGATTCACAACACCAGCT (SEQ ID NO: 2400) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 7
(SEQ ID NO: 2418)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAAGTGGAGGACTGCTACGAGAGAGAGGACGTGCAGAAGAAGACCTTCACC
AAGTGGGTGAACGCCCAGTTCAGCAAGTTCGGCAAGCAGCACATCGAGAACCTGTTCAGCGACCTGCAGGACGGC
AGAAGACTGCTGGACCTGCTGGAGGGCCTGACCGGCCAGAAGCTGCCTAAGGAGAAGGGCAGCACCAGAGTGCAC
GCTCTCAACAACGTGAACAAGGCCCTGAGAGTGCTGCAGAACAACAACGTGGACCTTGTGAACATCGGAAGCACC
GACATCGTGGATGGCAACCACAAGCTGACCCTGGGACTGATCTGGAACATCATCCTGCACTGGCAGGTGAAGAAC
GTCATGAAGAACATCATGGCCGGCCTGCAGCAGACCAACAGCGAAAAGATCCTGCTGAGCTGGGTCAGACAGAGC
ACCAGAAACTACCCTCAGGTGAACGTGATCAACTTCACCACCAGCTGGAGCGACGGCCTGGCCCTGAACGCCCTG
ATCCACAGCCACAGACCCGACCTGTTCGACTGGAACAGCGTGGTGTGCCAGCAGAGCGCCACCCAGAGACTGGAG
CACGCCTTCAACATCGCCAGATACCAGCTGGGCATCGAGAAGCTGCTGGACCCCGAGGACGTGGATACCACCTAC
CCCGACAAGAAGAGCATCCTGATGTACATCACCAGCCTGTTCCAGGTGCTGCCCCAGCAAGTGAGCATCGAGGCC
ATCCAGGAGGTGGAGATGCTGCCCAGACCTCCAAAGGTGACCAAGGAGGAGCACTTCCAGCTGCACCACCAGATG
CACTACAGCCAGCAGATCACCGTGAGCCTGGCCCAGGGCTACGAGAGAACCAGCAGCCCCAAGCCCAGATTCAAG
AGCTACGCCTACACCCAGGCCGCCTACGTGACCACCAGCGACCCCACCAGAAGCCCCTTCCCCAGCCAGCACCTG
GAGGCCCCCGAGGATAAGAGCTTCGGCAGCAGCCTGATGGAGAGCGAGGTGAACTTGGACAGATACCAGACCGCC
CTGGAGGAGGTGCTGAGCTGGCTGCTGAGCGCCGAGGACACCCTTCAGGCACAGGGCGAGATCAGCAACGACGTG
GAGGTGGTGAAGGACCAGTTCCACACCCACGAGGGCTACATGATGGACCTGACCGCCCACCAGGGCAGAGTGGGT
AACATCCTCCAGCTGGGAAGCAAGCTGATCGGCACCGGCAAGCTGAGCGAGGACGAGGAGACCGAGGTGCAAGAG
CAAATGAACCTGCTTAACAGCAGATGGGAGTGCCTGAGAGTGGCCAGCATGGAGAAGCAGAGCAACCTGCACAGA
GTGCTGATGGACCTGCAGAACCAGAAGCTGAAGGAGCTGAACGACTGGCTGACCAAGACCGAGGAGAGAACCAGA
AAGATGGAGGAGGAGCCCCTGGGCCCGGACCTGGAGGACCTGAAGCGGCAGGTGCAGCAGCACAAGGTCTTACAG
GAGGACCTGGAGCAGGAGCAAGTGAGAGTGAACAGCCTGACCCACATGGTGGTGGTGGTGGATGAGAGCAGCGGC
GACCACGCCACCGCCGCCCTGGAGGAGCAGCTGAAGGTGCTGGGCGACAGATGGGCCAACATCTGCAGATGGACC
GAGGACAGATGGGTTCTGCTGCAGGACATCCTGCTGAAGTGGCAGAGACTGACCGAGGAACAGTGCCTGTTCAGC
GCCTGGCTGAGTGAGAAGGAAGACGCTGTGAACAAGATCCACACCACCGGCTTCAAGGACCAGAACGAGATGCTG
AGCAGCCTGCAGAAGCTGGCCGTGCTGAAGGCAGACCTGGAGAAGAAAAAGCAGTCCATGGGAAAGCTGTACAGC
CTGAAGCAAGACCTGCTGAGCACCCTGAAGAACAAGAGCGTGACCCAGAAGACCGAGGCCTGGCTGGACAACTTC
GCCAGATGCTGGGACAACCTGGTTCAGAAGCTGGAGAAGAGCACCGCCCAGATCAGCCAGGCCGTGACCACCACC
CAGCCCAGCCTGACCCAGACCACCGTGATGGAGACCGTGACCACCGTGACCACCAGAGAGCAGATCCTGGTGAAG
CACGCCCAGGAGGAGCTGCCCCCTCCTCCCCCGCAAAAGAAGAGAACCCTGGAGAGACTGCAGGAGCTGCAGGAG
GCTACCGATGAGCTGGACCTGAAGCTGAGACAGGCCGAGGTGATCAAGGGCAGCTGGCAGCCCGTGGGTGACCTG
CTTATCGACAGCCTGCAGGACCACCTAGAGAAGGTGAAGGCCCTGAGAGGCGAGATCGCCCCTCTGAAGGAGAAC
GTGAGCCACGTGAACGACCTGGCCAGACAGCTGACCACCCTGGGAATCCAGCTGAGCCCCTACAACCTGAGCACC
CTGGAGGACCTGAACACGAGATGGAAGCTGCTGCAAGTGGCCGTGGAGGACAGAGTCAGACAGTTGCACGAGGCT
CACAGAGATTTCGGCCCCGCTAGTCAGCACTTCCTGAGCACCAGCGTGCAGGGCCCCTGGGAGAGAGCTATCAGC
CCAAACAAGGTGCCCTACTACATCAACCACGAGACCCAGACCACCTGCTGGGACCACCCCAAGATGACCGAGCTG
TACCAGAGCCTGGCAGACCTCAACAACGTGAGATTCAGCGCCTACAGAACCGCCATGAAGCTGAGAAGACTGCAG
AAGGCCCTGTGCCTGGACCTGCTGAGCCTGAGCGCCGCCTGCGACGCCCTGGACCAGCACAACCTGAAGCAGAAC
GACCAGCCCATGGACATCCTTCAGATCATCAACTGCCTGACCACCATCTACGACAGACTGGAGCAGGAACATAAC
AACCTGGTGAACGTGCCCCTGTGCGTGGACATGTGCCTGAACTGGCTGCTGAACGTGTACGACACCGGCAGAACA
GGCAGAATCAGAGTGCTGAGCTTCAAGACCGGCATCATCAGCCTGTGCAAGGCCCACCTGGAGGACAAGTACAGA
TACTTATTCAAGCAGGTGGCCAGCAGCACCGGCTTCTGCGATCAGAGAAGACTGGGCCTGCTGCTGCACGACAGC
ATCCAGATCCCCAGACAGCTGGGCGAAGTGGCCAGCTTCGGCGGCAGCAACATCGAGCCCAGCGTGAGAAGCTGC
TTCCAGTTCGCTAACAACAAGCCCGAGATCGAGGCCGCCCTGTTCTTGGACTGGATGAGACTGGAGCCCCAGAGC
ATGGTGTGGCTGCCCGTGCTGCACAGAGTGGCCGCCGCCGAGACCGCCAAGCACCAGGCCAAGTGTAACATCTGC
AAGGAGTGCCCAATCATCGGCTTCAGATACAGAAGCCTGAAGCACTTCAACTACGACATCTGCCAGAGCTGCTTC
TTCAGCGGCAGAGTGGCCAAGGGCCACAAGATGCACTACCCCATGGTGGAGTACTGCACCCCCACCACCAGCGGC
GAGGACGTGAGAGACTTCGCAAAGGTCCTGAAGAACAAGTTCAGAACCAAGAGATACTTCGCTAAGCACCCCAGA
ATGGGCTACCTGCCCGTGCAGACCGTGCTGGAGGGCGATAACATGGAGACCGACACCATGTGATAGATTCTCATA
CAGCTAGATAACCAAAGAAGATCAGTCATACAGCTAGATAACCAAAGAAGACGCAGGAATAAAATATCTTTATTT
TCATTACATCTGTGTGTTGGTTTTTTGTGTGAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCAGTGAGCGAG
CGAGCGCGC

| Nucleic acid | |
|---|---|
| cargo 7 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | TAGTTATTAATAGTAATCAATTACGGGGTCATTAGTTCATAGCCCATATATGGAGTTC |
| promoter | CGCGTTACATAACTTACGGTAAATGGCCCGCCTGGCTGACCGCCCAACGACCCCCGCC |
| | CATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGT |
| | CATCGCTATTACCATGCTAGACTAGCATGCTGCCCATGTAAGGAGGCAAGGCCTGGGG |
| | ACACCCGAGATGCCTGGTTATAATTAACCCAGACATGTGGCTGCCCCCCCCCCCCAAC |
| | ACCTGCTGCCTCTAAAAATAACCCTGCATGCCATGTTCCCGGCGAAGGGCCAGCTGTC |
| | CCCCGCCAGCTAGACTCAGCTCTCAGGGGCCCCTCCCTGGGGACAGCCCCTCCTGGCT |
| | AGTCACACCCTGTAGGCTCCTCTATATAACCCAGGGGCACAGGGGCTGCCCTCATTCT |
| | ACCACCACCTCCACAGCACAGACAGACACTCAGGAGCCAGCCAGC (SEQ ID NO: |
| | 2402) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAGGAAGTGGAGGACTGCTACGAGAGAGAGGACGTGCAGAAGAAGA |
| optimized | CCTTCACCAAGTGGGTGAACGCCCAGTTCAGCAAGTTCGGCAAGCAGCACATCGAGAA |
| microdystroph | CCTGTTCAGCGACCTGCAGGACGGCAGAAGACTGCTGGACCTGCTGGAGGGCCTGACC |
| in coding | GGCCAGAAGCTGCCTAAGGAGAAGGGCAGCACCAGAGTGCACGCTCTCAACAACGTGA |
| sequence | ACAAGGCCCTGAGAGTGCTGCAGAACAACAACGTGGACCTTGTGAACATCGGAAGCAC |
| | CGACATCGTGGATGGCAACCACAAGCTGACCCTGGGACTGATCTGGAACATCATCCTG |
| | CACTGGCAGGTGAAGAACGTCATGAAGAACATCATGGCCGGCCTGCAGCAGACCAACA |
| | GCGAAAAGATCCTGCTGAGCTGGGTCAGACAGAGCACCAGAAACTACCCTCAGGTGAA |
| | CGTGATCAACTTCACCACCAGCTGGAGCGACGGCCTGGCCCTGAACGCCCTGATCCAC |
| | AGCCACAGACCCGACCTGTTCGACTGGAACAGCGTGGTGTGCCAGCAGAGCGCCACCC |
| | AGAGACTGGAGCACGCCTTCAACATCGCCAGATACCAGCTGGGCATCGAGAAGCTGCT |
| | GGACCCCGAGGACGTGGATACCACCTACCCCGACAAGAAGAGCATCCTGATGTACATC |
| | ACCAGCCTGTTCCAGGTGCTGCCCCAGCAAGTGAGCATCGAGGCCATCCAGGAGGTGG |
| | AGATGCTGCCCAGACCTCCAAAGGTGACCAAGGAGGAGCACTTCCAGCTGCACCACCA |
| | GATGCACTACAGCCAGCAGATCACCGTGAGCCTGGCCCAGGGCTACGAGAGAACCAGC |
| | AGCCCCAAGCCCAGATTCAAGAGCTACGCCTACACCCAGGCCGCCTACGTGACCACCA |
| | GCGACCCCACCAGAAGCCCCTTCCCCAGCCAGCACCTGGAGGCCCCCGAGGATAAGAG |
| | CTTCGGCAGCAGCCTGATGGAGAGCGAGGTGAACTTGGACAGATACCAGACCGCCCTG |
| | GAGGAGGTGCTGAGCTGGCTGCTGAGCGCCGAGGACACCCTTCAGGCACAGGGCGAGA |
| | TCAGCAACGACGTGGAGGTGGTGAAGGACCAGTTCCACACCCACGAGGGCTACATGAT |
| | GGACCTGACCGCCCACCAGGGCAGAGTGGGTAACATCCTCCAGCTGGGAAGCAAGCTG |
| | ATCGGCACCGGCAAGCTGAGCGAGGACGAGGAGACCGAGGTGCAAGAGCAAATGAACC |
| | TGCTTAACAGCAGATGGGAGTGCCTGAGAGTGGCCAGCATGGAGAAGCAGAGCAACCT |
| | GCACAGAGTGCTGATGGACCTGCAGAACCAGAAGCTGAAGGAGCTGAACGACTGGCTG |
| | ACCAAGACCGAGGAGAGAACCAGAAAGATGGAGGAGGAGCCCCTGGGCCCGGACCTGG |
| | AGGACCTGAAGCGGCAGGTGCAGCAGCACAAGGTCTTACAGGAGGACCTGGAGCAGGA |
| | GCAAGTGAGAGTGAACAGCCTGACCCACATGGTGGTGGTGGTGGATGAGAGCAGCGGC |
| | GACCACGCCACCGCCGCCCTGGAGGAGCAGCTGAAGGTGCTGGGCGACAGATGGGCCA |
| | ACATCTGCAGATGGACCGAGGACAGATGGGTTCTGCTGCAGGACATCCTGCTGAAGTG |
| | GCAGAGACTGACCGAGGAACAGTGCCTGTTCAGCGCCTGGCTGAGTGAGAAGGAAGAC |
| | GCTGTGAACAAGATCCACACCACCGGCTTCAAGGACCAGAACGAGATGCTGAGCAGCC |
| | TGCAGAAGCTGGCCGTGCTGAAGGCAGACCTGGAGAAGAAAAAGCAGTCCATGGGAAA |
| | GCTGTACAGCCTGAAGCAAGACCTGCTGAGCACCCTGAAGAACAAGAGCGTGACCCAG |
| | AAGACCGAGGCCTGGCTGGACAACTTCGCCAGATGCTGGGACAACCTGGTTCAGAAGC |
| | TGGAGAAGAGCACCGCCCAGATCAGCCAGGCCGTGACCACCACCCAGCCCAGCCTGAC |
| | CCAGACCACCGTGATGGAGACCGTGACCACCGTGACCACCAGAGAGCAGATCCTGGTG |
| | AAGCACGCCCAGGAGGAGCTGCCCCCTCCTCCCCCGCAAAAGAAGAGAACCCTGGAGA |
| | GACTGCAGGAGCTGCAGGAGGCTACCGATGAGCTGGACCTGAAGCTGAGACAGGCCGA |
| | GGTGATCAAGGGCAGCTGGCAGCCCGTGGGTGACCTGCTTATCGACAGCCTGCAGGAC |
| | CACCTAGAGAAGGTGAAGGCCCTGAGAGGCGAGATCGCCCCTCTGAAGGAGAACGTGA |
| | GCCACGTGAACGACCTGGCCAGACAGCTGACCACCCTGGGAATCCAGCTGAGCCCCTA |
| | CAACCTGAGCACCCTGGAGGACCTGAACACGAGATGGAAGCTGCTGCAAGTGGCCGTG |
| | GAGGACAGAGTCAGACAGTTGCACGAGGCTCACAGAGATTTCGGCCCCGCTAGTCAGC |
| | ACTTCCTGAGCACCAGCGTGCAGGGCCCCTGGGAGAGAGCTATCAGCCCAAACAAGGT |
| | GCCCTACTACATCAACCACGAGACCCAGACCACCTGCTGGGACCACCCCAAGATGACC |
| | GAGCTGTACCAGAGCCTGGCAGACCTCAACAACGTGAGATTCAGCGCCTACAGAACCG |
| | CCATGAAGCTGAGAAGACTGCAGAAGGCCCTGTGCCTGGACCTGCTGAGCCTGAGCGC |
| | CGCCTGCGACGCCCTGGACCAGCACAACCTGAAGCAGAACGACCAGCCCATGGACATC |
| | CTTCAGATCATCAACTGCCTGACCACCATCTACGACAGACTGGAGCAGGAACATAACA |
| | ACCTGGTGAACGTGCCCCTGTGCGTGGACATGTGCCTGAACTGGCTGCTGAACGTGTA |
| | CGACACCGGCAGAACAGGCAGAATCAGAGTGCTGAGCTTCAAGACCGGCATCATCAGC |
| | CTGTGCAAGGCCCACCTGGAGGACAAGTACAGATACTTATTCAAGCAGGTGGCCAGCA |
| | GCACCGGCTTCTGCGATCAGAGAAGACTGGGCCTGCTGCTGCACGACAGCATCCAGAT |
| | CCCCAGACAGCTGGGCGAAGTGGCCAGCTTCGGCGGCAGCAACATCGAGCCCAGCGTG |
| | AGAAGCTGCTTCCAGTTCGCTAACAACAAGCCCGAGATCGAGGCCGCCCTGTTCTTGG |
| | ACTGGATGAGACTGGAGCCCCAGAGCATGGTGTGGCTGCCCGTGCTGCACAGAGTGGC |
| | CGCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGCT |
| | TCTTCAGCGGCAGAGTGGCCAAGGGCCACAAGATGCACTACCCCATGGTGGAGTACTG |
| | CACCCCCACCACCAGCGGCGAGGACGTGAGAGACTTCGCAAAGGTCCTGAAGAACAAG |
| | TTCAGAACCAAGAGATACTTCGCTAAGCACCCCAGAATGGGCTACCTGCCCGTGCAGA |
| | CCGTGCTGGAGGGCGATAACATGGAGACCGACACCATGTGA (SEQ ID NO: |
| | 2405) |
| First DRG de- | TCATACAGCTAGATAACCAAAGA (SEQ ID NO: 2401) |
| targeting | |
| miRNA binding | |
| site | |
| Second DRG | TCATACAGCTAGATAACCAAAGA (SEQ ID NO: 2401) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 8
(SEQ ID NO: 2419)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 8 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | CGTCTGTCTAATTCCAGCCAAGGGCATGGGGGAAAGGACAGAGCCCAGGGACCTGTGG |
| promoter | CCTATTCTCTGAGAGGACGCTGGGCTAATAATAGGAGGGGAGATTAGGGAGGGATTTG |
| | ACTGCATTGTTATCTCACAGGAATGGCGTTTGAGAGCCTTGCTTCCAAGCTCTTCCAA |
| | GAGTCATGCCACCCCCCTCCAATCTCTGTGGAAGTGCCCACTGCCTTTCCCGCTTCGT |
| | TCTCTTCTCTCTAAATCTGAGCCCACCCCACTCAGGGGTACAGGATCTGGTTCATTTA |
| | TCTCCCAGAATGATGCCTTGGGTTGCGTCCCCTCTAAACTCTGGCACATTCCAGCCCC |
| | TCTTTGGGAGAAAGAACATCTCTTCTTGCCCAGGCCCTAGTGAGACATGCATCGCAAG |
| | ACATGCTCCCCCCTTCCTCCCCGCTGGGCTCCAGCTGCTGCCAGCCCTCCAAGAAAGG |
| | AGAGGCCCTGAGTTGGGCTATTTTGGTATCTGGGGTGGGCACCCGCAGGGCTAAGGTT |
| | ACCTTGGTATGTTAAGGGCTCCCTGGGGCAGGACTATATAACCCCAGAGGGACTGCCC |
| | CATGCTGACTCCTTGCTTCTTTCCTGCTCGAGCCGCTGCCTTGCCCCCCGGAGACTGA |
| | AGACA (SEQ ID NO: 2403) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAGGAAGTGGAGGACTGCTACGAGAGAGAGGACGTGCAGAAGAAGA |
| optimized | CCTTCACCAAGTGGGTGAACGCCCAGTTCAGCAAGTTCGGCAAGCAGCACATCGAGAA |
| microdystroph | CCTGTTCAGCGACCTGCAGGACGGCAGAAGACTGCTGGACCTGCTGGAGGGCCTGACC |
| in coding | GGCCAGAAGCTGCCTAAGGAGAAGGGCAGCACCAGAGTGCACGCTCTCAACAACGTGA |
| sequence | ACAAGGCCCTGAGAGTGCTGCAGAACAACAACGTGGACCTTGTGAACATCGGAAGCAC |
| | CGACATCGTGGATGGCAACCACAAGCTGACCCTGGGACTGATCTGGAACATCATCCTG |
| | CACTGGCAGGTGAAGAACGTCATGAAGAACATCATGGCCGGCCTGCAGCAGACCAACA |
| | GCGAAAAGATCCTGCTGAGCTGGGTCAGACAGAGCACCAGAAACTACCCTCAGGTGAA |
| | CGTGATCAACTTCACCACCAGCTGGAGCGACGGCCTGGCCCTGAACGCCCTGATCCAC |
| | AGCCACAGACCCGACCTGTTCGACTGGAACAGCGTGGTGTGCCAGCAGAGCGCCACCC |
| | AGAGACTGGAGCACGCCTTCAACATCGCCAGATACCAGCTGGGCATCGAGAAGCTGCT |
| | GGACCCCGAGGACGTGGATACCACCTACCCCGACAAGAAGAGCATCCTGATGTACATC |
| | ACCAGCCTGTTCCAGGTGCTGCCCCAGCAAGTGAGCATCGAGGCCATCCAGGAGGTGG |
| | AGATGCTGCCCAGACCTCCAAAGGTGACCAAGGAGGAGCACTTCCAGCTGCACCACCA |
| | GATGCACTACAGCCAGCAGATCACCGTGAGCCTGGCCCAGGGCTACGAGAGAACCAGC |
| | AGCCCCAAGCCCAGATTCAAGAGCTACGCCTACACCCAGGCCGCCTACGTGACCACCA |
| | GCGACCCCACCAGAAGCCCCTTCCCCAGCCAGCACCTGGAGGCCCCCGAGGATAAGAG |
| | CTTCGGCAGCAGCCTGATGGAGAGCGAGGTGAACTTGGACAGATACCAGACCGCCCTG |
| | GAGGAGGTGCTGAGCTGGCTGCTGAGCGCCGAGGACACCCTTCAGGCACAGGGCGAGA |
| | TCAGCAACGACGTGGAGGTGGTGAAGGACCAGTTCCACACCCACGAGGGCTACATGAT |
| | GGACCTGACCGCCCACCAGGGCAGAGTGGGTAACATCCTCCAGCTGGGAAGCAAGCTG |
| | ATCGGCACCGGCAAGCTGAGCGAGGACGAGGAGACCGAGGTGCAAGAGCAAATGAACC |
| | TGCTTAACAGCAGATGGGAGTGCCTGAGAGTGGCCAGCATGGAGAAGCAGAGCAACCT |
| | GCACAGAGTGCTGATGGACCTGCAGAACCAGAAGCTGAAGGAGCTGAACGACTGGCTG |
| | ACCAAGACCGAGGAGAGAACCAGAAAGATGGAGGAGGAGCCCCTGGGCCCGGACCTGG |
| | AGGACCTGAAGCGGCAGGTGCAGCAGCACAAGGTCTTACAGGAGGACCTGGAGCAGGA |
| | GCAAGTGAGAGTGAACAGCCTGACCCACATGGTGGTGGTGGTGGATGAGAGCAGCGGC |
| | GACCACGCCACCGCCGCCCTGGAGGAGCAGCTGAAGGTGCTGGGCGACAGATGGGCCA |
| | ACATCTGCAGATGGACCGAGGACAGATGGGTTCTGCTGCAGGACATCCTGCTGAAGTG |
| | GCAGAGACTGACCGAGGAACAGTGCCTGTTCAGCGCCTGGCTGAGTGAGAAGGAAGAC |
| | GCTGTGAACAAGATCCACACCACCGGCTTCAAGGACCAGAACGAGATGCTGAGCAGCC |
| | TGCAGAAGCTGGCCGTGCTGAAGGCAGACCTGGAGAAGAAAAAGCAGTCCATGGGAAA |
| | GCTGTACAGCCTGAAGCAAGACCTGCTGAGCACCCTGAAGAACAAGAGCGTGACCCAG |
| | AAGACCGAGGCCTGGCTGGACAACTTCGCCAGATGCTGGGACAACCTGGTTCAGAAGC |
| | TGGAGAAGAGCACCGCCCAGATCAGCCAGGCCGTGACCACCACCCAGCCCAGCCTGAC |
| | CCAGACCACCGTGATGGAGACCGTGACCACCGTGACCACCAGAGAGCAGATCCTGGTG |
| | AAGCACGCCCAGGAGGAGCTGCCCCCTCCTCCCCCGCAAAAGAAGAGAACCCTGGAGA |
| | GACTGCAGGAGCTGCAGGAGGCTACCGATGAGCTGGACCTGAAGCTGAGACAGGCCGA |
| | GGTGATCAAGGGCAGCTGGCAGCCCGTGGGTGACCTGCTTATCGACAGCCTGCAGGAC |
| | CACCTAGAGAAGGTGAAGGCCCTGAGAGGCGAGATCGCCCCTCTGAAGGAGAACGTGA |
| | GCCACGTGAACGACCTGGCCAGACAGCTGACCACCCTGGGAATCCAGCTGAGCCCCTA |
| | CAACCTGAGCACCCTGGAGGACCTGAACACGAGATGGAAGCTGCTGCAAGTGGCCGTG |
| | GAGGACAGAGTCAGACAGTTGCACGAGGCTCACAGAGATTTCGGCCCCGCTAGTCAGC |
| | ACTTCCTGAGCACCAGCGTGCAGGGCCCCTGGGAGAGAGCTATCAGCCCAAACAAGGT |
| | GCCCTACTACATCAACCACGAGACCCAGACCACCTGCTGGGACCACCCCAAGATGACC |
| | GAGCTGTACCAGAGCCTGGCAGACCTCAACAACGTGAGATTCAGCGCCTACAGAACCG |
| | CCATGAAGCTGAGAAGACTGCAGAAGGCCCTGTGCCTGGACCTGCTGAGCCTGAGCGC |
| | CGCCTGCGACGCCCTGGACCAGCACAACCTGAAGCAGAACGACCAGCCCATGGACATC |
| | CTTCAGATCATCAACTGCCTGACCACCATCTACGACAGACTGGAGCAGGAACATAACA |
| | ACCTGGTGAACGTGCCCCTGTGCGTGGACATGTGCCTGAACTGGCTGCTGAACGTGTA |
| | CGACACCGGCAGAACAGGCAGAATCAGAGTGCTGAGCTTCAAGACCGGCATCATCAGC |
| | CTGTGCAAGGCCCACCTGGAGGACAAGTACAGATACTTATTCAAGCAGGTGGCCAGCA |
| | GCACCGGCTTCTGCGATCAGAGAAGACTGGGCCTGCTGCTGCACGACAGCATCCAGAT |
| | CCCCAGACAGCTGGGCGAAGTGGCCAGCTTCGGCGGCAGCAACATCGAGCCCAGCGTG |
| | AGAAGCTGCTTCCAGTTCGCTAACAACAAGCCCGAGATCGAGGCCGCCCTGTTCTTGG |
| | ACTGGATGAGACTGGAGCCCCAGAGCATGGTGTGGCTGCCCGTGCTGCACAGAGTGGC |
| | CGCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGCT |
| | TCTTCAGCGGCAGAGTGGCCAAGGGCCACAAGATGCACTACCCCATGGTGGAGTACTG |
| | CACCCCCACCACCAGCGGCGAGGACGTGAGAGACTTCGCAAAGGTCCTGAAGAACAAG |
| | TTCAGAACCAAGAGATACTTCGCTAAGCACCCCAGAATGGGCTACCTGCCCGTGCAGA |
| | CCGTGCTGGAGGGCGATAACATGGAGACCGACACCATGTGA (SEQ ID NO: |
| | 2405) |
| First DRG de- | TCATACAGCTAGATAACCAAAGA (SEQ ID NO: 2401) |
| targeting | |
| miRNA binding | |
| site | |
| Second DRG | TCATACAGCTAGATAACCAAAGA (SEQ ID NO: 2401) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 9
(SEQ ID NO: 2420)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAAGTGGAGGACTGCT
ACGAGAGAGAGGACGTGCAGAAGAAGACCTTCACCAAGTGGGTGAACGCCCAGTTCAGCAAGTTCGGCAAGCAGC
ACATCGAGAACCTGTTCAGCGACCTGCAGGACGGCAGAAGACTGCTGGACCTGCTGGAGGGCCTGACCGGCCAGA
AGCTGCCTAAGGAGAAGGGCAGCACCAGAGTGCACGCTCTCAACAACGTGAACAAGGCCCTGAGAGTGCTGCAGA
ACAACAACGTGGACCTTGTGAACATCGGAAGCACCGACATCGTGGATGGCAACCACAAGCTGACCCTGGGACTGA
TCTGGAACATCATCCTGCACTGGCAGGTGAAGAACGTCATGAAGAACATCATGGCCGGCCTGCAGCAGACCAACA
GCGAAAAGATCCTGCTGAGCTGGGTCAGACAGAGCACCAGAAACTACCCTCAGGTGAACGTGATCAACTTCACCA
CCAGCTGGAGCGACGGCCTGGCCCTGAACGCCCTGATCCACAGCCACAGACCCGACCTGTTCGACTGGAACAGCG
TGGTGTGCCAGCAGAGCGCCACCCAGAGACTGGAGCACGCCTTCAACATCGCCAGATACCAGCTGGGCATCGAGA
AGCTGCTGGACCCCGAGGACGTGGATACCACCTACCCCGACAAGAAGAGCATCCTGATGTACATCACCAGCCTGT
TCCAGGTGCTGCCCCAGCAAGTGAGCATCGAGGCCATCCAGGAGGTGGAGATGCTGCCCAGACCTCCAAAGGTGA
CCAAGGAGGAGCACTTCCAGCTGCACCACCAGATGCACTACAGCCAGCAGATCACCGTGAGCCTGGCCCAGGGCT
ACGAGAGAACCAGCAGCCCCAAGCCCAGATTCAAGAGCTACGCCTACACCCAGGCCGCCTACGTGACCACCAGCG
ACCCCACCAGAAGCCCCTTCCCCAGCCAGCACCTGGAGGCCCCCGAGGATAAGAGCTTCGGCAGCAGCCTGATGG
AGAGCGAGGTGAACTTGGACAGATACCAGACCGCCCTGGAGGAGGTGCTGAGCTGGCTGCTGAGCGCCGAGGACA
CCCTTCAGGCACAGGGCGAGATCAGCAACGACGTGGAGGTGGTGAAGGACCAGTTCCACACCCACGAGGGCTACA
TGATGGACCTGACCGCCCACCAGGGCAGAGTGGGTAACATCCTCCAGCTGGGAAGCAAGCTGATCGGCACCGGCA
AGCTGAGCGAGGACGAGGAGACCGAGGTGCAAGAGCAAATGAACCTGCTTAACAGCAGATGGGAGTGCCTGAGAG
TGGCCAGCATGGAGAAGCAGAGCAACCTGCACAGAGTGCTGATGGACCTGCAGAACCAGAAGCTGAAGGAGCTGA
ACGACTGGCTGACCAAGACCGAGGAGAGAACCAGAAAGATGGAGGAGGAGCCCCTGGGCCCGGACCTGGAGGACC
TGAAGCGGCAGGTGCAGCAGCACAAGGTCTTACAGGAGGACCTGGAGCAGGAGCAAGTGAGAGTGAACAGCCTGA
CCCACATGGTGGTGGTGGTGGATGAGAGCAGCGGCGACCACGCCACCGCCGCCCTGGAGGAGCAGCTGAAGGTGC
TGGGCGACAGATGGGCCAACATCTGCAGATGGACCGAGGACAGATGGGTTCTGCTGCAGGACATCCTGCTGAAGT
GGCAGAGACTGACCGAGGAACAGTGCCTGTTCAGCGCCTGGCTGAGTGAGAAGGAAGACGCTGTGAACAAGATCC
ACACCACCGGCTTCAAGGACCAGAACGAGATGCTGAGCAGCCTGCAGAAGCTGGCCGTGCTGAAGGCAGACCTGG
AGAAGAAAAAGCAGTCCATGGGAAAGCTGTACAGCCTGAAGCAAGACCTGCTGAGCACCCTGAAGAACAAGAGCG
TGACCCAGAAGACCGAGGCCTGGCTGGACAACTTCGCCAGATGCTGGGACAACCTGGTTCAGAAGCTGGAGAAGA
GCACCGCCCAGATCAGCCAGGCCGTGACCACCACCCAGCCCAGCCTGACCCAGACCACCGTGATGGAGACCGTGA
CCACCGTGACCACCAGAGAGCAGATCCTGGTGAAGCACGCCCAGGAGGAGCTGCCCCCTCCTCCCCCGCAAAAGA
AGAGAACCCTGGAGAGACTGCAGGAGCTGCAGGAGGCTACCGATGAGCTGGACCTGAAGCTGAGACAGGCCGAGG
TGATCAAGGGCAGCTGGCAGCCCGTGGGTGACCTGCTTATCGACAGCCTGCAGGACCACCTAGAGAAGGTGAAGG
CCCTGAGAGGCGAGATCGCCCCTCTGAAGGAGAACGTGAGCCACGTGAACGACCTGGCCAGACAGCTGACCACCC
TGGGAATCCAGCTGAGCCCCTACAACCTGAGCACCCTGGAGGACCTGAACACGAGATGGAAGCTGCTGCAAGTGG
CCGTGGAGGACAGAGTCAGACAGTTGCACGAGGCTCACAGAGATTTCGGCCCCGCTAGTCAGCACTTCCTGAGCA
CCAGCGTGCAGGGCCCCTGGGAGAGAGCTATCAGCCCAAACAAGGTGCCCTACTACATCAACCACGAGACCCAGA
CCACCTGCTGGGACCACCCCAAGATGACCGAGCTGTACCAGAGCCTGGCAGACCTCAACAACGTGAGATTCAGCG
CCTACAGAACCGCCATGAAGCTGAGAAGACTGCAGAAGGCCCTGTGCCTGGACCTGCTGAGCCTGAGCGCCGCCT
GCGACGCCCTGGACCAGCACAACCTGAAGCAGAACGACCAGCCCATGGACATCCTTCAGATCATCAACTGCCTGA
CCACCATCTACGACAGACTGGAGCAGGAACATAACAACCTGGTGAACGTGCCCCTGTGCGTGGACATGTGCCTGA
ACTGGCTGCTGAACGTGTACGACACCGGCAGAACAGGCAGAATCAGAGTGCTGAGCTTCAAGACCGGCATCATCA
GCCTGTGCAAGGCCCACCTGGAGGACAAGTACAGATACTTATTCAAGCAGGTGGCCAGCAGCACCGGCTTCTGCG
ATCAGAGAAGACTGGGCCTGCTGCTGCACGACAGCATCCAGATCCCCAGACAGCTGGGCGAAGTGGCCAGCTTCG
GCGGCAGCAACATCGAGCCCAGCGTGAGAAGCTGCTTCCAGTTCGCTAACAACAAGCCCGAGATCGAGGCCGCCC
TGTTCTTGGACTGGATGAGACTGGAGCCCCAGAGCATGGTGTGGCTGCCCGTGCTGCACAGAGTGGCCGCCGCCG
AGACCGCCAAGCACCAGGCCAAGTGTAACATCTGCAAGGAGTGCCCAATCATCGGCTTCAGATACAGAAGCCTGA
AGCACTTCAACTACGACATCTGCCAGAGCTGCTTCTTCAGCGGCAGAGTGGCCAAGGGCCACAAGATGCACTACC
CCATGGTGGAGTACTGCACCCCCACCACCAGCGGCGAGGACGTGAGAGACTTCGCAAAGGTCCTGAAGAACAAGT
TCAGAACCAAGAGATACTTCGCTAAGCACCCCAGAATGGGCTACCTGCCCGTGCAGACCGTGCTGGAGGGCGATA
ACATGGAGACCGACACCATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 9 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | CGTCTGTCTAATTCCAGCCAAGGGCATGGGGGAAAGGACAGAGCCCAGGGACCTGTGG |
| promoter | CCTATTCTCTGAGAGGACGCTGGGCTAATAATAGGAGGGGAGATTAGGGAGGGATTTG |
| | ACTGCATTGTTATCTCACAGGAATGGCGTTTGAGAGCCTTGCTTCCAAGCTCTTCCAA |
| | GAGTCATGCCACCCCCCTCCAATCTCTGTGGAAGTGCCCACTGCCTTTCCCGCTTCGT |
| | TCTCTTCTCTCGGTCCCATCCCTGACCCACTAGCCTGGTGCCGGGGGGAGACCAATTT |
| | CTGGAGGAAGCTCTGGGTACCCAGTCTTGCCCTAAGCCTGCCTCTGAACAGGCTGTTC |
| | TTGTTTATGACCCCCGTCCCAGCAGAATAGTGATTGCTCATCCCTGCATGTGATTTGA |
| | GAGGGCCAGTCTTTCAGGCCTGGAGGAAACTCAAGCCCCACCAGCCGCTTGGCATCGG |
| | AATGCTCAGCTGGTCCCCTCCTGGGGCTCATGTGACCAAGATCTAGCTATAAATATCA |
| | GAGGGGCCTCAGACCAAGACAGAATTCGGGCACCAGGAGAAGGAAGCCAACAGGATCC |
| | GACCCGGTGTTTTGTGACAAAGGCAAGACCCCCAGGTCTACTTAGAGCAACGTTAGTA |
| | GAGGA (SEQ ID NO: 2404) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAGGAAGTGGAGGACTGCTACGAGAGAGAGGACGTGCAGAAGAAGA |
| optimized | CCTTCACCAAGTGGGTGAACGCCCAGTTCAGCAAGTTCGGCAAGCAGCACATCGAGAA |
| microdystroph | CCTGTTCAGCGACCTGCAGGACGGCAGAAGACTGCTGGACCTGCTGGAGGGCCTGACC |
| in coding | GGCCAGAAGCTGCCTAAGGAGAAGGGCAGCACCAGAGTGCACGCTCTCAACAACGTGA |
| sequence | ACAAGGCCCTGAGAGTGCTGCAGAACAACAACGTGGACCTTGTGAACATCGGAAGCAC |
| | CGACATCGTGGATGGCAACCACAAGCTGACCCTGGGACTGATCTGGAACATCATCCTG |
| | CACTGGCAGGTGAAGAACGTCATGAAGAACATCATGGCCGGCCTGCAGCAGACCAACA |
| | GCGAAAAGATCCTGCTGAGCTGGGTCAGACAGAGCACCAGAAACTACCCTCAGGTGAA |
| | CGTGATCAACTTCACCACCAGCTGGAGCGACGGCCTGGCCCTGAACGCCCTGATCCAC |
| | AGCCACAGACCCGACCTGTTCGACTGGAACAGCGTGGTGTGCCAGCAGAGCGCCACCC |
| | AGAGACTGGAGCACGCCTTCAACATCGCCAGATACCAGCTGGGCATCGAGAAGCTGCT |
| | GGACCCCGAGGACGTGGATACCACCTACCCCGACAAGAAGAGCATCCTGATGTACATC |
| | ACCAGCCTGTTCCAGGTGCTGCCCCAGCAAGTGAGCATCGAGGCCATCCAGGAGGTGG |
| | AGATGCTGCCCAGACCTCCAAAGGTGACCAAGGAGGAGCACTTCCAGCTGCACCACCA |
| | GATGCACTACAGCCAGCAGATCACCGTGAGCCTGGCCCAGGGCTACGAGAGAACCAGC |
| | AGCCCCAAGCCCAGATTCAAGAGCTACGCCTACACCCAGGCCGCCTACGTGACCACCA |
| | GCGACCCCACCAGAAGCCCCTTCCCCAGCCAGCACCTGGAGGCCCCCGAGGATAAGAG |
| | CTTCGGCAGCAGCCTGATGGAGAGCGAGGTGAACTTGGACAGATACCAGACCGCCCTG |
| | GAGGAGGTGCTGAGCTGGCTGCTGAGCGCCGAGGACACCCTTCAGGCACAGGGCGAGA |
| | TCAGCAACGACGTGGAGGTGGTGAAGGACCAGTTCCACACCCACGAGGGCTACATGAT |
| | GGACCTGACCGCCCACCAGGGCAGAGTGGGTAACATCCTCCAGCTGGGAAGCAAGCTG |
| | ATCGGCACCGGCAAGCTGAGCGAGGACGAGGAGACCGAGGTGCAAGAGCAAATGAACC |
| | TGCTTAACAGCAGATGGGAGTGCCTGAGAGTGGCCAGCATGGAGAAGCAGAGCAACCT |
| | GCACAGAGTGCTGATGGACCTGCAGAACCAGAAGCTGAAGGAGCTGAACGACTGGCTG |
| | ACCAAGACCGAGGAGAGAACCAGAAAGATGGAGGAGGAGCCCCTGGGCCCGGACCTGG |
| | AGGACCTGAAGCGGCAGGTGCAGCAGCACAAGGTCTTACAGGAGGACCTGGAGCAGGA |
| | GCAAGTGAGAGTGAACAGCCTGACCCACATGGTGGTGGTGGTGGATGAGAGCAGCGGC |
| | GACCACGCCACCGCCGCCCTGGAGGAGCAGCTGAAGGTGCTGGGCGACAGATGGGCCA |
| | ACATCTGCAGATGGACCGAGGACAGATGGGTTCTGCTGCAGGACATCCTGCTGAAGTG |
| | GCAGAGACTGACCGAGGAACAGTGCCTGTTCAGCGCCTGGCTGAGTGAGAAGGAAGAC |
| | GCTGTGAACAAGATCCACACCACCGGCTTCAAGGACCAGAACGAGATGCTGAGCAGCC |
| | TGCAGAAGCTGGCCGTGCTGAAGGCAGACCTGGAGAAGAAAAAGCAGTCCATGGGAAA |
| | GCTGTACAGCCTGAAGCAAGACCTGCTGAGCACCCTGAAGAACAAGAGCGTGACCCAG |
| | AAGACCGAGGCCTGGCTGGACAACTTCGCCAGATGCTGGGACAACCTGGTTCAGAAGC |
| | TGGAGAAGAGCACCGCCCAGATCAGCCAGGCCGTGACCACCACCCAGCCCAGCCTGAC |
| | CCAGACCACCGTGATGGAGACCGTGACCACCGTGACCACCAGAGAGCAGATCCTGGTG |
| | AAGCACGCCCAGGAGGAGCTGCCCCCTCCTCCCCCGCAAAAGAAGAGAACCCTGGAGA |
| | GACTGCAGGAGCTGCAGGAGGCTACCGATGAGCTGGACCTGAAGCTGAGACAGGCCGA |
| | GGTGATCAAGGGCAGCTGGCAGCCCGTGGGTGACCTGCTTATCGACAGCCTGCAGGAC |
| | CACCTAGAGAAGGTGAAGGCCCTGAGAGGCGAGATCGCCCCTCTGAAGGAGAACGTGA |
| | GCCACGTGAACGACCTGGCCAGACAGCTGACCACCCTGGGAATCCAGCTGAGCCCCTA |
| | CAACCTGAGCACCCTGGAGGACCTGAACACGAGATGGAAGCTGCTGCAAGTGGCCGTG |
| | GAGGACAGAGTCAGACAGTTGCACGAGGCTCACAGAGATTTCGGCCCCGCTAGTCAGC |
| | ACTTCCTGAGCACCAGCGTGCAGGGCCCCTGGGAGAGAGCTATCAGCCCAAACAAGGT |
| | GCCCTACTACATCAACCACGAGACCCAGACCACCTGCTGGGACCACCCCAAGATGACC |
| | GAGCTGTACCAGAGCCTGGCAGACCTCAACAACGTGAGATTCAGCGCCTACAGAACCG |
| | CCATGAAGCTGAGAAGACTGCAGAAGGCCCTGTGCCTGGACCTGCTGAGCCTGAGCGC |
| | CGCCTGCGACGCCCTGGACCAGCACAACCTGAAGCAGAACGACCAGCCCATGGACATC |
| | CTTCAGATCATCAACTGCCTGACCACCATCTACGACAGACTGGAGCAGGAACATAACA |
| | ACCTGGTGAACGTGCCCCTGTGCGTGGACATGTGCCTGAACTGGCTGCTGAACGTGTA |
| | CGACACCGGCAGAACAGGCAGAATCAGAGTGCTGAGCTTCAAGACCGGCATCATCAGC |
| | CTGTGCAAGGCCCACCTGGAGGACAAGTACAGATACTTATTCAAGCAGGTGGCCAGCA |
| | GCACCGGCTTCTGCGATCAGAGAAGACTGGGCCTGCTGCTGCACGACAGCATCCAGAT |
| | CCCCAGACAGCTGGGCGAAGTGGCCAGCTTCGGCGGCAGCAACATCGAGCCCAGCGTG |
| | AGAAGCTGCTTCCAGTTCGCTAACAACAAGCCCGAGATCGAGGCCGCCCTGTTCTTGG |
| | ACTGGATGAGACTGGAGCCCCAGAGCATGGTGTGGCTGCCCGTGCTGCACAGAGTGGC |
| | CGCCGCCGAGACCGCCAAGCACCAGGCCAAGTGTAACATCTGCAAGGAGTGCCCAATC |
| | ATCGGCTTCAGATACAGAAGCCTGAAGCACTTCAACTACGACATCTGCCAGAGCTGCT |
| | TCTTCAGCGGCAGAGTGGCCAAGGGCCACAAGATGCACTACCCCATGGTGGAGTACTG |
| | CACCCCCACCACCAGCGGCGAGGACGTGAGAGACTTCGCAAAGGTCCTGAAGAACAAG |
| | TTCAGAACCAAGAGATACTTCGCTAAGCACCCCAGAATGGGCTACCTGCCCGTGCAGA |
| | CCGTGCTGGAGGGCGATAACATGGAGACCGACACCATGTGA (SEQ ID NO: |
| | 2405) |
| First DRG de- | TCATACAGCTAGATAACCAAAGA (SEQ ID NO: 2401) |
| targeting | |
| miRNA binding | |
| site | |
| Second DRG | TCATACAGCTAGATAACCAAAGA (SEQ ID NO: 2401) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 10
(SEQ ID NO: 2421)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAAGAGGTCGAGGACTGCTACGAGCGCGAAGATGTGCAGAAGAAAACTTTCACT
AAATGGGTGAATGCCCAGTTCTCTAAGTTCGGAAAGCAGCACATCGAAAACCTGTTTTCCGATCTGCAAGATGGC
CGGCGGCTGCTGGACCTGCTGGAGGGGCTGACCGGACAGAAGCTGCCTAAGGAGAAAGGATCTACCAGGGTCCAT
GCTCTGAATAACGTGAACAAGGCTCTGAGGGTGCTGCAGAACAATAACGTGGACCTGGTGAACATTGGTTCCACA
GACATCGTGGATGGAAACCACAAACTGACACTGGGACTGATCTGGAACATCATTCTGCATTGGCAAGTGAAGAAC
GTGATGAAGAACATCATGGCCGGACTGCAGCAGACGAACTCCGAGAAGATCCTGCTTAGCTGGGTGAGACAGAGT
ACCCGGAATTACCCACAGGTGAACGTGATCAATTTCACAACCAGCTGGTCAGACGGCCTGGCTCTGAATGCCCTG
ATTCATAGCCACCGGCCAGATCTGTTTGACTGGAACTCTGTCGTGTGTCAGCAGTCAGCCACCCAGAGGCTGGAG
CACGCCTTCAATATTGCCCGCTATCAGCTGGGGATTGAGAAACTGCTGGACCCCGAGGATGTGGACACAACATAC
CCTGACAAGAAGAGCATACTTATGTATATCACAAGCCTGTTCCAGGTGCTGCCACAGCAGGTCTCCATCGAGGCC
ATTCAAGAAGTGGAGATGCTGCCCAGGCCTCCTAAGGTCACCAAAGAGGAGCACTTTCAGCTCCATCATCAGATG
CACTACAGCCAGCAAATTACCGTGTCCCTGGCCCAGGGCTACGAGAGGACAAGTTCCCCCAAGCCTAGATTTAAG
AGCTATGCCTATACCCAGGCCGCCTACGTGACTACCTCCGATCCCACCAGGAGCCCATTTCCCAGCCAGCACCTG
GAGGCTCCCGAAGATAAGAGCTTTGGCAGCAGCCTGATGGAATCTGAGGTGAACCTGGACAGATACCAGACTGCG
CTGGAGGAGGTGCTGTCCTGGCTGCTGAGCGCCGAGGACACCCTGCAGGCTCAGGGCGAGATTAGTAATGATGTG
GAGGTGGTGAAGGATCAGTTTCACACTCACGAAGGCTACATGATGGACCTGACCGCCCACCAGGGCAGGGTGGGG
AACATTCTGCAGCTCGGGAGTAAGCTGATAGGGACCGGCAAACTGTCCGAGGATGAGGAAACCGAGGTGCAGGAG
CAGATGAACCTGCTTAACTCCCGGTGGGAGTGTCTGAGAGTGGCCAGCATGGAGAAGCAGAGCAATCTGCACAGA
GTGCTGATGGATCTGCAGAATCAGAAGCTGAAGGAACTGAATGATTGGCTCACAAAGACAGAGGAGAGGACTAGA
AAGATGGAGGAGGAGCCCCTGGGCCCTGACCTGGAGGACCTGAAGCGACAGGTGCAGCAGCACAAGGTGCTGCAG
GAGGATCTGGAGCAAGAACAAGTGAGGGTGAACTCTCTGACTCATATGGTTGTGGTGGTGGACGAGTCTTCCGGA
GACCACGCAACTGCCGCCCTCGAGGAGCAGCTGAAGGTGCTGGGCGATAGGTGGGCTAATATCTGTAGGTGGACC
GAGGACAGATGGGTGCTGCTGCAGGATATCCTGCTGAAATGGCAGCGCCTGACAGAAGAGCAGTGTCTGTTCAGC
GCATGGCTGAGCGAAAAGGAGGACGCTGTGAACAAGATCCACACTACAGGATTTAAAGACCAGAACGAGATGCTG
AGTAGCCTGCAGAAGCTGGCTGTGCTGAAGGCTGATTTGGAGAAAAAGAAGCAGAGCATGGGCAAGCTGTACTCC
CTGAAACAGGACCTGCTGAGCACCCTGAAGAATAAGTCCGTGACCCAGAAAACCGAAGCTTGGCTGGACAACTTC
GCGCGCTGCTGGGATAACCTGGTGCAGAAGCTGGAGAAGAGCACCGCCCAGATTTCTCAAGCAGTGACCACAACC
CAGCCCAGCCTGACCCAGACTACAGTAATGGAGACAGTCACTACCGTGACCACCCGGGAGCAGATCCTGGTGAAG
CACGCCCAGGAAGAGCTGCCGCCCCCACCACCCCAGAAAAAGCGGACCCTGGAGCGCCTGCAGGAGCTCCAGGAG
GCCACCGACGAACTGGATCTGAAGCTGAGGCAGGCCGAGGTGATTAAGGGATCCTGGCAGCCTGTGGGAGACCTG
CTGATCGACAGCCTGCAGGATCATCTGGAGAAAGTCAAAGCTCTGAGAGGGGAGATCGCCCCACTGAAGGAGAAC
GTGAGCCACGTGAATGACCTGGCTAGACAGCTGACCACCTTGGGCATTCAGCTGAGTCCTTACAATCTGTCAACT
CTGGAGGATCTGAACACCCGGTGGAAACTGCTGCAGGTGGCTGTGGAAGATCGGGTGCGCCAGTTACATGAGGCC
CACAGGGACTTTGGCCCAGCCTCACAGCACTTTCTGTCAACCTCTGTGCAAGGACCTTGGGAGAGAGCAATCAGC
CCAAACAAGGTGCCATATTATATCAATCATGAGACTCAGACAACTTGCTGGGATCACCCCAAGATGACAGAACTC
TATCAGTCACTCGCCGATCTCAATAATGTACGGTTCTCTGCCTACCGGACAGCTATGAAGCTGCGGAGGCTGCAG
AAGGCACTCTGTCTGGACCTGCTGAGTCTCTCTGCGGCCTGCGATGCCCTGGATCAACATAACCTGAAGCAGAAT
GATCAGCCTATGGACATCCTGCAGATCATTAACTGTCTCACCACAATCTACGATCGGCTGGAACAGGAACATAAC
AACCTGGTGAATGTGCCACTGTGTGTGGACATGTGCCTGAATTGGCTGCTCAACGTCTACGATACAGGCAGGACT
GGCAGGATCCGCGTGCTGAGCTTTAAAACCGGCATCATCTCTCTGTGCAAAGCCCACCTGGAGGACAAATATCGG
TACCTGTTTAAGCAGGTGGCCTCCAGCACAGGCTTCTGTGATCAGCGCCGGCTGGGCCTGCTGCTGCACGACAGC
ATTCAGATCCCTAGGCAGCTGGGCGAGGTGGCCTCCTTCGGCGGCTCAAACATTGAACCCAGCGTGAGATCTTGC
TTTCAGTTTGCAAATAATAAACCCGAGATCGAGGCCGCCCTGTTCCTGGATTGGATGAGACTGGAGCCTCAGAGT
ATGGTGTGGCTGCCAGTGCTGCATAGAGTGGCCGCCGCTGAGACGGCCAAGCACCAGGCAAAGTGCAATATCTGC
AAGGAATGCCCCATTATTGGCTTTCGATACCGGTCCTTGAAACACTTCAATTATGACATCTGCCAGTCATGCTTC
TTCAGCGGACGCGTGGCCAAGGGACACAAGATGCATTATCCTATGGTGGAATACTGCACTCCAACTACTTCCGGC
GAGGACGTGAGAGATTTCGCTAAAGTTCTTAAAAACAAATTTCGGACTAAGAGGTACTTCGCCAAGCACCCCCGC
ATGGGCTATCTGCCTGTGCAGACAGTGCTTGAAGGAGACAACATGGAAACCGATACAATGTGATAGATTCCAACA
AAATCACTGATGCTGGAAGATCAGCAACAAAATCACTGATGCTGGAAGACGCAGGAATAAAATATCTTTATTTTC
ATTACATCTGTGTGTTGGTTTTTTGTGTGAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGTGAGCGAGCG
AGCGCGC

| Nucleic acid | |
|---|---|
| cargo 10 | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | TAGTTATTAATAGTAATCAATTACGGGGTCATTAGTTCATAGCCCATATATGGAGTT |
| promoter | CCGCGTTACATAACTTACGGTAAATGGCCCGCCTGGCTGACCGCCCAACGACCCCCG |
| | CCCATTGACGTCAATAATGACGTATGTTCCCATAGTAACGCCAATAGGGACTTTCCA |
| | TTGACGTCAATGGGTGGAGTATTTACGGTAAACTGCCCACTTGGCAGTACATCAAGT |
| | GTATCATATGCCAAGTACGCCCCCTATTGACGTCAATGACGGTAAATGGCCCGCCTG |
| | GCATTATGCCCAGTACATGACCTTATGGGACTTTCCTACTTGGCAGTACATCTACGT |
| | ATTAGTCATCGCTATTACCATGCTAGACTAGCATGCTGCCCATGTAAGGAGGCAAGG |
| | CCTGGGGACACCCGAGATGCCTGGTTATAATTAACCCAGACATGTGGCTGCCCCCCC |
| | CCCCCAACACCTGCTGCCTCTAAAAATAACCCTGCATGCCATGTTCCCGGCGAAGGG |
| | CCAGCTGTCCCCCGCCAGCTAGACTCAGCTCTCAGGGGCCCCTCCCTGGGGACAGCC |
| | CCTCCTGGCTAGTCACACCCTGTAGGCTCCTCTATATAACCCAGGGGCACAGGGGCT |
| | GCCCTCATTCTACCACCACCTCCACAGCACAGACAGACACTCAGGAGCCAGCCAGC |
| | (SEQ ID NO: 2402) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAAGAGGTCGAGGACTGCTACGAGCGCGAAGATGTGCAGAAGAAA |
| optimized | ACTTTCACTAAATGGGTGAATGCCCAGTTCTCTAAGTTCGGAAAGCAGCACATCGAA |
| microdystroph | AACCTGTTTTCCGATCTGCAAGATGGCCGGCGGCTGCTGGACCTGCTGGAGGGGCTG |
| in coding | ACCGGACAGAAGCTGCCTAAGGAGAAAGGATCTACCAGGGTCCATGCTCTGAATAAC |
| sequence | GTGAACAAGGCTCTGAGGGTGCTGCAGAACAATAACGTGGACCTGGTGAACATTGGT |
| | TCCACAGACATCGTGGATGGAAACCACAAACTGACACTGGGACTGATCTGGAACATC |
| | ATTCTGCATTGGCAAGTGAAGAACGTGATGAAGAACATCATGGCCGGACTGCAGCAG |
| | ACGAACTCCGAGAAGATCCTGCTTAGCTGGGTGAGACAGAGTACCCGGAATTACCCA |
| | CAGGTGAACGTGATCAATTTCACAACCAGCTGGTCAGACGGCCTGGCTCTGAATGCC |
| | CTGATTCATAGCCACCGGCCAGATCTGTTTGACTGGAACTCTGTCGTGTGTCAGCAG |
| | TCAGCCACCCAGAGGCTGGAGCACGCCTTCAATATTGCCCGCTATCAGCTGGGGATT |
| | GAGAAACTGCTGGACCCCGAGGATGTGGACACAACATACCCTGACAAGAAGAGCATA |
| | CTTATGTATATCACAAGCCTGTTCCAGGTGCTGCCACAGCAGGTCTCCATCGAGGCC |
| | ATTCAAGAAGTGGAGATGCTGCCCAGGCCTCCTAAGGTCACCAAAGAGGAGCACTTT |
| | CAGCTCCATCATCAGATGCACTACAGCCAGCAAATTACCGTGTCCCTGGCCCAGGGC |
| | TACGAGAGGACAAGTTCCCCCAAGCCTAGATTTAAGAGCTATGCCTATACCCAGGCC |
| | GCCTACGTGACTACCTCCGATCCCACCAGGAGCCCATTTCCCAGCCAGCACCTGGAG |
| | GCTCCCGAAGATAAGAGCTTTGGCAGCAGCCTGATGGAATCTGAGGTGAACCTGGAC |
| | AGATACCAGACTGCGCTGGAGGAGGTGCTGTCCTGGCTGCTGAGCGCCGAGGACACC |
| | CTGCAGGCTCAGGGCGAGATTAGTAATGATGTGGAGGTGGTGAAGGATCAGTTTCAC |
| | ACTCACGAAGGCTACATGATGGACCTGACCGCCCACCAGGGCAGGGTGGGGAACATT |
| | CTGCAGCTCGGGAGTAAGCTGATAGGGACCGGCAAACTGTCCGAGGATGAGGAAACC |
| | GAGGTGCAGGAGCAGATGAACCTGCTTAACTCCCGGTGGGAGTGTCTGAGAGTGGCC |
| | AGCATGGAGAAGCAGAGCAATCTGCACAGAGTGCTGATGGATCTGCAGAATCAGAAG |
| | CTGAAGGAACTGAATGATTGGCTCACAAAGACAGAGGAGAGGACTAGAAAGATGGAG |
| | GAGGAGCCCCTGGGCCCTGACCTGGAGGACCTGAAGCGACAGGTGCAGCAGCACAAG |
| | GTGCTGCAGGAGGATCTGGAGCAAGAACAAGTGAGGGTGAACTCTCTGACTCATATG |
| | GTTGTGGTGGTGGACGAGTCTTCCGGAGACCACGCAACTGCCGCCCTCGAGGAGCAG |
| | CTGAAGGTGCTGGGCGATAGGTGGGCTAATATCTGTAGGTGGACCGAGGACAGATGG |
| | GTGCTGCTGCAGGATATCCTGCTGAAATGGCAGCGCCTGACAGAAGAGCAGTGTCTG |
| | TTCAGCGCATGGCTGAGCGAAAAGGAGGACGCTGTGAACAAGATCCACACTACAGGA |
| | TTTAAAGACCAGAACGAGATGCTGAGTAGCCTGCAGAAGCTGGCTGTGCTGAAGGCT |
| | GATTTGGAGAAAAAGAAGCAGAGCATGGGCAAGCTGTACTCCCTGAAACAGGACCTG |
| | CTGAGCACCCTGAAGAATAAGTCCGTGACCCAGAAAACCGAAGCTTGGCTGGACAAC |
| | TTCGCGCGCTGCTGGGATAACCTGGTGCAGAAGCTGGAGAAGAGCACCGCCCAGATT |
| | TCTCAAGCAGTGACCACAACCCAGCCCAGCCTGACCCAGACTACAGTAATGGAGACA |
| | GTCACTACCGTGACCACCCGGGAGCAGATCCTGGTGAAGCACGCCCAGGAAGAGCTG |
| | CCGCCCCCACCACCCCAGAAAAAGCGGACCCTGGAGCGCCTGCAGGAGCTCCAGGAG |
| | GCCACCGACGAACTGGATCTGAAGCTGAGGCAGGCCGAGGTGATTAAGGGATCCTGG |
| | CAGCCTGTGGGAGACCTGCTGATCGACAGCCTGCAGGATCATCTGGAGAAAGTCAAA |
| | GCTCTGAGAGGGGAGATCGCCCCACTGAAGGAGAACGTGAGCCACGTGAATGACCTG |
| | GCTAGACAGCTGACCACCTTGGGCATTCAGCTGAGTCCTTACAATCTGTCAACTCTG |
| | GAGGATCTGAACACCCGGTGGAAACTGCTGCAGGTGGCTGTGGAAGATCGGGTGCGC |
| | CAGTTACATGAGGCCCACAGGGACTTTGGCCCAGCCTCACAGCACTTTCTGTCAACC |
| | TCTGTGCAAGGACCTTGGGAGAGAGCAATCAGCCCAAACAAGGTGCCATATTATATC |
| | AATCATGAGACTCAGACAACTTGCTGGGATCACCCCAAGATGACAGAACTCTATCAG |
| | TCACTCGCCGATCTCAATAATGTACGGTTCTCTGCCTACCGGACAGCTATGAAGCTG |
| | CGGAGGCTGCAGAAGGCACTCTGTCTGGACCTGCTGAGTCTCTCTGCGGCCTGCGAT |
| | GCCCTGGATCAACATAACCTGAAGCAGAATGATCAGCCTATGGACATCCTGCAGATC |
| | ATTAACTGTCTCACCACAATCTACGATCGGCTGGAACAGGAACATAACAACCTGGTG |
| | AATGTGCCACTGTGTGTGGACATGTGCCTGAATTGGCTGCTCAACGTCTACGATACA |
| | GGCAGGACTGGCAGGATCCGCGTGCTGAGCTTTAAAACCGGCATCATCTCTCTGTGC |
| | AAAGCCCACCTGGAGGACAAATATCGGTACCTGTTTAAGCAGGTGGCCTCCAGCACA |
| | GGCTTCTGTGATCAGCGCCGGCTGGGCCTGCTGCTGCACGACAGCATTCAGATCCCT |
| | AGGCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGGAT |
| | TGGATGAGACTGGAGCCTCAGAGTATGGTGTGGCTGCCAGTGCTGCATAGAGTGGCC |
| | GCCGCTGAGACGGCCAAGCACCAGGCAAAGTGCAATATCTGCAAGGAATGCCCCATT |
| | ATTGGCTTTCGATACCGGTCCTTGAAACACTTCAATTATGACATCTGCCAGTCATGC |
| | TTCTTCAGCGGACGCGTGGCCAAGGGACACAAGATGCATTATCCTATGGTGGAATAC |
| | TGCACTCCAACTACTTCCGGCGAGGACGTGAGAGATTTCGCTAAAGTTCTTAAAAAC |
| | AAATTTCGGACTAAGAGGTACTTCGCCAAGCACCCCCGCATGGGCTATCTGCCTGTG |
| | CAGACAGTGCTTGAAGGAGACAACATGGAAACCGATACAATGTGA (SEQ ID NO: |
| | 2406) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 11
(SEQ ID NO: 2422)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 11 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | CGTCTGTCTAATTCCAGCCAAGGGCATGGGGGAAAGGACAGAGCCCAGGGACCTGTGG |
| promoter | CCTATTCTCTGAGAGGACGCTGGGCTAATAATAGGAGGGGAGATTAGGGAGGGATTTG |
| | ACTGCATTGTTATCTCACAGGAATGGCGTTTGAGAGCCTTGCTTCCAAGCTCTTCCAA |
| | GAGTCATGCCACCCCCCTCCAATCTCTGTGGAAGTGCCCACTGCCTTTCCCGCTTCGT |
| | TCTCTTCTCTCTAAATCTGAGCCCACCCCACTCAGGGGTACAGGATCTGGTTCATTTA |
| | TCTCCCAGAATGATGCCTTGGGTTGCGTCCCCTCTAAACTCTGGCACATTCCAGCCCC |
| | TCTTTGGGAGAAAGAACATCTCTTCTTGCCCAGGCCCTAGTGAGACATGCATCGCAAG |
| | ACATGCTCCCCCCTTCCTCCCCGCTGGGCTCCAGCTGCTGCCAGCCCTCCAAGAAAGG |
| | AGAGGCCCTGAGTTGGGCTATTTTGGTATCTGGGGTGGGCACCCGCAGGGCTAAGGTT |
| | ACCTTGGTATGTTAAGGGCTCCCTGGGGCAGGACTATATAACCCCAGAGGGACTGCCC |
| | CATGCTGACTCCTTGCTTCTTTCCTGCTCGAGCCGCTGCCTTGCCCCCCGGAGACTGA |
| | AGACA (SEQ ID NO: 2403) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAAGAGGTCGAGGACTGCTACGAGCGCGAAGATGTGCAGAAGAAAA |
| optimized | CTTTCACTAAATGGGTGAATGCCCAGTTCTCTAAGTTCGGAAAGCAGCACATCGAAAA |
| microdystroph | CCTGTTTTCCGATCTGCAAGATGGCCGGCGGCTGCTGGACCTGCTGGAGGGGCTGACC |
| in coding | GGACAGAAGCTGCCTAAGGAGAAAGGATCTACCAGGGTCCATGCTCTGAATAACGTGA |
| sequence | ACAAGGCTCTGAGGGTGCTGCAGAACAATAACGTGGACCTGGTGAACATTGGTTCCAC |
| | AGACATCGTGGATGGAAACCACAAACTGACACTGGGACTGATCTGGAACATCATTCTG |
| | CATTGGCAAGTGAAGAACGTGATGAAGAACATCATGGCCGGACTGCAGCAGACGAACT |
| | CCGAGAAGATCCTGCTTAGCTGGGTGAGACAGAGTACCCGGAATTACCCACAGGTGAA |
| | CGTGATCAATTTCACAACCAGCTGGTCAGACGGCCTGGCTCTGAATGCCCTGATTCAT |
| | AGCCACCGGCCAGATCTGTTTGACTGGAACTCTGTCGTGTGTCAGCAGTCAGCCACCC |
| | AGAGGCTGGAGCACGCCTTCAATATTGCCCGCTATCAGCTGGGGATTGAGAAACTGCT |
| | GGACCCCGAGGATGTGGACACAACATACCCTGACAAGAAGAGCATACTTATGTATATC |
| | ACAAGCCTGTTCCAGGTGCTGCCACAGCAGGTCTCCATCGAGGCCATTCAAGAAGTGG |
| | AGATGCTGCCCAGGCCTCCTAAGGTCACCAAAGAGGAGCACTTTCAGCTCCATCATCA |
| | GATGCACTACAGCCAGCAAATTACCGTGTCCCTGGCCCAGGGCTACGAGAGGACAAGT |
| | TCCCCCAAGCCTAGATTTAAGAGCTATGCCTATACCCAGGCCGCCTACGTGACTACCT |
| | CCGATCCCACCAGGAGCCCATTTCCCAGCCAGCACCTGGAGGCTCCCGAAGATAAGAG |
| | CTTTGGCAGCAGCCTGATGGAATCTGAGGTGAACCTGGACAGATACCAGACTGCGCTG |
| | GAGGAGGTGCTGTCCTGGCTGCTGAGCGCCGAGGACACCCTGCAGGCTCAGGGCGAGA |
| | TTAGTAATGATGTGGAGGTGGTGAAGGATCAGTTTCACACTCACGAAGGCTACATGAT |
| | GGACCTGACCGCCCACCAGGGCAGGGTGGGGAACATTCTGCAGCTCGGGAGTAAGCTG |
| | ATAGGGACCGGCAAACTGTCCGAGGATGAGGAAACCGAGGTGCAGGAGCAGATGAACC |
| | TGCTTAACTCCCGGTGGGAGTGTCTGAGAGTGGCCAGCATGGAGAAGCAGAGCAATCT |
| | GCACAGAGTGCTGATGGATCTGCAGAATCAGAAGCTGAAGGAACTGAATGATTGGCTC |
| | ACAAAGACAGAGGAGAGGACTAGAAAGATGGAGGAGGAGCCCCTGGGCCCTGACCTGG |
| | AGGACCTGAAGCGACAGGTGCAGCAGCACAAGGTGCTGCAGGAGGATCTGGAGCAAGA |
| | ACAAGTGAGGGTGAACTCTCTGACTCATATGGTTGTGGTGGTGGACGAGTCTTCCGGA |
| | GACCACGCAACTGCCGCCCTCGAGGAGCAGCTGAAGGTGCTGGGCGATAGGTGGGCTA |
| | ATATCTGTAGGTGGACCGAGGACAGATGGGTGCTGCTGCAGGATATCCTGCTGAAATG |
| | GCAGCGCCTGACAGAAGAGCAGTGTCTGTTCAGCGCATGGCTGAGCGAAAAGGAGGAC |
| | GCTGTGAACAAGATCCACACTACAGGATTTAAAGACCAGAACGAGATGCTGAGTAGCC |
| | TGCAGAAGCTGGCTGTGCTGAAGGCTGATTTGGAGAAAAAGAAGCAGAGCATGGGCAA |
| | GCTGTACTCCCTGAAACAGGACCTGCTGAGCACCCTGAAGAATAAGTCCGTGACCCAG |
| | AAAACCGAAGCTTGGCTGGACAACTTCGCGCGCTGCTGGGATAACCTGGTGCAGAAGC |
| | TGGAGAAGAGCACCGCCCAGATTTCTCAAGCAGTGACCACAACCCAGCCCAGCCTGAC |
| | CCAGACTACAGTAATGGAGACAGTCACTACCGTGACCACCCGGGAGCAGATCCTGGTG |
| | AAGCACGCCCAGGAAGAGCTGCCGCCCCCACCACCCCAGAAAAAGCGGACCCTGGAGC |
| | GCCTGCAGGAGCTCCAGGAGGCCACCGACGAACTGGATCTGAAGCTGAGGCAGGCCGA |
| | GGTGATTAAGGGATCCTGGCAGCCTGTGGGAGACCTGCTGATCGACAGCCTGCAGGAT |
| | CATCTGGAGAAAGTCAAAGCTCTGAGAGGGGAGATCGCCCCACTGAAGGAGAACGTGA |
| | GCCACGTGAATGACCTGGCTAGACAGCTGACCACCTTGGGCATTCAGCTGAGTCCTTA |
| | CAATCTGTCAACTCTGGAGGATCTGAACACCCGGTGGAAACTGCTGCAGGTGGCTGTG |
| | GAAGATCGGGTGCGCCAGTTACATGAGGCCCACAGGGACTTTGGCCCAGCCTCACAGC |
| | ACTTTCTGTCAACCTCTGTGCAAGGACCTTGGGAGAGAGCAATCAGCCCAAACAAGGT |
| | GCCATATTATATCAATCATGAGACTCAGACAACTTGCTGGGATCACCCCAAGATGACA |
| | GAACTCTATCAGTCACTCGCCGATCTCAATAATGTACGGTTCTCTGCCTACCGGACAG |
| | CTATGAAGCTGCGGAGGCTGCAGAAGGCACTCTGTCTGGACCTGCTGAGTCTCTCTGC |
| | GGCCTGCGATGCCCTGGATCAACATAACCTGAAGCAGAATGATCAGCCTATGGACATC |
| | CTGCAGATCATTAACTGTCTCACCACAATCTACGATCGGCTGGAACAGGAACATAACA |
| | ACCTGGTGAATGTGCCACTGTGTGTGGACATGTGCCTGAATTGGCTGCTCAACGTCTA |
| | CGATACAGGCAGGACTGGCAGGATCCGCGTGCTGAGCTTTAAAACCGGCATCATCTCT |
| | CTGTGCAAAGCCCACCTGGAGGACAAATATCGGTACCTGTTTAAGCAGGTGGCCTCCA |
| | GCACAGGCTTCTGTGATCAGCGCCGGCTGGGCCTGCTGCTGCACGACAGCATTCAGAT |
| | CCCTAGGCAGCTGGGCGAGGTGGCCTCCTTCGGCGGCTCAAACATTGAACCCAGCGTG |
| | AGATCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGTGGC |
| | CGCCGCTGAGACGGCCAAGCACCAGGCAAAGTGCAATATCTGCAAGGAATGCCCCATT |
| | ATTGGCTTTCGATACCGGTCCTTGAAACACTTCAATTATGACATCTGCCAGTCATGCT |
| | TCTTCAGCGGACGCGTGGCCAAGGGACACAAGATGCATTATCCTATGGTGGAATACTG |
| | CACTCCAACTACTTCCGGCGAGGACGTGAGAGATTTCGCTAAAGTTCTTAAAAACAAA |
| | TTTCGGACTAAGAGGTACTTCGCCAAGCACCCCCGCATGGGCTATCTGCCTGTGCAGA |
| | CAGTGCTTGAAGGAGACAACATGGAAACCGATACAATGTGA (SEQ ID NO: |
| | 2406) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 12
(SEQ ID NO: 2423)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 12 | |
| annotated | Sequence |
| AAV2 ITR | GCGCGCTCGCTCGCTCACTGAGGCCGCCCGGGCAAAGCCCGGGCGTCGGGCGACCTT |
| | TGGTCGCCCGGCCTCAGTGAGCGAGCGAGCGCGCAGAGAGGGAGTGGCCAACTCCAT |
| | CACTAGGGGTTCCT (SEQ ID NO: 2408) |
| Novel | CGTCTGTCTAATTCCAGCCAAGGGCATGGGGGAAAGGACAGAGCCCAGGGACCTGTG |
| promoter | GCCTATTCTCTGAGAGGACGCTGGGCTAATAATAGGAGGGGAGATTAGGGAGGGATT |
| | TGACTGCATTGTTATCTCACAGGAATGGCGTTTGAGAGCCTTGCTTCCAAGCTCTTC |
| | CAAGAGTCATGCCACCCCCCTCCAATCTCTGTGGAAGTGCCCACTGCCTTTCCCGCT |
| | TCGTTCTCTTCTCTCGGTCCCATCCCTGACCCACTAGCCTGGTGCCGGGGGGAGACC |
| | AATTTCTGGAGGAAGCTCTGGGTACCCAGTCTTGCCCTAAGCCTGCCTCTGAACAGG |
| | CTGTTCTTGTTTATGACCCCCGTCCCAGCAGAATAGTGATTGCTCATCCCTGCATGT |
| | GATTTGAGAGGGCCAGTCTTTCAGGCCTGGAGGAAACTCAAGCCCCACCAGCCGCTT |
| | GGCATCGGAATGCTCAGCTGGTCCCCTCCTGGGGCTCATGTGACCAAGATCTAGCTA |
| | TAAATATCAGAGGGGCCTCAGACCAAGACAGAATTCGGGCACCAGGAGAAGGAAGCC |
| | AACAGGATCCGACCCGGTGTTTTGTGACAAAGGCAAGACCCCCAGGTCTACTTAGAG |
| | CAACGTTAGTAGAGGA (SEQ ID NO: 2404) |
| Intron | GTAAGTATCAAGGTTACAAGACAGGTTTAAGGAGACCAATAGAAACTGGGCTTGTCG |
| | AGACAGAGAAGACTCTTGCGTTTCTGATAGGCACCTATTGGTCTTACTGACATCCAC |
| | TTTGCCTTTCTCTCCACAG (SEQ ID NO: 2409) |
| Codon | ATGCTGTGGTGGGAAGAGGTCGAGGACTGCTACGAGCGCGAAGATGTGCAGAAGAAA |
| optimized | ACTTTCACTAAATGGGTGAATGCCCAGTTCTCTAAGTTCGGAAAGCAGCACATCGAA |
| microdystroph | AACCTGTTTTCCGATCTGCAAGATGGCCGGCGGCTGCTGGACCTGCTGGAGGGGCTG |
| in coding | ACCGGACAGAAGCTGCCTAAGGAGAAAGGATCTACCAGGGTCCATGCTCTGAATAAC |
| sequence | GTGAACAAGGCTCTGAGGGTGCTGCAGAACAATAACGTGGACCTGGTGAACATTGGT |
| | TCCACAGACATCGTGGATGGAAACCACAAACTGACACTGGGACTGATCTGGAACATC |
| | ATTCTGCATTGGCAAGTGAAGAACGTGATGAAGAACATCATGGCCGGACTGCAGCAG |
| | ACGAACTCCGAGAAGATCCTGCTTAGCTGGGTGAGACAGAGTACCCGGAATTACCCA |
| | CAGGTGAACGTGATCAATTTCACAACCAGCTGGTCAGACGGCCTGGCTCTGAATGCC |
| | CTGATTCATAGCCACCGGCCAGATCTGTTTGACTGGAACTCTGTCGTGTGTCAGCAG |
| | TCAGCCACCCAGAGGCTGGAGCACGCCTTCAATATTGCCCGCTATCAGCTGGGGATT |
| | GAGAAACTGCTGGACCCCGAGGATGTGGACACAACATACCCTGACAAGAAGAGCATA |
| | CTTATGTATATCACAAGCCTGTTCCAGGTGCTGCCACAGCAGGTCTCCATCGAGGCC |
| | ATTCAAGAAGTGGAGATGCTGCCCAGGCCTCCTAAGGTCACCAAAGAGGAGCACTTT |
| | CAGCTCCATCATCAGATGCACTACAGCCAGCAAATTACCGTGTCCCTGGCCCAGGGC |
| | TACGAGAGGACAAGTTCCCCCAAGCCTAGATTTAAGAGCTATGCCTATACCCAGGCC |
| | GCCTACGTGACTACCTCCGATCCCACCAGGAGCCCATTTCCCAGCCAGCACCTGGAG |
| | GCTCCCGAAGATAAGAGCTTTGGCAGCAGCCTGATGGAATCTGAGGTGAACCTGGAC |
| | AGATACCAGACTGCGCTGGAGGAGGTGCTGTCCTGGCTGCTGAGCGCCGAGGACACC |
| | CTGCAGGCTCAGGGCGAGATTAGTAATGATGTGGAGGTGGTGAAGGATCAGTTTCAC |
| | ACTCACGAAGGCTACATGATGGACCTGACCGCCCACCAGGGCAGGGTGGGGAACATT |
| | CTGCAGCTCGGGAGTAAGCTGATAGGGACCGGCAAACTGTCCGAGGATGAGGAAACC |
| | GAGGTGCAGGAGCAGATGAACCTGCTTAACTCCCGGTGGGAGTGTCTGAGAGTGGCC |
| | AGCATGGAGAAGCAGAGCAATCTGCACAGAGTGCTGATGGATCTGCAGAATCAGAAG |
| | CTGAAGGAACTGAATGATTGGCTCACAAAGACAGAGGAGAGGACTAGAAAGATGGAG |
| | GAGGAGCCCCTGGGCCCTGACCTGGAGGACCTGAAGCGACAGGTGCAGCAGCACAAG |
| | GTGCTGCAGGAGGATCTGGAGCAAGAACAAGTGAGGGTGAACTCTCTGACTCATATG |
| | GTTGTGGTGGTGGACGAGTCTTCCGGAGACCACGCAACTGCCGCCCTCGAGGAGCAG |
| | CTGAAGGTGCTGGGCGATAGGTGGGCTAATATCTGTAGGTGGACCGAGGACAGATGG |
| | GTGCTGCTGCAGGATATCCTGCTGAAATGGCAGCGCCTGACAGAAGAGCAGTGTCTG |
| | TTCAGCGCATGGCTGAGCGAAAAGGAGGACGCTGTGAACAAGATCCACACTACAGGA |
| | TTTAAAGACCAGAACGAGATGCTGAGTAGCCTGCAGAAGCTGGCTGTGCTGAAGGCT |
| | GATTTGGAGAAAAAGAAGCAGAGCATGGGCAAGCTGTACTCCCTGAAACAGGACCTG |
| | CTGAGCACCCTGAAGAATAAGTCCGTGACCCAGAAAACCGAAGCTTGGCTGGACAAC |
| | TTCGCGCGCTGCTGGGATAACCTGGTGCAGAAGCTGGAGAAGAGCACCGCCCAGATT |
| | TCTCAAGCAGTGACCACAACCCAGCCCAGCCTGACCCAGACTACAGTAATGGAGACA |
| | GTCACTACCGTGACCACCCGGGAGCAGATCCTGGTGAAGCACGCCCAGGAAGAGCTG |
| | CCGCCCCCACCACCCCAGAAAAAGCGGACCCTGGAGCGCCTGCAGGAGCTCCAGGAG |
| | GCCACCGACGAACTGGATCTGAAGCTGAGGCAGGCCGAGGTGATTAAGGGATCCTGG |
| | CAGCCTGTGGGAGACCTGCTGATCGACAGCCTGCAGGATCATCTGGAGAAAGTCAAA |
| | GCTCTGAGAGGGGAGATCGCCCCACTGAAGGAGAACGTGAGCCACGTGAATGACCTG |
| | GCTAGACAGCTGACCACCTTGGGCATTCAGCTGAGTCCTTACAATCTGTCAACTCTG |
| | GAGGATCTGAACACCCGGTGGAAACTGCTGCAGGTGGCTGTGGAAGATCGGGTGCGC |
| | CAGTTACATGAGGCCCACAGGGACTTTGGCCCAGCCTCACAGCACTTTCTGTCAACC |
| | TCTGTGCAAGGACCTTGGGAGAGAGCAATCAGCCCAAACAAGGTGCCATATTATATC |
| | AATCATGAGACTCAGACAACTTGCTGGGATCACCCCAAGATGACAGAACTCTATCAG |
| | TCACTCGCCGATCTCAATAATGTACGGTTCTCTGCCTACCGGACAGCTATGAAGCTG |
| | CGGAGGCTGCAGAAGGCACTCTGTCTGGACCTGCTGAGTCTCTCTGCGGCCTGCGAT |
| | GCCCTGGATCAACATAACCTGAAGCAGAATGATCAGCCTATGGACATCCTGCAGATC |
| | ATTAACTGTCTCACCACAATCTACGATCGGCTGGAACAGGAACATAACAACCTGGTG |
| | AATGTGCCACTGTGTGTGGACATGTGCCTGAATTGGCTGCTCAACGTCTACGATACA |
| | GGCAGGACTGGCAGGATCCGCGTGCTGAGCTTTAAAACCGGCATCATCTCTCTGTGC |
| | AAAGCCCACCTGGAGGACAAATATCGGTACCTGTTTAAGCAGGTGGCCTCCAGCACA |
| | GGCTTCTGTGATCAGCGCCGGCTGGGCCTGCTGCTGCACGACAGCATTCAGATCCCT |
| | AGGCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGGAT |
| | TGGATGAGACTGGAGCCTCAGAGTATGGTGTGGCTGCCAGTGCTGCATAGAGTGGCC |
| | GCCGCTGAGACGGCCAAGCACCAGGCAAAGTGCAATATCTGCAAGGAATGCCCCATT |
| | ATTGGCTTTCGATACCGGTCCTTGAAACACTTCAATTATGACATCTGCCAGTCATGC |
| | TTCTTCAGCGGACGCGTGGCCAAGGGACACAAGATGCATTATCCTATGGTGGAATAC |
| | TGCACTCCAACTACTTCCGGCGAGGACGTGAGAGATTTCGCTAAAGTTCTTAAAAAC |
| | AAATTTCGGACTAAGAGGTACTTCGCCAAGCACCCCCGCATGGGCTATCTGCCTGTG |
| | CAGACAGTGCTTGAAGGAGACAACATGGAAACCGATACAATGTGA (SEQ ID NO: |
| | 2406) |
| First DRG de- | CAACAAAATCACTGATGCTGGA (SEQ ID NO: 2399) |
| targeting | |
| miRNA binding | |
| site | |
| Second DRG | CAACAAAATCACTGATGCTGGA (SEQ ID NO: 2399) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGCCTCAGTGA |
| | GCGAGCGAGCGCGC (SEQ ID NO: 2411) |

Nucleic acid 13
(SEQ ID NO: 2424)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAAGAGGTCGAGGACTGCTACGAGCGCGAAGATGTGCAGAAGAAAACTTTCACT
AAATGGGTGAATGCCCAGTTCTCTAAGTTCGGAAAGCAGCACATCGAAAACCTGTTTTCCGATCTGCAAGATGGC
CGGCGGCTGCTGGACCTGCTGGAGGGGCTGACCGGACAGAAGCTGCCTAAGGAGAAAGGATCTACCAGGGTCCAT
GCTCTGAATAACGTGAACAAGGCTCTGAGGGTGCTGCAGAACAATAACGTGGACCTGGTGAACATTGGTTCCACA
GACATCGTGGATGGAAACCACAAACTGACACTGGGACTGATCTGGAACATCATTCTGCATTGGCAAGTGAAGAAC
GTGATGAAGAACATCATGGCCGGACTGCAGCAGACGAACTCCGAGAAGATCCTGCTTAGCTGGGTGAGACAGAGT
ACCCGGAATTACCCACAGGTGAACGTGATCAATTTCACAACCAGCTGGTCAGACGGCCTGGCTCTGAATGCCCTG
ATTCATAGCCACCGGCCAGATCTGTTTGACTGGAACTCTGTCGTGTGTCAGCAGTCAGCCACCCAGAGGCTGGAG
CACGCCTTCAATATTGCCCGCTATCAGCTGGGGATTGAGAAACTGCTGGACCCCGAGGATGTGGACACAACATAC
CCTGACAAGAAGAGCATACTTATGTATATCACAAGCCTGTTCCAGGTGCTGCCACAGCAGGTCTCCATCGAGGCC
ATTCAAGAAGTGGAGATGCTGCCCAGGCCTCCTAAGGTCACCAAAGAGGAGCACTTTCAGCTCCATCATCAGATG
CACTACAGCCAGCAAATTACCGTGTCCCTGGCCCAGGGCTACGAGAGGACAAGTTCCCCCAAGCCTAGATTTAAG
AGCTATGCCTATACCCAGGCCGCCTACGTGACTACCTCCGATCCCACCAGGAGCCCATTTCCCAGCCAGCACCTG
GAGGCTCCCGAAGATAAGAGCTTTGGCAGCAGCCTGATGGAATCTGAGGTGAACCTGGACAGATACCAGACTGCG
CTGGAGGAGGTGCTGTCCTGGCTGCTGAGCGCCGAGGACACCCTGCAGGCTCAGGGCGAGATTAGTAATGATGTG
GAGGTGGTGAAGGATCAGTTTCACACTCACGAAGGCTACATGATGGACCTGACCGCCCACCAGGGCAGGGTGGGG
AACATTCTGCAGCTCGGGAGTAAGCTGATAGGGACCGGCAAACTGTCCGAGGATGAGGAAACCGAGGTGCAGGAG
CAGATGAACCTGCTTAACTCCCGGTGGGAGTGTCTGAGAGTGGCCAGCATGGAGAAGCAGAGCAATCTGCACAGA
GTGCTGATGGATCTGCAGAATCAGAAGCTGAAGGAACTGAATGATTGGCTCACAAAGACAGAGGAGAGGACTAGA
AAGATGGAGGAGGAGCCCCTGGGCCCTGACCTGGAGGACCTGAAGCGACAGGTGCAGCAGCACAAGGTGCTGCAG
GAGGATCTGGAGCAAGAACAAGTGAGGGTGAACTCTCTGACTCATATGGTTGTGGTGGTGGACGAGTCTTCCGGA
GACCACGCAACTGCCGCCCTCGAGGAGCAGCTGAAGGTGCTGGGCGATAGGTGGGCTAATATCTGTAGGTGGACC
GAGGACAGATGGGTGCTGCTGCAGGATATCCTGCTGAAATGGCAGCGCCTGACAGAAGAGCAGTGTCTGTTCAGC
GCATGGCTGAGCGAAAAGGAGGACGCTGTGAACAAGATCCACACTACAGGATTTAAAGACCAGAACGAGATGCTG
AGTAGCCTGCAGAAGCTGGCTGTGCTGAAGGCTGATTTGGAGAAAAAGAAGCAGAGCATGGGCAAGCTGTACTCC
CTGAAACAGGACCTGCTGAGCACCCTGAAGAATAAGTCCGTGACCCAGAAAACCGAAGCTTGGCTGGACAACTTC
GCGCGCTGCTGGGATAACCTGGTGCAGAAGCTGGAGAAGAGCACCGCCCAGATTTCTCAAGCAGTGACCACAACC
CAGCCCAGCCTGACCCAGACTACAGTAATGGAGACAGTCACTACCGTGACCACCCGGGAGCAGATCCTGGTGAAG
CACGCCCAGGAAGAGCTGCCGCCCCCACCACCCCAGAAAAAGCGGACCCTGGAGCGCCTGCAGGAGCTCCAGGAG
GCCACCGACGAACTGGATCTGAAGCTGAGGCAGGCCGAGGTGATTAAGGGATCCTGGCAGCCTGTGGGAGACCTG
CTGATCGACAGCCTGCAGGATCATCTGGAGAAAGTCAAAGCTCTGAGAGGGGAGATCGCCCCACTGAAGGAGAAC
GTGAGCCACGTGAATGACCTGGCTAGACAGCTGACCACCTTGGGCATTCAGCTGAGTCCTTACAATCTGTCAACT
CTGGAGGATCTGAACACCCGGTGGAAACTGCTGCAGGTGGCTGTGGAAGATCGGGTGCGCCAGTTACATGAGGCC
CACAGGGACTTTGGCCCAGCCTCACAGCACTTTCTGTCAACCTCTGTGCAAGGACCTTGGGAGAGAGCAATCAGC
CCAAACAAGGTGCCATATTATATCAATCATGAGACTCAGACAACTTGCTGGGATCACCCCAAGATGACAGAACTC
TATCAGTCACTCGCCGATCTCAATAATGTACGGTTCTCTGCCTACCGGACAGCTATGAAGCTGCGGAGGCTGCAG
AAGGCACTCTGTCTGGACCTGCTGAGTCTCTCTGCGGCCTGCGATGCCCTGGATCAACATAACCTGAAGCAGAAT
GATCAGCCTATGGACATCCTGCAGATCATTAACTGTCTCACCACAATCTACGATCGGCTGGAACAGGAACATAAC
AACCTGGTGAATGTGCCACTGTGTGTGGACATGTGCCTGAATTGGCTGCTCAACGTCTACGATACAGGCAGGACT
GGCAGGATCCGCGTGCTGAGCTTTAAAACCGGCATCATCTCTCTGTGCAAAGCCCACCTGGAGGACAAATATCGG
TACCTGTTTAAGCAGGTGGCCTCCAGCACAGGCTTCTGTGATCAGCGCCGGCTGGGCCTGCTGCTGCACGACAGC
ATTCAGATCCCTAGGCAGCTGGGCGAGGTGGCCTCCTTCGGCGGCTCAAACATTGAACCCAGCGTGAGATCTTGC
TTTCAGTTTGCAAATAATAAACCCGAGATCGAGGCCGCCCTGTTCCTGGATTGGATGAGACTGGAGCCTCAGAGT
ATGGTGTGGCTGCCAGTGCTGCATAGAGTGGCCGCCGCTGAGACGGCCAAGCACCAGGCAAAGTGCAATATCTGC
AAGGAATGCCCCATTATTGGCTTTCGATACCGGTCCTTGAAACACTTCAATTATGACATCTGCCAGTCATGCTTC
TTCAGCGGACGCGTGGCCAAGGGACACAAGATGCATTATCCTATGGTGGAATACTGCACTCCAACTACTTCCGGC
GAGGACGTGAGAGATTTCGCTAAAGTTCTTAAAAACAAATTTCGGACTAAGAGGTACTTCGCCAAGCACCCCCGC
ATGGGCTATCTGCCTGTGCAGACAGTGCTTGAAGGAGACAACATGGAAACCGATACAATGTGATAGATTCCGGCC
TGATTCACAACACCAGCTAGATCAGCGGCCTGATTCACAACACCAGCTAGACGCAGGAATAAAATATCTTTATTT
TCATTACATCTGTGTGTTGGTTTTTTGTGTGAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCAGTGAGCGAG
CGAGCGCGC

| Nucleic acid | |
|---|---|
| cargo 13 | |
| annotated | Sequence |
| AAV2 ITR | GCGCGCTCGCTCGCTCACTGAGGCCGCCCGGGCAAAGCCCGGGCGTCGGGCGACCTTT |
| | GGTCGCCCGGCCTCAGTGAGCGAGCGAGCGCGCAGAGAGGGAGTGGCCAACTCCATCA |
| | CTAGGGGTTCCT (SEQ ID NO: 2408) |
| Novel | TAGTTATTAATAGTAATCAATTACGGGGTCATTAGTTCATAGCCCATATATGGAGTTC |
| promoter | CGCGTTACATAACTTACGGTAAATGGCCCGCCTGGCTGACCGCCCAACGACCCCCGCC |
| | CATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGT |
| | CATCGCTATTACCATGCTAGACTAGCATGCTGCCCATGTAAGGAGGCAAGGCCTGGGG |
| | ACACCCGAGATGCCTGGTTATAATTAACCCAGACATGTGGCTGCCCCCCCCCCCCAAC |
| | ACCTGCTGCCTCTAAAAATAACCCTGCATGCCATGTTCCCGGCGAAGGGCCAGCTGTC |
| | CCCCGCCAGCTAGACTCAGCTCTCAGGGGCCCCTCCCTGGGGACAGCCCCTCCTGGCT |
| | AGTCACACCCTGTAGGCTCCTCTATATAACCCAGGGGCACAGGGGCTGCCCTCATTCT |
| | ACCACCACCTCCACAGCACAGACAGACACTCAGGAGCCAGCCAGC (SEQ ID NO: |
| | 2402) |
| Intron | GTAAGTATCAAGGTTACAAGACAGGTTTAAGGAGACCAATAGAAACTGGGCTTGTCGA |
| | GACAGAGAAGACTCTTGCGTTTCTGATAGGCACCTATTGGTCTTACTGACATCCACTT |
| | TGCCTTTCTCTCCACAG (SEQ ID NO: 2409) |
| Codon | ATGCTGTGGTGGGAAGAGGTCGAGGACTGCTACGAGCGCGAAGATGTGCAGAAGAAAA |
| optimized | CTTTCACTAAATGGGTGAATGCCCAGTTCTCTAAGTTCGGAAAGCAGCACATCGAAAA |
| microdystrophin | CCTGTTTTCCGATCTGCAAGATGGCCGGCGGCTGCTGGACCTGCTGGAGGGGCTGACC |
| coding | GGACAGAAGCTGCCTAAGGAGAAAGGATCTACCAGGGTCCATGCTCTGAATAACGTGA |
| sequence | ACAAGGCTCTGAGGGTGCTGCAGAACAATAACGTGGACCTGGTGAACATTGGTTCCAC |
| | AGACATCGTGGATGGAAACCACAAACTGACACTGGGACTGATCTGGAACATCATTCTG |
| | CATTGGCAAGTGAAGAACGTGATGAAGAACATCATGGCCGGACTGCAGCAGACGAACT |
| | CCGAGAAGATCCTGCTTAGCTGGGTGAGACAGAGTACCCGGAATTACCCACAGGTGAA |
| | CGTGATCAATTTCACAACCAGCTGGTCAGACGGCCTGGCTCTGAATGCCCTGATTCAT |
| | AGCCACCGGCCAGATCTGTTTGACTGGAACTCTGTCGTGTGTCAGCAGTCAGCCACCC |
| | AGAGGCTGGAGCACGCCTTCAATATTGCCCGCTATCAGCTGGGGATTGAGAAACTGCT |
| | GGACCCCGAGGATGTGGACACAACATACCCTGACAAGAAGAGCATACTTATGTATATC |
| | ACAAGCCTGTTCCAGGTGCTGCCACAGCAGGTCTCCATCGAGGCCATTCAAGAAGTGG |
| | AGATGCTGCCCAGGCCTCCTAAGGTCACCAAAGAGGAGCACTTTCAGCTCCATCATCA |
| | GATGCACTACAGCCAGCAAATTACCGTGTCCCTGGCCCAGGGCTACGAGAGGACAAGT |
| | TCCCCCAAGCCTAGATTTAAGAGCTATGCCTATACCCAGGCCGCCTACGTGACTACCT |
| | CCGATCCCACCAGGAGCCCATTTCCCAGCCAGCACCTGGAGGCTCCCGAAGATAAGAG |
| | CTTTGGCAGCAGCCTGATGGAATCTGAGGTGAACCTGGACAGATACCAGACTGCGCTG |
| | GAGGAGGTGCTGTCCTGGCTGCTGAGCGCCGAGGACACCCTGCAGGCTCAGGGCGAGA |
| | TTAGTAATGATGTGGAGGTGGTGAAGGATCAGTTTCACACTCACGAAGGCTACATGAT |
| | GGACCTGACCGCCCACCAGGGCAGGGTGGGGAACATTCTGCAGCTCGGGAGTAAGCTG |
| | ATAGGGACCGGCAAACTGTCCGAGGATGAGGAAACCGAGGTGCAGGAGCAGATGAACC |
| | TGCTTAACTCCCGGTGGGAGTGTCTGAGAGTGGCCAGCATGGAGAAGCAGAGCAATCT |
| | GCACAGAGTGCTGATGGATCTGCAGAATCAGAAGCTGAAGGAACTGAATGATTGGCTC |
| | ACAAAGACAGAGGAGAGGACTAGAAAGATGGAGGAGGAGCCCCTGGGCCCTGACCTGG |
| | AGGACCTGAAGCGACAGGTGCAGCAGCACAAGGTGCTGCAGGAGGATCTGGAGCAAGA |
| | ACAAGTGAGGGTGAACTCTCTGACTCATATGGTTGTGGTGGTGGACGAGTCTTCCGGA |
| | GACCACGCAACTGCCGCCCTCGAGGAGCAGCTGAAGGTGCTGGGCGATAGGTGGGCTA |
| | ATATCTGTAGGTGGACCGAGGACAGATGGGTGCTGCTGCAGGATATCCTGCTGAAATG |
| | GCAGCGCCTGACAGAAGAGCAGTGTCTGTTCAGCGCATGGCTGAGCGAAAAGGAGGAC |
| | GCTGTGAACAAGATCCACACTACAGGATTTAAAGACCAGAACGAGATGCTGAGTAGCC |
| | TGCAGAAGCTGGCTGTGCTGAAGGCTGATTTGGAGAAAAAGAAGCAGAGCATGGGCAA |
| | GCTGTACTCCCTGAAACAGGACCTGCTGAGCACCCTGAAGAATAAGTCCGTGACCCAG |
| | AAAACCGAAGCTTGGCTGGACAACTTCGCGCGCTGCTGGGATAACCTGGTGCAGAAGC |
| | TGGAGAAGAGCACCGCCCAGATTTCTCAAGCAGTGACCACAACCCAGCCCAGCCTGAC |
| | CCAGACTACAGTAATGGAGACAGTCACTACCGTGACCACCCGGGAGCAGATCCTGGTG |
| | AAGCACGCCCAGGAAGAGCTGCCGCCCCCACCACCCCAGAAAAAGCGGACCCTGGAGC |
| | GCCTGCAGGAGCTCCAGGAGGCCACCGACGAACTGGATCTGAAGCTGAGGCAGGCCGA |
| | GGTGATTAAGGGATCCTGGCAGCCTGTGGGAGACCTGCTGATCGACAGCCTGCAGGAT |
| | CATCTGGAGAAAGTCAAAGCTCTGAGAGGGGAGATCGCCCCACTGAAGGAGAACGTGA |
| | GCCACGTGAATGACCTGGCTAGACAGCTGACCACCTTGGGCATTCAGCTGAGTCCTTA |
| | CAATCTGTCAACTCTGGAGGATCTGAACACCCGGTGGAAACTGCTGCAGGTGGCTGTG |
| | GAAGATCGGGTGCGCCAGTTACATGAGGCCCACAGGGACTTTGGCCCAGCCTCACAGC |
| | ACTTTCTGTCAACCTCTGTGCAAGGACCTTGGGAGAGAGCAATCAGCCCAAACAAGGT |
| | GCCATATTATATCAATCATGAGACTCAGACAACTTGCTGGGATCACCCCAAGATGACA |
| | GAACTCTATCAGTCACTCGCCGATCTCAATAATGTACGGTTCTCTGCCTACCGGACAG |
| | CTATGAAGCTGCGGAGGCTGCAGAAGGCACTCTGTCTGGACCTGCTGAGTCTCTCTGC |
| | GGCCTGCGATGCCCTGGATCAACATAACCTGAAGCAGAATGATCAGCCTATGGACATC |
| | CTGCAGATCATTAACTGTCTCACCACAATCTACGATCGGCTGGAACAGGAACATAACA |
| | ACCTGGTGAATGTGCCACTGTGTGTGGACATGTGCCTGAATTGGCTGCTCAACGTCTA |
| | CGATACAGGCAGGACTGGCAGGATCCGCGTGCTGAGCTTTAAAACCGGCATCATCTCT |
| | CTGTGCAAAGCCCACCTGGAGGACAAATATCGGTACCTGTTTAAGCAGGTGGCCTCCA |
| | GCACAGGCTTCTGTGATCAGCGCCGGCTGGGCCTGCTGCTGCACGACAGCATTCAGAT |
| | CCCTAGGCAGCTGGGCGAGGTGGCCTCCTTCGGCGGCTCAAACATTGAACCCAGCGTG |
| | AGATCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGTGGC |
| | CGCCGCTGAGACGGCCAAGCACCAGGCAAAGTGCAATATCTGCAAGGAATGCCCCATT |
| | ATTGGCTTTCGATACCGGTCCTTGAAACACTTCAATTATGACATCTGCCAGTCATGCT |
| | TCTTCAGCGGACGCGTGGCCAAGGGACACAAGATGCATTATCCTATGGTGGAATACTG |
| | CACTCCAACTACTTCCGGCGAGGACGTGAGAGATTTCGCTAAAGTTCTTAAAAACAAA |
| | TTTCGGACTAAGAGGTACTTCGCCAAGCACCCCCGCATGGGCTATCTGCCTGTGCAGA |
| | CAGTGCTTGAAGGAGACAACATGGAAACCGATACAATGTGA (SEQ ID NO: |
| | 2406) |
| First DRG de- | CGGCCTGATTCACAACACCAGCT (SEQ ID NO: 2400) |
| targeting | |
| miRNA binding | |
| site | |
| Second DRG | CGGCCTGATTCACAACACCAGCT (SEQ ID NO: 2400) |
| de-targeting | |
| miRNA binding | |
| site | |
| Short polyA | AGGAATAAAATATCTTTATTTTCATTACATCTGTGTGTTGGTTTTTTGTGTG (SEQ |
| | ID NO: 2410) |
| AAV2 ITR | AGGAACCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCAGTGAGC |
| | GAGCGAGCGCGC (SEQ ID NO: 2411) |

Nucleic acid 14
(SEQ ID NO: 2425)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 14 | |
| annotated | Sequence |
| AAV2 ITR | GCGCGCTCGCTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGAGTGGCCAACT |
| | CCATCACTAGGGGTTCCT (SEQ ID NO: 2408) |
| Novel promoter | CGTCTGTCTAATTCCAGCCAAGGGCATGGGGGAAAGGACAGAGCCCAGGGACCTG |
| | TGGCCTATTCTCTGAGAGGACGCTGGGCTAATAATAGGAGGGGAGATTAGGGAGG |
| | GATTTGACTGCATTGTTATCTCACAGGAATGGCGTTTGAGAGCCTTGCTTCCAAG |
| | CTCTTCCAAGAGTCATGCCACCCCCCTCCAATCTCTGTGGAAGTGCCCACTGCCT |
| | TTCCCGCTTCGTTCTCTTCTCTCTAAATCTGAGCCCACCCCACTCAGGGGTACAG |
| | GATCTGGTTCATTTATCTCCCAGAATGATGCCTTGGGTTGCGTCCCCTCTAAACT |
| | CTGGCACATTCCAGCCCCTCTTTGGGAGAAAGAACATCTCTTCTTGCCCAGGCCC |
| | TAGTGAGACATGCATCGCAAGACATGCTCCCCCCTTCCTCCCCGCTGGGCTCCAG |
| | CTGCTGCCAGCCCTCCAAGAAAGGAGAGGCCCTGAGTTGGGCTATTTTGGTATCT |
| | GGGGTGGGCACCCGCAGGGCTAAGGTTACCTTGGTATGTTAAGGGCTCCCTGGGG |
| | CAGGACTATATAACCCCAGAGGGACTGCCCCATGCTGACTCCTTGCTTCTTTCCT |
| | GCTCGAGCCGCTGCCTTGCCCCCCGGAGACTGAAGACA (SEQ ID NO: |
| | 2403) |
| Intron | GTAAGTATCAAGGTTACAAGACAGGTTTAAGGAGACCAATAGAAACTGGGCTTGT |
| | CGAGACAGAGAAGACTCTTGCGTTTCTGATAGGCACCTATTGGTCTTACTGACAT |
| | CCACTTTGCCTTTCTCTCCACAG (SEQ ID NO: 2409) |
| Codon optimized | ATGCTGTGGTGGGAAGAGGTCGAGGACTGCTACGAGCGCGAAGATGTGCAGAAGA |
| microdystrophin | AAACTTTCACTAAATGGGTGAATGCCCAGTTCTCTAAGTTCGGAAAGCAGCACAT |
| coding sequence | CGAAAACCTGTTTTCCGATCTGCAAGATGGCCGGCGGCTGCTGGACCTGCTGGAG |
| | GGGCTGACCGGACAGAAGCTGCCTAAGGAGAAAGGATCTACCAGGGTCCATGCTC |
| | TGAATAACGTGAACAAGGCTCTGAGGGTGCTGCAGAACAATAACGTGGACCTGGT |
| | GAACATTGGTTCCACAGACATCGTGGATGGAAACCACAAACTGACACTGGGACTG |
| | ATCTGGAACATCATTCTGCATTGGCAAGTGAAGAACGTGATGAAGAACATCATGG |
| | CCGGACTGCAGCAGACGAACTCCGAGAAGATCCTGCTTAGCTGGGTGAGACAGAG |
| | TACCCGGAATTACCCACAGGTGAACGTGATCAATTTCACAACCAGCTGGTCAGAC |
| | GGCCTGGCTCTGAATGCCCTGATTCATAGCCACCGGCCAGATCTGTTTGACTGGA |
| | ACTCTGTCGTGTGTCAGCAGTCAGCCACCCAGAGGCTGGAGCACGCCTTCAATAT |
| | TGCCCGCTATCAGCTGGGGATTGAGAAACTGCTGGACCCCGAGGATGTGGACACA |
| | ACATACCCTGACAAGAAGAGCATACTTATGTATATCACAAGCCTGTTCCAGGTGC |
| | TGCCACAGCAGGTCTCCATCGAGGCCATTCAAGAAGTGGAGATGCTGCCCAGGCC |
| | TCCTAAGGTCACCAAAGAGGAGCACTTTCAGCTCCATCATCAGATGCACTACAGC |
| | CAGCAAATTACCGTGTCCCTGGCCCAGGGCTACGAGAGGACAAGTTCCCCCAAGC |
| | CTAGATTTAAGAGCTATGCCTATACCCAGGCCGCCTACGTGACTACCTCCGATCC |
| | CACCAGGAGCCCATTTCCCAGCCAGCACCTGGAGGCTCCCGAAGATAAGAGCTTT |
| | GGCAGCAGCCTGATGGAATCTGAGGTGAACCTGGACAGATACCAGACTGCGCTGG |
| | AGGAGGTGCTGTCCTGGCTGCTGAGCGCCGAGGACACCCTGCAGGCTCAGGGCGA |
| | GATTAGTAATGATGTGGAGGTGGTGAAGGATCAGTTTCACACTCACGAAGGCTAC |
| | ATGATGGACCTGACCGCCCACCAGGGCAGGGTGGGGAACATTCTGCAGCTCGGGA |
| | GTAAGCTGATAGGGACCGGCAAACTGTCCGAGGATGAGGAAACCGAGGTGCAGGA |
| | GCAGATGAACCTGCTTAACTCCCGGTGGGAGTGTCTGAGAGTGGCCAGCATGGAG |
| | AAGCAGAGCAATCTGCACAGAGTGCTGATGGATCTGCAGAATCAGAAGCTGAAGG |
| | AACTGAATGATTGGCTCACAAAGACAGAGGAGAGGACTAGAAAGATGGAGGAGGA |
| | GCCCCTGGGCCCTGACCTGGAGGACCTGAAGCGACAGGTGCAGCAGCACAAGGTG |
| | CTGCAGGAGGATCTGGAGCAAGAACAAGTGAGGGTGAACTCTCTGACTCATATGG |
| | TTGTGGTGGTGGACGAGTCTTCCGGAGACCACGCAACTGCCGCCCTCGAGGAGCA |
| | GCTGAAGGTGCTGGGCGATAGGTGGGCTAATATCTGTAGGTGGACCGAGGACAGA |
| | TGGGTGCTGCTGCAGGATATCCTGCTGAAATGGCAGCGCCTGACAGAAGAGCAGT |
| | GTCTGTTCAGCGCATGGCTGAGCGAAAAGGAGGACGCTGTGAACAAGATCCACAC |
| | TACAGGATTTAAAGACCAGAACGAGATGCTGAGTAGCCTGCAGAAGCTGGCTGTG |
| | CTGAAGGCTGATTTGGAGAAAAAGAAGCAGAGCATGGGCAAGCTGTACTCCCTGA |
| | AACAGGACCTGCTGAGCACCCTGAAGAATAAGTCCGTGACCCAGAAAACCGAAGC |
| | TTGGCTGGACAACTTCGCGCGCTGCTGGGATAACCTGGTGCAGAAGCTGGAGAAG |
| | AGCACCGCCCAGATTTCTCAAGCAGTGACCACAACCCAGCCCAGCCTGACCCAGA |
| | CTACAGTAATGGAGACAGTCACTACCGTGACCACCCGGGAGCAGATCCTGGTGAA |
| | GCACGCCCAGGAAGAGCTGCCGCCCCCACCACCCCAGAAAAAGCGGACCCTGGAG |
| | CGCCTGCAGGAGCTCCAGGAGGCCACCGACGAACTGGATCTGAAGCTGAGGCAGG |
| | CCGAGGTGATTAAGGGATCCTGGCAGCCTGTGGGAGACCTGCTGATCGACAGCCT |
| | GCAGGATCATCTGGAGAAAGTCAAAGCTCTGAGAGGGGAGATCGCCCCACTGAAG |
| | GAGAACGTGAGCCACGTGAATGACCTGGCTAGACAGCTGACCACCTTGGGCATTC |
| | AGCTGAGTCCTTACAATCTGTCAACTCTGGAGGATCTGAACACCCGGTGGAAACT |
| | GCTGCAGGTGGCTGTGGAAGATCGGGTGCGCCAGTTACATGAGGCCCACAGGGAC |
| | TTTGGCCCAGCCTCACAGCACTTTCTGTCAACCTCTGTGCAAGGACCTTGGGAGA |
| | GAGCAATCAGCCCAAACAAGGTGCCATATTATATCAATCATGAGACTCAGACAAC |
| | TTGCTGGGATCACCCCAAGATGACAGAACTCTATCAGTCACTCGCCGATCTCAAT |
| | AATGTACGGTTCTCTGCCTACCGGACAGCTATGAAGCTGCGGAGGCTGCAGAAGG |
| | CACTCTGTCTGGACCTGCTGAGTCTCTCTGCGGCCTGCGATGCCCTGGATCAACA |
| | TAACCTGAAGCAGAATGATCAGCCTATGGACATCCTGCAGATCATTAACTGTCTC |
| | ACCACAATCTACGATCGGCTGGAACAGGAACATAACAACCTGGTGAATGTGCCAC |
| | TGTGTGTGGACATGTGCCTGAATTGGCTGCTCAACGTCTACGATACAGGCAGGAC |
| | TGGCAGGATCCGCGTGCTGAGCTTTAAAACCGGCATCATCTCTCTGTGCAAAGCC |
| | CACCTGGAGGACAAATATCGGTACCTGTTTAAGCAGGTGGCCTCCAGCACAGGCT |
| | TCTGTGATCAGCGCCGGCTGGGCCTGCTGCTGCACGACAGCATTCAGATCCCTAG |
| | GCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGT |
| | GGCCGCCGCTGAGACGGCCAAGCACCAGGCAAAGTGCAATATCTGCAAGGAATGC |
| | CCCATTATTGGCTTTCGATACCGGTCCTTGAAACACTTCAATTATGACATCTGCC |
| | AGTCATGCTTCTTCAGCGGACGCGTGGCCAAGGGACACAAGATGCATTATCCTAT |
| | GGTGGAATACTGCACTCCAACTACTTCCGGCGAGGACGTGAGAGATTTCGCTAAA |
| | GTTCTTAAAAACAAATTTCGGACTAAGAGGTACTTCGCCAAGCACCCCCGCATGG |
| | GCTATCTGCCTGTGCAGACAGTGCTTGAAGGAGACAACATGGAAACCGATACAAT |
| | GTGA (SEQ ID NO: 2406) |
| First DRG de- | CAACAAAATCACTGATGCTGGA (SEQ ID NO: 2400) |
| targeting miRNA | |
| binding site | |
| Second DRG de- | CAACAAAATCACTGATGCTGGA (SEQ ID NO: 2400) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 2411) |

Nucleic acid 15
(SEQ ID NO: 2426)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 15 | |
| annotated | Sequence |
| AAV2 ITR | GCGCGCTCGCTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGAGTGGCCAACT |
| | CCATCACTAGGGGTTCCT (SEQ ID NO: 2408) |
| Novel promoter | CGTCTGTCTAATTCCAGCCAAGGGCATGGGGGAAAGGACAGAGCCCAGGGACCTG |
| | TGGCCTATTCTCTGAGAGGACGCTGGGCTAATAATAGGAGGGGAGATTAGGGAGG |
| | GATTTGACTGCATTGTTATCTCACAGGAATGGCGTTTGAGAGCCTTGCTTCCAAG |
| | CTCTTCCAAGAGTCATGCCACCCCCCTCCAATCTCTGTGGAAGTGCCCACTGCCT |
| | TTCCCGCTTCGTTCTCTTCTCTCGGTCCCATCCCTGACCCACTAGCCTGGTGCCG |
| | GGGGGAGACCAATTTCTGGAGGAAGCTCTGGGTACCCAGTCTTGCCCTAAGCCTG |
| | CCTCTGAACAGGCTGTTCTTGTTTATGACCCCCGTCCCAGCAGAATAGTGATTGC |
| | TCATCCCTGCATGTGATTTGAGAGGGCCAGTCTTTCAGGCCTGGAGGAAACTCAA |
| | GCCCCACCAGCCGCTTGGCATCGGAATGCTCAGCTGGTCCCCTCCTGGGGCTCAT |
| | GTGACCAAGATCTAGCTATAAATATCAGAGGGGCCTCAGACCAAGACAGAATTCG |
| | GGCACCAGGAGAAGGAAGCCAACAGGATCCGACCCGGTGTTTTGTGACAAAGGCA |
| | AGACCCCCAGGTCTACTTAGAGCAACGTTAGTAGAGGA (SEQ ID NO: |
| | 2404) |
| Intron | GTAAGTATCAAGGTTACAAGACAGGTTTAAGGAGACCAATAGAAACTGGGCTTGT |
| | CGAGACAGAGAAGACTCTTGCGTTTCTGATAGGCACCTATTGGTCTTACTGACAT |
| | CCACTTTGCCTTTCTCTCCACAG (SEQ ID NO: 2409) |
| Codon optimized | ATGCTGTGGTGGGAAGAGGTCGAGGACTGCTACGAGCGCGAAGATGTGCAGAAGA |
| microdystrophin | AAACTTTCACTAAATGGGTGAATGCCCAGTTCTCTAAGTTCGGAAAGCAGCACAT |
| coding sequence | CGAAAACCTGTTTTCCGATCTGCAAGATGGCCGGCGGCTGCTGGACCTGCTGGAG |
| | GGGCTGACCGGACAGAAGCTGCCTAAGGAGAAAGGATCTACCAGGGTCCATGCTC |
| | TGAATAACGTGAACAAGGCTCTGAGGGTGCTGCAGAACAATAACGTGGACCTGGT |
| | GAACATTGGTTCCACAGACATCGTGGATGGAAACCACAAACTGACACTGGGACTG |
| | ATCTGGAACATCATTCTGCATTGGCAAGTGAAGAACGTGATGAAGAACATCATGG |
| | CCGGACTGCAGCAGACGAACTCCGAGAAGATCCTGCTTAGCTGGGTGAGACAGAG |
| | TACCCGGAATTACCCACAGGTGAACGTGATCAATTTCACAACCAGCTGGTCAGAC |
| | GGCCTGGCTCTGAATGCCCTGATTCATAGCCACCGGCCAGATCTGTTTGACTGGA |
| | ACTCTGTCGTGTGTCAGCAGTCAGCCACCCAGAGGCTGGAGCACGCCTTCAATAT |
| | TGCCCGCTATCAGCTGGGGATTGAGAAACTGCTGGACCCCGAGGATGTGGACACA |
| | ACATACCCTGACAAGAAGAGCATACTTATGTATATCACAAGCCTGTTCCAGGTGC |
| | TGCCACAGCAGGTCTCCATCGAGGCCATTCAAGAAGTGGAGATGCTGCCCAGGCC |
| | TCCTAAGGTCACCAAAGAGGAGCACTTTCAGCTCCATCATCAGATGCACTACAGC |
| | CAGCAAATTACCGTGTCCCTGGCCCAGGGCTACGAGAGGACAAGTTCCCCCAAGC |
| | CTAGATTTAAGAGCTATGCCTATACCCAGGCCGCCTACGTGACTACCTCCGATCC |
| | CACCAGGAGCCCATTTCCCAGCCAGCACCTGGAGGCTCCCGAAGATAAGAGCTTT |
| | GGCAGCAGCCTGATGGAATCTGAGGTGAACCTGGACAGATACCAGACTGCGCTGG |
| | AGGAGGTGCTGTCCTGGCTGCTGAGCGCCGAGGACACCCTGCAGGCTCAGGGCGA |
| | GATTAGTAATGATGTGGAGGTGGTGAAGGATCAGTTTCACACTCACGAAGGCTAC |
| | ATGATGGACCTGACCGCCCACCAGGGCAGGGTGGGGAACATTCTGCAGCTCGGGA |
| | GTAAGCTGATAGGGACCGGCAAACTGTCCGAGGATGAGGAAACCGAGGTGCAGGA |
| | GCAGATGAACCTGCTTAACTCCCGGTGGGAGTGTCTGAGAGTGGCCAGCATGGAG |
| | AAGCAGAGCAATCTGCACAGAGTGCTGATGGATCTGCAGAATCAGAAGCTGAAGG |
| | AACTGAATGATTGGCTCACAAAGACAGAGGAGAGGACTAGAAAGATGGAGGAGGA |
| | GCCCCTGGGCCCTGACCTGGAGGACCTGAAGCGACAGGTGCAGCAGCACAAGGTG |
| | CTGCAGGAGGATCTGGAGCAAGAACAAGTGAGGGTGAACTCTCTGACTCATATGG |
| | TTGTGGTGGTGGACGAGTCTTCCGGAGACCACGCAACTGCCGCCCTCGAGGAGCA |
| | GCTGAAGGTGCTGGGCGATAGGTGGGCTAATATCTGTAGGTGGACCGAGGACAGA |
| | TGGGTGCTGCTGCAGGATATCCTGCTGAAATGGCAGCGCCTGACAGAAGAGCAGT |
| | GTCTGTTCAGCGCATGGCTGAGCGAAAAGGAGGACGCTGTGAACAAGATCCACAC |
| | TACAGGATTTAAAGACCAGAACGAGATGCTGAGTAGCCTGCAGAAGCTGGCTGTG |
| | CTGAAGGCTGATTTGGAGAAAAAGAAGCAGAGCATGGGCAAGCTGTACTCCCTGA |
| | AACAGGACCTGCTGAGCACCCTGAAGAATAAGTCCGTGACCCAGAAAACCGAAGC |
| | TTGGCTGGACAACTTCGCGCGCTGCTGGGATAACCTGGTGCAGAAGCTGGAGAAG |
| | AGCACCGCCCAGATTTCTCAAGCAGTGACCACAACCCAGCCCAGCCTGACCCAGA |
| | CTACAGTAATGGAGACAGTCACTACCGTGACCACCCGGGAGCAGATCCTGGTGAA |
| | GCACGCCCAGGAAGAGCTGCCGCCCCCACCACCCCAGAAAAAGCGGACCCTGGAG |
| | CGCCTGCAGGAGCTCCAGGAGGCCACCGACGAACTGGATCTGAAGCTGAGGCAGG |
| | CCGAGGTGATTAAGGGATCCTGGCAGCCTGTGGGAGACCTGCTGATCGACAGCCT |
| | GCAGGATCATCTGGAGAAAGTCAAAGCTCTGAGAGGGGAGATCGCCCCACTGAAG |
| | GAGAACGTGAGCCACGTGAATGACCTGGCTAGACAGCTGACCACCTTGGGCATTC |
| | AGCTGAGTCCTTACAATCTGTCAACTCTGGAGGATCTGAACACCCGGTGGAAACT |
| | GCTGCAGGTGGCTGTGGAAGATCGGGTGCGCCAGTTACATGAGGCCCACAGGGAC |
| | TTTGGCCCAGCCTCACAGCACTTTCTGTCAACCTCTGTGCAAGGACCTTGGGAGA |
| | GAGCAATCAGCCCAAACAAGGTGCCATATTATATCAATCATGAGACTCAGACAAC |
| | TTGCTGGGATCACCCCAAGATGACAGAACTCTATCAGTCACTCGCCGATCTCAAT |
| | AATGTACGGTTCTCTGCCTACCGGACAGCTATGAAGCTGCGGAGGCTGCAGAAGG |
| | CACTCTGTCTGGACCTGCTGAGTCTCTCTGCGGCCTGCGATGCCCTGGATCAACA |
| | TAACCTGAAGCAGAATGATCAGCCTATGGACATCCTGCAGATCATTAACTGTCTC |
| | ACCACAATCTACGATCGGCTGGAACAGGAACATAACAACCTGGTGAATGTGCCAC |
| | TGTGTGTGGACATGTGCCTGAATTGGCTGCTCAACGTCTACGATACAGGCAGGAC |
| | TGGCAGGATCCGCGTGCTGAGCTTTAAAACCGGCATCATCTCTCTGTGCAAAGCC |
| | CACCTGGAGGACAAATATCGGTACCTGTTTAAGCAGGTGGCCTCCAGCACAGGCT |
| | TCTGTGATCAGCGCCGGCTGGGCCTGCTGCTGCACGACAGCATTCAGATCCCTAG |
| | GCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGT |
| | GGCCGCCGCTGAGACGGCCAAGCACCAGGCAAAGTGCAATATCTGCAAGGAATGC |
| | CCCATTATTGGCTTTCGATACCGGTCCTTGAAACACTTCAATTATGACATCTGCC |
| | AGTCATGCTTCTTCAGCGGACGCGTGGCCAAGGGACACAAGATGCATTATCCTAT |
| | GGTGGAATACTGCACTCCAACTACTTCCGGCGAGGACGTGAGAGATTTCGCTAAA |
| | GTTCTTAAAAACAAATTTCGGACTAAGAGGTACTTCGCCAAGCACCCCCGCATGG |
| | GCTATCTGCCTGTGCAGACAGTGCTTGAAGGAGACAACATGGAAACCGATACAAT |
| | GTGA |
| | (SEQ ID NO: 2406) |
| First DRG de- | CGGCCTGATTCACAACACCAGCT (SEQ ID NO: 2400) |
| targeting miRNA | |
| binding site | |
| Second DRG de- | CGGCCTGATTCACAACACCAGCT (SEQ ID NO: 2400) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 2411) |

Nucleic acid 16
(SEQ ID NO: 2427)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAAGAGGTCGAGGACTGCTACGAGCGCGAAGATGTGCAGAAGAAAACTTTCACT
AAATGGGTGAATGCCCAGTTCTCTAAGTTCGGAAAGCAGCACATCGAAAACCTGTTTTCCGATCTGCAAGATGGC
CGGCGGCTGCTGGACCTGCTGGAGGGGCTGACCGGACAGAAGCTGCCTAAGGAGAAAGGATCTACCAGGGTCCAT
GCTCTGAATAACGTGAACAAGGCTCTGAGGGTGCTGCAGAACAATAACGTGGACCTGGTGAACATTGGTTCCACA
GACATCGTGGATGGAAACCACAAACTGACACTGGGACTGATCTGGAACATCATTCTGCATTGGCAAGTGAAGAAC
GTGATGAAGAACATCATGGCCGGACTGCAGCAGACGAACTCCGAGAAGATCCTGCTTAGCTGGGTGAGACAGAGT
ACCCGGAATTACCCACAGGTGAACGTGATCAATTTCACAACCAGCTGGTCAGACGGCCTGGCTCTGAATGCCCTG
ATTCATAGCCACCGGCCAGATCTGTTTGACTGGAACTCTGTCGTGTGTCAGCAGTCAGCCACCCAGAGGCTGGAG
CACGCCTTCAATATTGCCCGCTATCAGCTGGGGATTGAGAAACTGCTGGACCCCGAGGATGTGGACACAACATAC
CCTGACAAGAAGAGCATACTTATGTATATCACAAGCCTGTTCCAGGTGCTGCCACAGCAGGTCTCCATCGAGGCC
ATTCAAGAAGTGGAGATGCTGCCCAGGCCTCCTAAGGTCACCAAAGAGGAGCACTTTCAGCTCCATCATCAGATG
CACTACAGCCAGCAAATTACCGTGTCCCTGGCCCAGGGCTACGAGAGGACAAGTTCCCCCAAGCCTAGATTTAAG
AGCTATGCCTATACCCAGGCCGCCTACGTGACTACCTCCGATCCCACCAGGAGCCCATTTCCCAGCCAGCACCTG
GAGGCTCCCGAAGATAAGAGCTTTGGCAGCAGCCTGATGGAATCTGAGGTGAACCTGGACAGATACCAGACTGCG
CTGGAGGAGGTGCTGTCCTGGCTGCTGAGCGCCGAGGACACCCTGCAGGCTCAGGGCGAGATTAGTAATGATGTG
GAGGTGGTGAAGGATCAGTTTCACACTCACGAAGGCTACATGATGGACCTGACCGCCCACCAGGGCAGGGTGGGG
AACATTCTGCAGCTCGGGAGTAAGCTGATAGGGACCGGCAAACTGTCCGAGGATGAGGAAACCGAGGTGCAGGAG
CAGATGAACCTGCTTAACTCCCGGTGGGAGTGTCTGAGAGTGGCCAGCATGGAGAAGCAGAGCAATCTGCACAGA
GTGCTGATGGATCTGCAGAATCAGAAGCTGAAGGAACTGAATGATTGGCTCACAAAGACAGAGGAGAGGACTAGA
AAGATGGAGGAGGAGCCCCTGGGCCCTGACCTGGAGGACCTGAAGCGACAGGTGCAGCAGCACAAGGTGCTGCAG
GAGGATCTGGAGCAAGAACAAGTGAGGGTGAACTCTCTGACTCATATGGTTGTGGTGGTGGACGAGTCTTCCGGA
GACCACGCAACTGCCGCCCTCGAGGAGCAGCTGAAGGTGCTGGGCGATAGGTGGGCTAATATCTGTAGGTGGACC
GAGGACAGATGGGTGCTGCTGCAGGATATCCTGCTGAAATGGCAGCGCCTGACAGAAGAGCAGTGTCTGTTCAGC
GCATGGCTGAGCGAAAAGGAGGACGCTGTGAACAAGATCCACACTACAGGATTTAAAGACCAGAACGAGATGCTG
AGTAGCCTGCAGAAGCTGGCTGTGCTGAAGGCTGATTTGGAGAAAAAGAAGCAGAGCATGGGCAAGCTGTACTCC
CTGAAACAGGACCTGCTGAGCACCCTGAAGAATAAGTCCGTGACCCAGAAAACCGAAGCTTGGCTGGACAACTTC
GCGCGCTGCTGGGATAACCTGGTGCAGAAGCTGGAGAAGAGCACCGCCCAGATTTCTCAAGCAGTGACCACAACC
CAGCCCAGCCTGACCCAGACTACAGTAATGGAGACAGTCACTACCGTGACCACCCGGGAGCAGATCCTGGTGAAG
CACGCCCAGGAAGAGCTGCCGCCCCCACCACCCCAGAAAAAGCGGACCCTGGAGCGCCTGCAGGAGCTCCAGGAG
GCCACCGACGAACTGGATCTGAAGCTGAGGCAGGCCGAGGTGATTAAGGGATCCTGGCAGCCTGTGGGAGACCTG
CTGATCGACAGCCTGCAGGATCATCTGGAGAAAGTCAAAGCTCTGAGAGGGGAGATCGCCCCACTGAAGGAGAAC
GTGAGCCACGTGAATGACCTGGCTAGACAGCTGACCACCTTGGGCATTCAGCTGAGTCCTTACAATCTGTCAACT
CTGGAGGATCTGAACACCCGGTGGAAACTGCTGCAGGTGGCTGTGGAAGATCGGGTGCGCCAGTTACATGAGGCC
CACAGGGACTTTGGCCCAGCCTCACAGCACTTTCTGTCAACCTCTGTGCAAGGACCTTGGGAGAGAGCAATCAGC
CCAAACAAGGTGCCATATTATATCAATCATGAGACTCAGACAACTTGCTGGGATCACCCCAAGATGACAGAACTC
TATCAGTCACTCGCCGATCTCAATAATGTACGGTTCTCTGCCTACCGGACAGCTATGAAGCTGCGGAGGCTGCAG
AAGGCACTCTGTCTGGACCTGCTGAGTCTCTCTGCGGCCTGCGATGCCCTGGATCAACATAACCTGAAGCAGAAT
GATCAGCCTATGGACATCCTGCAGATCATTAACTGTCTCACCACAATCTACGATCGGCTGGAACAGGAACATAAC
AACCTGGTGAATGTGCCACTGTGTGTGGACATGTGCCTGAATTGGCTGCTCAACGTCTACGATACAGGCAGGACT
GGCAGGATCCGCGTGCTGAGCTTTAAAACCGGCATCATCTCTCTGTGCAAAGCCCACCTGGAGGACAAATATCGG
TACCTGTTTAAGCAGGTGGCCTCCAGCACAGGCTTCTGTGATCAGCGCCGGCTGGGCCTGCTGCTGCACGACAGC
ATTCAGATCCCTAGGCAGCTGGGCGAGGTGGCCTCCTTCGGCGGCTCAAACATTGAACCCAGCGTGAGATCTTGC
TTTCAGTTTGCAAATAATAAACCCGAGATCGAGGCCGCCCTGTTCCTGGATTGGATGAGACTGGAGCCTCAGAGT
ATGGTGTGGCTGCCAGTGCTGCATAGAGTGGCCGCCGCTGAGACGGCCAAGCACCAGGCAAAGTGCAATATCTGC
AAGGAATGCCCCATTATTGGCTTTCGATACCGGTCCTTGAAACACTTCAATTATGACATCTGCCAGTCATGCTTC
TTCAGCGGACGCGTGGCCAAGGGACACAAGATGCATTATCCTATGGTGGAATACTGCACTCCAACTACTTCCGGC
GAGGACGTGAGAGATTTCGCTAAAGTTCTTAAAAACAAATTTCGGACTAAGAGGTACTTCGCCAAGCACCCCCGC
ATGGGCTATCTGCCTGTGCAGACAGTGCTTGAAGGAGACAACATGGAAACCGATACAATGTGATAGATTCTCATA
CAGCTAGATAACCAAAGAAGATCAGTCATACAGCTAGATAACCAAAGA
AGACGCAGGAATAAAATATCTTTATTTTCATTACATCTGTGTGTTGGTTTTTTGTGTGAGGAACCCCTAGTGATG
GAGTTGGCCACTCCCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGC

| Nucleic acid | |
|---|---|
| cargo 16 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | TAGTTATTAATAGTAATCAATTACGGGGTCATTAGTTCATAGCCCATATATGGAGT |
| | TCCGCGTTACATAACTTACGGTAAATGGCCCGCCTGGCTGACCGCCCAACGACCCC |
| | CGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATC |
| | AAGTGTATCATATGCCAAGTACGCCCCCTATTGACGTCAATGACGGTAAATGGCCC |
| | GCCTGGCATTATGCCCAGTACATGACCTTATGGGACTTTCCTACTTGGCAGTACAT |
| | CTACGTATTAGTCATCGCTATTACCATGCTAGACTAGCATGCTGCCCATGTAAGGA |
| | GGCAAGGCCTGGGGACACCCGAGATGCCTGGTTATAATTAACCCAGACATGTGGCT |
| | GCCCCCCCCCCCCAACACCTGCTGCCTCTAAAAATAACCCTGCATGCCATGTTCCC |
| | GGCGAAGGGCCAGCTGTCCCCCGCCAGCTAGACTCAGCTCTCAGGGGCCCCTCCCT |
| | GGGGACAGCCCCTCCTGGCTAGTCACACCCTGTAGGCTCCTCTATATAACCCAGGG |
| | GCACAGGGGCTGCCCTCATTCTACCACCACCTCCACAGCACAGACAGACACTCAGG |
| | AGCCAGCCAGC (SEQ ID NO: 2402) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon | ATGCTGTGGTGGGAAGAGGTCGAGGACTGCTACGAGCGCGAAGATGTGCAGAAGAA |
| optimized | AACTTTCACTAAATGGGTGAATGCCCAGTTCTCTAAGTTCGGAAAGCAGCACATCG |
| microdystrophin | AAAACCTGTTTTCCGATCTGCAAGATGGCCGGCGGCTGCTGGACCTGCTGGAGGGG |
| coding | CTGACCGGACAGAAGCTGCCTAAGGAGAAAGGATCTACCAGGGTCCATGCTCTGAA |
| sequence | TAACGTGAACAAGGCTCTGAGGGTGCTGCAGAACAATAACGTGGACCTGGTGAACA |
| | TTGGTTCCACAGACATCGTGGATGGAAACCACAAACTGACACTGGGACTGATCTGG |
| | AACATCATTCTGCATTGGCAAGTGAAGAACGTGATGAAGAACATCATGGCCGGACT |
| | GCAGCAGACGAACTCCGAGAAGATCCTGCTTAGCTGGGTGAGACAGAGTACCCGGA |
| | ATTACCCACAGGTGAACGTGATCAATTTCACAACCAGCTGGTCAGACGGCCTGGCT |
| | CTGAATGCCCTGATTCATAGCCACCGGCCAGATCTGTTTGACTGGAACTCTGTCGT |
| | GTGTCAGCAGTCAGCCACCCAGAGGCTGGAGCACGCCTTCAATATTGCCCGCTATC |
| | AGCTGGGGATTGAGAAACTGCTGGACCCCGAGGATGTGGACACAACATACCCTGAC |
| | AAGAAGAGCATACTTATGTATATCACAAGCCTGTTCCAGGTGCTGCCACAGCAGGT |
| | CTCCATCGAGGCCATTCAAGAAGTGGAGATGCTGCCCAGGCCTCCTAAGGTCACCA |
| | AAGAGGAGCACTTTCAGCTCCATCATCAGATGCACTACAGCCAGCAAATTACCGTG |
| | TCCCTGGCCCAGGGCTACGAGAGGACAAGTTCCCCCAAGCCTAGATTTAAGAGCTA |
| | TGCCTATACCCAGGCCGCCTACGTGACTACCTCCGATCCCACCAGGAGCCCATTTC |
| | CCAGCCAGCACCTGGAGGCTCCCGAAGATAAGAGCTTTGGCAGCAGCCTGATGGAA |
| | TCTGAGGTGAACCTGGACAGATACCAGACTGCGCTGGAGGAGGTGCTGTCCTGGCT |
| | GCTGAGCGCCGAGGACACCCTGCAGGCTCAGGGCGAGATTAGTAATGATGTGGAGG |
| | TGGTGAAGGATCAGTTTCACACTCACGAAGGCTACATGATGGACCTGACCGCCCAC |
| | CAGGGCAGGGTGGGGAACATTCTGCAGCTCGGGAGTAAGCTGATAGGGACCGGCAA |
| | ACTGTCCGAGGATGAGGAAACCGAGGTGCAGGAGCAGATGAACCTGCTTAACTCCC |
| | GGTGGGAGTGTCTGAGAGTGGCCAGCATGGAGAAGCAGAGCAATCTGCACAGAGTG |
| | CTGATGGATCTGCAGAATCAGAAGCTGAAGGAACTGAATGATTGGCTCACAAAGAC |
| | AGAGGAGAGGACTAGAAAGATGGAGGAGGAGCCCCTGGGCCCTGACCTGGAGGACC |
| | TGAAGCGACAGGTGCAGCAGCACAAGGTGCTGCAGGAGGATCTGGAGCAAGAACAA |
| | GTGAGGGTGAACTCTCTGACTCATATGGTTGTGGTGGTGGACGAGTCTTCCGGAGA |
| | CCACGCAACTGCCGCCCTCGAGGAGCAGCTGAAGGTGCTGGGCGATAGGTGGGCTA |
| | ATATCTGTAGGTGGACCGAGGACAGATGGGTGCTGCTGCAGGATATCCTGCTGAAA |
| | TGGCAGCGCCTGACAGAAGAGCAGTGTCTGTTCAGCGCATGGCTGAGCGAAAAGGA |
| | GGACGCTGTGAACAAGATCCACACTACAGGATTTAAAGACCAGAACGAGATGCTGA |
| | GTAGCCTGCAGAAGCTGGCTGTGCTGAAGGCTGATTTGGAGAAAAAGAAGCAGAGC |
| | ATGGGCAAGCTGTACTCCCTGAAACAGGACCTGCTGAGCACCCTGAAGAATAAGTC |
| | CGTGACCCAGAAAACCGAAGCTTGGCTGGACAACTTCGCGCGCTGCTGGGATAACC |
| | TGGTGCAGAAGCTGGAGAAGAGCACCGCCCAGATTTCTCAAGCAGTGACCACAACC |
| | CAGCCCAGCCTGACCCAGACTACAGTAATGGAGACAGTCACTACCGTGACCACCCG |
| | GGAGCAGATCCTGGTGAAGCACGCCCAGGAAGAGCTGCCGCCCCCACCACCCCAGA |
| | AAAAGCGGACCCTGGAGCGCCTGCAGGAGCTCCAGGAGGCCACCGACGAACTGGAT |
| | CTGAAGCTGAGGCAGGCCGAGGTGATTAAGGGATCCTGGCAGCCTGTGGGAGACCT |
| | GCTGATCGACAGCCTGCAGGATCATCTGGAGAAAGTCAAAGCTCTGAGAGGGGAGA |
| | TCGCCCCACTGAAGGAGAACGTGAGCCACGTGAATGACCTGGCTAGACAGCTGACC |
| | ACCTTGGGCATTCAGCTGAGTCCTTACAATCTGTCAACTCTGGAGGATCTGAACAC |
| | CCGGTGGAAACTGCTGCAGGTGGCTGTGGAAGATCGGGTGCGCCAGTTACATGAGG |
| | CCCACAGGGACTTTGGCCCAGCCTCACAGCACTTTCTGTCAACCTCTGTGCAAGGA |
| | CCTTGGGAGAGAGCAATCAGCCCAAACAAGGTGCCATATTATATCAATCATGAGAC |
| | TCAGACAACTTGCTGGGATCACCCCAAGATGACAGAACTCTATCAGTCACTCGCCG |
| | ATCTCAATAATGTACGGTTCTCTGCCTACCGGACAGCTATGAAGCTGCGGAGGCTG |
| | CAGAAGGCACTCTGTCTGGACCTGCTGAGTCTCTCTGCGGCCTGCGATGCCCTGGA |
| | TCAACATAACCTGAAGCAGAATGATCAGCCTATGGACATCCTGCAGATCATTAACT |
| | GTCTCACCACAATCTACGATCGGCTGGAACAGGAACATAACAACCTGGTGAATGTG |
| | CCACTGTGTGTGGACATGTGCCTGAATTGGCTGCTCAACGTCTACGATACAGGCAG |
| | GACTGGCAGGATCCGCGTGCTGAGCTTTAAAACCGGCATCATCTCTCTGTGCAAAG |
| | CCCACCTGGAGGACAAATATCGGTACCTGTTTAAGCAGGTGGCCTCCAGCACAGGC |
| | TTCTGTGATCAGCGCCGGCTGGGCCTGCTGCTGCACGACAGCATTCAGATCCCTAG |
| | GCAGCTGGGCGAGGTGGCCTCCTTCGGCGGCTCAAACATTGAACCCAGCGTGAGAT |
| | CTTGCTTTCAGTTTGCAAATAATAAACCCGAGATCGAGGCCGCCCTGTTCCTGGAT |
| | TGGATGAGACTGGAGCCTCAGAGTATGGTGTGGCTGCCAGTGCTGCATAGAGTGGC |
| | CGCCGCTGAGACGGCCAAGCACCAGGCAAAGTGCAATATCTGCAAGGAATGCCCCA |
| | TTATTGGCTTTCGATACCGGTCCTTGAAACACTTCAATTATGACATCTGCCAGTCA |
| | TGCTTCTTCAGCGGACGCGTGGCCAAGGGACACAAGATGCATTATCCTATGGTGGA |
| | ATACTGCACTCCAACTACTTCCGGCGAGGACGTGAGAGATTTCGCTAAAGTTCTTA |
| | AAAACAAATTTCGGACTAAGAGGTACTTCGCCAAGCACCCCCGCATGGGCTATCTG |
| | CCTGTGCAGACAGTGCTTGAAGGAGACAACATGGAAACCGATACAATGTGA (SEQ |
| | ID NO: 2406) |
| First DRG de- | TCATACAGCTAGATAACCAAAGA (SEQ ID NO: 2401) |
| targeting | |
| miRNA binding | |
| site | |
| Second DRG de- | TCATACAGCTAGATAACCAAAGA (SEQ ID NO: 2401) |
| targeting | |
| miRNA binding | |
| site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 17
(SEQ ID NO: 2428)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 17 | |
| annotated | Sequence |
| AAV2 ITR | GCGCGCTCGCTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGAGTGGCCAACT |
| | CCATCACTAGGGGTTCCT (SEQ ID NO: 2408) |
| Novel promoter | CGTCTGTCTAATTCCAGCCAAGGGCATGGGGGAAAGGACAGAGCCCAGGGACCTG |
| | TGGCCTATTCTCTGAGAGGACGCTGGGCTAATAATAGGAGGGGAGATTAGGGAGG |
| | GATTTGACTGCATTGTTATCTCACAGGAATGGCGTTTGAGAGCCTTGCTTCCAAG |
| | CTCTTCCAAGAGTCATGCCACCCCCCTCCAATCTCTGTGGAAGTGCCCACTGCCT |
| | TTCCCGCTTCGTTCTCTTCTCTCTAAATCTGAGCCCACCCCACTCAGGGGTACAG |
| | GATCTGGTTCATTTATCTCCCAGAATGATGCCTTGGGTTGCGTCCCCTCTAAACT |
| | CTGGCACATTCCAGCCCCTCTTTGGGAGAAAGAACATCTCTTCTTGCCCAGGCCC |
| | TAGTGAGACATGCATCGCAAGACATGCTCCCCCCTTCCTCCCCGCTGGGCTCCAG |
| | CTGCTGCCAGCCCTCCAAGAAAGGAGAGGCCCTGAGTTGGGCTATTTTGGTATCT |
| | GGGGTGGGCACCCGCAGGGCTAAGGTTACCTTGGTATGTTAAGGGCTCCCTGGGG |
| | CAGGACTATATAACCCCAGAGGGACTGCCCCATGCTGACTCCTTGCTTCTTTCCT |
| | GCTCGAGCCGCTGCCTTGCCCCCCGGAGACTGAAGACA (SEQ ID NO: |
| | 2403) |
| Intron | GTAAGTATCAAGGTTACAAGACAGGTTTAAGGAGACCAATAGAAACTGGGCTTGT |
| | CGAGACAGAGAAGACTCTTGCGTTTCTGATAGGCACCTATTGGTCTTACTGACAT |
| | CCACTTTGCCTTTCTCTCCACAG (SEQ ID NO: 2409) |
| Codon optimized | ATGCTGTGGTGGGAAGAGGTCGAGGACTGCTACGAGCGCGAAGATGTGCAGAAGA |
| microdystrophin | AAACTTTCACTAAATGGGTGAATGCCCAGTTCTCTAAGTTCGGAAAGCAGCACAT |
| coding sequence | CGAAAACCTGTTTTCCGATCTGCAAGATGGCCGGCGGCTGCTGGACCTGCTGGAG |
| | GGGCTGACCGGACAGAAGCTGCCTAAGGAGAAAGGATCTACCAGGGTCCATGCTC |
| | TGAATAACGTGAACAAGGCTCTGAGGGTGCTGCAGAACAATAACGTGGACCTGGT |
| | GAACATTGGTTCCACAGACATCGTGGATGGAAACCACAAACTGACACTGGGACTG |
| | ATCTGGAACATCATTCTGCATTGGCAAGTGAAGAACGTGATGAAGAACATCATGG |
| | CCGGACTGCAGCAGACGAACTCCGAGAAGATCCTGCTTAGCTGGGTGAGACAGAG |
| | TACCCGGAATTACCCACAGGTGAACGTGATCAATTTCACAACCAGCTGGTCAGAC |
| | GGCCTGGCTCTGAATGCCCTGATTCATAGCCACCGGCCAGATCTGTTTGACTGGA |
| | ACTCTGTCGTGTGTCAGCAGTCAGCCACCCAGAGGCTGGAGCACGCCTTCAATAT |
| | TGCCCGCTATCAGCTGGGGATTGAGAAACTGCTGGACCCCGAGGATGTGGACACA |
| | ACATACCCTGACAAGAAGAGCATACTTATGTATATCACAAGCCTGTTCCAGGTGC |
| | TGCCACAGCAGGTCTCCATCGAGGCCATTCAAGAAGTGGAGATGCTGCCCAGGCC |
| | TCCTAAGGTCACCAAAGAGGAGCACTTTCAGCTCCATCATCAGATGCACTACAGC |
| | CAGCAAATTACCGTGTCCCTGGCCCAGGGCTACGAGAGGACAAGTTCCCCCAAGC |
| | CTAGATTTAAGAGCTATGCCTATACCCAGGCCGCCTACGTGACTACCTCCGATCC |
| | CACCAGGAGCCCATTTCCCAGCCAGCACCTGGAGGCTCCCGAAGATAAGAGCTTT |
| | GGCAGCAGCCTGATGGAATCTGAGGTGAACCTGGACAGATACCAGACTGCGCTGG |
| | AGGAGGTGCTGTCCTGGCTGCTGAGCGCCGAGGACACCCTGCAGGCTCAGGGCGA |
| | GATTAGTAATGATGTGGAGGTGGTGAAGGATCAGTTTCACACTCACGAAGGCTAC |
| | ATGATGGACCTGACCGCCCACCAGGGCAGGGTGGGGAACATTCTGCAGCTCGGGA |
| | GTAAGCTGATAGGGACCGGCAAACTGTCCGAGGATGAGGAAACCGAGGTGCAGGA |
| | GCAGATGAACCTGCTTAACTCCCGGTGGGAGTGTCTGAGAGTGGCCAGCATGGAG |
| | AAGCAGAGCAATCTGCACAGAGTGCTGATGGATCTGCAGAATCAGAAGCTGAAGG |
| | AACTGAATGATTGGCTCACAAAGACAGAGGAGAGGACTAGAAAGATGGAGGAGGA |
| | GCCCCTGGGCCCTGACCTGGAGGACCTGAAGCGACAGGTGCAGCAGCACAAGGTG |
| | CTGCAGGAGGATCTGGAGCAAGAACAAGTGAGGGTGAACTCTCTGACTCATATGG |
| | TTGTGGTGGTGGACGAGTCTTCCGGAGACCACGCAACTGCCGCCCTCGAGGAGCA |
| | GCTGAAGGTGCTGGGCGATAGGTGGGCTAATATCTGTAGGTGGACCGAGGACAGA |
| | TGGGTGCTGCTGCAGGATATCCTGCTGAAATGGCAGCGCCTGACAGAAGAGCAGT |
| | GTCTGTTCAGCGCATGGCTGAGCGAAAAGGAGGACGCTGTGAACAAGATCCACAC |
| | TACAGGATTTAAAGACCAGAACGAGATGCTGAGTAGCCTGCAGAAGCTGGCTGTG |
| | CTGAAGGCTGATTTGGAGAAAAAGAAGCAGAGCATGGGCAAGCTGTACTCCCTGA |
| | AACAGGACCTGCTGAGCACCCTGAAGAATAAGTCCGTGACCCAGAAAACCGAAGC |
| | TTGGCTGGACAACTTCGCGCGCTGCTGGGATAACCTGGTGCAGAAGCTGGAGAAG |
| | AGCACCGCCCAGATTTCTCAAGCAGTGACCACAACCCAGCCCAGCCTGACCCAGA |
| | CTACAGTAATGGAGACAGTCACTACCGTGACCACCCGGGAGCAGATCCTGGTGAA |
| | GCACGCCCAGGAAGAGCTGCCGCCCCCACCACCCCAGAAAAAGCGGACCCTGGAG |
| | CGCCTGCAGGAGCTCCAGGAGGCCACCGACGAACTGGATCTGAAGCTGAGGCAGG |
| | CCGAGGTGATTAAGGGATCCTGGCAGCCTGTGGGAGACCTGCTGATCGACAGCCT |
| | GCAGGATCATCTGGAGAAAGTCAAAGCTCTGAGAGGGGAGATCGCCCCACTGAAG |
| | GAGAACGTGAGCCACGTGAATGACCTGGCTAGACAGCTGACCACCTTGGGCATTC |
| | AGCTGAGTCCTTACAATCTGTCAACTCTGGAGGATCTGAACACCCGGTGGAAACT |
| | GCTGCAGGTGGCTGTGGAAGATCGGGTGCGCCAGTTACATGAGGCCCACAGGGAC |
| | TTTGGCCCAGCCTCACAGCACTTTCTGTCAACCTCTGTGCAAGGACCTTGGGAGA |
| | GAGCAATCAGCCCAAACAAGGTGCCATATTATATCAATCATGAGACTCAGACAAC |
| | TTGCTGGGATCACCCCAAGATGACAGAACTCTATCAGTCACTCGCCGATCTCAAT |
| | AATGTACGGTTCTCTGCCTACCGGACAGCTATGAAGCTGCGGAGGCTGCAGAAGG |
| | CACTCTGTCTGGACCTGCTGAGTCTCTCTGCGGCCTGCGATGCCCTGGATCAACA |
| | TAACCTGAAGCAGAATGATCAGCCTATGGACATCCTGCAGATCATTAACTGTCTC |
| | ACCACAATCTACGATCGGCTGGAACAGGAACATAACAACCTGGTGAATGTGCCAC |
| | TGTGTGTGGACATGTGCCTGAATTGGCTGCTCAACGTCTACGATACAGGCAGGAC |
| | TGGCAGGATCCGCGTGCTGAGCTTTAAAACCGGCATCATCTCTCTGTGCAAAGCC |
| | CACCTGGAGGACAAATATCGGTACCTGTTTAAGCAGGTGGCCTCCAGCACAGGCT |
| | TCTGTGATCAGCGCCGGCTGGGCCTGCTGCTGCACGACAGCATTCAGATCCCTAG |
| | GCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGT |
| | GGCCGCCGCTGAGACGGCCAAGCACCAGGCAAAGTGCAATATCTGCAAGGAATGC |
| | CCCATTATTGGCTTTCGATACCGGTCCTTGAAACACTTCAATTATGACATCTGCC |
| | AGTCATGCTTCTTCAGCGGACGCGTGGCCAAGGGACACAAGATGCATTATCCTAT |
| | GGTGGAATACTGCACTCCAACTACTTCCGGCGAGGACGTGAGAGATTTCGCTAAA |
| | GTTCTTAAAAACAAATTTCGGACTAAGAGGTACTTCGCCAAGCACCCCCGCATGG |
| | GCTATCTGCCTGTGCAGACAGTGCTTGAAGGAGACAACATGGAAACCGATACAAT |
| | GTGA (SEQ ID NO: 2406) |
| First DRG de- | TCATACAGCTAGATAACCAAAGA (SEQ ID NO: 2401) |
| targeting miRNA | |
| binding site | |
| Second DRG de- | TCATACAGCTAGATAACCAAAGA (SEQ ID NO: 2401) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 2411) |

Nucleic acid 18
(SEQ ID NO: 2429)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAAGAGGTCGAGGACTGCT
ACGAGCGCGAAGATGTGCAGAAGAAAACTTTCACTAAATGGGTGAATGCCCAGTTCTCTAAGTTCGGAAAGCAGC
ACATCGAAAACCTGTTTTCCGATCTGCAAGATGGCCGGCGGCTGCTGGACCTGCTGGAGGGGCTGACCGGACAGA
AGCTGCCTAAGGAGAAAGGATCTACCAGGGTCCATGCTCTGAATAACGTGAACAAGGCTCTGAGGGTGCTGCAGA
ACAATAACGTGGACCTGGTGAACATTGGTTCCACAGACATCGTGGATGGAAACCACAAACTGACACTGGGACTGA
TCTGGAACATCATTCTGCATTGGCAAGTGAAGAACGTGATGAAGAACATCATGGCCGGACTGCAGCAGACGAACT
CCGAGAAGATCCTGCTTAGCTGGGTGAGACAGAGTACCCGGAATTACCCACAGGTGAACGTGATCAATTTCACAA
CCAGCTGGTCAGACGGCCTGGCTCTGAATGCCCTGATTCATAGCCACCGGCCAGATCTGTTTGACTGGAACTCTG
TCGTGTGTCAGCAGTCAGCCACCCAGAGGCTGGAGCACGCCTTCAATATTGCCCGCTATCAGCTGGGGATTGAGA
AACTGCTGGACCCCGAGGATGTGGACACAACATACCCTGACAAGAAGAGCATACTTATGTATATCACAAGCCTGT
TCCAGGTGCTGCCACAGCAGGTCTCCATCGAGGCCATTCAAGAAGTGGAGATGCTGCCCAGGCCTCCTAAGGTCA
CCAAAGAGGAGCACTTTCAGCTCCATCATCAGATGCACTACAGCCAGCAAATTACCGTGTCCCTGGCCCAGGGCT
ACGAGAGGACAAGTTCCCCCAAGCCTAGATTTAAGAGCTATGCCTATACCCAGGCCGCCTACGTGACTACCTCCG
ATCCCACCAGGAGCCCATTTCCCAGCCAGCACCTGGAGGCTCCCGAAGATAAGAGCTTTGGCAGCAGCCTGATGG
AATCTGAGGTGAACCTGGACAGATACCAGACTGCGCTGGAGGAGGTGCTGTCCTGGCTGCTGAGCGCCGAGGACA
CCCTGCAGGCTCAGGGCGAGATTAGTAATGATGTGGAGGTGGTGAAGGATCAGTTTCACACTCACGAAGGCTACA
TGATGGACCTGACCGCCCACCAGGGCAGGGTGGGGAACATTCTGCAGCTCGGGAGTAAGCTGATAGGGACCGGCA
AACTGTCCGAGGATGAGGAAACCGAGGTGCAGGAGCAGATGAACCTGCTTAACTCCCGGTGGGAGTGTCTGAGAG
TGGCCAGCATGGAGAAGCAGAGCAATCTGCACAGAGTGCTGATGGATCTGCAGAATCAGAAGCTGAAGGAACTGA
ATGATTGGCTCACAAAGACAGAGGAGAGGACTAGAAAGATGGAGGAGGAGCCCCTGGGCCCTGACCTGGAGGACC
TGAAGCGACAGGTGCAGCAGCACAAGGTGCTGCAGGAGGATCTGGAGCAAGAACAAGTGAGGGTGAACTCTCTGA
CTCATATGGTTGTGGTGGTGGACGAGTCTTCCGGAGACCACGCAACTGCCGCCCTCGAGGAGCAGCTGAAGGTGC
TGGGCGATAGGTGGGCTAATATCTGTAGGTGGACCGAGGACAGATGGGTGCTGCTGCAGGATATCCTGCTGAAAT
GGCAGCGCCTGACAGAAGAGCAGTGTCTGTTCAGCGCATGGCTGAGCGAAAAGGAGGACGCTGTGAACAAGATCC
ACACTACAGGATTTAAAGACCAGAACGAGATGCTGAGTAGCCTGCAGAAGCTGGCTGTGCTGAAGGCTGATTTGG
AGAAAAAGAAGCAGAGCATGGGCAAGCTGTACTCCCTGAAACAGGACCTGCTGAGCACCCTGAAGAATAAGTCCG
TGACCCAGAAAACCGAAGCTTGGCTGGACAACTTCGCGCGCTGCTGGGATAACCTGGTGCAGAAGCTGGAGAAGA
GCACCGCCCAGATTTCTCAAGCAGTGACCACAACCCAGCCCAGCCTGACCCAGACTACAGTAATGGAGACAGTCA
CTACCGTGACCACCCGGGAGCAGATCCTGGTGAAGCACGCCCAGGAAGAGCTGCCGCCCCCACCACCCCAGAAAA
AGCGGACCCTGGAGCGCCTGCAGGAGCTCCAGGAGGCCACCGACGAACTGGATCTGAAGCTGAGGCAGGCCGAGG
TGATTAAGGGATCCTGGCAGCCTGTGGGAGACCTGCTGATCGACAGCCTGCAGGATCATCTGGAGAAAGTCAAAG
CTCTGAGAGGGGAGATCGCCCCACTGAAGGAGAACGTGAGCCACGTGAATGACCTGGCTAGACAGCTGACCACCT
TGGGCATTCAGCTGAGTCCTTACAATCTGTCAACTCTGGAGGATCTGAACACCCGGTGGAAACTGCTGCAGGTGG
CTGTGGAAGATCGGGTGCGCCAGTTACATGAGGCCCACAGGGACTTTGGCCCAGCCTCACAGCACTTTCTGTCAA
CCTCTGTGCAAGGACCTTGGGAGAGAGCAATCAGCCCAAACAAGGTGCCATATTATATCAATCATGAGACTCAGA
CAACTTGCTGGGATCACCCCAAGATGACAGAACTCTATCAGTCACTCGCCGATCTCAATAATGTACGGTTCTCTG
CCTACCGGACAGCTATGAAGCTGCGGAGGCTGCAGAAGGCACTCTGTCTGGACCTGCTGAGTCTCTCTGCGGCCT
GCGATGCCCTGGATCAACATAACCTGAAGCAGAATGATCAGCCTATGGACATCCTGCAGATCATTAACTGTCTCA
CCACAATCTACGATCGGCTGGAACAGGAACATAACAACCTGGTGAATGTGCCACTGTGTGTGGACATGTGCCTGA
ATTGGCTGCTCAACGTCTACGATACAGGCAGGACTGGCAGGATCCGCGTGCTGAGCTTTAAAACCGGCATCATCT
CTCTGTGCAAAGCCCACCTGGAGGACAAATATCGGTACCTGTTTAAGCAGGTGGCCTCCAGCACAGGCTTCTGTG
ATCAGCGCCGGCTGGGCCTGCTGCTGCACGACAGCATTCAGATCCCTAGGCAGCTGGGCGAGGTGGCCTCCTTCG
GCGGCTCAAACATTGAACCCAGCGTGAGATCTTGCTTTCAGTTTGCAAATAATAAACCCGAGATCGAGGCCGCCC
TGTTCCTGGATTGGATGAGACTGGAGCCTCAGAGTATGGTGTGGCTGCCAGTGCTGCATAGAGTGGCCGCCGCTG
AGACGGCCAAGCACCAGGCAAAGTGCAATATCTGCAAGGAATGCCCCATTATTGGCTTTCGATACCGGTCCTTGA
AACACTTCAATTATGACATCTGCCAGTCATGCTTCTTCAGCGGACGCGTGGCCAAGGGACACAAGATGCATTATC
CTATGGTGGAATACTGCACTCCAACTACTTCCGGCGAGGACGTGAGAGATTTCGCTAAAGTTCTTAAAAACAAAT
TTCGGACTAAGAGGTACTTCGCCAAGCACCCCCGCATGGGCTATCTGCCTGTGCAGACAGTGCTTGAAGGAGACA
ACATGGAAACCGATACAATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 18 | |
| annotated | Sequence |
| AAV2 ITR | GCGCGCTCGCTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGAGTGGCCAACT |
| | CCATCACTAGGGGTTCCT (SEQ ID NO: 2408) |
| Novel promoter | CGTCTGTCTAATTCCAGCCAAGGGCATGGGGGAAAGGACAGAGCCCAGGGACCTG |
| | TGGCCTATTCTCTGAGAGGACGCTGGGCTAATAATAGGAGGGGAGATTAGGGAGG |
| | GATTTGACTGCATTGTTATCTCACAGGAATGGCGTTTGAGAGCCTTGCTTCCAAG |
| | CTCTTCCAAGAGTCATGCCACCCCCCTCCAATCTCTGTGGAAGTGCCCACTGCCT |
| | TTCCCGCTTCGTTCTCTTCTCTCGGTCCCATCCCTGACCCACTAGCCTGGTGCCG |
| | GGGGGAGACCAATTTCTGGAGGAAGCTCTGGGTACCCAGTCTTGCCCTAAGCCTG |
| | CCTCTGAACAGGCTGTTCTTGTTTATGACCCCCGTCCCAGCAGAATAGTGATTGC |
| | TCATCCCTGCATGTGATTTGAGAGGGCCAGTCTTTCAGGCCTGGAGGAAACTCAA |
| | GCCCCACCAGCCGCTTGGCATCGGAATGCTCAGCTGGTCCCCTCCTGGGGCTCAT |
| | GTGACCAAGATCTAGCTATAAATATCAGAGGGGCCTCAGACCAAGACAGAATTCG |
| | GGCACCAGGAGAAGGAAGCCAACAGGATCCGACCCGGTGTTTTGTGACAAAGGCA |
| | AGACCCCCAGGTCTACTTAGAGCAACGTTAGTAGAGGA (SEQ ID NO: |
| | 2404) |
| Intron | GTAAGTATCAAGGTTACAAGACAGGTTTAAGGAGACCAATAGAAACTGGGCTTGT |
| | CGAGACAGAGAAGACTCTTGCGTTTCTGATAGGCACCTATTGGTCTTACTGACAT |
| | CCACTTTGCCTTTCTCTCCACAG (SEQ ID NO: 2409) |
| Codon optimized | ATGCTGTGGTGGGAAGAGGTCGAGGACTGCTACGAGCGCGAAGATGTGCAGAAGA |
| microdystrophin | AAACTTTCACTAAATGGGTGAATGCCCAGTTCTCTAAGTTCGGAAAGCAGCACAT |
| coding sequence | CGAAAACCTGTTTTCCGATCTGCAAGATGGCCGGCGGCTGCTGGACCTGCTGGAG |
| | GGGCTGACCGGACAGAAGCTGCCTAAGGAGAAAGGATCTACCAGGGTCCATGCTC |
| | TGAATAACGTGAACAAGGCTCTGAGGGTGCTGCAGAACAATAACGTGGACCTGGT |
| | GAACATTGGTTCCACAGACATCGTGGATGGAAACCACAAACTGACACTGGGACTG |
| | ATCTGGAACATCATTCTGCATTGGCAAGTGAAGAACGTGATGAAGAACATCATGG |
| | CCGGACTGCAGCAGACGAACTCCGAGAAGATCCTGCTTAGCTGGGTGAGACAGAG |
| | TACCCGGAATTACCCACAGGTGAACGTGATCAATTTCACAACCAGCTGGTCAGAC |
| | GGCCTGGCTCTGAATGCCCTGATTCATAGCCACCGGCCAGATCTGTTTGACTGGA |
| | ACTCTGTCGTGTGTCAGCAGTCAGCCACCCAGAGGCTGGAGCACGCCTTCAATAT |
| | TGCCCGCTATCAGCTGGGGATTGAGAAACTGCTGGACCCCGAGGATGTGGACACA |
| | ACATACCCTGACAAGAAGAGCATACTTATGTATATCACAAGCCTGTTCCAGGTGC |
| | TGCCACAGCAGGTCTCCATCGAGGCCATTCAAGAAGTGGAGATGCTGCCCAGGCC |
| | TCCTAAGGTCACCAAAGAGGAGCACTTTCAGCTCCATCATCAGATGCACTACAGC |
| | CAGCAAATTACCGTGTCCCTGGCCCAGGGCTACGAGAGGACAAGTTCCCCCAAGC |
| | CTAGATTTAAGAGCTATGCCTATACCCAGGCCGCCTACGTGACTACCTCCGATCC |
| | CACCAGGAGCCCATTTCCCAGCCAGCACCTGGAGGCTCCCGAAGATAAGAGCTTT |
| | GGCAGCAGCCTGATGGAATCTGAGGTGAACCTGGACAGATACCAGACTGCGCTGG |
| | AGGAGGTGCTGTCCTGGCTGCTGAGCGCCGAGGACACCCTGCAGGCTCAGGGCGA |
| | GATTAGTAATGATGTGGAGGTGGTGAAGGATCAGTTTCACACTCACGAAGGCTAC |
| | ATGATGGACCTGACCGCCCACCAGGGCAGGGTGGGGAACATTCTGCAGCTCGGGA |
| | GTAAGCTGATAGGGACCGGCAAACTGTCCGAGGATGAGGAAACCGAGGTGCAGGA |
| | GCAGATGAACCTGCTTAACTCCCGGTGGGAGTGTCTGAGAGTGGCCAGCATGGAG |
| | AAGCAGAGCAATCTGCACAGAGTGCTGATGGATCTGCAGAATCAGAAGCTGAAGG |
| | AACTGAATGATTGGCTCACAAAGACAGAGGAGAGGACTAGAAAGATGGAGGAGGA |
| | GCCCCTGGGCCCTGACCTGGAGGACCTGAAGCGACAGGTGCAGCAGCACAAGGTG |
| | CTGCAGGAGGATCTGGAGCAAGAACAAGTGAGGGTGAACTCTCTGACTCATATGG |
| | TTGTGGTGGTGGACGAGTCTTCCGGAGACCACGCAACTGCCGCCCTCGAGGAGCA |
| | GCTGAAGGTGCTGGGCGATAGGTGGGCTAATATCTGTAGGTGGACCGAGGACAGA |
| | TGGGTGCTGCTGCAGGATATCCTGCTGAAATGGCAGCGCCTGACAGAAGAGCAGT |
| | GTCTGTTCAGCGCATGGCTGAGCGAAAAGGAGGACGCTGTGAACAAGATCCACAC |
| | TACAGGATTTAAAGACCAGAACGAGATGCTGAGTAGCCTGCAGAAGCTGGCTGTG |
| | CTGAAGGCTGATTTGGAGAAAAAGAAGCAGAGCATGGGCAAGCTGTACTCCCTGA |
| | AACAGGACCTGCTGAGCACCCTGAAGAATAAGTCCGTGACCCAGAAAACCGAAGC |
| | TTGGCTGGACAACTTCGCGCGCTGCTGGGATAACCTGGTGCAGAAGCTGGAGAAG |
| | AGCACCGCCCAGATTTCTCAAGCAGTGACCACAACCCAGCCCAGCCTGACCCAGA |
| | CTACAGTAATGGAGACAGTCACTACCGTGACCACCCGGGAGCAGATCCTGGTGAA |
| | GCACGCCCAGGAAGAGCTGCCGCCCCCACCACCCCAGAAAAAGCGGACCCTGGAG |
| | CGCCTGCAGGAGCTCCAGGAGGCCACCGACGAACTGGATCTGAAGCTGAGGCAGG |
| | CCGAGGTGATTAAGGGATCCTGGCAGCCTGTGGGAGACCTGCTGATCGACAGCCT |
| | GCAGGATCATCTGGAGAAAGTCAAAGCTCTGAGAGGGGAGATCGCCCCACTGAAG |
| | GAGAACGTGAGCCACGTGAATGACCTGGCTAGACAGCTGACCACCTTGGGCATTC |
| | AGCTGAGTCCTTACAATCTGTCAACTCTGGAGGATCTGAACACCCGGTGGAAACT |
| | GCTGCAGGTGGCTGTGGAAGATCGGGTGCGCCAGTTACATGAGGCCCACAGGGAC |
| | TTTGGCCCAGCCTCACAGCACTTTCTGTCAACCTCTGTGCAAGGACCTTGGGAGA |
| | GAGCAATCAGCCCAAACAAGGTGCCATATTATATCAATCATGAGACTCAGACAAC |
| | TTGCTGGGATCACCCCAAGATGACAGAACTCTATCAGTCACTCGCCGATCTCAAT |
| | AATGTACGGTTCTCTGCCTACCGGACAGCTATGAAGCTGCGGAGGCTGCAGAAGG |
| | CACTCTGTCTGGACCTGCTGAGTCTCTCTGCGGCCTGCGATGCCCTGGATCAACA |
| | TAACCTGAAGCAGAATGATCAGCCTATGGACATCCTGCAGATCATTAACTGTCTC |
| | ACCACAATCTACGATCGGCTGGAACAGGAACATAACAACCTGGTGAATGTGCCAC |
| | TGTGTGTGGACATGTGCCTGAATTGGCTGCTCAACGTCTACGATACAGGCAGGAC |
| | TGGCAGGATCCGCGTGCTGAGCTTTAAAACCGGCATCATCTCTCTGTGCAAAGCC |
| | CACCTGGAGGACAAATATCGGTACCTGTTTAAGCAGGTGGCCTCCAGCACAGGCT |
| | TCTGTGATCAGCGCCGGCTGGGCCTGCTGCTGCACGACAGCATTCAGATCCCTAG |
| | GCAGCTGGGCGAGGTGGCCTCCTTCGGCGGCTCAAACATTGAACCCAGCGTGAGA |
| | TCTTGCTTTCAGTTTGCAAATAATAAACCCGAGATCGAGGCCGCCCTGTTCCTGG |
| | ATTGGATGAGACTGGAGCCTCAGAGTATGGTGTGGCTGCCAGTGCTGCATAGAGT |
| | GGCCGCCGCTGAGACGGCCAAGCACCAGGCAAAGTGCAATATCTGCAAGGAATGC |
| | CCCATTATTGGCTTTCGATACCGGTCCTTGAAACACTTCAATTATGACATCTGCC |
| | AGTCATGCTTCTTCAGCGGACGCGTGGCCAAGGGACACAAGATGCATTATCCTAT |
| | GGTGGAATACTGCACTCCAACTACTTCCGGCGAGGACGTGAGAGATTTCGCTAAA |
| | GTTCTTAAAAACAAATTTCGGACTAAGAGGTACTTCGCCAAGCACCCCCGCATGG |
| | GCTATCTGCCTGTGCAGACAGTGCTTGAAGGAGACAACATGGAAACCGATACAAT |
| | GTGA (SEQ ID NO: 2406) |
| First DRG de- | TCATACAGCTAGATAACCAAAGA (SEQ ID NO: 2401) |
| targeting miRNA | |
| binding site | |
| Second DRG de- | TCATACAGCTAGATAACCAAAGA (SEQ ID NO: 2401) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 2411) |

Nucleic acid 19
(SEQ ID NO: 2430)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAGGTGGAGGATTGCTACGAGAGAGAGGACGTGCAGAAGAAAACCTTCACC
AAATGGGTGAACGCCCAGTTCAGCAAGTTTGGCAAGCAGCACATCGAGAACCTGTTCAGCGATCTGCAGGACGGC
CGCAGGCTGCTGGATCTGCTGGAGGGTCTGACCGGCCAGAAACTGCCCAAAGAGAAGGGATCAACCAGGGTGCAC
GCCCTGAATAATGTGAACAAGGCCCTGAGAGTGCTGCAAAATAATAATGTGGATCTGGTGAACATCGGATCTACC
GATATTGTGGACGGCAACCATAAGCTGACTCTGGGCCTGATCTGGAATATCATCCTGCACTGGCAGGTGAAAAAT
GTGATGAAAAACATCATGGCCGGGCTGCAGCAGACCAACTCTGAGAAGATCCTGCTGAGCTGGGTGAGGCAGTCT
ACAAGAAATTACCCCCAGGTGAACGTGATCAATTTCACCACCAGCTGGTCCGACGGGCTGGCTCTGAATGCCCTG
ATTCACAGCCACCGGCCCGACCTGTTCGACTGGAATTCCGTGGTGTGCCAGCAGAGCGCCACACAGAGACTGGAG
CACGCTTTCAATATCGCTCGGTATCAGCTGGGCATCGAGAAGCTGCTGGACCCCGAGGACGTGGACACTACTTAC
CCCGATAAAAAGAGCATCCTGATGTATATCACCTCCCTGTTCCAAGTGCTGCCCCAGCAGGTCTCCATCGAGGCC
ATCCAGGAGGTGGAAATGCTGCCCCGGCCCCCAAAGGTGACAAAGGAAGAACACTTCCAGCTCCACCACCAGATG
CACTACTCTCAGCAGATCACCGTGAGCCTGGCCCAGGGGTACGAGCGGACCTCTTCTCCAAAGCCCCGCTTCAAG
AGCTATGCCTATACCCAGGCCGCCTATGTGACCACCTCCGATCCCACCCGCTCACCCTTCCCTTCTCAACACCTG
GAAGCCCCAGAGGACAAGTCCTTCGGCAGTTCTCTGATGGAGTCTGAGGTGAACCTGGATAGATATCAGACTGCC
CTGGAGGAGGTGCTGTCTTGGCTGCTCTCCGCCGAGGACACCCTGCAGGCACAGGGCGAGATCTCCAACGATGTG
GAGGTGGTGAAGGACCAGTTCCACACACACGAGGGCTATATGATGGACCTGACTGCCCATCAGGGCAGAGTGGGC
AATATCCTGCAGCTGGGTTCCAAACTGATAGGCACCGGAAAGCTGAGCGAAGACGAGGAGACCGAGGTGCAGGAG
CAGATGAATCTGCTGAACTCCCGGTGGGAGTGCCTGCGAGTCGCCAGCATGGAAAAGCAGAGCAACCTGCACCGC
GTGCTCATGGATCTGCAGAATCAGAAGCTGAAGGAGCTGAATGACTGGCTGACCAAGACCGAAGAGCGCACCAGG
AAGATGGAGGAGGAGCCACTGGGCCCCGATCTGGAGGACCTGAAAAGACAGGTGCAGCAGCATAAAGTGCTGCAG
GAGGACCTGGAGCAGGAACAAGTGAGAGTGAATTCTCTGACACACATGGTGGTGGTGGTGGACGAGAGCAGCGGC
GACCACGCAACAGCCGCCCTGGAGGAGCAGCTGAAAGTGCTGGGAGACCGGTGGGCCAACATTTGCCGGTGGACC
GAGGACCGCTGGGTGCTGCTGCAGGACATTCTGCTGAAGTGGCAGCGGCTGACCGAAGAGCAGTGCCTGTTCTCC
GCCTGGCTGAGCGAGAAGGAGGACGCCGTGAATAAGATCCACACCACCGGGTTCAAGGACCAGAATGAGATGCTG
TCCAGCCTGCAGAAGCTGGCCGTGCTGAAGGCCGATCTGGAAAAGAAGAAGCAGAGCATGGGAAAGCTGTATAGC
CTGAAGCAGGACCTGCTGTCTACTCTGAAAAACAAGTCAGTGACCCAGAAGACCGAGGCCTGGCTGGACAACTTC
GCCCGCTGCTGGGACAACCTGGTGCAGAAGCTGGAAAAGAGCACCGCCCAGATCAGCCAGGCAGTGACTACCACA
CAGCCCTCCCTGACACAGACTACTGTGATGGAGACCGTGACAACAGTGACCACAAGGGAGCAGATCCTGGTGAAG
CACGCCCAGGAAGAACTGCCCCCTCCACCCCCTCAGAAGAAGAGAACTCTGGAAAGACTGCAGGAGCTGCAGGAG
GCCACTGACGAGCTGGACCTGAAGCTGCGGCAGGCCGAGGTGATTAAGGGCAGCTGGCAGCCTGTGGGCGATCTG
CTGATCGACAGCCTGCAGGACCACCTGGAGAAGGTGAAAGCCCTGCGGGGCGAGATCGCCCCTCTGAAGGAGAAC
GTCAGCCATGTGAATGACCTGGCCAGACAGCTGACCACACTGGGGATTCAGCTGTCTCCTTACAATCTGTCAACC
CTGGAGGACCTGAACACTCGCTGGAAGCTGCTGCAGGTGGCCGTGGAGGACAGGGTGCGGCAGCTGCACGAAGCC
CATCGCGACTTTGGGCCCGCCTCTCAGCACTTTCTGAGCACCTCCGTGCAGGGACCCTGGGAGCGGGCAATCAGC
CCAAACAAGGTGCCATACTACATCAACCACGAGACCCAGACCACCTGTTGGGATCACCCCAAGATGACAGAGCTG
TACCAGTCTCTGGCTGACCTGAACAACGTGCGGTTCTCAGCCTACAGGACCGCCATGAAGCTGCGGAGACTGCAG
AAGGCCCTGTGCCTGGATCTGCTGAGCCTCTCCGCTGCTTGTGACGCCCTGGATCAGCACAACCTGAAACAGAAC
GACCAGCCAATGGACATCCTGCAGATTATTAATTGCCTGACAACCATCTATGATCGGCTGGAACAGGAGCACAAT
AATCTGGTGAACGTGCCACTGTGCGTGGACATGTGCCTGAATTGGCTGCTGAACGTGTACGACACAGGACGGACC
GGCAGAATCAGAGTGCTGTCTTTTAAGACCGGGATCATCAGCCTGTGCAAAGCCCACCTGGAGGACAAGTACCGG
TATCTGTTCAAACAAGTGGCCTCCAGCACCGGCTTTTGCGACCAGCGCAGGCTGGGCCTGCTGCTCCACGACTCC
ATCCAGATCCCTAGACAGCTGGGCGAGGTGGCTTCTTTTGGCGGATCCAATATCGAGCCCAGTGTGCGGAGCTGT
TTTCAGTTTGCTAATAATAAGCCTGAGATCGAGGCCGCTCTGTTCCTGGACTGGATGAGACTGGAGCCCCAGTCC
ATGGTGTGGCTGCCAGTGCTGCACAGGGTCGCCGCCGCAGAGACTGCCAAGCACCAGGCCAAGTGCAACATCTGC
AAAGAATGCCCAATCATTGGCTTTAGATACCGGAGCCTCAAACACTTTAACTACGACATCTGCCAGAGCTGCTTC
TTCTCCGGCCGGGTGGCCAAGGGCCACAAGATGCACTACCCCATGGTGGAGTACTGTACCCCAACCACTTCCGGC
GAGGACGTGAGAGATTTCGCCAAGGTGCTGAAGAACAAGTTCCGGACAAAGAGATACTTTGCAAAACACCCAAGA
ATGGGCTACCTGCCAGTGCAGACCGTGCTGGAGGGGGACAATATGGAGACAGACACAATGTGATAGATTCCAACA
AAATCACTGATGCTGGAAGATCAGCAACAAAATCACTGATGCTGGAAGACGCAGGAATAAAATATCTTTATTTTC
ATTACATCTGTGTGTTGGTTTTTTGTGTGAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGTGAGCGAGCG
AGCGCGC

| Nucleic acid | |
|---|---|
| cargo 19 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | TAGTTATTAATAGTAATCAATTACGGGGTCATTAGTTCATAGCCCATATATGGAGT |
| | TCCGCGTTACATAACTTACGGTAAATGGCCCGCCTGGCTGACCGCCCAACGACCCC |
| | CGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATC |
| | AAGTGTATCATATGCCAAGTACGCCCCCTATTGACGTCAATGACGGTAAATGGCCC |
| | GCCTGGCATTATGCCCAGTACATGACCTTATGGGACTTTCCTACTTGGCAGTACAT |
| | CTACGTATTAGTCATCGCTATTACCATGCTAGACTAGCATGCTGCCCATGTAAGGA |
| | GGCAAGGCCTGGGGACACCCGAGATGCCTGGTTATAATTAACCCAGACATGTGGCT |
| | GCCCCCCCCCCCCAACACCTGCTGCCTCTAAAAATAACCCTGCATGCCATGTTCCC |
| | GGCGAAGGGCCAGCTGTCCCCCGCCAGCTAGACTCAGCTCTCAGGGGCCCCTCCCT |
| | GGGGACAGCCCCTCCTGGCTAGTCACACCCTGTAGGCTCCTCTATATAACCCAGGG |
| | GCACAGGGGCTGCCCTCATTCTACCACCACCTCCACAGCACAGACAGACACTCAGG |
| | AGCCAGCCAGC (SEQ ID NO: 2402) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | CAACAAAATCACTGATGCTGGA (SEQ ID NO: 2399) |
| targeting miRNA | |
| binding site | |
| Second DRG de- | CAACAAAATCACTGATGCTGGA (SEQ ID NO: 2399) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 20
(SEQ ID NO: 2431)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | Sequence |
|---|---|
| cargo 20 | |
| annotated | |
| AAV2 ITR | GCGCGCTCGCTCGCTCACTGAGGCCGCCCGGGCAAAGCCCGGGCGTCGGGCGACC |
| | TTTGGTCGCCCGGCCTCAGTGAGCGAGCGAGCGCGCAGAGAGGGAGTGGCCAACT |
| | CCATCACTAGGGGTTCCT (SEQ ID NO: 2408) |
| Novel promoter | CGTCTGTCTAATTCCAGCCAAGGGCATGGGGGAAAGGACAGAGCCCAGGGACCTG |
| | TGGCCTATTCTCTGAGAGGACGCTGGGCTAATAATAGGAGGGGAGATTAGGGAGG |
| | GATTTGACTGCATTGTTATCTCACAGGAATGGCGTTTGAGAGCCTTGCTTCCAAG |
| | CTCTTCCAAGAGTCATGCCACCCCCCTCCAATCTCTGTGGAAGTGCCCACTGCCT |
| | TTCCCGCTTCGTTCTCTTCTCTCTAAATCTGAGCCCACCCCACTCAGGGGTACAG |
| | GATCTGGTTCATTTATCTCCCAGAATGATGCCTTGGGTTGCGTCCCCTCTAAACT |
| | CTGGCACATTCCAGCCCCTCTTTGGGAGAAAGAACATCTCTTCTTGCCCAGGCCC |
| | TAGTGAGACATGCATCGCAAGACATGCTCCCCCCTTCCTCCCCGCTGGGCTCCAG |
| | CTGCTGCCAGCCCTCCAAGAAAGGAGAGGCCCTGAGTTGGGCTATTTTGGTATCT |
| | GGGGTGGGCACCCGCAGGGCTAAGGTTACCTTGGTATGTTAAGGGCTCCCTGGGG |
| | CAGGACTATATAACCCCAGAGGGACTGCCCCATGCTGACTCCTTGCTTCTTTCCT |
| | GCTCGAGCCGCTGCCTTGCCCCCCGGAGACTGAAGACA (SEQ ID NO: |
| | 2403) |
| Intron | GTAAGTATCAAGGTTACAAGACAGGTTTAAGGAGACCAATAGAAACTGGGCTTGT |
| | CGAGACAGAGAAGACTCTTGCGTTTCTGATAGGCACCTATTGGTCTTACTGACAT |
| | CCACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGA |
| microdystrophin | AAACCTTCACCAAATGGGTGAACGCCCAGTTCAGCAAGTTTGGCAAGCAGCACAT |
| coding sequence | CGAGAACCTGTTCAGCGATCTGCAGGACGGCCGCAGGCTGCTGGATCTGCTGGAG |
| | GGTCTGACCGGCCAGAAACTGCCCAAAGAGAAGGGATCAACCAGGGTGCACGCCC |
| | TGAATAATGTGAACAAGGCCCTGAGAGTGCTGCAAAATAATAATGTGGATCTGGT |
| | GAACATCGGATCTACCGATATTGTGGACGGCAACCATAAGCTGACTCTGGGCCTG |
| | ATCTGGAATATCATCCTGCACTGGCAGGTGAAAAATGTGATGAAAAACATCATGG |
| | CCGGGCTGCAGCAGACCAACTCTGAGAAGATCCTGCTGAGCTGGGTGAGGCAGTC |
| | TACAAGAAATTACCCCCAGGTGAACGTGATCAATTTCACCACCAGCTGGTCCGAC |
| | GGGCTGGCTCTGAATGCCCTGATTCACAGCCACCGGCCCGACCTGTTCGACTGGA |
| | ATTCCGTGGTGTGCCAGCAGAGCGCCACACAGAGACTGGAGCACGCTTTCAATAT |
| | CGCTCGGTATCAGCTGGGCATCGAGAAGCTGCTGGACCCCGAGGACGTGGACACT |
| | ACTTACCCCGATAAAAAGAGCATCCTGATGTATATCACCTCCCTGTTCCAAGTGC |
| | TGCCCCAGCAGGTCTCCATCGAGGCCATCCAGGAGGTGGAAATGCTGCCCCGGCC |
| | CCCAAAGGTGACAAAGGAAGAACACTTCCAGCTCCACCACCAGATGCACTACTCT |
| | CAGCAGATCACCGTGAGCCTGGCCCAGGGGTACGAGCGGACCTCTTCTCCAAAGC |
| | CCCGCTTCAAGAGCTATGCCTATACCCAGGCCGCCTATGTGACCACCTCCGATCC |
| | CACCCGCTCACCCTTCCCTTCTCAACACCTGGAAGCCCCAGAGGACAAGTCCTTC |
| | GGCAGTTCTCTGATGGAGTCTGAGGTGAACCTGGATAGATATCAGACTGCCCTGG |
| | AGGAGGTGCTGTCTTGGCTGCTCTCCGCCGAGGACACCCTGCAGGCACAGGGCGA |
| | GATCTCCAACGATGTGGAGGTGGTGAAGGACCAGTTCCACACACACGAGGGCTAT |
| | ATGATGGACCTGACTGCCCATCAGGGCAGAGTGGGCAATATCCTGCAGCTGGGTT |
| | CCAAACTGATAGGCACCGGAAAGCTGAGCGAAGACGAGGAGACCGAGGTGCAGGA |
| | GCAGATGAATCTGCTGAACTCCCGGTGGGAGTGCCTGCGAGTCGCCAGCATGGAA |
| | AAGCAGAGCAACCTGCACCGCGTGCTCATGGATCTGCAGAATCAGAAGCTGAAGG |
| | AGCTGAATGACTGGCTGACCAAGACCGAAGAGCGCACCAGGAAGATGGAGGAGGA |
| | GCCACTGGGCCCCGATCTGGAGGACCTGAAAAGACAGGTGCAGCAGCATAAAGTG |
| | CTGCAGGAGGACCTGGAGCAGGAACAAGTGAGAGTGAATTCTCTGACACACATGG |
| | TGGTGGTGGTGGACGAGAGCAGCGGCGACCACGCAACAGCCGCCCTGGAGGAGCA |
| | GCTGAAAGTGCTGGGAGACCGGTGGGCCAACATTTGCCGGTGGACCGAGGACCGC |
| | TGGGTGCTGCTGCAGGACATTCTGCTGAAGTGGCAGCGGCTGACCGAAGAGCAGT |
| | GCCTGTTCTCCGCCTGGCTGAGCGAGAAGGAGGACGCCGTGAATAAGATCCACAC |
| | CACCGGGTTCAAGGACCAGAATGAGATGCTGTCCAGCCTGCAGAAGCTGGCCGTG |
| | CTGAAGGCCGATCTGGAAAAGAAGAAGCAGAGCATGGGAAAGCTGTATAGCCTGA |
| | AGCAGGACCTGCTGTCTACTCTGAAAAACAAGTCAGTGACCCAGAAGACCGAGGC |
| | CTGGCTGGACAACTTCGCCCGCTGCTGGGACAACCTGGTGCAGAAGCTGGAAAAG |
| | AGCACCGCCCAGATCAGCCAGGCAGTGACTACCACACAGCCCTCCCTGACACAGA |
| | CTACTGTGATGGAGACCGTGACAACAGTGACCACAAGGGAGCAGATCCTGGTGAA |
| | GCACGCCCAGGAAGAACTGCCCCCTCCACCCCCTCAGAAGAAGAGAACTCTGGAA |
| | AGACTGCAGGAGCTGCAGGAGGCCACTGACGAGCTGGACCTGAAGCTGCGGCAGG |
| | CCGAGGTGATTAAGGGCAGCTGGCAGCCTGTGGGCGATCTGCTGATCGACAGCCT |
| | GCAGGACCACCTGGAGAAGGTGAAAGCCCTGCGGGGCGAGATCGCCCCTCTGAAG |
| | GAGAACGTCAGCCATGTGAATGACCTGGCCAGACAGCTGACCACACTGGGGATTC |
| | AGCTGTCTCCTTACAATCTGTCAACCCTGGAGGACCTGAACACTCGCTGGAAGCT |
| | GCTGCAGGTGGCCGTGGAGGACAGGGTGCGGCAGCTGCACGAAGCCCATCGCGAC |
| | TTTGGGCCCGCCTCTCAGCACTTTCTGAGCACCTCCGTGCAGGGACCCTGGGAGC |
| | GGGCAATCAGCCCAAACAAGGTGCCATACTACATCAACCACGAGACCCAGACCAC |
| | CTGTTGGGATCACCCCAAGATGACAGAGCTGTACCAGTCTCTGGCTGACCTGAAC |
| | AACGTGCGGTTCTCAGCCTACAGGACCGCCATGAAGCTGCGGAGACTGCAGAAGG |
| | CCCTGTGCCTGGATCTGCTGAGCCTCTCCGCTGCTTGTGACGCCCTGGATCAGCA |
| | CAACCTGAAACAGAACGACCAGCCAATGGACATCCTGCAGATTATTAATTGCCTG |
| | ACAACCATCTATGATCGGCTGGAACAGGAGCACAATAATCTGGTGAACGTGCCAC |
| | TGTGCGTGGACATGTGCCTGAATTGGCTGCTGAACGTGTACGACACAGGACGGAC |
| | CGGCAGAATCAGAGTGCTGTCTTTTAAGACCGGGATCATCAGCCTGTGCAAAGCC |
| | CACCTGGAGGACAAGTACCGGTATCTGTTCAAACAAGTGGCCTCCAGCACCGGCT |
| | TTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGG |
| | AGCTGTTTTCAGTTTGCTAATAATAAGCCTGAGATCGAGGCCGCTCTGTTCCTGG |
| | ACTGGATGAGACTGGAGCCCCAGTCCATGGTGTGGCTGCCAGTGCTGCACAGGGT |
| | CGCCGCCGCAGAGACTGCCAAGCACCAGGCCAAGTGCAACATCTGCAAAGAATGC |
| | CCAATCATTGGCTTTAGATACCGGAGCCTCAAACACTTTAACTACGACATCTGCC |
| | AGAGCTGCTTCTTCTCCGGCCGGGTGGCCAAGGGCCACAAGATGCACTACCCCAT |
| | GGTGGAGTACTGTACCCCAACCACTTCCGGCGAGGACGTGAGAGATTTCGCCAAG |
| | GTGCTGAAGAACAAGTTCCGGACAAAGAGATACTTTGCAAAACACCCAAGAATGG |
| | GCTACCTGCCAGTGCAGACCGTGCTGGAGGGGGACAATATGGAGACAGACACAAT |
| | GTGA |
| | (SEQ ID NO: 2407) |
| First DRG de- | CAACAAAATCACTGATGCTGGA (SEQ ID NO: 2399) |
| targeting miRNA | |
| binding site | |
| Second DRG de- | CAACAAAATCACTGATGCTGGA (SEQ ID NO: 2399) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 2411) |

Nucleic acid 21
(SEQ ID NO: 2432)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCCAACAAAATCACTGATGCTGGAAGATCAGCAACAAAATCACTGAT
GCTGGAAGACGCAGGAATAAAATATCTTTATTTTCATTACATCTGTGTGTTGGTTTTTTGTGTGAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 21 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | CGTCTGTCTAATTCCAGCCAAGGGCATGGGGGAAAGGACAGAGCCCAGGGACCTGT |
| | GGCCTATTCTCTGAGAGGACGCTGGGCTAATAATAGGAGGGGAGATTAGGGAGGGA |
| | TTTGACTGCATTGTTATCTCACAGGAATGGCGTTTGAGAGCCTTGCTTCCAAGCTC |
| | TTCCAAGAGTCATGCCACCCCCCTCCAATCTCTGTGGAAGTGCCCACTGCCTTTCC |
| | CGCTTCGTTCTCTTCTCTCGGTCCCATCCCTGACCCACTAGCCTGGTGCCGGGGGG |
| | AGACCAATTTCTGGAGGAAGCTCTGGGTACCCAGTCTTGCCCTAAGCCTGCCTCTG |
| | AACAGGCTGTTCTTGTTTATGACCCCCGTCCCAGCAGAATAGTGATTGCTCATCCC |
| | TGCATGTGATTTGAGAGGGCCAGTCTTTCAGGCCTGGAGGAAACTCAAGCCCCACC |
| | AGCCGCTTGGCATCGGAATGCTCAGCTGGTCCCCTCCTGGGGCTCATGTGACCAAG |
| | ATCTAGCTATAAATATCAGAGGGGCCTCAGACCAAGACAGAATTCGGGCACCAGGA |
| | GAAGGAAGCCAACAGGATCCGACCCGGTGTTTTGTGACAAAGGCAAGACCCCCAGG |
| | TCTACTTAGAGCAACGTTAGTAGAGGA (SEQ ID NO: 2404) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | CAACAAAATCACTGATGCTGGA (SEQ ID NO: 2399) |
| targeting miRNA | |
| binding site | |
| Second DRG de- | CAACAAAATCACTGATGCTGGA (SEQ ID NO: 2399) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 22
(SEQ ID NO: 2433)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAGGTGGAGGATTGCTACGAGAGAGAGGACGTGCAGAAGAAAACCTTCACC
AAATGGGTGAACGCCCAGTTCAGCAAGTTTGGCAAGCAGCACATCGAGAACCTGTTCAGCGATCTGCAGGACGGC
CGCAGGCTGCTGGATCTGCTGGAGGGTCTGACCGGCCAGAAACTGCCCAAAGAGAAGGGATCAACCAGGGTGCAC
GCCCTGAATAATGTGAACAAGGCCCTGAGAGTGCTGCAAAATAATAATGTGGATCTGGTGAACATCGGATCTACC
GATATTGTGGACGGCAACCATAAGCTGACTCTGGGCCTGATCTGGAATATCATCCTGCACTGGCAGGTGAAAAAT
GTGATGAAAAACATCATGGCCGGGCTGCAGCAGACCAACTCTGAGAAGATCCTGCTGAGCTGGGTGAGGCAGTCT
ACAAGAAATTACCCCCAGGTGAACGTGATCAATTTCACCACCAGCTGGTCCGACGGGCTGGCTCTGAATGCCCTG
ATTCACAGCCACCGGCCCGACCTGTTCGACTGGAATTCCGTGGTGTGCCAGCAGAGCGCCACACAGAGACTGGAG
CACGCTTTCAATATCGCTCGGTATCAGCTGGGCATCGAGAAGCTGCTGGACCCCGAGGACGTGGACACTACTTAC
CCCGATAAAAAGAGCATCCTGATGTATATCACCTCCCTGTTCCAAGTGCTGCCCCAGCAGGTCTCCATCGAGGCC
ATCCAGGAGGTGGAAATGCTGCCCCGGCCCCCAAAGGTGACAAAGGAAGAACACTTCCAGCTCCACCACCAGATG
CACTACTCTCAGCAGATCACCGTGAGCCTGGCCCAGGGGTACGAGCGGACCTCTTCTCCAAAGCCCCGCTTCAAG
AGCTATGCCTATACCCAGGCCGCCTATGTGACCACCTCCGATCCCACCCGCTCACCCTTCCCTTCTCAACACCTG
GAAGCCCCAGAGGACAAGTCCTTCGGCAGTTCTCTGATGGAGTCTGAGGTGAACCTGGATAGATATCAGACTGCC
CTGGAGGAGGTGCTGTCTTGGCTGCTCTCCGCCGAGGACACCCTGCAGGCACAGGGCGAGATCTCCAACGATGTG
GAGGTGGTGAAGGACCAGTTCCACACACACGAGGGCTATATGATGGACCTGACTGCCCATCAGGGCAGAGTGGGC
AATATCCTGCAGCTGGGTTCCAAACTGATAGGCACCGGAAAGCTGAGCGAAGACGAGGAGACCGAGGTGCAGGAG
CAGATGAATCTGCTGAACTCCCGGTGGGAGTGCCTGCGAGTCGCCAGCATGGAAAAGCAGAGCAACCTGCACCGC
GTGCTCATGGATCTGCAGAATCAGAAGCTGAAGGAGCTGAATGACTGGCTGACCAAGACCGAAGAGCGCACCAGG
AAGATGGAGGAGGAGCCACTGGGCCCCGATCTGGAGGACCTGAAAAGACAGGTGCAGCAGCATAAAGTGCTGCAG
GAGGACCTGGAGCAGGAACAAGTGAGAGTGAATTCTCTGACACACATGGTGGTGGTGGTGGACGAGAGCAGCGGC
GACCACGCAACAGCCGCCCTGGAGGAGCAGCTGAAAGTGCTGGGAGACCGGTGGGCCAACATTTGCCGGTGGACC
GAGGACCGCTGGGTGCTGCTGCAGGACATTCTGCTGAAGTGGCAGCGGCTGACCGAAGAGCAGTGCCTGTTCTCC
GCCTGGCTGAGCGAGAAGGAGGACGCCGTGAATAAGATCCACACCACCGGGTTCAAGGACCAGAATGAGATGCTG
TCCAGCCTGCAGAAGCTGGCCGTGCTGAAGGCCGATCTGGAAAAGAAGAAGCAGAGCATGGGAAAGCTGTATAGC
CTGAAGCAGGACCTGCTGTCTACTCTGAAAAACAAGTCAGTGACCCAGAAGACCGAGGCCTGGCTGGACAACTTC
GCCCGCTGCTGGGACAACCTGGTGCAGAAGCTGGAAAAGAGCACCGCCCAGATCAGCCAGGCAGTGACTACCACA
CAGCCCTCCCTGACACAGACTACTGTGATGGAGACCGTGACAACAGTGACCACAAGGGAGCAGATCCTGGTGAAG
CACGCCCAGGAAGAACTGCCCCCTCCACCCCCTCAGAAGAAGAGAACTCTGGAAAGACTGCAGGAGCTGCAGGAG
GCCACTGACGAGCTGGACCTGAAGCTGCGGCAGGCCGAGGTGATTAAGGGCAGCTGGCAGCCTGTGGGCGATCTG
CTGATCGACAGCCTGCAGGACCACCTGGAGAAGGTGAAAGCCCTGCGGGGCGAGATCGCCCCTCTGAAGGAGAAC
GTCAGCCATGTGAATGACCTGGCCAGACAGCTGACCACACTGGGGATTCAGCTGTCTCCTTACAATCTGTCAACC
CTGGAGGACCTGAACACTCGCTGGAAGCTGCTGCAGGTGGCCGTGGAGGACAGGGTGCGGCAGCTGCACGAAGCC
CATCGCGACTTTGGGCCCGCCTCTCAGCACTTTCTGAGCACCTCCGTGCAGGGACCCTGGGAGCGGGCAATCAGC
CCAAACAAGGTGCCATACTACATCAACCACGAGACCCAGACCACCTGTTGGGATCACCCCAAGATGACAGAGCTG
TACCAGTCTCTGGCTGACCTGAACAACGTGCGGTTCTCAGCCTACAGGACCGCCATGAAGCTGCGGAGACTGCAG
AAGGCCCTGTGCCTGGATCTGCTGAGCCTCTCCGCTGCTTGTGACGCCCTGGATCAGCACAACCTGAAACAGAAC
GACCAGCCAATGGACATCCTGCAGATTATTAATTGCCTGACAACCATCTATGATCGGCTGGAACAGGAGCACAAT
AATCTGGTGAACGTGCCACTGTGCGTGGACATGTGCCTGAATTGGCTGCTGAACGTGTACGACACAGGACGGACC
GGCAGAATCAGAGTGCTGTCTTTTAAGACCGGGATCATCAGCCTGTGCAAAGCCCACCTGGAGGACAAGTACCGG
TATCTGTTCAAACAAGTGGCCTCCAGCACCGGCTTTTGCGACCAGCGCAGGCTGGGCCTGCTGCTCCACGACTCC
ATCCAGATCCCTAGACAGCTGGGCGAGGTGGCTTCTTTTGGCGGATCCAATATCGAGCCCAGTGTGCGGAGCTGT
TTTCAGTTTGCTAATAATAAGCCTGAGATCGAGGCCGCTCTGTTCCTGGACTGGATGAGACTGGAGCCCCAGTCC
ATGGTGTGGCTGCCAGTGCTGCACAGGGTCGCCGCCGCAGAGACTGCCAAGCACCAGGCCAAGTGCAACATCTGC
AAAGAATGCCCAATCATTGGCTTTAGATACCGGAGCCTCAAACACTTTAACTACGACATCTGCCAGAGCTGCTTC
TTCTCCGGCCGGGTGGCCAAGGGCCACAAGATGCACTACCCCATGGTGGAGTACTGTACCCCAACCACTTCCGGC
GAGGACGTGAGAGATTTCGCCAAGGTGCTGAAGAACAAGTTCCGGACAAAGAGATACTTTGCAAAACACCCAAGA
ATGGGCTACCTGCCAGTGCAGACCGTGCTGGAGGGGGACAATATGGAGACAGACACAATGTGATAGATTCCGGCC
TGATTCACAACACCAGCTAGATCAGCGGCCTGATTCACAACACCAGCTAGACGCAGGAATAAAATATCTTTATTT
TCATTACATCTGTGTGTTGGTTTTTTGTGTGAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCAGTGAGCGAG
CGAGCGCGC

| Nucleic acid | |
|---|---|
| cargo 22 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | TAGTTATTAATAGTAATCAATTACGGGGTCATTAGTTCATAGCCCATATATGGAGT |
| | TCCGCGTTACATAACTTACGGTAAATGGCCCGCCTGGCTGACCGCCCAACGACCCC |
| | CGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATC |
| | AAGTGTATCATATGCCAAGTACGCCCCCTATTGACGTCAATGACGGTAAATGGCCC |
| | GCCTGGCATTATGCCCAGTACATGACCTTATGGGACTTTCCTACTTGGCAGTACAT |
| | CTACGTATTAGTCATCGCTATTACCATGCTAGACTAGCATGCTGCCCATGTAAGGA |
| | GGCAAGGCCTGGGGACACCCGAGATGCCTGGTTATAATTAACCCAGACATGTGGCT |
| | GCCCCCCCCCCCCAACACCTGCTGCCTCTAAAAATAACCCTGCATGCCATGTTCCC |
| | GGCGAAGGGCCAGCTGTCCCCCGCCAGCTAGACTCAGCTCTCAGGGGCCCCTCCCT |
| | GGGGACAGCCCCTCCTGGCTAGTCACACCCTGTAGGCTCCTCTATATAACCCAGGG |
| | GCACAGGGGCTGCCCTCATTCTACCACCACCTCCACAGCACAGACAGACACTCAGG |
| | AGCCAGCCAGC (SEQ ID NO: 2402) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | CGGCCTGATTCACAACACCAGCT (SEQ ID NO: 2400) |
| targeting miRNA | |
| binding site | |
| Second DRG de- | CGGCCTGATTCACAACACCAGCT (SEQ ID NO: 2400) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 23
(SEQ ID NO: 2434)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 23 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | CGTCTGTCTAATTCCAGCCAAGGGCATGGGGGAAAGGACAGAGCCCAGGGACCTGT |
| | GGCCTATTCTCTGAGAGGACGCTGGGCTAATAATAGGAGGGGAGATTAGGGAGGGA |
| | TTTGACTGCATTGTTATCTCACAGGAATGGCGTTTGAGAGCCTTGCTTCCAAGCTC |
| | TTCCAAGAGTCATGCCACCCCCCTCCAATCTCTGTGGAAGTGCCCACTGCCTTTCC |
| | CGCTTCGTTCTCTTCTCTCTAAATCTGAGCCCACCCCACTCAGGGGTACAGGATCT |
| | GGTTCATTTATCTCCCAGAATGATGCCTTGGGTTGCGTCCCCTCTAAACTCTGGCA |
| | CATTCCAGCCCCTCTTTGGGAGAAAGAACATCTCTTCTTGCCCAGGCCCTAGTGAG |
| | ACATGCATCGCAAGACATGCTCCCCCCTTCCTCCCCGCTGGGCTCCAGCTGCTGCC |
| | AGCCCTCCAAGAAAGGAGAGGCCCTGAGTTGGGCTATTTTGGTATCTGGGGTGGGC |
| | ACCCGCAGGGCTAAGGTTACCTTGGTATGTTAAGGGCTCCCTGGGGCAGGACTATA |
| | TAACCCCAGAGGGACTGCCCCATGCTGACTCCTTGCTTCTTTCCTGCTCGAGCCGC |
| | TGCCTTGCCCCCCGGAGACTGAAGACA (SEQ ID NO: 2403) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | CAACAAAATCACTGATGCTGGA (SEQ ID NO: 2400) |
| targeting miRNA | |
| binding site | |
| Second DRG de- | CAACAAAATCACTGATGCTGGA (SEQ ID NO: 2400) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 24
(SEQ ID NO: 2435)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCCGGCCTGATTCACAACACCAGCTAGATCAGCGGCCTGATTCACAA
CACCAGCTAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 24 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | CGTCTGTCTAATTCCAGCCAAGGGCATGGGGGAAAGGACAGAGCCCAGGGACCTGT |
| | GGCCTATTCTCTGAGAGGACGCTGGGCTAATAATAGGAGGGGAGATTAGGGAGGGA |
| | TTTGACTGCATTGTTATCTCACAGGAATGGCGTTTGAGAGCCTTGCTTCCAAGCTC |
| | TTCCAAGAGTCATGCCACCCCCCTCCAATCTCTGTGGAAGTGCCCACTGCCTTTCC |
| | CGCTTCGTTCTCTTCTCTCGGTCCCATCCCTGACCCACTAGCCTGGTGCCGGGGGG |
| | AGACCAATTTCTGGAGGAAGCTCTGGGTACCCAGTCTTGCCCTAAGCCTGCCTCTG |
| | AACAGGCTGTTCTTGTTTATGACCCCCGTCCCAGCAGAATAGTGATTGCTCATCCC |
| | TGCATGTGATTTGAGAGGGCCAGTCTTTCAGGCCTGGAGGAAACTCAAGCCCCACC |
| | AGCCGCTTGGCATCGGAATGCTCAGCTGGTCCCCTCCTGGGGCTCATGTGACCAAG |
| | ATCTAGCTATAAATATCAGAGGGGCCTCAGACCAAGACAGAATTCGGGCACCAGGA |
| | GAAGGAAGCCAACAGGATCCGACCCGGTGTTTTGTGACAAAGGCAAGACCCCCAGG |
| | TCTACTTAGAGCAACGTTAGTAGAGGA (SEQ ID NO: 2404) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | CGGCCTGATTCACAACACCAGCT (SEQ ID NO: 2400) |
| targeting miRNA | |
| binding site | |
| Second DRG de- | CGGCCTGATTCACAACACCAGCT (SEQ ID NO: 2400) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 25
(SEQ ID NO: 2436)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTTAGTTATTAATAGTAATCAATT
ACGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGACCTTATGGGACTTTCCT
ACTTGGCAGTACATCTACGTATTAGTCATCGCTATTACCATGCTAGACTAGCATGCTGCCCATGTAAGGAGGCAA
GGCCTGGGGACACCCGAGATGCCTGGTTATAATTAACCCAGACATGTGGCTGCCCCCCCCCCCCAACACCTGCTG
CCTCTAAAAATAACCCTGCATGCCATGTTCCCGGCGAAGGGCCAGCTGTCCCCCGCCAGCTAGACTCAGCTCTCA
GGGGCCCCTCCCTGGGGACAGCCCCTCCTGGCTAGTCACACCCTGTAGGCTCCTCTATATAACCCAGGGGCACAG
GGGCTGCCCTCATTCTACCACCACCTCCACAGCACAGACAGACACTCAGGAGCCAGCCAGCCAGCCCAGGTACCT
GGGCAGGTAAGTATCAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAGGTGTCCAGACT
AGTGCCACCATGCTGTGGTGGGAGGAGGTGGAGGATTGCTACGAGAGAGAGGACGTGCAGAAGAAAACCTTCACC
AAATGGGTGAACGCCCAGTTCAGCAAGTTTGGCAAGCAGCACATCGAGAACCTGTTCAGCGATCTGCAGGACGGC
CGCAGGCTGCTGGATCTGCTGGAGGGTCTGACCGGCCAGAAACTGCCCAAAGAGAAGGGATCAACCAGGGTGCAC
GCCCTGAATAATGTGAACAAGGCCCTGAGAGTGCTGCAAAATAATAATGTGGATCTGGTGAACATCGGATCTACC
GATATTGTGGACGGCAACCATAAGCTGACTCTGGGCCTGATCTGGAATATCATCCTGCACTGGCAGGTGAAAAAT
GTGATGAAAAACATCATGGCCGGGCTGCAGCAGACCAACTCTGAGAAGATCCTGCTGAGCTGGGTGAGGCAGTCT
ACAAGAAATTACCCCCAGGTGAACGTGATCAATTTCACCACCAGCTGGTCCGACGGGCTGGCTCTGAATGCCCTG
ATTCACAGCCACCGGCCCGACCTGTTCGACTGGAATTCCGTGGTGTGCCAGCAGAGCGCCACACAGAGACTGGAG
CACGCTTTCAATATCGCTCGGTATCAGCTGGGCATCGAGAAGCTGCTGGACCCCGAGGACGTGGACACTACTTAC
CCCGATAAAAAGAGCATCCTGATGTATATCACCTCCCTGTTCCAAGTGCTGCCCCAGCAGGTCTCCATCGAGGCC
ATCCAGGAGGTGGAAATGCTGCCCCGGCCCCCAAAGGTGACAAAGGAAGAACACTTCCAGCTCCACCACCAGATG
CACTACTCTCAGCAGATCACCGTGAGCCTGGCCCAGGGGTACGAGCGGACCTCTTCTCCAAAGCCCCGCTTCAAG
AGCTATGCCTATACCCAGGCCGCCTATGTGACCACCTCCGATCCCACCCGCTCACCCTTCCCTTCTCAACACCTG
GAAGCCCCAGAGGACAAGTCCTTCGGCAGTTCTCTGATGGAGTCTGAGGTGAACCTGGATAGATATCAGACTGCC
CTGGAGGAGGTGCTGTCTTGGCTGCTCTCCGCCGAGGACACCCTGCAGGCACAGGGCGAGATCTCCAACGATGTG
GAGGTGGTGAAGGACCAGTTCCACACACACGAGGGCTATATGATGGACCTGACTGCCCATCAGGGCAGAGTGGGC
AATATCCTGCAGCTGGGTTCCAAACTGATAGGCACCGGAAAGCTGAGCGAAGACGAGGAGACCGAGGTGCAGGAG
CAGATGAATCTGCTGAACTCCCGGTGGGAGTGCCTGCGAGTCGCCAGCATGGAAAAGCAGAGCAACCTGCACCGC
GTGCTCATGGATCTGCAGAATCAGAAGCTGAAGGAGCTGAATGACTGGCTGACCAAGACCGAAGAGCGCACCAGG
AAGATGGAGGAGGAGCCACTGGGCCCCGATCTGGAGGACCTGAAAAGACAGGTGCAGCAGCATAAAGTGCTGCAG
GAGGACCTGGAGCAGGAACAAGTGAGAGTGAATTCTCTGACACACATGGTGGTGGTGGTGGACGAGAGCAGCGGC
GACCACGCAACAGCCGCCCTGGAGGAGCAGCTGAAAGTGCTGGGAGACCGGTGGGCCAACATTTGCCGGTGGACC
GAGGACCGCTGGGTGCTGCTGCAGGACATTCTGCTGAAGTGGCAGCGGCTGACCGAAGAGCAGTGCCTGTTCTCC
GCCTGGCTGAGCGAGAAGGAGGACGCCGTGAATAAGATCCACACCACCGGGTTCAAGGACCAGAATGAGATGCTG
TCCAGCCTGCAGAAGCTGGCCGTGCTGAAGGCCGATCTGGAAAAGAAGAAGCAGAGCATGGGAAAGCTGTATAGC
CTGAAGCAGGACCTGCTGTCTACTCTGAAAAACAAGTCAGTGACCCAGAAGACCGAGGCCTGGCTGGACAACTTC
GCCCGCTGCTGGGACAACCTGGTGCAGAAGCTGGAAAAGAGCACCGCCCAGATCAGCCAGGCAGTGACTACCACA
CAGCCCTCCCTGACACAGACTACTGTGATGGAGACCGTGACAACAGTGACCACAAGGGAGCAGATCCTGGTGAAG
CACGCCCAGGAAGAACTGCCCCCTCCACCCCCTCAGAAGAAGAGAACTCTGGAAAGACTGCAGGAGCTGCAGGAG
GCCACTGACGAGCTGGACCTGAAGCTGCGGCAGGCCGAGGTGATTAAGGGCAGCTGGCAGCCTGTGGGCGATCTG
CTGATCGACAGCCTGCAGGACCACCTGGAGAAGGTGAAAGCCCTGCGGGGCGAGATCGCCCCTCTGAAGGAGAAC
GTCAGCCATGTGAATGACCTGGCCAGACAGCTGACCACACTGGGGATTCAGCTGTCTCCTTACAATCTGTCAACC
CTGGAGGACCTGAACACTCGCTGGAAGCTGCTGCAGGTGGCCGTGGAGGACAGGGTGCGGCAGCTGCACGAAGCC
CATCGCGACTTTGGGCCCGCCTCTCAGCACTTTCTGAGCACCTCCGTGCAGGGACCCTGGGAGCGGGCAATCAGC
CCAAACAAGGTGCCATACTACATCAACCACGAGACCCAGACCACCTGTTGGGATCACCCCAAGATGACAGAGCTG
TACCAGTCTCTGGCTGACCTGAACAACGTGCGGTTCTCAGCCTACAGGACCGCCATGAAGCTGCGGAGACTGCAG
AAGGCCCTGTGCCTGGATCTGCTGAGCCTCTCCGCTGCTTGTGACGCCCTGGATCAGCACAACCTGAAACAGAAC
GACCAGCCAATGGACATCCTGCAGATTATTAATTGCCTGACAACCATCTATGATCGGCTGGAACAGGAGCACAAT
AATCTGGTGAACGTGCCACTGTGCGTGGACATGTGCCTGAATTGGCTGCTGAACGTGTACGACACAGGACGGACC
GGCAGAATCAGAGTGCTGTCTTTTAAGACCGGGATCATCAGCCTGTGCAAAGCCCACCTGGAGGACAAGTACCGG
TATCTGTTCAAACAAGTGGCCTCCAGCACCGGCTTTTGCGACCAGCGCAGGCTGGGCCTGCTGCTCCACGACTCC
ATCCAGATCCCTAGACAGCTGGGCGAGGTGGCTTCTTTTGGCGGATCCAATATCGAGCCCAGTGTGCGGAGCTGT
TTTCAGTTTGCTAATAATAAGCCTGAGATCGAGGCCGCTCTGTTCCTGGACTGGATGAGACTGGAGCCCCAGTCC
ATGGTGTGGCTGCCAGTGCTGCACAGGGTCGCCGCCGCAGAGACTGCCAAGCACCAGGCCAAGTGCAACATCTGC
AAAGAATGCCCAATCATTGGCTTTAGATACCGGAGCCTCAAACACTTTAACTACGACATCTGCCAGAGCTGCTTC
TTCTCCGGCCGGGTGGCCAAGGGCCACAAGATGCACTACCCCATGGTGGAGTACTGTACCCCAACCACTTCCGGC
GAGGACGTGAGAGATTTCGCCAAGGTGCTGAAGAACAAGTTCCGGACAAAGAGATACTTTGCAAAACACCCAAGA
ATGGGCTACCTGCCAGTGCAGACCGTGCTGGAGGGGGACAATATGGAGACAGACACAATGTGATAGATTCTCATA
CAGCTAGATAACCAAAGAAGATCAGTCATACAGCTAGATAACCAAAGAAGACGCAGGAATAAAATATCTTTATTT
TCATTACATCTGTGTGTTGGTTTTTTGTGTGAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTCAGTGAGCGAG
CGAGCGCGC

| Nucleic acid | |
|---|---|
| cargo 25 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | TAGTTATTAATAGTAATCAATTACGGGGTCATTAGTTCATAGCCCATATATGGAGT |
| | TCCGCGTTACATAACTTACGGTAAATGGCCCGCCTGGCTGACCGCCCAACGACCCC |
| | CGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATC |
| | AAGTGTATCATATGCCAAGTACGCCCCCTATTGACGTCAATGACGGTAAATGGCCC |
| | GCCTGGCATTATGCCCAGTACATGACCTTATGGGACTTTCCTACTTGGCAGTACAT |
| | CTACGTATTAGTCATCGCTATTACCATGCTAGACTAGCATGCTGCCCATGTAAGGA |
| | GGCAAGGCCTGGGGACACCCGAGATGCCTGGTTATAATTAACCCAGACATGTGGCT |
| | GCCCCCCCCCCCCAACACCTGCTGCCTCTAAAAATAACCCTGCATGCCATGTTCCC |
| | GGCGAAGGGCCAGCTGTCCCCCGCCAGCTAGACTCAGCTCTCAGGGGCCCCTCCCT |
| | GGGGACAGCCCCTCCTGGCTAGTCACACCCTGTAGGCTCCTCTATATAACCCAGGG |
| | GCACAGGGGCTGCCCTCATTCTACCACCACCTCCACAGCACAGACAGACACTCAGG |
| | AGCCAGCCAGC (SEQ ID NO: 2402) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | TCATACAGCTAGATAACCAAAGA (SEQ ID NO: 2401) |
| targeting miRNA | |
| binding site | |
| Second DRG de- | TCATACAGCTAGATAACCAAAGA (SEQ ID NO: 2401) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 26
(SEQ ID NO: 2437)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCTAAA
TCTGAGCCCACCCCACTCAGGGGTACAGGATCTGGTTCATTTATCTCCCAGAATGATGCCTTGGGTTGCGTCCCC
TCTAAACTCTGGCACATTCCAGCCCCTCTTTGGGAGAAAGAACATCTCTTCTTGCCCAGGCCCTAGTGAGACATG
CATCGCAAGACATGCTCCCCCCTTCCTCCCCGCTGGGCTCCAGCTGCTGCCAGCCCTCCAAGAAAGGAGAGGCCC
TGAGTTGGGCTATTTTGGTATCTGGGGTGGGCACCCGCAGGGCTAAGGTTACCTTGGTATGTTAAGGGCTCCCTG
GGGCAGGACTATATAACCCCAGAGGGACTGCCCCATGCTGACTCCTTGCTTCTTTCCTGCTCGAGCCGCTGCCTT
GCCCCCCGGAGACTGAAGACACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 26 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | CGTCTGTCTAATTCCAGCCAAGGGCATGGGGGAAAGGACAGAGCCCAGGGACCTGT |
| | GGCCTATTCTCTGAGAGGACGCTGGGCTAATAATAGGAGGGGAGATTAGGGAGGGA |
| | TTTGACTGCATTGTTATCTCACAGGAATGGCGTTTGAGAGCCTTGCTTCCAAGCTC |
| | TTCCAAGAGTCATGCCACCCCCCTCCAATCTCTGTGGAAGTGCCCACTGCCTTTCC |
| | CGCTTCGTTCTCTTCTCTCTAAATCTGAGCCCACCCCACTCAGGGGTACAGGATCT |
| | GGTTCATTTATCTCCCAGAATGATGCCTTGGGTTGCGTCCCCTCTAAACTCTGGCA |
| | CATTCCAGCCCCTCTTTGGGAGAAAGAACATCTCTTCTTGCCCAGGCCCTAGTGAG |
| | ACATGCATCGCAAGACATGCTCCCCCCTTCCTCCCCGCTGGGCTCCAGCTGCTGCC |
| | AGCCCTCCAAGAAAGGAGAGGCCCTGAGTTGGGCTATTTTGGTATCTGGGGTGGGC |
| | ACCCGCAGGGCTAAGGTTACCTTGGTATGTTAAGGGCTCCCTGGGGCAGGACTATA |
| | TAACCCCAGAGGGACTGCCCCATGCTGACTCCTTGCTTCTTTCCTGCTCGAGCCGC |
| | TGCCTTGCCCCCCGGAGACTGAAGACA (SEQ ID NO: 2403) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | TCATACAGCTAGATAACCAAAGA (SEQ ID NO: 2401) |
| targeting miRNA | |
| binding site | |
| Second DRG de- | TCATACAGCTAGATAACCAAAGA (SEQ ID NO: 2401) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

Nucleic acid 27
(SEQ ID NO: 2438)
GCGCGCTCGCTCGCTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTCGTCTGTCTAATTCCAGCCAAG
GGCATGGGGGAAAGGACAGAGCCCAGGGACCTGTGGCCTATTCTCTGAGAGGACGCTGGGCTAATAATAGGAGGG
GAGATTAGGGAGGGATTTGACTGCATTGTTATCTCACAGGAATGGCGTTTGAGAGCCTTGCTTCCAAGCTCTTCC
AAGAGTCATGCCACCCCCCTCCAATCTCTGTGGAAGTGCCCACTGCCTTTCCCGCTTCGTTCTCTTCTCTCGGTC
CCATCCCTGACCCACTAGCCTGGTGCCGGGGGGAGACCAATTTCTGGAGGAAGCTCTGGGTACCCAGTCTTGCCC
TAAGCCTGCCTCTGAACAGGCTGTTCTTGTTTATGACCCCCGTCCCAGCAGAATAGTGATTGCTCATCCCTGCAT
GTGATTTGAGAGGGCCAGTCTTTCAGGCCTGGAGGAAACTCAAGCCCCACCAGCCGCTTGGCATCGGAATGCTCA
GCTGGTCCCCTCCTGGGGCTCATGTGACCAAGATCTAGCTATAAATATCAGAGGGGCCTCAGACCAAGACAGAAT
TCGGGCACCAGGAGAAGGAAGCCAACAGGATCCGACCCGGTGTTTTGTGACAAAGGCAAGACCCCCAGGTCTACT
TAGAGCAACGTTAGTAGAGGACAGCCCAGGTACCTGGGCAGGTAAGTATCAAGGTTACAAGACAGGTTTAAGGAG
ACCAATAGAAACTGGGCTTGTCGAGACAGAGAAGACTCTTGCGTTTCTGATAGGCACCTATTGGTCTTACTGACA
TCCACTTTGCCTTTCTCTCCACAGGTGTCCAGACTAGTGCCACCATGCTGTGGTGGGAGGAGGTGGAGGATTGCT
ACGAGAGAGAGGACGTGCAGAAGAAAACCTTCACCAAATGGGTGAACGCCCAGTTCAGCAAGTTTGGCAAGCAGC
ACATCGAGAACCTGTTCAGCGATCTGCAGGACGGCCGCAGGCTGCTGGATCTGCTGGAGGGTCTGACCGGCCAGA
AACTGCCCAAAGAGAAGGGATCAACCAGGGTGCACGCCCTGAATAATGTGAACAAGGCCCTGAGAGTGCTGCAAA
ATAATAATGTGGATCTGGTGAACATCGGATCTACCGATATTGTGGACGGCAACCATAAGCTGACTCTGGGCCTGA
TCTGGAATATCATCCTGCACTGGCAGGTGAAAAATGTGATGAAAAACATCATGGCCGGGCTGCAGCAGACCAACT
CTGAGAAGATCCTGCTGAGCTGGGTGAGGCAGTCTACAAGAAATTACCCCCAGGTGAACGTGATCAATTTCACCA
CCAGCTGGTCCGACGGGCTGGCTCTGAATGCCCTGATTCACAGCCACCGGCCCGACCTGTTCGACTGGAATTCCG
TGGTGTGCCAGCAGAGCGCCACACAGAGACTGGAGCACGCTTTCAATATCGCTCGGTATCAGCTGGGCATCGAGA
AGCTGCTGGACCCCGAGGACGTGGACACTACTTACCCCGATAAAAAGAGCATCCTGATGTATATCACCTCCCTGT
TCCAAGTGCTGCCCCAGCAGGTCTCCATCGAGGCCATCCAGGAGGTGGAAATGCTGCCCCGGCCCCCAAAGGTGA
CAAAGGAAGAACACTTCCAGCTCCACCACCAGATGCACTACTCTCAGCAGATCACCGTGAGCCTGGCCCAGGGGT
ACGAGCGGACCTCTTCTCCAAAGCCCCGCTTCAAGAGCTATGCCTATACCCAGGCCGCCTATGTGACCACCTCCG
ATCCCACCCGCTCACCCTTCCCTTCTCAACACCTGGAAGCCCCAGAGGACAAGTCCTTCGGCAGTTCTCTGATGG
AGTCTGAGGTGAACCTGGATAGATATCAGACTGCCCTGGAGGAGGTGCTGTCTTGGCTGCTCTCCGCCGAGGACA
CCCTGCAGGCACAGGGCGAGATCTCCAACGATGTGGAGGTGGTGAAGGACCAGTTCCACACACACGAGGGCTATA
TGATGGACCTGACTGCCCATCAGGGCAGAGTGGGCAATATCCTGCAGCTGGGTTCCAAACTGATAGGCACCGGAA
AGCTGAGCGAAGACGAGGAGACCGAGGTGCAGGAGCAGATGAATCTGCTGAACTCCCGGTGGGAGTGCCTGCGAG
TCGCCAGCATGGAAAAGCAGAGCAACCTGCACCGCGTGCTCATGGATCTGCAGAATCAGAAGCTGAAGGAGCTGA
ATGACTGGCTGACCAAGACCGAAGAGCGCACCAGGAAGATGGAGGAGGAGCCACTGGGCCCCGATCTGGAGGACC
TGAAAAGACAGGTGCAGCAGCATAAAGTGCTGCAGGAGGACCTGGAGCAGGAACAAGTGAGAGTGAATTCTCTGA
CACACATGGTGGTGGTGGTGGACGAGAGCAGCGGCGACCACGCAACAGCCGCCCTGGAGGAGCAGCTGAAAGTGC
TGGGAGACCGGTGGGCCAACATTTGCCGGTGGACCGAGGACCGCTGGGTGCTGCTGCAGGACATTCTGCTGAAGT
GGCAGCGGCTGACCGAAGAGCAGTGCCTGTTCTCCGCCTGGCTGAGCGAGAAGGAGGACGCCGTGAATAAGATCC
ACACCACCGGGTTCAAGGACCAGAATGAGATGCTGTCCAGCCTGCAGAAGCTGGCCGTGCTGAAGGCCGATCTGG
AAAAGAAGAAGCAGAGCATGGGAAAGCTGTATAGCCTGAAGCAGGACCTGCTGTCTACTCTGAAAAACAAGTCAG
TGACCCAGAAGACCGAGGCCTGGCTGGACAACTTCGCCCGCTGCTGGGACAACCTGGTGCAGAAGCTGGAAAAGA
GCACCGCCCAGATCAGCCAGGCAGTGACTACCACACAGCCCTCCCTGACACAGACTACTGTGATGGAGACCGTGA
CAACAGTGACCACAAGGGAGCAGATCCTGGTGAAGCACGCCCAGGAAGAACTGCCCCCTCCACCCCCTCAGAAGA
AGAGAACTCTGGAAAGACTGCAGGAGCTGCAGGAGGCCACTGACGAGCTGGACCTGAAGCTGCGGCAGGCCGAGG
TGATTAAGGGCAGCTGGCAGCCTGTGGGCGATCTGCTGATCGACAGCCTGCAGGACCACCTGGAGAAGGTGAAAG
CCCTGCGGGGCGAGATCGCCCCTCTGAAGGAGAACGTCAGCCATGTGAATGACCTGGCCAGACAGCTGACCACAC
TGGGGATTCAGCTGTCTCCTTACAATCTGTCAACCCTGGAGGACCTGAACACTCGCTGGAAGCTGCTGCAGGTGG
CCGTGGAGGACAGGGTGCGGCAGCTGCACGAAGCCCATCGCGACTTTGGGCCCGCCTCTCAGCACTTTCTGAGCA
CCTCCGTGCAGGGACCCTGGGAGCGGGCAATCAGCCCAAACAAGGTGCCATACTACATCAACCACGAGACCCAGA
CCACCTGTTGGGATCACCCCAAGATGACAGAGCTGTACCAGTCTCTGGCTGACCTGAACAACGTGCGGTTCTCAG
CCTACAGGACCGCCATGAAGCTGCGGAGACTGCAGAAGGCCCTGTGCCTGGATCTGCTGAGCCTCTCCGCTGCTT
GTGACGCCCTGGATCAGCACAACCTGAAACAGAACGACCAGCCAATGGACATCCTGCAGATTATTAATTGCCTGA
CAACCATCTATGATCGGCTGGAACAGGAGCACAATAATCTGGTGAACGTGCCACTGTGCGTGGACATGTGCCTGA
ATTGGCTGCTGAACGTGTACGACACAGGACGGACCGGCAGAATCAGAGTGCTGTCTTTTAAGACCGGGATCATCA
GCCTGTGCAAAGCCCACCTGGAGGACAAGTACCGGTATCTGTTCAAACAAGTGGCCTCCAGCACCGGCTTTTGCG
ACCAGCGCAGGCTGGGCCTGCTGCTCCACGACTCCATCCAGATCCCTAGACAGCTGGGCGAGGTGGCTTCTTTTG
GCGGATCCAATATCGAGCCCAGTGTGCGGAGCTGTTTTCAGTTTGCTAATAATAAGCCTGAGATCGAGGCCGCTC
TGTTCCTGGACTGGATGAGACTGGAGCCCCAGTCCATGGTGTGGCTGCCAGTGCTGCACAGGGTCGCCGCCGCAG
AGACTGCCAAGCACCAGGCCAAGTGCAACATCTGCAAAGAATGCCCAATCATTGGCTTTAGATACCGGAGCCTCA
AACACTTTAACTACGACATCTGCCAGAGCTGCTTCTTCTCCGGCCGGGTGGCCAAGGGCCACAAGATGCACTACC
CCATGGTGGAGTACTGTACCCCAACCACTTCCGGCGAGGACGTGAGAGATTTCGCCAAGGTGCTGAAGAACAAGT
TCCGGACAAAGAGATACTTTGCAAAACACCCAAGAATGGGCTACCTGCCAGTGCAGACCGTGCTGGAGGGGGACA
ATATGGAGACAGACACAATGTGATAGATTCTCATACAGCTAGATAACCAAAGAAGATCAGTCATACAGCTAGATA
ACCAAAGAAGACGCAGGAATAAAATATCTTTATTTTCATTACATCTGTGTGTTGGTTTTTTGTGTGAGGAACCCC
TAGTGATGGAGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGC

| Nucleic acid | |
|---|---|
| cargo 27 | |
| annotated | Sequence |
| AAV2 ITR | GCGCGCTCGCTCGCTCACTGAGGCCGCCCGGGCAAAGCCCGGGCGTCGGGCGACCT |
| | TTGGTCGCCCGGCCTCAGTGAGCGAGCGAGCGCGCAGAGAGGGAGTGGCCAACTCC |
| | ATCACTAGGGGTTCCT (SEQ ID NO: 2408) |
| Novel promoter | CGTCTGTCTAATTCCAGCCAAGGGCATGGGGGAAAGGACAGAGCCCAGGGACCTGT |
| | GGCCTATTCTCTGAGAGGACGCTGGGCTAATAATAGGAGGGGAGATTAGGGAGGGA |
| | TTTGACTGCATTGTTATCTCACAGGAATGGCGTTTGAGAGCCTTGCTTCCAAGCTC |
| | TTCCAAGAGTCATGCCACCCCCCTCCAATCTCTGTGGAAGTGCCCACTGCCTTTCC |
| | CGCTTCGTTCTCTTCTCTCGGTCCCATCCCTGACCCACTAGCCTGGTGCCGGGGGG |
| | AGACCAATTTCTGGAGGAAGCTCTGGGTACCCAGTCTTGCCCTAAGCCTGCCTCTG |
| | AACAGGCTGTTCTTGTTTATGACCCCCGTCCCAGCAGAATAGTGATTGCTCATCCC |
| | TGCATGTGATTTGAGAGGGCCAGTCTTTCAGGCCTGGAGGAAACTCAAGCCCCACC |
| | AGCCGCTTGGCATCGGAATGCTCAGCTGGTCCCCTCCTGGGGCTCATGTGACCAAG |
| | ATCTAGCTATAAATATCAGAGGGGCCTCAGACCAAGACAGAATTCGGGCACCAGGA |
| | GAAGGAAGCCAACAGGATCCGACCCGGTGTTTTGTGACAAAGGCAAGACCCCCAGG |
| | TCTACTTAGAGCAACGTTAGTAGAGGA (SEQ ID NO: 2404) |
| Intron | GTAAGTATCAAGGTTACAAGACAGGTTTAAGGAGACCAATAGAAACTGGGCTTGTC |
| | GAGACAGAGAAGACTCTTGCGTTTCTGATAGGCACCTATTGGTCTTACTGACATCC |
| | ACTTTGCCTTTCTCTCCACAG (SEQ ID NO: 2409) |
| Codon optimized | ATGCTGTGGTGGGAGGAGGTGGAGGATTGCTACGAGAGAGAGGACGTGCAGAAGAA |
| microdystrophin | AACCTTCACCAAATGGGTGAACGCCCAGTTCAGCAAGTTTGGCAAGCAGCACATCG |
| coding sequence | AGAACCTGTTCAGCGATCTGCAGGACGGCCGCAGGCTGCTGGATCTGCTGGAGGGT |
| | CTGACCGGCCAGAAACTGCCCAAAGAGAAGGGATCAACCAGGGTGCACGCCCTGAA |
| | TAATGTGAACAAGGCCCTGAGAGTGCTGCAAAATAATAATGTGGATCTGGTGAACA |
| | TCGGATCTACCGATATTGTGGACGGCAACCATAAGCTGACTCTGGGCCTGATCTGG |
| | AATATCATCCTGCACTGGCAGGTGAAAAATGTGATGAAAAACATCATGGCCGGGCT |
| | GCAGCAGACCAACTCTGAGAAGATCCTGCTGAGCTGGGTGAGGCAGTCTACAAGAA |
| | ATTACCCCCAGGTGAACGTGATCAATTTCACCACCAGCTGGTCCGACGGGCTGGCT |
| | CTGAATGCCCTGATTCACAGCCACCGGCCCGACCTGTTCGACTGGAATTCCGTGGT |
| | GTGCCAGCAGAGCGCCACACAGAGACTGGAGCACGCTTTCAATATCGCTCGGTATC |
| | AGCTGGGCATCGAGAAGCTGCTGGACCCCGAGGACGTGGACACTACTTACCCCGAT |
| | AAAAAGAGCATCCTGATGTATATCACCTCCCTGTTCCAAGTGCTGCCCCAGCAGGT |
| | CTCCATCGAGGCCATCCAGGAGGTGGAAATGCTGCCCCGGCCCCCAAAGGTGACAA |
| | AGGAAGAACACTTCCAGCTCCACCACCAGATGCACTACTCTCAGCAGATCACCGTG |
| | AGCCTGGCCCAGGGGTACGAGCGGACCTCTTCTCCAAAGCCCCGCTTCAAGAGCTA |
| | TGCCTATACCCAGGCCGCCTATGTGACCACCTCCGATCCCACCCGCTCACCCTTCC |
| | CTTCTCAACACCTGGAAGCCCCAGAGGACAAGTCCTTCGGCAGTTCTCTGATGGAG |
| | TCTGAGGTGAACCTGGATAGATATCAGACTGCCCTGGAGGAGGTGCTGTCTTGGCT |
| | GCTCTCCGCCGAGGACACCCTGCAGGCACAGGGCGAGATCTCCAACGATGTGGAGG |
| | TGGTGAAGGACCAGTTCCACACACACGAGGGCTATATGATGGACCTGACTGCCCAT |
| | CAGGGCAGAGTGGGCAATATCCTGCAGCTGGGTTCCAAACTGATAGGCACCGGAAA |
| | GCTGAGCGAAGACGAGGAGACCGAGGTGCAGGAGCAGATGAATCTGCTGAACTCCC |
| | GGTGGGAGTGCCTGCGAGTCGCCAGCATGGAAAAGCAGAGCAACCTGCACCGCGTG |
| | CTCATGGATCTGCAGAATCAGAAGCTGAAGGAGCTGAATGACTGGCTGACCAAGAC |
| | CGAAGAGCGCACCAGGAAGATGGAGGAGGAGCCACTGGGCCCCGATCTGGAGGACC |
| | TGAAAAGACAGGTGCAGCAGCATAAAGTGCTGCAGGAGGACCTGGAGCAGGAACAA |
| | GTGAGAGTGAATTCTCTGACACACATGGTGGTGGTGGTGGACGAGAGCAGCGGCGA |
| | CCACGCAACAGCCGCCCTGGAGGAGCAGCTGAAAGTGCTGGGAGACCGGTGGGCCA |
| | ACATTTGCCGGTGGACCGAGGACCGCTGGGTGCTGCTGCAGGACATTCTGCTGAAG |
| | TGGCAGCGGCTGACCGAAGAGCAGTGCCTGTTCTCCGCCTGGCTGAGCGAGAAGGA |
| | GGACGCCGTGAATAAGATCCACACCACCGGGTTCAAGGACCAGAATGAGATGCTGT |
| | CCAGCCTGCAGAAGCTGGCCGTGCTGAAGGCCGATCTGGAAAAGAAGAAGCAGAGC |
| | ATGGGAAAGCTGTATAGCCTGAAGCAGGACCTGCTGTCTACTCTGAAAAACAAGTC |
| | AGTGACCCAGAAGACCGAGGCCTGGCTGGACAACTTCGCCCGCTGCTGGGACAACC |
| | TGGTGCAGAAGCTGGAAAAGAGCACCGCCCAGATCAGCCAGGCAGTGACTACCACA |
| | CAGCCCTCCCTGACACAGACTACTGTGATGGAGACCGTGACAACAGTGACCACAAG |
| | GGAGCAGATCCTGGTGAAGCACGCCCAGGAAGAACTGCCCCCTCCACCCCCTCAGA |
| | AGAAGAGAACTCTGGAAAGACTGCAGGAGCTGCAGGAGGCCACTGACGAGCTGGAC |
| | CTGAAGCTGCGGCAGGCCGAGGTGATTAAGGGCAGCTGGCAGCCTGTGGGCGATCT |
| | GCTGATCGACAGCCTGCAGGACCACCTGGAGAAGGTGAAAGCCCTGCGGGGCGAGA |
| | TCGCCCCTCTGAAGGAGAACGTCAGCCATGTGAATGACCTGGCCAGACAGCTGACC |
| | ACACTGGGGATTCAGCTGTCTCCTTACAATCTGTCAACCCTGGAGGACCTGAACAC |
| | TCGCTGGAAGCTGCTGCAGGTGGCCGTGGAGGACAGGGTGCGGCAGCTGCACGAAG |
| | CCCATCGCGACTTTGGGCCCGCCTCTCAGCACTTTCTGAGCACCTCCGTGCAGGGA |
| | CCCTGGGAGCGGGCAATCAGCCCAAACAAGGTGCCATACTACATCAACCACGAGAC |
| | CCAGACCACCTGTTGGGATCACCCCAAGATGACAGAGCTGTACCAGTCTCTGGCTG |
| | ACCTGAACAACGTGCGGTTCTCAGCCTACAGGACCGCCATGAAGCTGCGGAGACTG |
| | CAGAAGGCCCTGTGCCTGGATCTGCTGAGCCTCTCCGCTGCTTGTGACGCCCTGGA |
| | TCAGCACAACCTGAAACAGAACGACCAGCCAATGGACATCCTGCAGATTATTAATT |
| | GCCTGACAACCATCTATGATCGGCTGGAACAGGAGCACAATAATCTGGTGAACGTG |
| | CCACTGTGCGTGGACATGTGCCTGAATTGGCTGCTGAACGTGTACGACACAGGACG |
| | GACCGGCAGAATCAGAGTGCTGTCTTTTAAGACCGGGATCATCAGCCTGTGCAAAG |
| | CCCACCTGGAGGACAAGTACCGGTATCTGTTCAAACAAGTGGCCTCCAGCACCGGC |
| | TTTTGCGACCAGCGCAGGCTGGGCCTGCTGCTCCACGACTCCATCCAGATCCCTAG |
| | ACAGCTGGGCGAGGTGGCTTCTTTTGGCGGATCCAATATCGAGCCCAGTGTGCGGA |
| | GCTGTTTTCAGTTTGCTAATAATAAGCCTGAGATCGAGGCCGCTCTGTTCCTGGAC |
| | TGGATGAGACTGGAGCCCCAGTCCATGGTGTGGCTGCCAGTGCTGCACAGGGTCGC |
| | CGCCGCAGAGACTGCCAAGCACCAGGCCAAGTGCAACATCTGCAAAGAATGCCCAA |
| | TCATTGGCTTTAGATACCGGAGCCTCAAACACTTTAACTACGACATCTGCCAGAGC |
| | TGCTTCTTCTCCGGCCGGGTGGCCAAGGGCCACAAGATGCACTACCCCATGGTGGA |
| | GTACTGTACCCCAACCACTTCCGGCGAGGACGTGAGAGATTTCGCCAAGGTGCTGA |
| | AGAACAAGTTCCGGACAAAGAGATACTTTGCAAAACACCCAAGAATGGGCTACCTG |
| | CCAGTGCAGACCGTGCTGGAGGGGGACAATATGGAGACAGACACAATGTGA |
| | (SEQ ID NO: 2407) |
| First DRG de- | TCATACAGCTAGATAACCAAAGA (SEQ ID NO: 2401) |
| targeting miRNA | |
| binding site | |
| Second DRG de- | TCATACAGCTAGATAACCAAAGA (SEQ ID NO: 2401) |
| targeting miRNA | |
| binding site | |
| Short polyA | AGGAATAAAATATCTTTATTTTCATTACATCTGTGTGTTGGTTTTTTGTGTG |
| | (SEQ ID NO: 2410) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 2411) |

### Example 2: Materials and Methods

The following Materials and Methods were used in experiments described herein.

Mice: The following mice were purchased from the Jackson laboratories: B10-mdx (JAX, #001801) and DBA/2J-mdx (JAX, 013141). Mice were injected retro-orbitally with Composition 1 at the indicated dose.

Cynomolgus Macaques: Juvenile male non-human primates were injected intravenously with Composition 1 at the indicated dose.

Microdystrophin Expression: Microdystrophin mRNA was quantified using a standard taqman assay. nhpRPL30 mRNA was used as the housekeeping control for quantification of microdystrophin expression. Microdystrophin protein was quantified using an automated capillary-based immunoassay (JESS™ automated western blotting). Protein was extracted from tissues and cultured cells. The mDys-FLAG protein expression was evaluated using an anti-dystrophin antibody at 1:10 dilution (Cat #MANHINGE2A, Developmental Studies Hybridoma Bank, University of Iowa). The amount of the mDys-FLAG expressed in tissues was calculated by interpolation to the full-length dystrophin standard curve derived from a pool of normal human heart or skeletal muscle samples that were diluted in dystrophin-deficient mdx mouse skeletal muscle to achieve levels of 100, 75, 50, 25, 0% of normal dystrophin. The relative intensity of the full-length and mDys-FLAG protein was normalized to alpha actinin-2 (ACTN2) using an anti-ACTN2 antibody at 1:800 dilution, which was used as a loading control (Abcam Cat #ab68167).

Vector Genome Quantification: For the vector genome quantification experiments, total DNA was extracted from tissue samples and DNA concentration was determined by Qubit dsDNA high-sensitivity assay kit (Thermo Fisher Scientific) using the QUBIT™ 4.0 Fluorometer (ThemoFisher Scientific). DNA was diluted to 0.5 ng/μL.

The VCN was quantified by digital droplet polymerase chain reaction (ddPCR) using ddPCR Supermix for Probes (no dUTP) (Bio-Rad Laboratories, Inc.) and an assay targeting a region in the viral genome. The VCN was normalized by the number of diploid genomes using the endogenous NHP telomerase gene (nhpTERT). All primers and probes were synthesized by Thermo Fisher Scientific. The ddPCR was performed using the QX200 ddPCR system (Bio-Rad Laboratories, Inc).

Immunofluorescence: Cryosections (10 mm in thickness) were sectioned from isopentane-frozen tissue and used for immunofluorescence staining to assess the level of the mDys-FLAG in a subset set of skeletal and heart tissues. The mDys-FLAG protein expression was examined with a rabbit monoclonal antibody against the FLAG peptide (Cat #14793S, 1:100, Cell Signaling Technology) and was visualized using Alexa Fluor 546 conjugated goat anti-rabbit IgG (H+L) antibody (Catalog #A-11035, 1:100, Thermo Fisher Scientific). The percentage of mDys-positive cells was quantified from digitalized FLAG immunostaining images using Biodock, an AI imaging software platform.

Mouse Muscle Physiology Analysis: The mechanical properties of the EDL muscle were assessed using an ex-vivo assay to evaluate the isometric, eccentric, and passive properties. The assay was performed using an isolated muscle force system (Aurora Scientific) according to published protocols, except that the EDL muscle was strained at 15% of the optimal muscle length during the eccentric contraction cycle (Hakim, Li, and Duan 2011; Hakim, Grange, and Duan 2011; Hakim, Wasala, and Duan 2013).

Statistical Analysis: Data are presented as means±standard deviation (SD). Statistical significance from the mdx-vehicle group was determined with one-way ANOVA analysis for the grip strength, ex-vivo assay (anatomical properties, isometric force, and viscous properties of the EDL muscle), and morphological analysis, or Two-way ANOVA analysis for the eccentric contraction profile and elastic properties. All statistical analyses were performed using GraphPad PRISM software version 10.2.3. P<0.05 was considered statistically significant.

### Example 3: Administration of Composition 1 to B10-Mdx Mice Resulted in Widespread Microdystrophin Expression

A composition comprising a vector and transgene of the invention, referred to as Composition 1 was engineered, comprising the AAV capsid MyoAAV-LD B and a cargo comprising the novel promoter, codon optimized microdystrophin, a FLAG peptide tag, and two DRG de-targeting miRNA binding sites (two binding sites for miR-338-3p). The cargo nucleic acid sequence for Composition 1 is provided as SEQ ID NO: 2430 (SEQ ID NO: 2430 does not include the FLAG tag sequence). The FLAG tag in Composition 1 is fused to the C-terminal of microdystrophin and is used to detect FLAG tagged microdystrophin. Composition 1 includes the cargo nucleic acid sequence (SEQ ID NO: 2430) and the AAV capsid protein MyoAAV-LD B, which is described above in Example 1.

The Reference AAV composition comprised an AAVrh74 capsid, an MHCK7 promoter, microdystrophin, and a FLAG peptide tag. The Reference AAV composition is a surrogate of SRP-9001, which is a microdystrophin gene replacement therapy approved by the Food and Drug Administration (FDA). Compositions were administered into 8 weeks old B10-mdx mice, which is a mouse model of DMD. B10-mdx mice were injected with vehicle (n=8) as a negative control. Mice were necropsied 6 weeks post-injection. Microdystrophin expression was detected and quantified using whole-tissue fluorescent imaging and a capillary-based immunoassay.

Administration is detailed in the table below:

| Test Article | No. of Animals | Dose | |
|---|---|---|---|
| Composition 1 | 8 | 4E+13 | vg/kg |
| Reference AAV composition | 8 | 1.33E+14 | vg/kg |

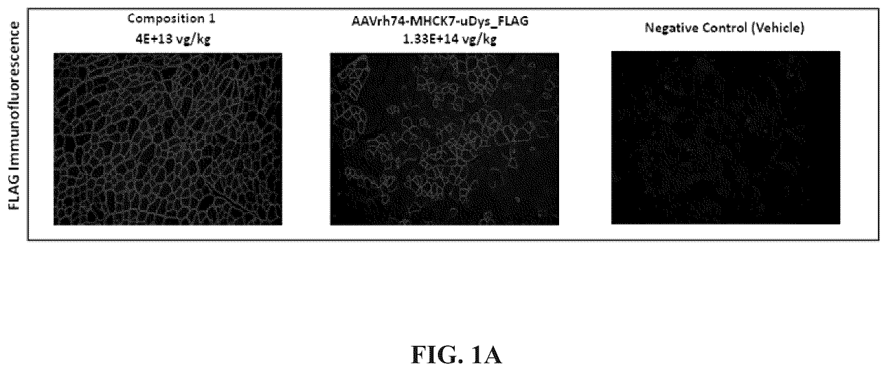

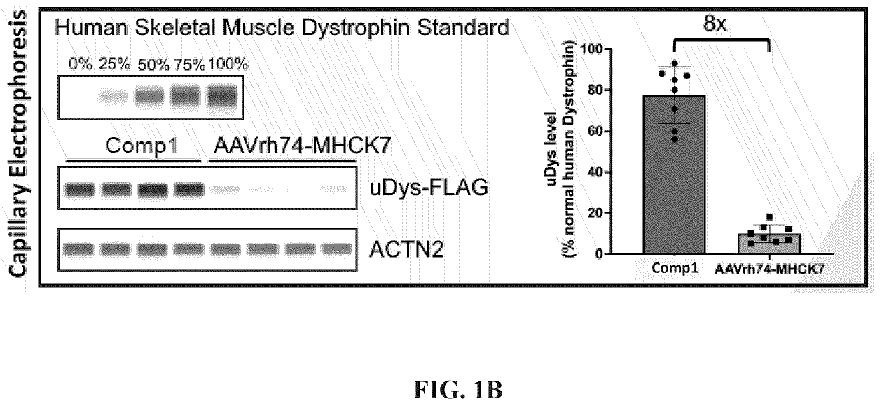

FIG. 1A shows images of FLAG immunofluorescence of microdystrophin expression in B10-mdx mouse quadriceps following administration of Composition 1 or the Reference AAV composition (AAVrh74-MHCK7-uDys-FLAG). FIG. 1B shows results from capillary electrophoresis quantifying microdystrophin expression in mouse quadriceps following administration of Composition 1 or the Reference AAV composition.

Administration of Composition 1 resulted in 8 times higher microdystrophin protein expression in quadriceps of dystrophic mice compared to the Reference AAV composition (FIGS. 1A-1B).

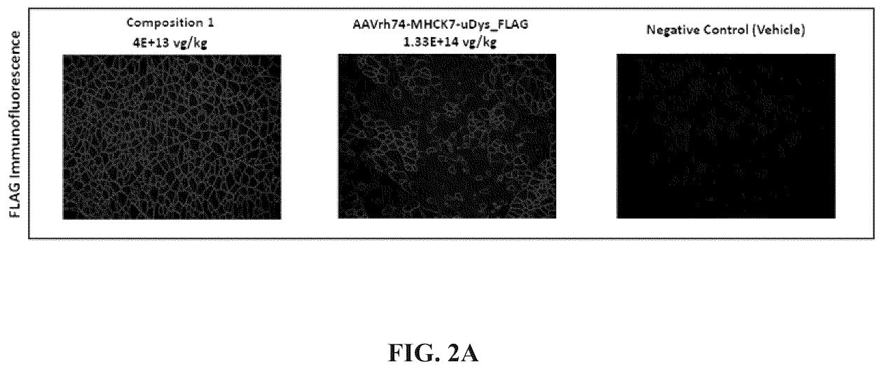

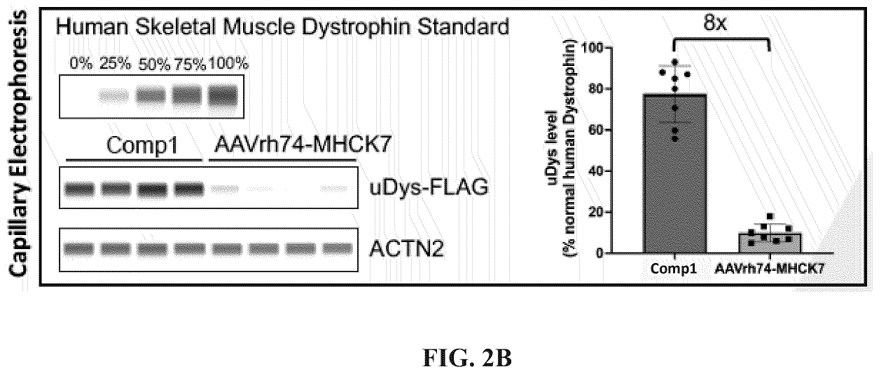

FIG. 2A shows images of FLAG immunofluorescence of microdystrophin expression in B10-mdx mouse triceps following administration of Composition 1 or the Reference AAV composition. FIG. 2B shows results from capillary electrophoresis quantifying microdystrophin expression in B10-mdx mouse triceps following administration of Composition 1 or the Reference AAV composition.

Administration of Composition 1 resulted in 8 times higher microdystrophin protein expression in triceps of dystrophic mice compared to the Reference AAV composition (FIGS. 2A-2B).

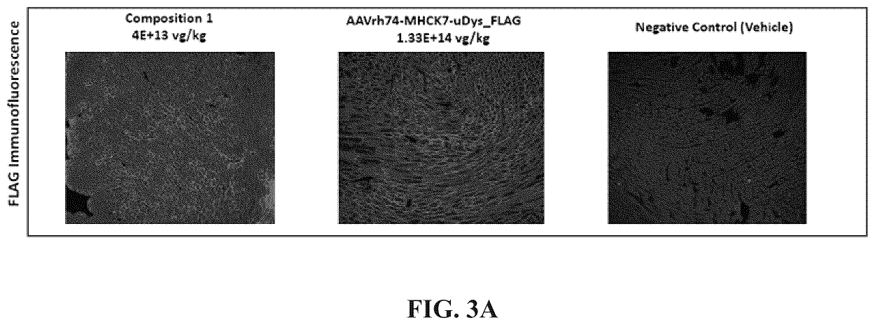

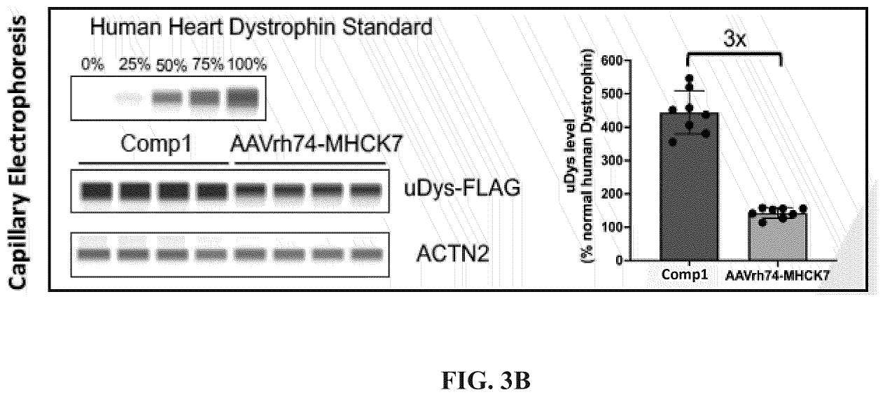

FIG. 3A shows images of FLAG immunofluorescence of microdystrophin expression in B10-mdx mouse heart following administration of Composition 1 or the Reference AAV composition. FIG. 3B shows results from capillary electrophoresis quantifying microdystrophin expression in B10-mdx mouse hearts following administration of Composition 1 or the Reference AAV composition.

Administration of Composition 1 resulted in 3 times higher microdystrophin protein expression in heart muscle of dystrophic mice compared to the Reference AAV composition (FIGS. 3A-3B).

Taken together, these results demonstrate that Composition 1-injected mice had greater expression of microdystrophin in each muscle group tested compared to Reference AAV composition-injected mice. This observed increase in microdystrophin expression could be achieved using a lower dose of Composition 1 (4E+13 vg/kg) than Reference AAV composition (1.33E+14 vg/kg).

### Example 4: Administration of Composition 1 to B10-Mdx Mice Resulted in Restoration of Muscle Function

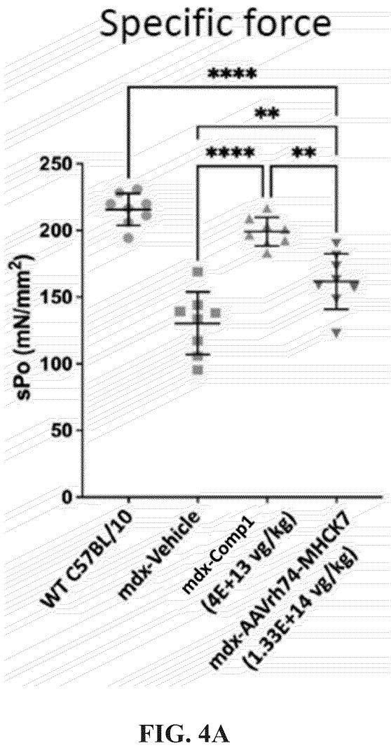

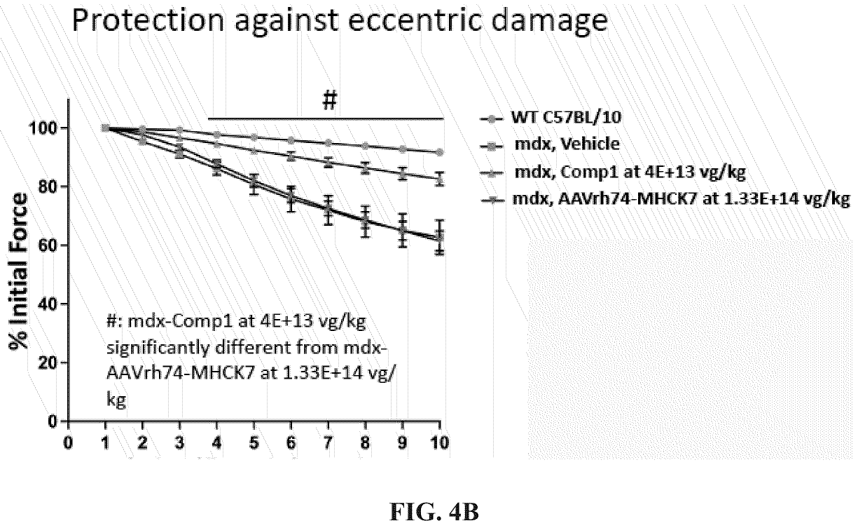

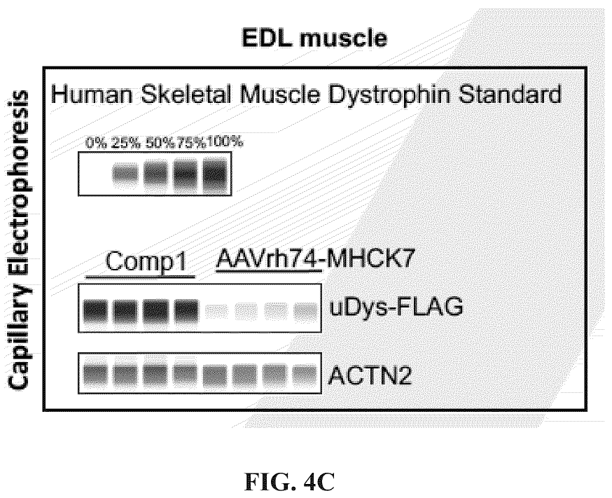

Muscle physiology of mice injected with Composition 1 or Reference AAV composition was analyzed. Specific force (FIG. 4A), protection of muscle against eccentric contraction (FIG. 4B), and microdystrophin expression (FIG. 4C) of extensor digitorum longus (EDL) muscle was analyzed. Protection of dystrophic skeletal muscle against eccentric damage was shown to be dependent on microdystrophin expression levels (FIGS. 4A-4C). EDL muscle was analyzed in mice 6 weeks after injection.

FIG. 4A shows a graph of specific force following administration of Composition 1 or the Reference AAV composition. FIG. 4B shows a graph of protection against eccentric damage following administration of Composition 1 or the Reference AAV composition. FIG. 4C shows results from capillary electrophoresis from EDL muscle following administration of Composition 1 or the Reference AAV composition. The level of microdystrophin expression was determined by interpolation using a dystrophin standard curve derived from normal human skeletal muscle samples.

Composition 1 outperformed the Reference AAV composition, reverting specific force back to wild type levels (FIG. 4A). Composition 1 administration resulted in protection of the EDL muscle of B10-mdx mice against eccentric contraction (FIG. 4B). Notably, the Reference AAV failed to outperform the vehicle control in protecting against eccentric damage (FIG. 4B). Microdystrophin expression in EDL muscles of mice administered Composition 1 was confirmed (FIG. 4C), while little to no microdystrophin expression was detected in mice administered Reference AAV composition (FIG. 4C).

Taken together, these results demonstrate that muscles from Composition 1-injected mice had higher levels of microdystrophin expression, recovered significantly greater specific force, and were more protected from damage compared to muscles from Reference AAV composition-injected animals. Notably, the observed increase in microdystrophin expression and restoration of muscle function could be achieved using a lower dose of Composition 1 (4E+13 vg/kg) than the dose of Reference AAV composition (1.33E+14 vg/kg).

### Example 5: Administration of Composition 1 to Non-Human Primates (NHPs) Resulted in Effective Microdystrophin Expression in Heart and Skeletal Muscle

Composition 1 was further benchmarked against Reference AAV composition in NHPs, specifically cynomolgus macaques. Tissues were collected 28 days post-injection, and microdystrophin expression was detected on the sarcolemma using immunofluorescence and quantified using a capillary-based immunoassay. The level of microdystrophin expression was determined by interpolation using a dystrophin standard curve derived from normal human heart or skeletal muscle samples.

Administration is detailed in the table below:

| Test Article | No. of Animals | Dose | |
|---|---|---|---|
| Composition 1 | 3 | 4E+13 | vg/kg |
| Reference AAV composition | 3 | 1.33E+14 | vg/kg |

### NHP Heart:

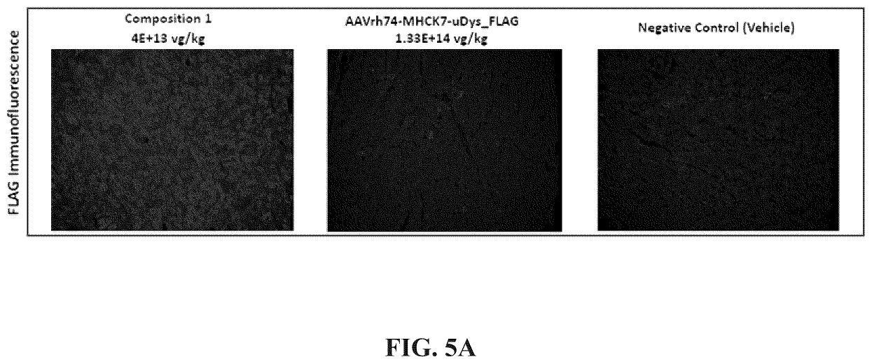

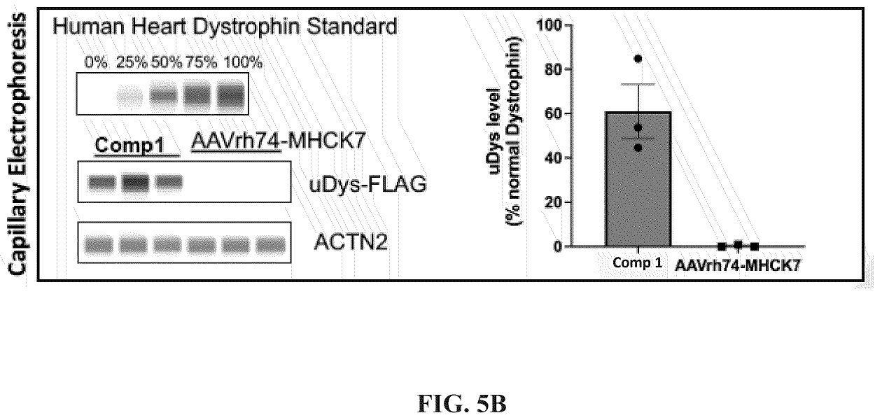

FIG. 5A shows images of FLAG immunofluorescence of microdystrophin expression in NHP heart left ventricles following administration of Composition 1 or the Reference AAV composition. FIG. 5B shows results from capillary electrophoresis quantifying microdystrophin expression in NHP heart left ventricles following administration of Composition 1 or the Reference AAV composition.

Administration of Composition 1 to NHPs resulted in ˜60% microdystrophin protein expression in NHP heart left ventricles as compared to normal human heart dystrophin expression (FIG. 5B). The microdystrophin protein expression levels after injection of Reference AAV composition were below the limit of detection in NHP heart left ventricles (FIGS. 5A-5B).

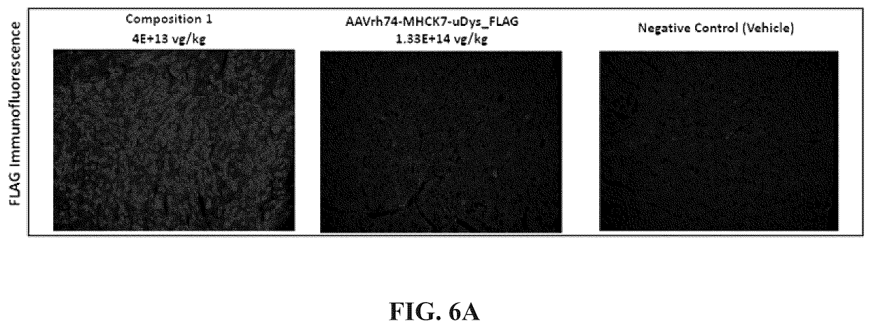

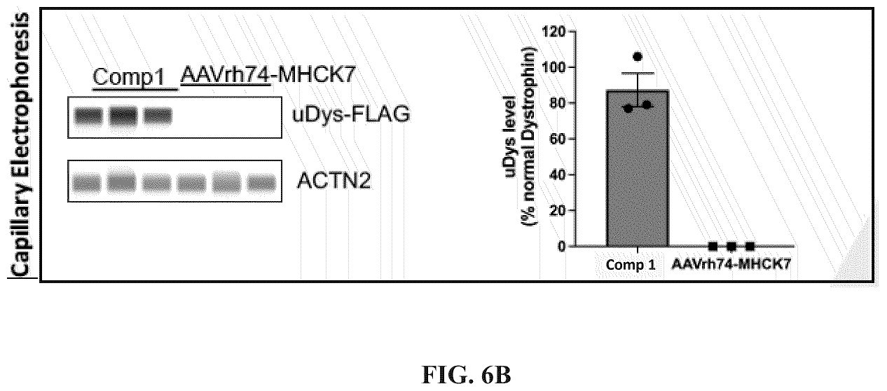

FIG. 6A shows images of FLAG immunofluorescence of microdystrophin expression in NHP heart right ventricles following administration of Composition 1 or the Reference AAV composition. FIG. 6B shows results from capillary electrophoresis quantifying microdystrophin expression in NHP heart right ventricles following administration of Composition 1 or the Reference AAV composition.

Administration of Composition 1 to NHPs resulted in ˜80% microdystrophin protein expression in NHP heart right ventricles as compared to normal dystrophin expression (FIG. 6B, see FIG. 5B for human heart dystrophin standard). The microdystrophin protein levels after injection of Reference AAV composition was below the limit of detection in NHP heart right ventricles (FIGS. 6A-6B).

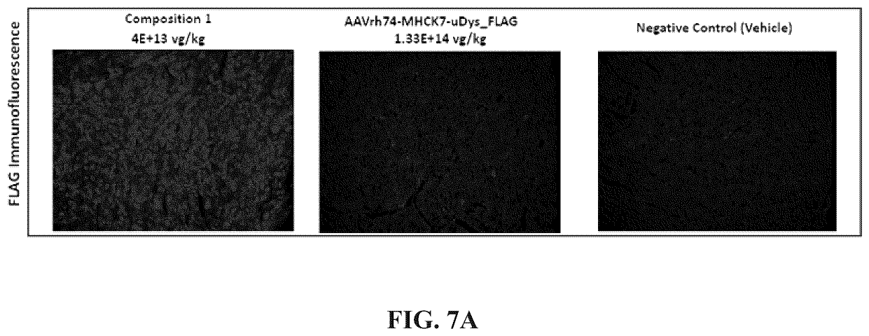

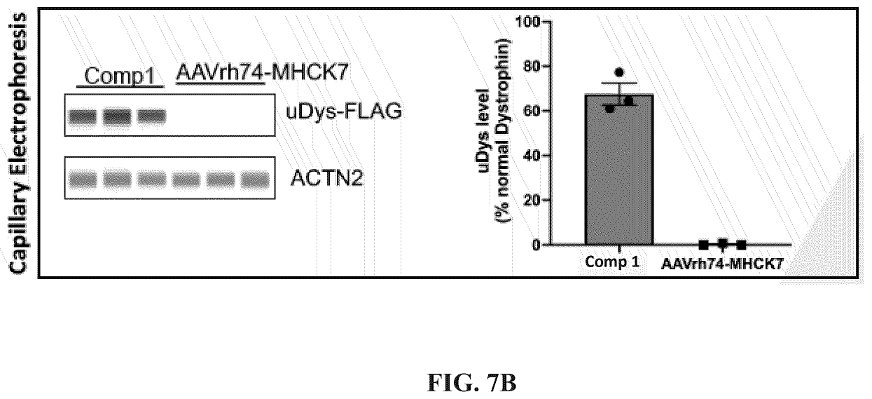

FIG. 7A shows images of FLAG immunofluorescence of microdystrophin expression in NHP heart interventricular septum following administration of Composition 1 or the Reference AAV composition. FIG. 7B shows results from capillary electrophoresis quantifying microdystrophin expression in NHP heart interventricular septum following administration of Composition 1 or the Reference AAV composition.

Administration of Composition 1 to NHPs resulted in ˜70% microdystrophin protein expression in NHP heart interventricular septum as compared to normal dystrophin expression (FIG. 7B, see FIG. 5B for human heart dystrophin standard). The microdystrophin protein levels after injection of Reference AAV composition was below the limit of detection in NHP heart interventricular septum (FIGS. 7A-7B).

Taken together, these results demonstrate that Composition 1-injected NHPs had greater expression of microdystrophin in each heart muscle group tested as compared to Reference AAV composition-injected NHPs. This observed increase in microdystrophin expression could be achieved using a lower dose of Composition 1 (4E+13 vg/kg) than Reference AAV composition (1.33E+14 vg/kg).

### NHP Skeletal Muscle:

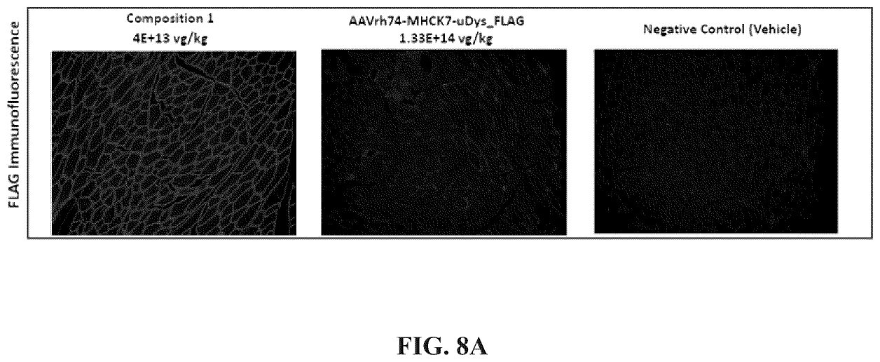

FIG. 8A shows images of FLAG immunofluorescence of microdystrophin expression in NHP gastrocnemius following administration of Composition 1 or the Reference AAV composition. FIG. 8B shows results from capillary electrophoresis quantifying microdystrophin expression in NHP gastrocnemius following administration of Composition 1 or the Reference AAV composition.

Administration of Composition 1 to NHPs resulted in ˜55% microdystrophin protein expression in NHP gastrocnemius as compared to normal dystrophin expression (FIG. 8B). The microdystrophin protein levels after injection of Reference AAV composition was below the limit of detection in NHP gastrocnemius muscle (FIGS. 8A-8B).

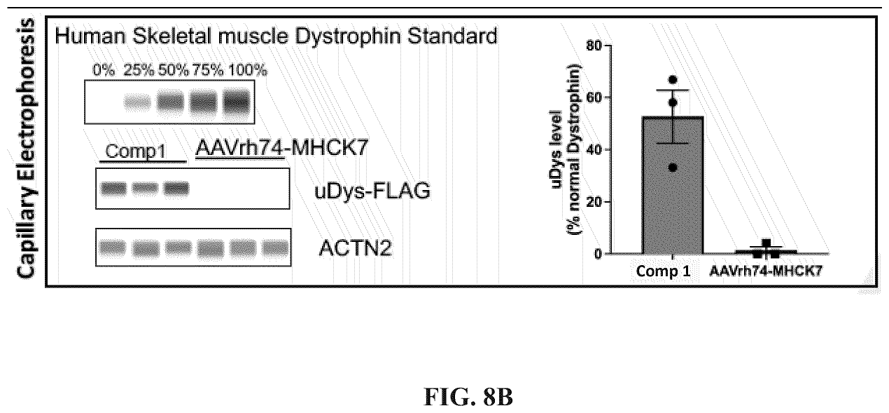

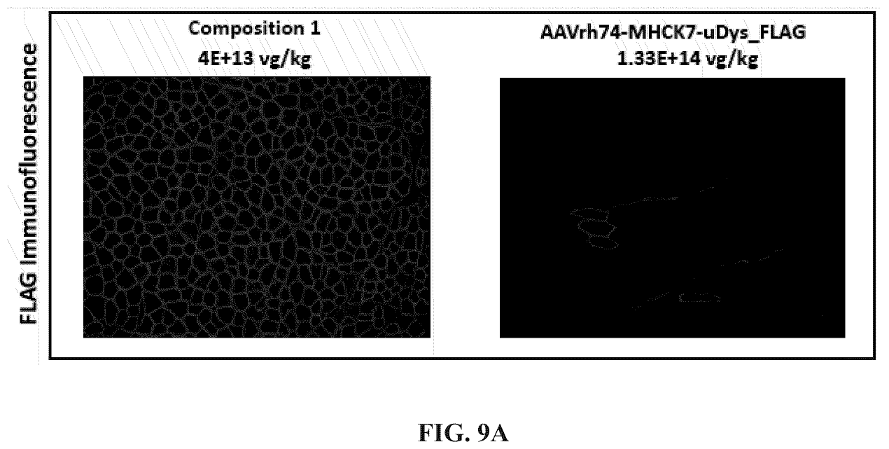

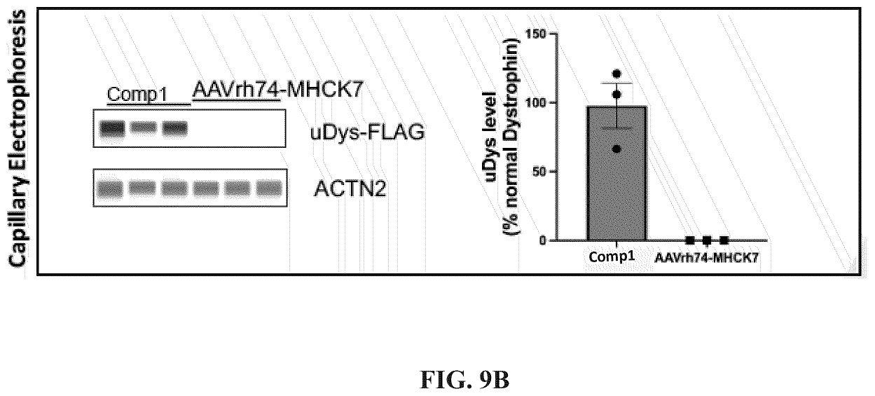

FIG. 9A shows images of FLAG immunofluorescence of microdystrophin expression in NHP pectoralis major following administration of Composition 1 or the Reference AAV composition. FIG. 9B shows results from capillary electrophoresis quantifying microdystrophin expression in NHP pectoralis major following administration of Composition 1 or the Reference AAV composition.

Administration of Composition 1 resulted in ˜100% microdystrophin protein expression as compared to normal dystrophin expression (FIG. 9B, see FIG. 8B for human skeletal muscle dystrophin standard). The microdystrophin protein levels after injection of Reference AAV composition were below the limit of detection in NHP pectoralis major muscle (FIGS. 9A-9B).

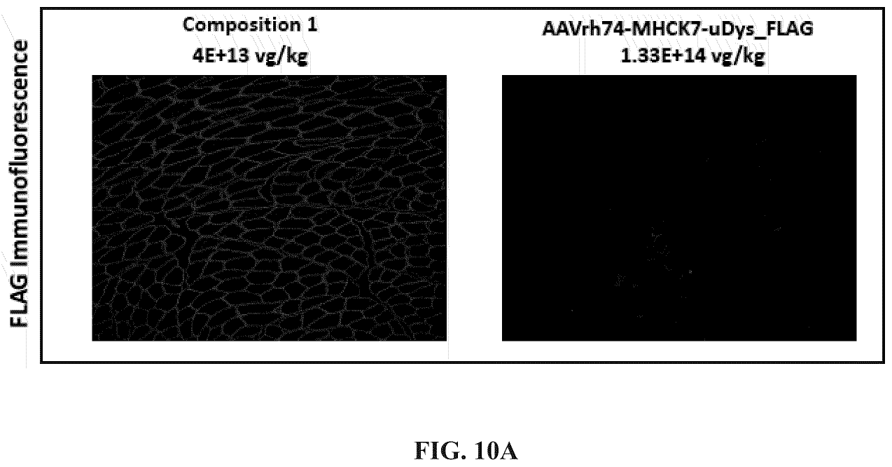

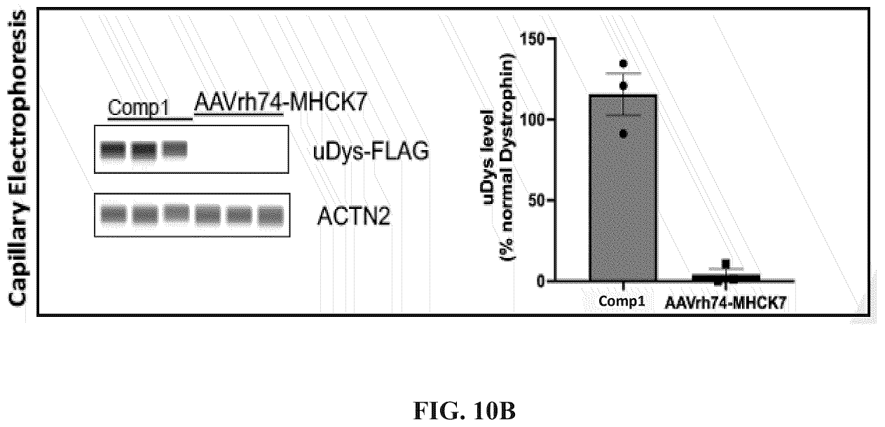

FIG. 10A shows images of FLAG immunofluorescence of microdystrophin expression in NHP diaphragm following administration of Composition 1 or the Reference AAV composition. FIG. 10B shows results from capillary electrophoresis quantifying microdystrophin expression in NHP diaphragm following administration of Composition 1 or the Reference AAV composition.

Administration of Composition 1 resulted ˜120% transgene protein expression as compared to normal dystrophin expression (FIG. 10B, see FIG. 8B for human skeletal muscle dystrophin standard). The Reference AAV composition results resulted in less than 5% transgene expression in NHP diaphragm (FIGS. 10A-10B).

Taken together, these results demonstrate that Composition 1-injected NHPs had greater expression of microdystrophin in each skeletal muscle group tested as compared to Reference AAV composition-injected NHPs. This observed increase in microdystrophin expression in skeletal muscle could be achieved using a lower dose of Composition 1 (4E+13 vg/kg) than Reference AAV composition (1.33E+14 vg/kg).

### Liver Transduction

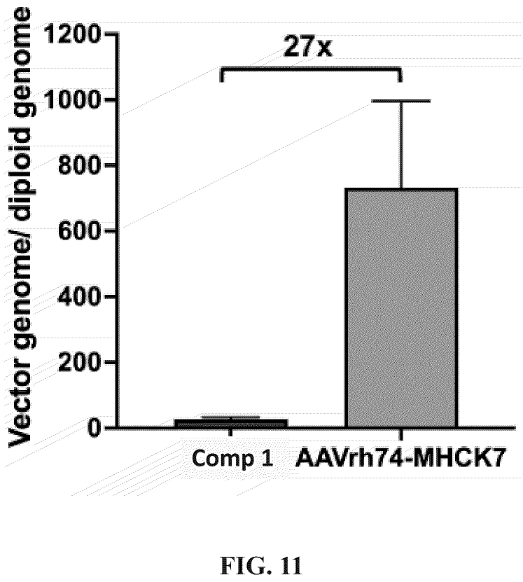

The liver is a major site of transduction by naturally occurring AAV capsids and a target of toxicity associated with administration of AAV vectors. Therefore, the abundance of liver genomes in the Composition 1-injected NHPs and the Reference AAV composition-injected NHPs was analyzed. FIG. 11 shows a graph of vector genome detected in the liver of NHPs injected with Composition 1 or Reference AAV composition.

Composition 1-injected NHPs had a 27 times lower vector genome per diploid genome in the liver compared to the Reference AAV composition-injected NHPs (FIG. 11).

Taken together, these results demonstrate that treatment with Composition 1 can achieve high levels of microdystrophin expression in heart and skeletal muscles of NHPs with lower transduction of liver cells.

### Example 6: Systemic Administration of Composition 1 Results in Dose-Dependent Improvement of Muscle Function in a Severe DMD Mouse Model and Results in High and Uniform Microdystrophin Expression in the Heart and Skeletal Muscles of Non-Human Primates

Duchenne muscular dystrophy (DMD) is a fatal disease, and the most common muscular dystrophy, with 20,000 boys diagnosed worldwide each year. DMD is caused by loss of the dystrophin protein, an essential component of the dystrophin-glycoprotein complex that preserves myofiber membrane integrity during contraction. Adeno-associated virus (AAV) mediated microdystrophin (mDys) gene replacement therapy is a promising approach to treat DMD. Unfortunately, clinical stage AAV-mDys therapies produce low, nonuniform mDys expression in skeletal muscles, with no clinical data supporting mDys expression in the heart, leading to suboptimal clinical efficacy and limited potential to address cardiomyopathy, a major cause of mortality in DMD. Engineered capsids together with regulatory element technologies of were used to develop the lead candidates of the invention, a novel AAV-mDys therapy with the potential to be safer and more effective. The lead candidate expresses a codon-optimized mDys selectively in cardiac and skeletal muscles by using a skeletal muscle/heart-tropic and liver de-targeted capsid (MyoAAV-LD B) and novel regulatory elements. A FLAG tag was added at the C-terminus of the mDys protein to facilitate immunofluorescent detection. To evaluate safety, efficacy, and mDys expression, 8-week-old male D2-mdx mice were injected intravenously with the lead candidate at 1E13, 2E13, and 4E13 vg/kg, and were evaluated 7 weeks later. The lead candidate produced a significant dose-dependent improvement in muscle pathology and fibrosis, grip strength, and mechanical properties of the extensor digitorum longus muscle ex vivo. Maximal efficacy occurred at 4E13 vg/kg, associated with mDys expression in nearly all myofibers at protein levels between 62 and 83% of normal human dystrophin levels. Furthermore, the lead candidate was well tolerated and did not induce toxicity in any treated mice.

To translate these findings to large animals and to guide human dose selection, biodistribution and safety of the lead candidate was evaluated in juvenile male non-human primates (NHPs). Animals received a single intravenous dose of 4E13 vg/kg, and tissues were collected 28- and 90-days post-dose. The lead candidate was well tolerated, had no effects on chemistry, hematology, or histopathology up to 90 days, and produced robust, uniform mDys expression in almost all skeletal muscle fibers and cardiomyocytes. The average levels of mDys protein in heart and skeletal muscles relative to normal human dystrophin were 72% and 66%, respectively. Further, the expression of the mDys transgene was highly selective for skeletal muscle and heart, with no transgene expression detected in off-target tissues.

In summary, the results illustrate the need for high mDys expression in the majority of myofibers to produce maximal efficacy, and for the first time demonstrate quantitative expression of mDys protein in NHP muscle at levels that are associated with maximal efficacy in a DMD mouse model. These results provide compelling preclinical data to support further development of Composition 1 to treat DMD.

### Example 7: Administration of Composition 1 Results in Dose-Dependent Microdystrophin Expression in a Severe DMD Mouse Model

To evaluate safety, efficacy, and microdystrophin expression of Composition 1 (described in Example 3), 8-week-old male D2-mdx mice, which is a severe mouse model of DMD, were injected intravenously with Composition 1 at 1E13, 2E13, or 4E13 vg/kg. Mice were evaluated 7 weeks and 22 weeks post-injection. WT mice and D2-mdx mice were injected with vehicle as a control. Microdystrophin expression was detected by FLAG immunofluorescence and quantified using capillary-based immunoassay.

Treatment with Composition 1 did not induce cardiotoxicity or hepatotoxicity in D2-mdx mice (data not shown). This demonstrates that Composition 1 can be administered with little to no damage or dysfunction to the heart and the liver.

FIG. 12 shows images of FLAG immunofluorescence of microdystrophin expression in D2-mdx mouse diaphragm, quadriceps, heart, and extensor digitorum longus (EDL) tissue at 7 weeks post injection following administration of Composition 1.

Immunofluorescence analysis of mouse tissues showed that Composition 1 treatment resulted in dose-dependent expression of microdystrophin in each of the muscles examined (EDL, diaphragm, quadriceps, and heart) (FIG. 12).

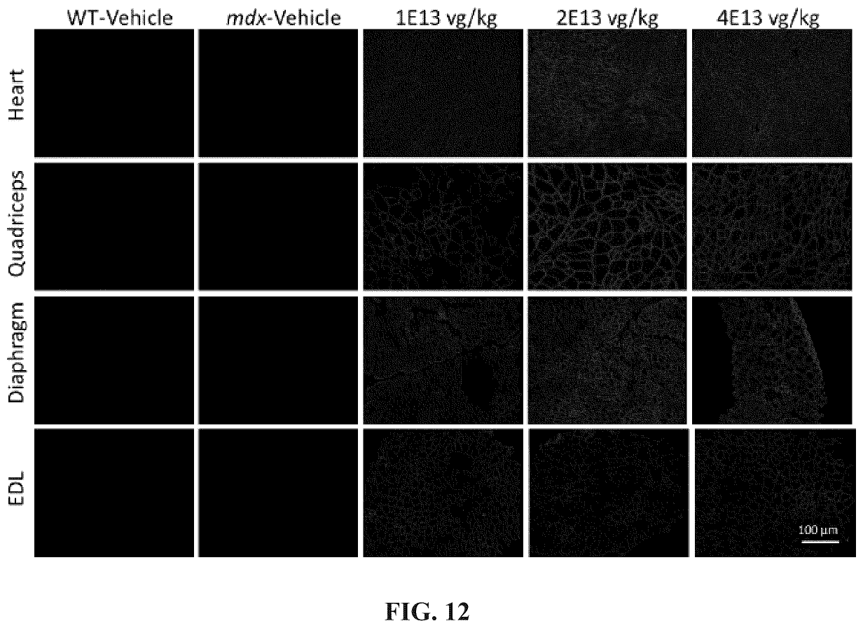

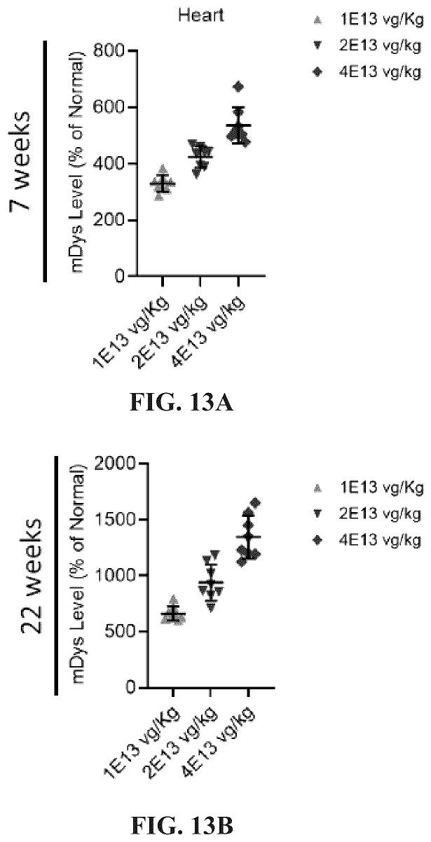

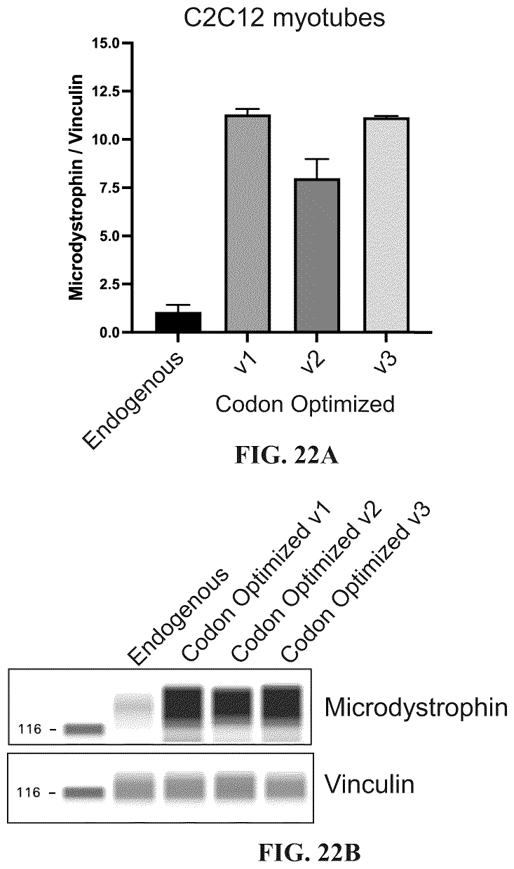

FIGS. 13A-13B shows results from capillary electrophoresis quantifying microdystrophin expression in D2-mdx mouse heart tissue at 7 weeks (FIG. 13A) and 22 weeks (FIG. 13B) following administration of Composition 1.

The examined heart tissues showed supra-physiological microdystrophin protein levels, exceeding 300% of normal in the low-dose group, and up to 535% in the high-dose group at 7 weeks post injection (FIG. 13A). At 22 weeks post dosing, the microdystrophin protein level in the heart was ˜2.2-fold higher than the 7-week time point across each of the tested doses (FIGS. 13A-13B).

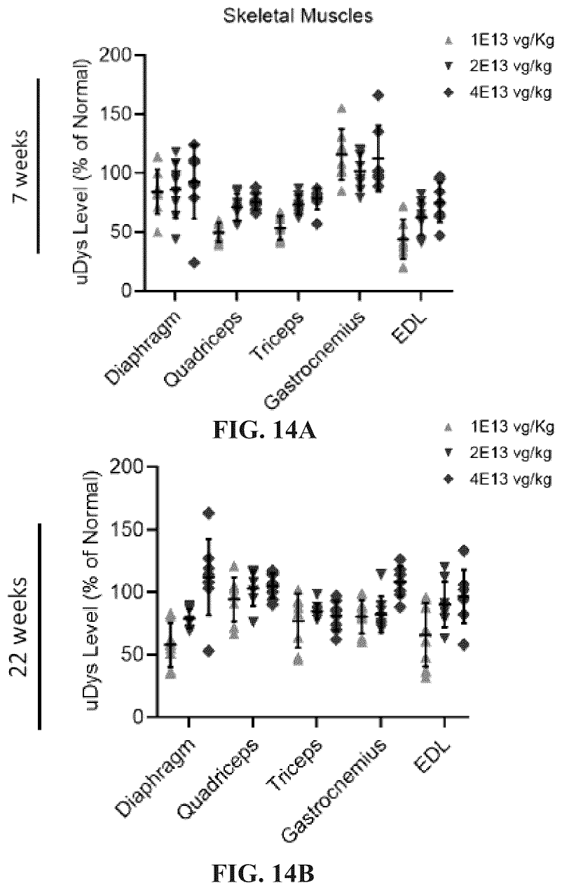

FIGS. 14A-14B shows results from capillary electrophoresis quantifying microdystrophin expression in D2-mdx mouse skeletal muscle tissue at 7 weeks (FIG. 14A) and 22 weeks (FIG. 14B) following administration of Composition 1.

In skeletal muscle, microdystrophin protein level was, on average, 70% of normal in the low-dose group and was, on average, 83% of normal in the mid and high-dose groups at 7 weeks post-dosing (FIG. 14A). By 22 weeks, the microdystrophin protein level in skeletal muscles increased minimally by 1.2-fold across all doses (FIGS. 14A-14B). At 22 weeks post-injection, the percent of microdystrophin-FLAG positive myofibers in EDL and quadriceps was similar, with 76%, 88%, and 93% expression in the low, mid, and high doses, respectively (FIG. 14B).

Taken together, these results demonstrate that treatment with Composition 1 restored microdystrophin protein levels in heart and skeletal muscles in a severe DMD mouse model, without inducing cardiotoxicity or hepatotoxicity.

### Example 8: Administration of Composition 1 Increased Body and Tibialis Anterior (TA) Muscle Weights in a Severe DMD Mouse Model

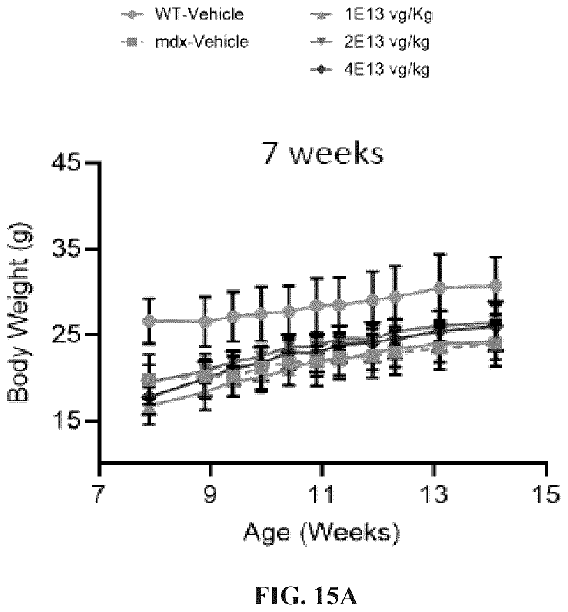

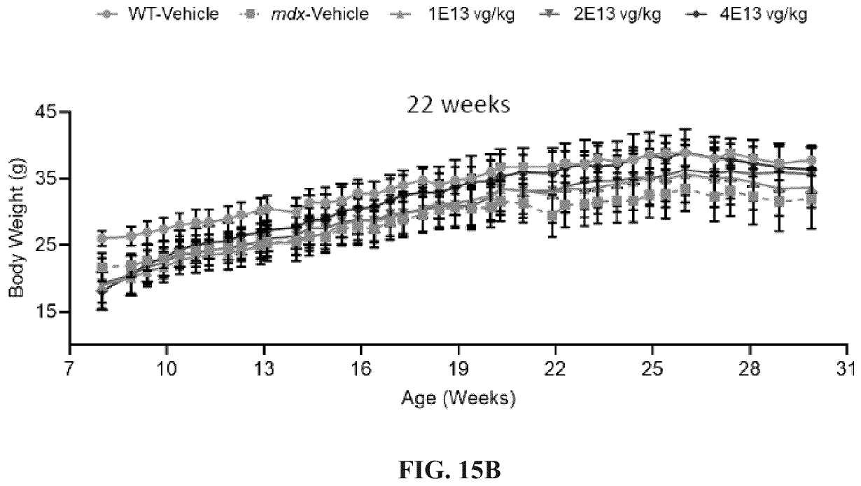

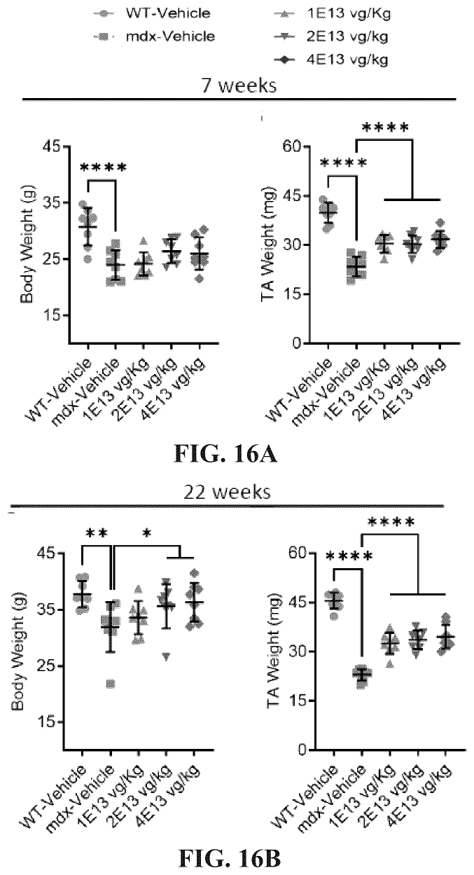

Composition 1 (described in Example 3) was intravenously injected into 8-week-old male D2-mdx mice, a severe DMD mouse model, at a dose of 1E13, 2E13, or 4E13 vg/kg. WT mice and D2-mdx mice were injected with vehicle as a control. Body weight of mice was measured pre-dose and monitored twice weekly throughout the 7 weeks and 22 weeks study cohorts (FIGS. 15A-15B). Body weight and TA muscle weight were measured at 7 weeks and 22 weeks post-injection (FIGS. 16A-16B).

FIGS. 15A-15B show graphs of body weight of D2-mdx mouse over 7 weeks (FIG. 15A) and 22 weeks (FIG. 15B) following administration of Composition 1. After about 16 weeks, body weights of mice administered the high dose of Composition 1 were comparable to wild type mice (FIGS. 15A-15B).

FIGS. 16A-16B show graphs of body weight and TA weight of D2-mdx mouse at 7 weeks (FIG. 16A) and at 22 weeks (FIG. 16B) following administration of Composition 1.

Mice injected with the mid or high dose of Composition 1 showed a significant increase in body weight at 22 weeks post-dosing (FIG. 16B, left panel). Composition 1-injected mice at each of the tested doses showed a significant increase in TA weight after 7 weeks (FIG. 16A, right panel) and 22 weeks. (FIG. 16B, right panel).

Taken together, these results demonstrate that treatment with Composition 1 increased body and TA muscle weights in a severe DMD mouse model. The observed increase in body and TA muscle weights could be achieved using low (1E13 vg/kg), middle (2E13 vg/kg), and high (4E13 vg/kg) doses of Composition 1.

### Example 9: Administration of Composition 1 Improved the Mechanical Properties of Extensor Digitorum Longus (EDL) Muscle in a Severe DMD Mouse Model

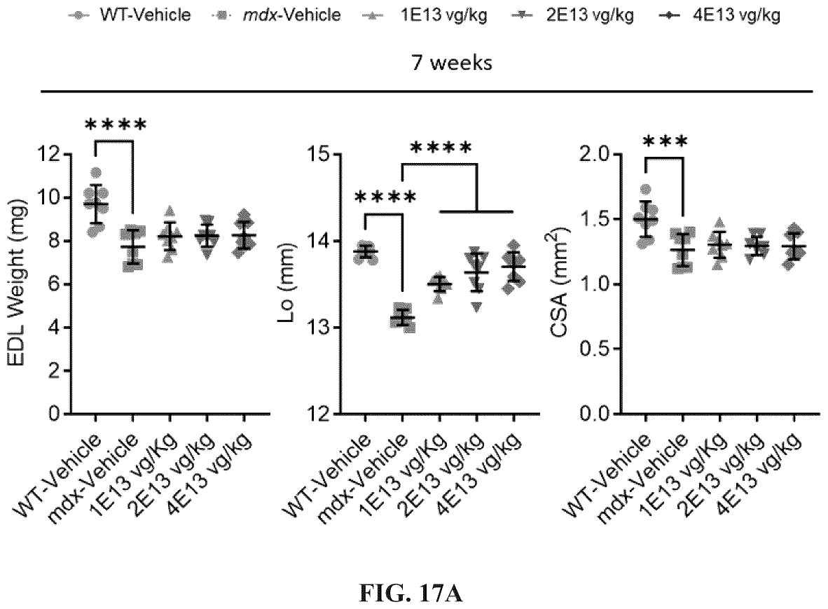

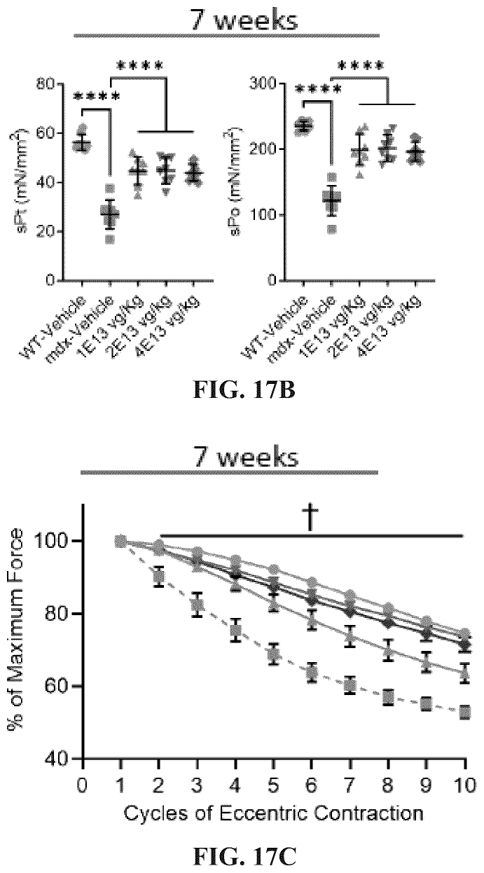

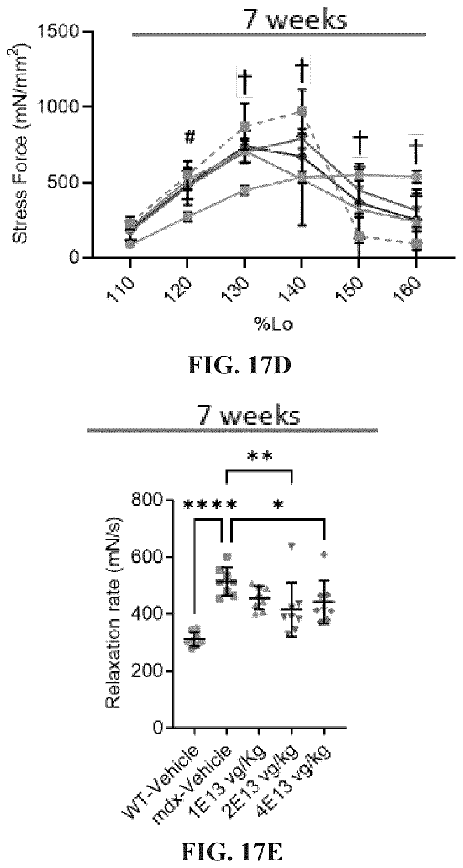

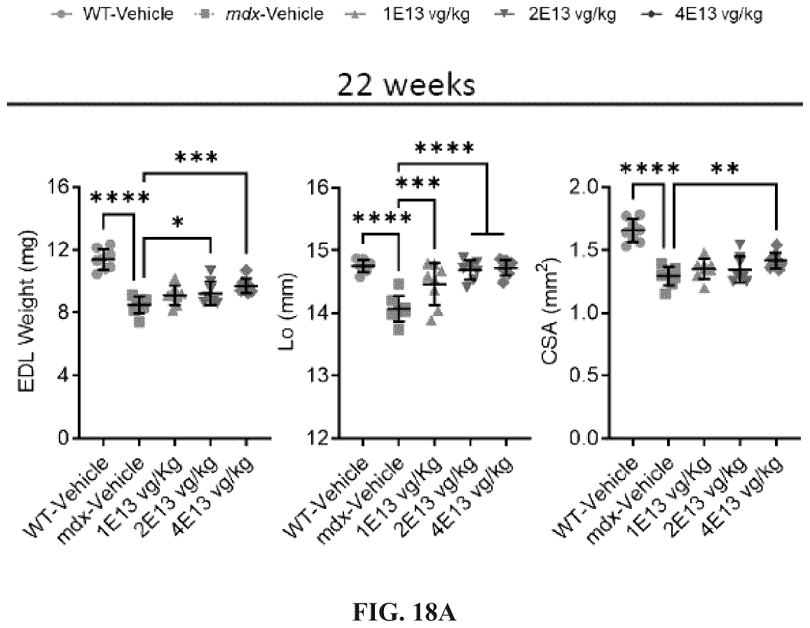

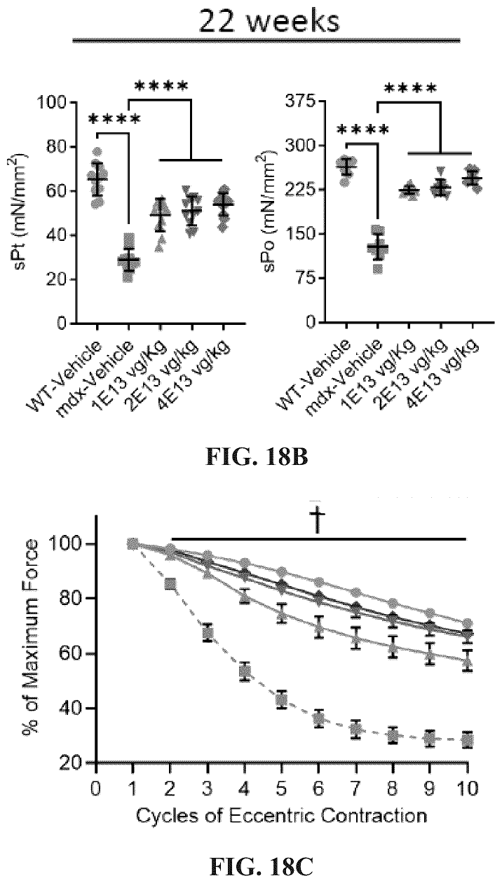

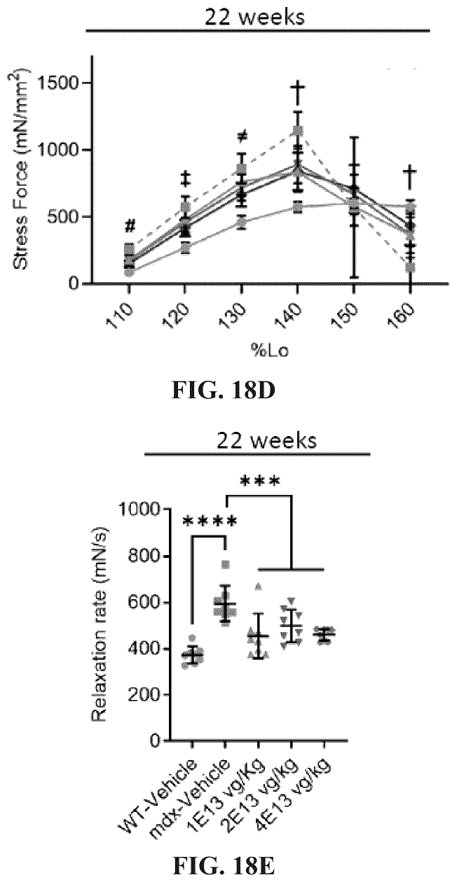

Composition 1 (described in Example 3) was intravenously injected into 8-week-old male D2-mdx mice, a severe DMD mouse model, at a dose of 1E13, 2E13, or 4E13 vg/kg. WT mice and D2-mdx mice were injected with vehicle as a control. The anatomical properties (FIG. 17A and FIG. 18A), muscle contractility (FIG. 17B and FIG. 18B), protection against eccentric contraction (FIG. 17C and FIG. 18C), elastic properties (FIG. 17D and FIG. 18D), and viscous properties (FIG. 17E and FIG. 18E) of EDL muscle was analyzed at 7 weeks and 22 weeks post-injection.

FIG. 17A and FIG. 18A show graphs of anatomical measurements of EDL muscle, including cross-section area (CSA) and optimal length (Lo), in D2-mdx mouse at 7 weeks (FIG. 17A) or 22 weeks (FIG. 18A) post-injection of Composition 1.

FIG. 17B and FIG. 18B show graphs of quantitative measurements of muscle contractility in EDL muscle, including specific twitch force (Pt) and tetanic force (Po), in D2-mdx mouse at 7 weeks (FIG. 17B) or 12 weeks (FIG. 18B) post-injection of Composition 1.

FIG. 17C and FIG. 18C show a graph of eccentric contraction of EDL muscle in D2-mdx mice at 7 weeks (FIG. 17C) or 12 weeks (FIG. 18C) post-injection of Composition 1.

FIG. 17D and FIG. 18D show a graph of the stress-strain relationship, evaluating elasticity, of EDL muscle in D2-mdx mice at 7 weeks (FIG. 17D) or 12 weeks (FIG. 18D) post-injection of Composition 1.

FIG. 17E and FIG. 18E shows a graph of the stress relaxation rate, evaluation the viscous properties of muscle, of EDL muscle in D2-mdx mice at 7 weeks (FIG. 17E) or 12 weeks (FIG. 18E) post-injection of Composition 1.

Taken together, these results demonstrate that treatment of D2-mdx mice with Composition 1 improved the contractile properties of the EDL muscle in a dose-dependent manner and provided long-term protection of muscle function up to 22 weeks post-dosing. The treatment with Composition 1 was fully efficacious at the mid-dose of 2E13 vg/kg. However, further improvements in many mechanical parameters (e.g., EDL weight, optimal length, specific twitch force) were noted in the high-dose group (4E13 vg/kg).

### Example 10: Administration of Composition 1 Resulted in High and Selective Microdystrophin Expression in Heart and Skeletal Muscles of Non-Human Primates (NHPs)

Biodistribution and safety of the Composition 1 was evaluated in juvenile male non-human primates (NHPs). Animals received a single intravenous dose of 4E13 vg/kg of Composition 1, and tissues were collected 28- and 90-days post-injection. Microdystrophin-FLAG mRNA levels were assessed in heart, skeletal muscles, and off-target tissues. Expression of microdystrophin was analyzed by FLAG immunostaining.

Composition 1 was safe and well tolerated, with no treatment-related effects in clinical chemistry, hematology, and histopathology, including liver and DRG up to 90 days post-injection (data not shown).

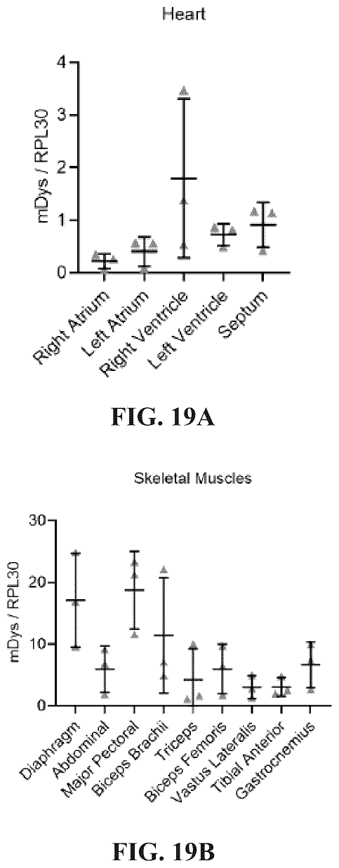

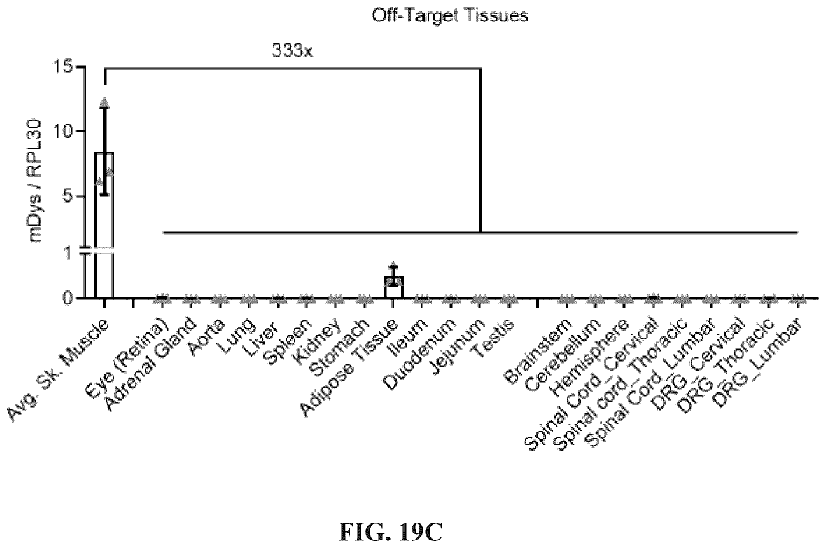

FIG. 19A-19C show graphs of results quantifying transgene mRNA levels in NHP heart, skeletal muscle, and off-target tissues 28 days post administration of Composition 1. The transcript copy was normalized to nhpRPL30 (n=3/group). These results demonstrate that treatment of NHPs with Composition 1 results in high and selective expression of microdystrophin in NHP heart and skeletal muscles, with little to no expression in off-target tissues.

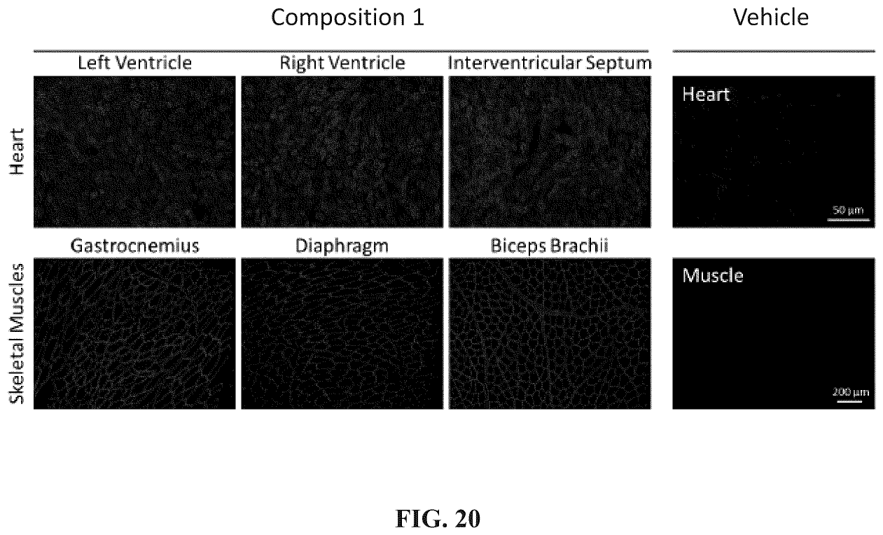

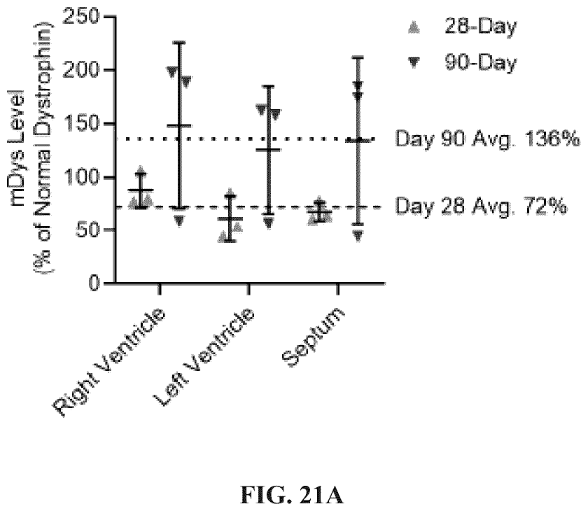

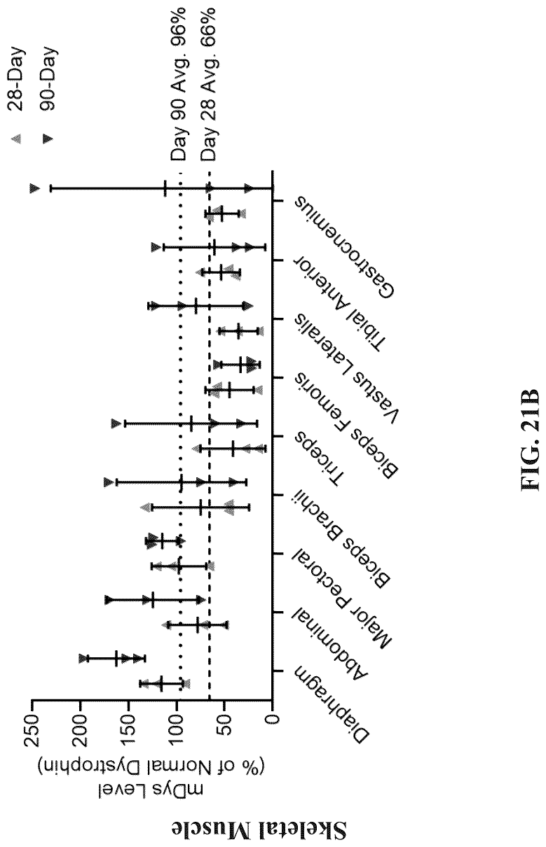

FIG. 20 shows FLAG immunofluorescence images of microdystrophin expression in NHP heart and skeletal muscles 28 days post administration of Composition 1. FIGS. 21A-21B show graphs of results from capillary electrophoresis quantifying microdystrophin expression in NHP heart and skeletal muscles at 28 days and 90 days post administration of Composition 1. The amount of the microdystrophin expressed in tissues was calculated by interpolation to the full-length dystrophin standard curve derived from normal human heart or skeletal muscle samples.

Treatment with Composition 1 resulted in high microdystrophin protein expression in skeletal muscles and the cardiomyocytes. After 28 days, treatment with Composition 1 resulted in robust microdystrophin protein expression levels in heart and skeletal muscles that were, on average, 72% and 66% of normal full-length dystrophin, respectively (FIGS. 21A-21B). Microdystrophin expression levels increased after 90 days post-injection, with microdystrophin protein expression levels in heart and skeletal muscles that were, on average, 136% and 96% of normal full-length dystrophin, respectively (FIGS. 21A-21B).

Taken together, these results demonstrate that a single intravenous dose of Composition 1 was safe and well tolerated in NHPs up to 90 days post injection and resulted in high microdystrophin transcript and protein expression in skeletal muscles and the heart.

The Examples described herein use Composition 1 as an example composition for treating DMD using the recombinant nucleic acids, vectors, and viral particles described herein.

### Example 11: Codon Optimization Increased Expression of Microdystrophin

Three different codon optimized microdystrophin sequences (referred to as v1, v2, and v3) were generated and the expression of microdystrophin from the codon optimized sequences was tested in C2C12 derived myotubes and human primary myotubes. Microdystrophin expression was detected and quantified using a capillary-based immunoassay. The level of microdystrophin expressed from codon optimized microdystrophin sequences was compared to the level of microdystrophin expressed from an endogenous coding sequence of dystrophin. Vinculin was used as a loading control.

FIGS. 22A-22B show results from capillary electrophoresis analysis of microdystrophin expression from an endogenous coding sequence or a codon optimized microdystrophin sequence (v1, v2, or v3) in C2C12 derived myotubes.

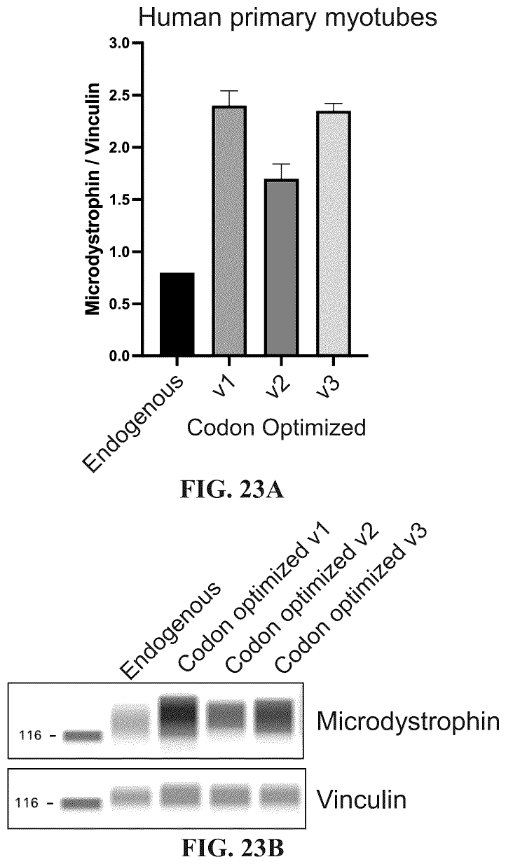

FIGS. 23A-23B show results from capillary electrophoresis analysis of microdystrophin expression from an endogenous coding sequence or a codon optimized microdystrophin sequence (v1, v2, or v3) in human primary myotubes.

In both C2C12 derived myotubes (FIGS. 22A-22B) and human primary myotubes (FIGS. 23A-23B), the level of expression of microdystrophin from each of the codon optimized microdystrophin sequences (v1, v2, v3) was significantly greater than the level of microdystrophin expressed from the endogenous coding sequence.

These results demonstrate that microdystrophin expression can be significantly increased using a codon optimized microdystrophin sequence (v1, v2, or v3) compared to the endogenous microdystrophin sequence.

### Example 12: Administration of MyoAAV-LD a Resulted in Higher Microdystrophin Expression Compared to AAV9 in NHPs

To investigate the muscle transduction profile of MyoAAV-LD A after systemic delivery, NHPs were injected with either MyoAAV-LD A- or AAV9-CBh-uDys-FLAG, and microdystrophin transgene expression in different muscle tissues of the injected NHPs was examined. CBh-uDys-FLAG includes a C-terminal FLAG tagged microdystrophin (uDys) under the control of a hybrid CBA promoter (CBh). Gastrocnemius muscle from NHPs injected with vehicle (saline) was used as a negative control.

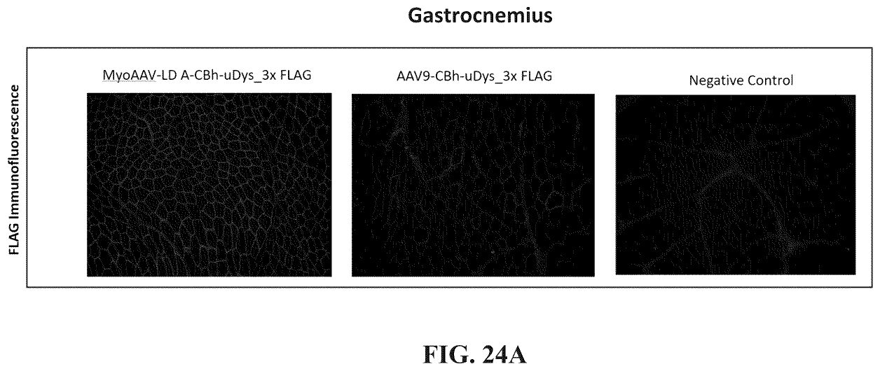

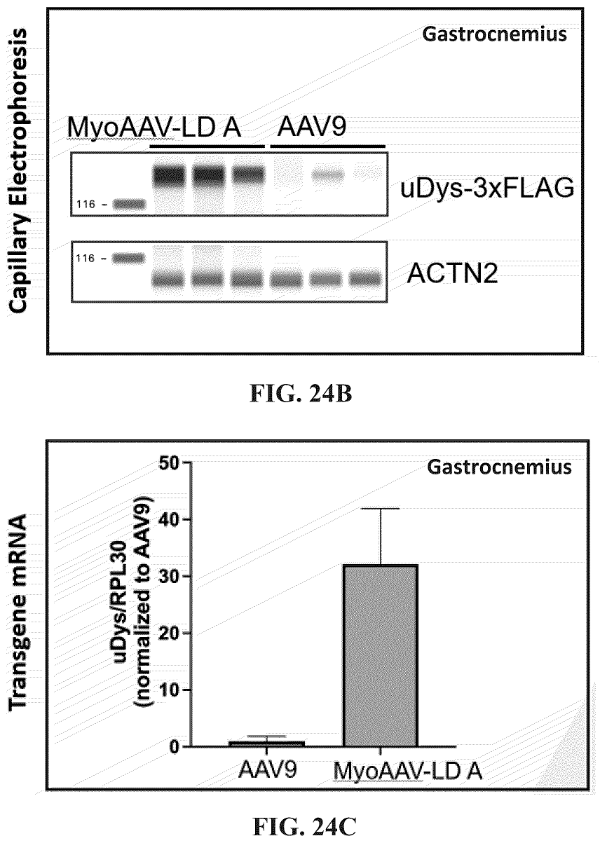

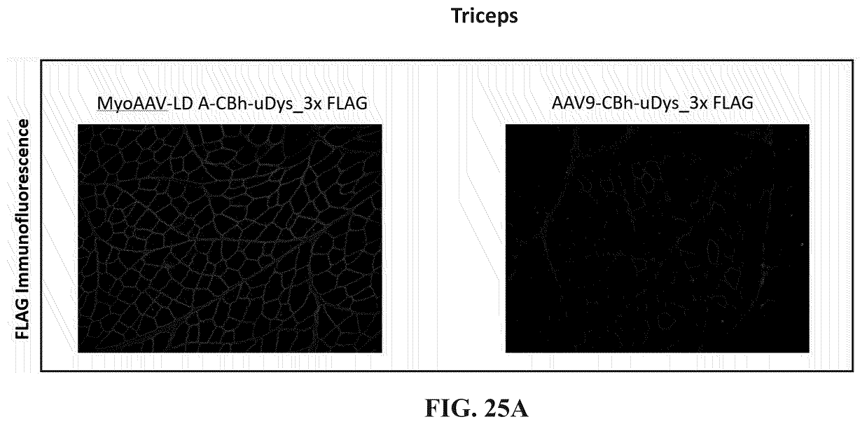

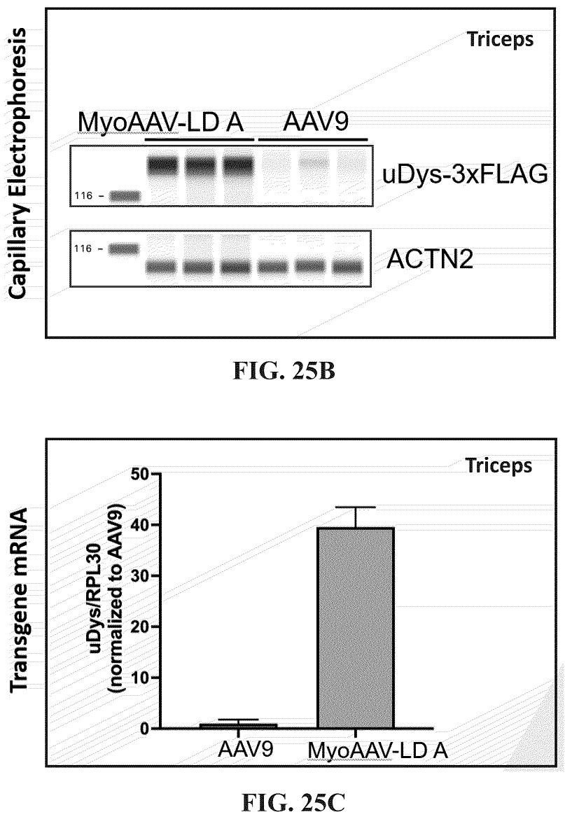

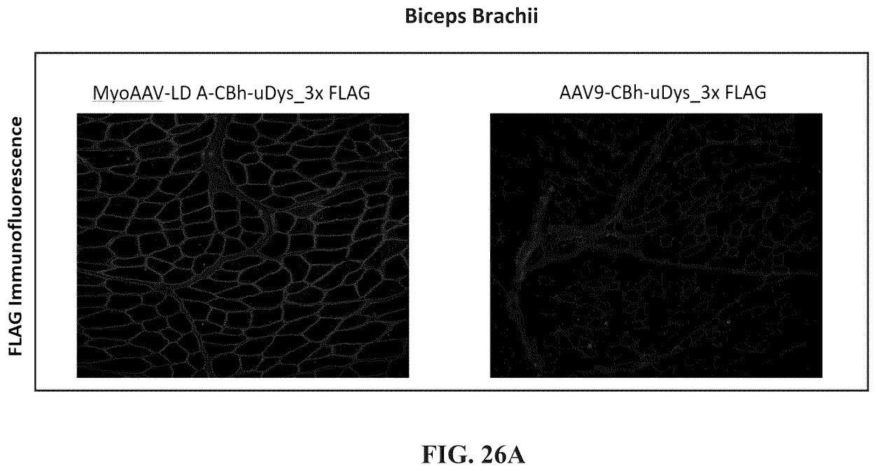

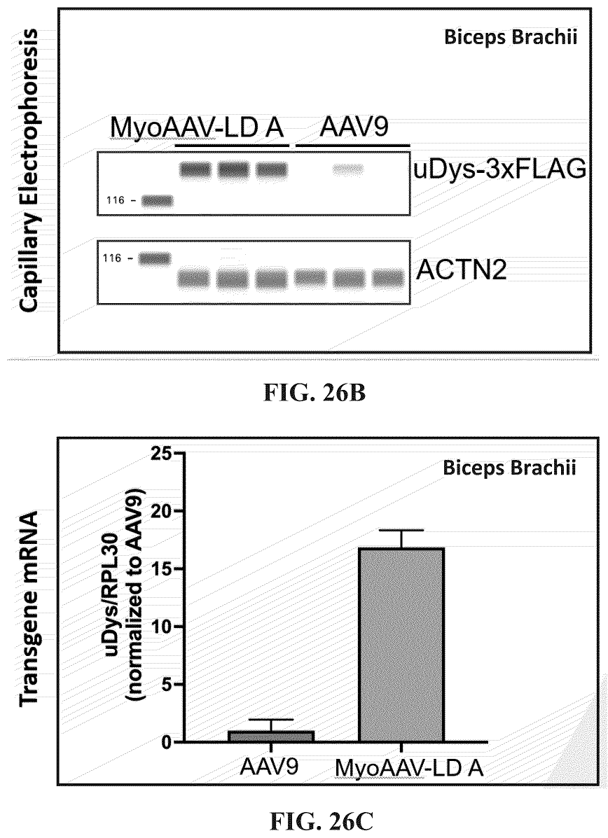

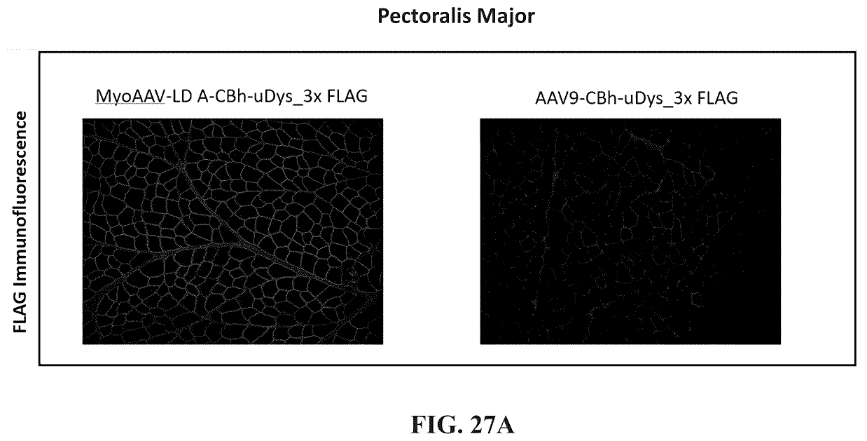

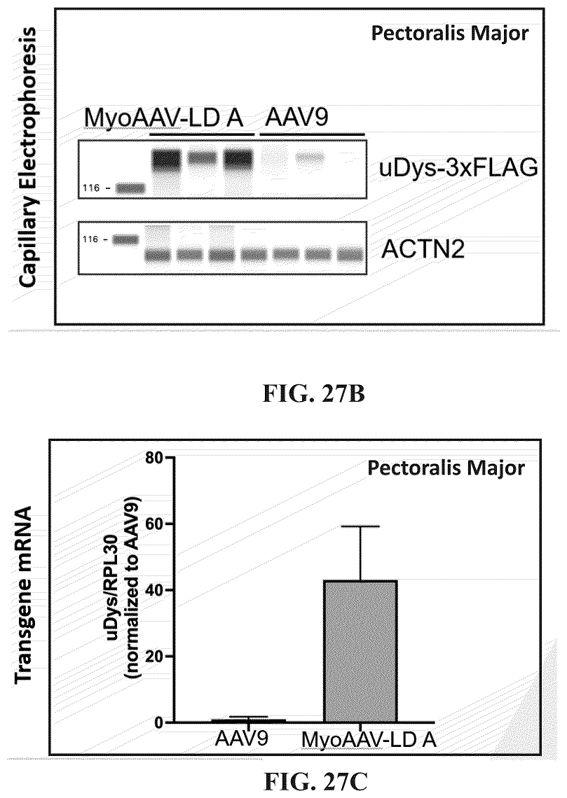

Microdystrophin expression was detected using immunofluorescence and quantified using a capillary-based immunoassay. Microdystrophin mRNA was quantified using a standard taqman assay. nhpRPL30 mRNA was used as the housekeeping control for quantification of microdystrophin expression. Microdystrophin expression was assessed in skeletal muscles including gastrocnemius (FIGS. 24A-24C), triceps (FIGS. 25A-25C), biceps brachii (FIGS. 26A-26C), and pectoralis major (FIGS. 27A-27C).

Fluorescence imaging of harvested tissues revealed far greater fluorescence intensity in muscles of NHP injected with MyoAAV-LD A—as compared to AAV9-injected NHP (FIG. 24A, FIG. 25A, FIG. 26A, and FIG. 27A). Strong microdystrophin transgene expression in muscle fibers of MyoAAV-LD A-injected NHP was further confirmed by capillary electrophoresis (FIG. 24B, FIG. 25B, FIG. 26B, and FIG. 27B). Quantification of microdystrophin mRNA in different skeletal muscles of NHP revealed 15 to 40 times higher microdystrophin transgene expression in muscles of MyoAAV-LD A-injected NHPs compared to AAV9-injected NHPs (FIG. 24C, FIG. 25C, FIG. 26C, and FIG. 27C).

Taken together, these results demonstrate that MyoAAV-LD A can transduce skeletal muscles with significantly higher efficiency that wild type AAV9.

### Example 13: Administration of DMD Tool Compound Restores Muscle Force and Protects the Muscle Against Eccentric Damage in B10-Mdx Mice

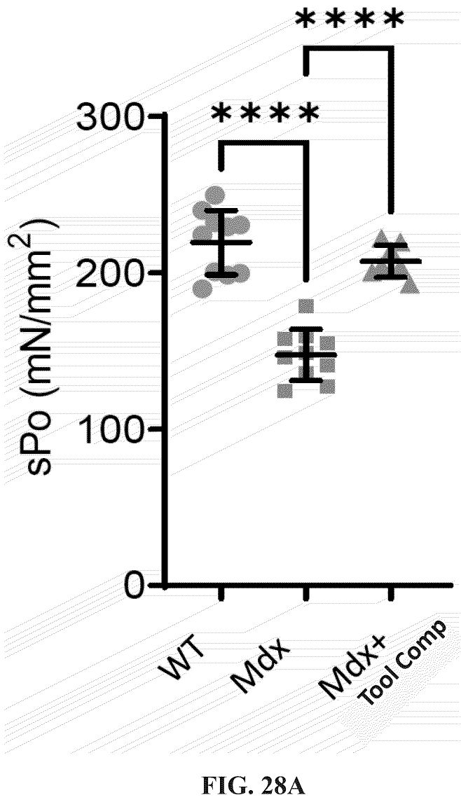

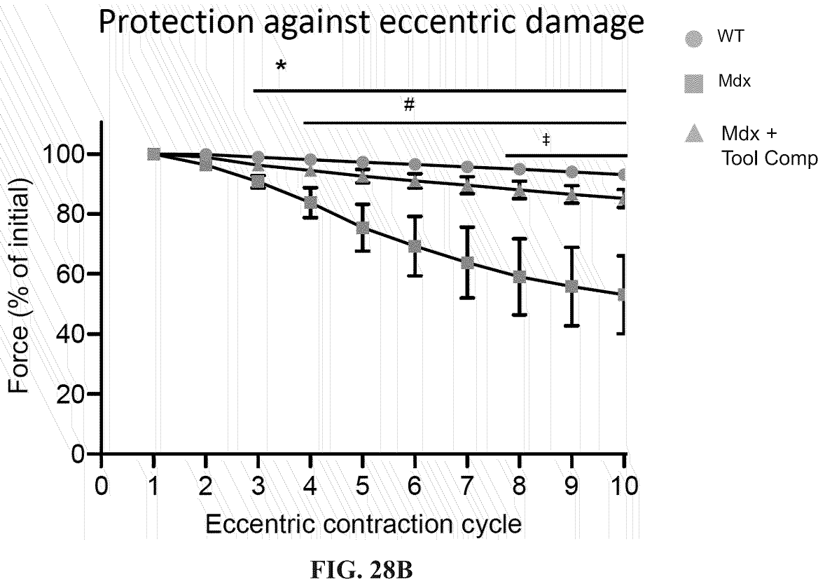

Muscle physiology, including specific force (FIG. 28A) and protection of muscle against eccentric contraction (FIG. 28B), was analyzed in mice injected with a DMD tool compound that includes MyoAAV-LD B-uDys under the control of a muscle specific promoter. 8 weeks old B10-mdx mice were injected with 2E+13 vg/kg of the DMD tool compound, and EDL muscle was analyzed 6 weeks after injection.

Administration of the DMD tool compound reverted specific force back to wild type levels (FIG. 28A) and resulted in protection of the EDL muscle against eccentric contraction (FIG. 28B). These results demonstrate that delivery of microdystrophin to muscle tissues via MyoAAV-LD B can restore muscle strength and protect muscle against damage in a mouse model of DMD.

### CONCLUSION

AAV compositions of the invention (combining an engineered liver de-targeted MyoAAV capsid comprising the amino acid sequence RGDR (SEQ ID NO: 2440), a novel promoter of the invention, and a codon optimized microdystrophin sequence of the invention) result in expression of microdystrophin in mice and NHPs at rates far superior to a Reference AAVrh74 capsid comprising a muscle specific promoter (MHCK7) and a transgene encoding microdystrophin, while also reducing the presence of AAV genomes in the liver. Notably, the Reference AAV capsid resulted in very low microdystrophin expression in NHP skeletal muscles and undetectable expression in the NHP heart, while compositions of the invention resulted in uniform microdystrophin expression throughout NHP heart and skeletal muscle tissues with levels comparable to endogenous dystrophin levels.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A recombinant nucleic acid comprising:
a muscle specific promoter;
a sequence encoding microdystrophin; and
at least one dorsal root ganglia (DRG) de-targeting miRNA binding site.

2. The recombinant nucleic acid of claim 1, wherein the muscle specific promoter comprises a CK8 promoter, a desmin promoter, a Mb promoter, a MCK promoter, a MHCK7 promoter, a skeletal muscle alpha actin promoter, or a TTNI2 promoter.

3. The recombinant nucleic acid of claim 1, wherein the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404.

4.-6. (canceled)

7. The recombinant nucleic acid of claim 1, wherein the sequence encoding microdystrophin encodes a microdystrophin protein comprising or consisting of an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 2439.

8. (canceled)

9. The recombinant nucleic acid of claim 1, wherein the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407.

10.-12. (canceled)

13. The recombinant nucleic acid of claim 1, wherein the at least one DRG de-targeting miRNA binding site comprises a binding site for miR-338, miR-138, or miR-9.

14. The recombinant nucleic acid of claim 1, wherein the at least one DRG de-targeting miRNA binding site comprises a binding site for miR-338-3p, miR-138-5p, or miR-9-5p.

15. The recombinant nucleic acid of claim 1, wherein the at least one DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401.

16.-20. (canceled)

21. The recombinant nucleic acid of claim 1, wherein the recombinant nucleic acid comprises, optionally from 5′ to 3′:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401.

22. The recombinant nucleic acid of claim 1, wherein the recombinant nucleic acid is selected from:
(a) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(b) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(c) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(d) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(e) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(f) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(g) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(h) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(i) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(j) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(k) a recombinant nucleic acid comprising:
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(l) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(m) a recombinant nucleic acid comprising:
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(n) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(o) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(p) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(q) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(r) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(s) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(t) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(u) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
(v) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(w) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(x) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
(y) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
(z) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401; and
(aa) a recombinant nucleic acid comprising:
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407; and
the at least one DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401.

23. The recombinant nucleic acid of claim 1, wherein the recombinant nucleic acid is selected from:
(a) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2399;
(b) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2399;
(c) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2399;
(d) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2400;
(e) a recombinant nucleic acid comprising:
the promoter comprising or consisting of the nucleic acid sequence of SEQ ID the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2400;
(f) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2400;
(g) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2401;
(h) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2401;
(i) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2405; and
the at least one DRG de-targeting miRNA binding site comprising or consisting of the nucleic acid sequence of SEQ ID NO: 2401;
(j) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2399;
(k) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2399;
(l) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2399;
(m) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2400;
(n) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2400;
(o) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2400;
(p) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2401;
(q) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2401;
(r) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2406; and
the at least one DRG de-targeting miRNA binding site comprising or consisting of the nucleic acid sequence of SEQ ID NO: 2401;
(s) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2399;
(t) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2399;
(u) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2399;
(v) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2400;
(w) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2400;
(x) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2400;
(y) a recombinant nucleic acid comprising:
the promoter comprising or consisting of the nucleic acid sequence of SEQ ID NO: 2402;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2401;
(z) a recombinant nucleic acid comprising:
the promoter comprising or consisting of the nucleic acid sequence of SEQ ID NO: 2403;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2401; and
(aa) a recombinant nucleic acid comprising:
the promoter comprising or consisting of the nucleic acid sequence of SEQ ID NO: 2404;
the sequence encoding microdystrophin comprising or consisting of the nucleic acid sequence of SEQ ID NO: 2407; and
the at least one DRG de-targeting miRNA binding site comprising or consisting of the nucleic acid sequence of SEQ ID NO: 2401.

24. The recombinant nucleic acid of claim 1, further comprising an intron.

25.-27. (canceled)

28. The recombinant nucleic acid of claim 1, further comprising a polyadenylation (PolyA) signal.

29.-31. (canceled)

32. The recombinant nucleic acid of claim 1, further comprising a 5′ inverted terminal repeat (ITR) and a 3′ ITR.

33.-37. (canceled)

38. The recombinant nucleic acid of claim 1, wherein the recombinant nucleic acid comprises, optionally from 5′ to 3′:
a 5′ ITR;
a promoter;
an intron;
a sequence encoding microdystrophin;
a first DRG de-targeting miRNA binding site;
a second DRG de-targeting miRNA binding site;
a PolyA signal; and
a 3′ ITR.

39. The recombinant nucleic acid of claim 38, wherein:
the 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404;
the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407;
the first DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401;
the second DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-2401;
the PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411.

40. The recombinant nucleic acid of claim 38, wherein the recombinant nucleic acid is selected from:
(a) a recombinant nucleic acid comprising:
the 5′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405; and
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(b) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(c) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(d) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2402;
the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 92%, at least 93%, at least 94%, at least 95%, at least 96%, at least 97%, at least 98%, or at least 99% identical to the nucleic acid sequence of SEQ ID NO: 2411;
(e) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(f) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(g) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2411;
(h) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(i) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2405;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(j) a recombinant nucleic acid comprising:
the 5′ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(k) a recombinant nucleic acid comprising:
the 5′ comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(l) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(m) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(n) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2411;
(o) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(p) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(q) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2411;
(r) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2406;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(s) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(t) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(u) a recombinant nucleic acid comprising:
the 5′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2399;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(v) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(w) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(x) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2400;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(y) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2402;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411;
(z) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2403;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2411; and
(aa) a recombinant nucleic acid comprising:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2408;
the promoter comprising or consisting of a nucleic acid sequence that is at least least 92%, at least 93%, at least 94%, at least 95%, at least 96%, at least 97%, at least 98%, or at least 99% identical to the nucleic acid sequence of SEQ ID NO: 2404;
the intron comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2409;
the sequence encoding microdystrophin comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2407;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2401;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2410; and
the 3′ ITR comprising or consisting of a nucleic acid sequence that is at least 85%, 92%, at least 93%, at least 94%, at least 95%, at least 96%, at least 97%, at least 98%, or at least 99% identical to the nucleic acid sequence of SEQ ID NO: 2411.

41. The recombinant nucleic acid of claim 38, wherein the recombinant nucleic acid is selected from:
(a) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(b) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(c) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(d) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprises or consists of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(e) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(f) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprises or consists of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(g) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2401;
the first DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(h) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(i) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprises or consists of the nucleic acid sequence of SEQ ID NO: 2409;
the sequence encoding microdystrophin comprising or consisting of the nucleic acid sequence of SEQ ID NO: 2405;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(j) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprises or consists of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(k) a recombinant nucleic acid comprising:
the 5′ comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(l) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(m) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(n) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(o) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(p) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(q) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(r) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2406;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(s) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(t) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(u) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2399;
the second DRG de-targeting miRNA binding site comprising or consisting of the nucleic acid sequence of SEQ ID NO: 2399;
the PolyA signal comprising or consisting of the nucleic acid sequence of SEQ ID NO: 2410; and
the 3′ ITR comprising or consisting of the nucleic acid sequence of SEQ ID NO: 2411;
(v) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(w) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(x) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2400;
the second DRG de-targeting miRNA binding site comprising or consisting of the nucleic acid sequence of SEQ ID NO: 2400;
the PolyA signal comprising or consisting of the nucleic acid sequence of SEQ ID NO: 2410; and
the 3′ ITR comprising or consisting of the nucleic acid sequence of SEQ ID NO: 2411;
(y) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2402;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411;
(z) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2403;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411; and
(aa) a recombinant nucleic acid comprising:
the 5′ ITR comprising or consisting of the nucleic acid sequence of SEQ ID NO: 2408;
the promoter comprising or consisting of the nucleic acid sequence of SEQ ID NO: 2404;
the intron comprising or consisting of the nucleic acid sequence of SEQ ID NO: 2409;
the sequence encoding microdystrophin comprising or consisting of the nucleic acid sequence of SEQ ID NO: 2407;
the first DRG de-targeting miRNA binding site comprising or consisting of the nucleic acid sequence of SEQ ID NO: 2401;
the second DRG de-targeting miRNA binding site comprising or consisting of the nucleic acid sequence of SEQ ID NO: 2401;
the PolyA signal comprising or consisting of the nucleic acid sequence of SEQ ID NO: 2410; and
the 3′ ITR comprising or consisting of the nucleic acid sequence of SEQ ID NO: 2411.

42. The recombinant nucleic acid of claim 1, wherein the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2412-2438.

43. (canceled)

44. A recombinant nucleic acid comprising a promoter, wherein the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2402-2404.

45.-46. (canceled)

47. A recombinant nucleic acid comprising a codon optimized sequence encoding microdystrophin that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2405-2407.

48.-49. (canceled)

50. An adeno-associated virus (AAV) particle comprising the recombinant nucleic acid of claim 1 and a capsid protein.

51. The AAV particle of claim 50, wherein the capsid protein is derived from a serotype selected from AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, and AAV7m8, or a derivative, hybrid or chimeric serotype thereof.

52.-53. (canceled)

54. The AAV particle of claim 50, wherein the capsid protein comprises an amino acid sequence of formula (I):
(I)
X₁NX₂X₃X₄RGDRX₅X₆L,
wherein each of X₁-X₆ is any amino acid.

55. (canceled)

56. The AAV particle of claim 54, wherein, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L is inserted after amino acid 455.

57.-59. (canceled)

60. The AAV particle of claim 54, wherein the amino acid sequence of formula (I) comprises or consists of any one of SEQ ID NOs: 2-800.

61. (canceled)

62. The AAV particle of claim 54, wherein the capsid protein further comprises a deletion of amino acid G267 relative to a wild type AAV9 vector.

63.-64. (canceled)

65. A method of treating Duchenne muscular dystrophy (DMD) in a subject in need thereof, the method comprising administering to the subject an effective amount of the recombinant nucleic acid of claim 1.

66. The method of claim 65, wherein the subject is a human subject.

67. The method of claim 65, wherein the subject has at least one mutation in the dystrophin gene.

68.-72. (canceled)
